SEQUENCE LISTING (128 sequences)

SEQ ID NO: 1 - protein, 546 aa, synthetic construct
  REGION 1..546: note = Hendra F
MATQEVRLKCLLCGIIVLVLSLEGLGILHYEKLSKIGLVKGITRKYKIKSNPLTKDIVIKMIPNVSNVSKCTGTVMENYKSRLTGILSPIKGAIELYNNNTHDLVGDVKLAGVVMAGIAIGIATAAQITAGVALYEAMKNADNINKLKSSIESTNEAVVKLQETAEKTVYVLTALQDYINTNLVPTIDQISCKQTELALDLALSKYLSDLLFVFGPNLQDPVSNSMTIQAISQAFGGNYETLLRTLGYATEDFDDLLESDSIAGQIVYVDLSSYYIIVRVYFPILTEIQQAYVQELLPVSFNNDNSEWISIVPNFVLIRNTLISNIEVKYCLITKKSVICNQDYATPMTASVRECLTGSTDKCPRELVVSSHVPRFALSGGVLFANCISVTCQCQTTGRAISQSGEQTLLMIDNTTCTTVVLGNIIISLGKYLGSINYNSESIAVGPPVYTDKVDISSQISSMNQSLQQSKDYIKEAQKILDTVNPSLISMLSMIILYVLSIAALCIGLITFISFVIVEKKRGNYSRLDDRQVRPVSNGDLYYIGT

SEQ ID NO: 2 - protein, 546 aa, synthetic construct
  REGION 1..546: note = Nipah F0
MVVILDKRCYCNLLILILMISECSVGILHYEKLSKIGLVKGVTRKYKIKSNPLTKDIVIKMIPNVSNMSQCTGSVMENYKTRLNGILTPIKGALEIYKNNTHDLVGDVRLAGVIMAGVAIGIATAAQITAGVALYEAMKNADNINKLKSSIESTNEAVVKLQETAEKTVYVLTALQDYINTNLVPTIDKISCKQTELSLDLALSKYLSDLLFVFGPNLQDPVSNSMTIQAISQAFGGNYETLLRTLGYATEDFDDLLESDSITGQIIYVDLSSYYIIVRVYFPILTEIQQAYIQELLPVSFNNDNSEWISIVPNFILVRNTLISNIEIGFCLITKRSVICNQDYATPMTNNMRECLTGSTEKCPRELVVSSHVPRFALSNGVLFANCISVTCQCQTTGRAISQSGEQTLLMIDNTTCPTAVLGNVIISLGKYLGSVNYNSEGIAIGPPVFTDKVDISSQISSMNQSLQQSKDYIKEAQRLLDTVNPSLISMLSMIILYVLSIASLCIGLITFISFIIVEKKRNTYSRLEDRRVRPTSSGDLYYIGT

SEQ ID NO: 3 - protein, 557 aa, synthetic construct
  REGION 1..557: note = CedV F
MSNKRTTVLIIISYTLFYLNNAAIVGFDFDKLNKIGVVQGRVLNYKIKGDPMTKDLVLKFIPNIVNITECVREPLSRYNETVRRLLLPIHNMLGLYLNNTNAKMTGLMIAGVIMGGIAIGIATAAQITAGFALYEAKKNTENIQKLTDSIMKTQDSIDKLTDSVGTSILILNKLQTYINNQLVPNLELLSCRQNKIEFDLMLTKYLVDLMTVIGPNINNPVNKDMTIQSLSLLFDGNYDIMMSELGYTPQDFLDLIESKSITGQIIYVDMENLYVVIRTYLPTLIEVPDAQIYEFNKITMSSNGGEYLSTIPNFILIRGNYMSNIDVATCYMTKASVICNQDYSLPMSQNLRSCYQGETEYCPVEAVIASHSPRFALTNGVIFANCINTICRCQDNGKTITQNINQFVSMIDNSTCNDVMVDKFTIKVGKYMGRKDINNINIQIGPQIIIDKVDLSNEINKMNQSLKDSIFYLREAKRILDSVNISLISPSVQLFLIIISVLSFIILLIIIVYLYCKSKHSYKYNKFIDDPDYYNDYKRERINGKASKSNNIYYVGD

SEQ ID NO: 4 - protein, 545 aa, synthetic construct
  REGION 1..545: note = Mojiang F
MALNKNMFSSLFLGYLLVYATTVQSSIHYDSLSKVGVIKGLTYNYKIKGSPSTKLMVVKLIPNIDSVKNCTQKQYDEYKNLVRKALEPVKMAIDTMLNNVKSGNNKYRFAGAIMAGVALGVATAATVTAGIALHRSNENAQAIANMKSAIQNTNEAVKQLQLANKQTLAVIDTIRGEINNNIIPVINQLSCDTIGLSVGIRLTQYYSEIITAFGPALQNPVNTRITIQAISSVFNGNFDELLKIMGYTSGDLYEILHSELIRGNIIDVDVDAGYIALEIEFPNLTLVPNAVVQELMPISYNIDGDEWVTLVPRFVLTRTTLLSNIDTSRCTITDSSVICDNDYALPMSHELIGCLQGDTSKCAREKVVSSYVPKFALSDGLVYANCLNTICRCMDTDTPISQSLGATVSLLDNKRCSVYQVGDVLISVGSYLGDGEYNADNVELGPPIVIDKIDIGNQLAGINQTLQEAEDYIEKSEEFLKGVNPSIITLGSMVVLYIFMILIAIVSVIALVLSIKLTVKGNVVRQQFTYTQHVPSMENINYVSH

SEQ ID NO: 5 - protein, 662 aa, synthetic construct
  REGION 1..662: note = Bat PV F
MKKKTDNPTISKRGHNHSRGIKSRALLRETDNYSNGLIVENLVRNCHHPSKNNLNYTKTQKRDSTIPYRVEERKGHYPKIKHLIDKSYKHIKRGKRRNGHNGNIITIILLLILILKTQMSEGAIHYETLSKIGLIKGITREYKVKGTPSSKDIVIKLIPNVTGLNKCTNISMENYKEQLDKILIPINNIIELYANSTKSAPGNARFAGVIIAGVALGVAAAAQITAGIALHEARQNAERINLLKDSISATNNAVAELQEATGGIVNVITGMQDYINTNLVPQIDKLQCSQIKTALDISLSQYYSEILTVFGPNLQNPVTTSMSIQAISQSFGGNIDLLLNLLGYTANDLLDLLESKSITGQITYINLEHYFMVIRVYYPIMTTISNAYVQELIKISFNVDGSEWVSLVPSYILIRNSYLSNIDISECLITKNSVICRHDFAMPMSYTLKECLTGDTEKCPREAVVTSYVPRFAISGGVIYANCLSTTCQCYQTGKVIAQDGSQTLMMIDNQTCSIVRIEEILISTGKYLGSQEYNTMHVSVGNPVFTDKLDITSQISNINQSIEQSKFYLDKSKAILDKINLNLIGSVPISILFIIAILSLILSIITFVIVMIIVRRYNKYTPLINSDPSSRRSTIQDVYIIPNPGEHSIRSAARSIDRDRD

SEQ ID NO: 6 - protein, 520 aa, synthetic construct
  REGION 1..520: note = Hendra virus F Protein, Without signal sequence
ILHYEKLSKIGLVKGITRKYKIKSNPLTKDIVIKMIPNVSNVSKCTGTVMENYKSRLTGILSPIKGAIELYNNNTHDLVGDVKLAGVVMAGIAIGIATAAQITAGVALYEAMKNADNINKLKSSIESTNEAVVKLQETAEKTVYVLTALQDYINTNLVPTIDQISCKQTELALDLALSKYLSDLLFVFGPNLQDPVSNSMTIQAISQAFGGNYETLLRTLGYATEDFDDLLESDSIAGQIVYVDLSSYYIIVRVYFPILTEIQQAYVQELLPVSFNNDNSEWISIVPNFVLIRNTLISNIEVKYCLITKKSVICNQDYATPMTASVRECLTGSTDKCPRELVVSSHVPRFALSGGVLFANCISVTCQCQTTGRAISQSGEQTLLMIDNTTCTTVVLGNIIISLGKYLGSINYNSESIAVGPPVYTDKVDISSQISSMNQSLQQSKDYIKEAQKILDTVNPSLISMLSMIILYVLSIAALCIGLITFISFVIVEKKRGNYSRLDDRQVRPVSNGDLYYIGT

SEQ ID NO: 7 - protein, 520 aa, synthetic construct
  REGION 1..520: note = Nipah virus F Protein, without signal sequence
ILHYEKLSKIGLVKGVTRKYKIKSNPLTKDIVIKMIPNVSNMSQCTGSVMENYKTRLNGILTPIKGALEIYKNNTHDLVGDVRLAGVIMAGVAIGIATAAQITAGVALYEAMKNADNINKLKSSIESTNEAVVKLQETAEKTVYVLTALQDYINTNLVPTIDKISCKQTELSLDLALSKYLSDLLFVFGPNLQDPVSNSMTIQAISQAFGGNYETLLRTLGYATEDFDDLLESDSITGQIIYVDLSSYYIIVRVYFPILTEIQQAYIQELLPVSFNNDNSEWISIVPNFILVRNTLISNIEIGFCLITKRSVICNQDYATPMTNNMRECLTGSTEKCPRELVVSSHVPRFALSNGVLFANCISVTCQCQTTGRAISQSGEQTLLMIDNTTCPTAVLGNVIISLGKYLGSVNYNSEGIAIGPPVFTDKVDISSQISSMNQSLQQSKDYIKEAQRLLDTVNPSLISMLSMIILYVLSIASLCIGLITFISFIIVEKKRNTYSRLEDRRVRPTSSGDLYYIGT

SEQ ID NO: 8 - protein, 551 aa, synthetic construct
  REGION 1..551: note = Cedar Virus F Protein, without signal sequence
TVLIIISYTLFYLNNAAIVGFDFDKLNKIGVVQGRVLNYKIKGDPMTKDLVLKFIPNIVNITECVREPLSRYNETVRRLLLPIHNMLGLYLNNTNAKMTGLMIAGVIMGGIAIGIATAAQITAGFALYEAKKNTENIQKLTDSIMKTQDSIDKLTDSVGTSILILNKLQTYINNQLVPNLELLSCRQNKIEFDLMLTKYLVDLMTVIGPNINNPVNKDMTIQSLSLLFDGNYDIMMSELGYTPQDFLDLIESKSITGQIIYVDMENLYVVIRTYLPTLIEVPDAQIYEFNKITMSSNGGEYLSTIPNFILIRGNYMSNIDVATCYMTKASVICNQDYSLPMSQNLRSCYQGETEYCPVEAVIASHSPRFALTNGVIFANCINTICRCQDNGKTITQNINQFVSMIDNSTCNDVMVDKFTIKVGKYMGRKDINNINIQIGPQIIIDKVDLSNEINKMNQSLKDSIFYLREAKRILDSVNISLISPSVQLFLIIISVLSFIILLIIIVYLYCKSKHSYKYNKFIDDPDYYNDYKRERINGKASKSNNIYYVGD

SEQ ID NO: 9 - protein, 519 aa, synthetic construct
  REGION 1..519: note = Mojiang virus, Tongguan 1 F Protein, without signal sequence
IHYDSLSKVGVIKGLTYNYKIKGSPSTKLMVVKLIPNIDSVKNCTQKQYDEYKNLVRKALEPVKMAIDTMLNNVKSGNNKYRFAGAIMAGVALGVATAATVTAGIALHRSNENAQAIANMKSAIQNTNEAVKQLQLANKQTLAVIDTIRGEINNNIIPVINQLSCDTIGLSVGIRLTQYYSEIITAFGPALQNPVNTRITIQAISSVFNGNFDELLKIMGYTSGDLYEILHSELIRGNIIDVDVDAGYIALEIEFPNLTLVPNAVVQELMPISYNIDGDEWVTLVPRFVLTRTTLLSNIDTSRCTITDSSVICDNDYALPMSHELIGCLQGDTSKCAREKVVSSYVPKFALSDGLVYANCLNTICRCMDTDTPISQSLGATVSLLDNKRCSVYQVGDVLISVGSYLGDGEYNADNVELGPPIVIDKIDIGNQLAGINQTLQEAEDYIEKSEEFLKGVNPSIITLGSMVVLYIFMILIAIVSVIALVLSIKLTVKGNVVRQQFTYTQHVPSMENINYVSH

SEQ ID NO: 10 - protein, 640 aa, synthetic construct
  REGION 1..640: note = Bat Paramyxovirus F Protein, without signal sequence
SRALLRETDNYSNGLIVENLVRNCHHPSKNNLNYTKTQKRDSTIPYRVEERKGHYPKIKHLIDKSYKHIKRGKRRNGHNGNIITIILLLILILKTQMSEGAIHYETLSKIGLIKGITREYKVKGTPSSKDIVIKLIPNVTGLNKCTNISMENYKEQLDKILIPINNIIELYANSTKSAPGNARFAGVIIAGVALGVAAAAQITAGIALHEARQNAERINLLKDSISATNNAVAELQEATGGIVNVITGMQDYINTNLVPQIDKLQCSQIKTALDISLSQYYSEILTVFGPNLQNPVTTSMSIQAISQSFGGNIDLLLNLLGYTANDLLDLLESKSITGQITYINLEHYFMVIRVYYPIMTTISNAYVQELIKISFNVDGSEWVSLVPSYILIRNSYLSNIDISECLITKNSVICRHDFAMPMSYTLKECLTGDTEKCPREAVVTSYVPRFAISGGVIYANCLSTTCQCYQTGKVIAQDGSQTLMMIDNQTCSIVRIEEILISTGKYLGSQEYNTMHVSVGNPVFTDKLDITSQISNINQSIEQSKFYLDKSKAILDKINLNLIGSVPISILFIIAILSLILSIITFVIVMIIVRRYNKYTPLINSDPSSRRSTIQDVYIIPNPGEHSIRSAARSIDRDRD

SEQ ID NO: 11 - protein, 524 aa, synthetic construct
  REGION 1..524: note = Truncated NiV fusion glycoprotein (FcDelta22) at cytoplasmic tail (with signal sequence)
MVVILDKRCYCNLLILILMISECSVGILHYEKLSKIGLVKGVTRKYKIKSNPLTKDIVIKMIPNVSNMSQCTGSVMENYKTRLNGILTPIKGALEIYKNNTHDLVGDVRLAGVIMAGVAIGIATAAQITAGVALYEAMKNADNINKLKSSIESTNEAVVKLQETAEKTVYVLTALQDYINTNLVPTIDKISCKQTELSLDLALSKYLSDLLFVFGPNLQDPVSNSMTIQAISQAFGGNYETLLRTLGYATEDFDDLLESDSITGQIIYVDLSSYYIIVRVYFPILTEIQQAYIQELLPVSFNNDNSEWISIVPNFILVRNTLISNIEIGFCLITKRSVICNQDYATPMTNNMRECLTGSTEKCPRELVVSSHVPRFALSNGVLFANCISVTCQCQTTGRAISQSGEQTLLMIDNTTCPTAVLGNVIISLGKYLGSVNYNSEGIAIGPPVFTDKVDISSQISSMNQSLQQSKDYIKEAQRLLDTVNPSLISMLSMIILYVLSIASLCIGLITFISFIIVEKKRNT

SEQ ID NO: 12 - protein, 498 aa, synthetic construct
  REGION 1..498: note = Truncated mature NiV fusion glycoprotein (FcDelta22) at cytoplasmic tail
ILHYEKLSKIGLVKGVTRKYKIKSNPLTKDIVIKMIPNVSNMSQCTGSVMENYKTRLNGILTPIKGALEIYKNNTHDLVGDVRLAGVIMAGVAIGIATAAQITAGVALYEAMKNADNINKLKSSIESTNEAVVKLQETAEKTVYVLTALQDYINTNLVPTIDKISCKQTELSLDLALSKYLSDLLFVFGPNLQDPVSNSMTIQAISQAFGGNYETLLRTLGYATEDFDDLLESDSITGQIIYVDLSSYYIIVRVYFPILTEIQQAYIQELLPVSFNNDNSEWISIVPNFILVRNTLISNIEIGFCLITKRSVICNQDYATPMTNNMRECLTGSTEKCPRELVVSSHVPRFALSNGVLFANCISVTCQCQTTGRAISQSGEQTLLMIDNTTCPTAVLGNVIISLGKYLGSVNYNSEGIAIGPPVFTDKVDISSQISSMNQSLQQSKDYIKEAQRLLDTVNPSLISMLSMIILYVLSIASLCIGLITFISFIIVEKKRNT

SEQ ID NO: 13 - protein, 604 aa, synthetic construct
  REGION 1..604: note = Hendra virus G protein Uniprot O89343
MMADSKLVSLNNNLSGKIKDQGKVIKNYYGTMDIKKINDGLLDSKILGAFNTVIALLGSIIIIVMNIMIIQNYTRTTDNQALIKESLQSVQQQIKALTDKIGTEIGPKVSLIDTSSTITIPANIGLLGSKISQSTSSINENVNDKCKFTLPPLKIHECNISCPNPLPFREYRPISQGVSDLVGLPNQICLQKTTSTILKPRLISYTLPINTREGVCITDPLLAVDNGFFAYSHLEKIGSCTRGIAKQRIIGVGEVLDRGDKVPSMFMTNVWTPPNPSTIHHCSSTYHEDFYYTLCAVSHVGDPILNSTSWTESLSLIRLAVRPKSDSGDYNQKYIAITKVERGKYDKVMPYGPSGIKQGDTLYFPAVGFLPRTEFQYNDSNCPIIHCKYSKAENCRLSMGVNSKSHYILRSGLLKYNLSLGGDIILQFIEIADNRLTIGSPSKIYNSLGQPVFYQASYSWDTMIKLGDVDTVDPLRVQWRNNSVISRPGQSQCPRFNVCPEVCWEGTYNDAFLIDRLNWVSAGVYLNSNQTAENPVFAVFKDNEILYQVPLAEDDTNAQKTITDCFLLENVIWCISLVEIYDTGDSVIRPKLFAVKIPAQCSES

SEQ ID NO: 14 - protein, 602 aa, synthetic construct
  REGION 1..602: note = Uniprot Q9IH62
MPAENKKVRFENTTSDKGKIPSKVIKSYYGTMDIKKINEGLLDSKILSAFNTVIALLGSIVIIVMNIMIIQNYTRSTDNQAVIKDALQGIQQQIKGLADKIGTEIGPKVSLIDTSSTITIPANIGLLGSKISQSTASINENVNEKCKFTLPPLKIHECNISCPNPLPFREYRPQTEGVSNLVGLPNNICLQKTSNQILKPKLISYTLPVVGQSGTCITDPLLAMDEGYFAYSHLERIGSCSRGVSKQRIIGVGEVLDRGDEVPSLFMTNVWTPPNPNTVYHCSAVYNNEFYYVLCAVSTVGDPILNSTYWSGSLMMTRLAVKPKSNGGGYNQHQLALRSIEKGRYDKVMPYGPSGIKQGDTLYFPAVGFLVRTEFKYNDSNCPITKCQYSKPENCRLSMGIRPNSHYILRSGLLKYNLSDGENPKVVFIEISDQRLSIGSPSKIYDSLGQPVFYQASFSWDTMIKFGDVLTVNPLVVNWRNNTVISRPGQSQCPRFNTCPEICWEGVYNDAFLIDRINWISAGVFLDSNQTAENPVFTVFKDNEILYRAQLASEDTNAQKTITNCFLLKNKIWCISLVEIYDTGDNVIRPKLFAVKIPEQCT

SEQ ID NO: 15 - protein, 622 aa, synthetic construct
  REGION 1..622: note = Cedar virus attachment glycoprotein
MLSQLQKNYLDNSNQQGDKMNNPDKKLSVNFNPLELDKGQKDLNKSYYVKNKNYNVSNLLNESLHDIKFCIYCIFSLLIIITIINIITISIVITRLKVHEENNGMESPNLQSIQDSLSSLTNMINTEITPRIGILVTATSVTLSSSINYVGTKTNQLVNELKDYITKSCGFKVPELKLHECNISCADPKISKSAMYSTNAYAELAGPPKIFCKSVSKDPDFRLKQIDYVIPVQQDRSICMNNPLLDISDGFFTYIHYEGINSCKKSDSFKVLLSHGEIVDRGDYRPSLYLLSSHYHPYSMQVINCVPVTCNQSSFVFCHISNNTKTLDNSDYSSDEYYITYFNGIDRPKTKKIPINNMTADNRYIHFTFSGGGGVCLGEEFIIPVTTVINTDVFTHDYCESFNCSVQTGKSLKEICSESLRSPTNSSRYNLNGIMIISQNNMTDFKIQLNGITYNKLSFGSPGRLSKTLGQVLYYQSSMSWDTYLKAGFVEKWKPFTPNWMNNTVISRPNQGNCPRYHKCPEICYGGTYNDIAPLDLGKDMYVSVILDSDQLAENPEITVFNSTTILYKERVSKDELNTRSTTTSCFLFLDEPWCISVLETNRFNGKSIRPEIYSYKIPKYC

SEQ ID NO: 16 - protein, 632 aa, synthetic construct
  REGION 1..632: note = Bat Paramyxovirus Eid_hel G
MPQKTVEFINMNSPLERGVSTLSDKKTLNQSKITKQGYFGLGSHSERNWKKQKNQNDHYMTVSTMILEILVVLGIMFNLIVLTMVYYQNDNINQRMAELTSNITVLNLNLNQLTNKIQREIIPRITLIDTATTITIPSAITYILATLTTRISELLPSINQKCEFKTPTLVLNDCRINCTPPLNPSDGVKMSSLATNLVAHGPSPCRNFSSVPTIYYYRIPGLYNRTALDERCILNPRLTISSTKFAYVHSEYDKNCTRGFKYYELMTFGEILEGPEKEPRMFSRSFYSPTNAVNYHSCTPIVTVNEGYFLCLECTSSDPLYKANLSNSTFHLVILRHNKDEKIVSMPSFNLSTDQEYVQIIPAEGGGTAESGNLYFPCIGRLLHKRVTHPLCKKSNCSRTDDESCLKSYYNQGSPQHQVVNCLIRIRNAQRDNPTWDVITVDLTNTYPGSRSRIFGSFSKPMLYQSSVSWHTLLQVAEITDLDKYQLDWLDTPYISRPGGSECPFGNYCPTVCWEGTYNDVYSLTPNNDLFVTVYLKSEQVAENPYFAIFSRDQILKEFPLDAWISSARTTTISCFMFNNEIWCIAALEITRLNDDIIRPIYYSFWLPTDCRTPYPHTGKMTRVPLRSTYNY

SEQ ID NO: 17 - protein, 625 aa, synthetic construct
  REGION 1..625: note = Mojiang virus G
MATNRDNTITSAEVSQEDKVKKYYGVETAEKVADSISGNKVFILMNTLLILTGAIITITLNITNLTAAKSQQNMLKIIQDDVNAKLEMFVNLDQLVKGEIKPKVSLINTAVSVSIPGQISNLQTKFLQKYVYLEESITKQCTCNPLSGIFPTSGPTYPPTDKPDDDTTDDDKVDTTIKPIEYPKPDGCNRTGDHFTMEPGANFYTVPNLGPASSNSDECYTNPSFSIGSSIYMFSQEIRKTDCTAGEILSIQIVLGRIVDKGQQGPQASPLLVWAVPNPKIINSCAVAAGDEMGWVLCSVTLTAASGEPIPHMFDGFWLYKLEPDTEVVSYRITGYAYLLDKQYDSVFIGKGGGIQKGNDLYFQMYGLSRNRQSFKALCEHGSCLGTGGGGYQVLCDRAVMSFGSEESLITNAYLKVNDLASGKPVIIGQTFPPSDSYKGSNGRMYTIGDKYGLYLAPSSWNRYLRFGITPDISVRSTTWLKSQDPIMKILSTCTNTDRDMCPEICNTRGYQDIFPLSEDSEYYTYIGITPNNGGTKNFVAVRDSDGHIASIDILQNYYSITSATISCFMYKDEIWCIAITEGKKQKDNPQRIYAHSYKIRQMCYNMKSATVTVGNAKNITIRRY

SEQ ID NO: 18 - protein, 622 aa, synthetic construct; identical to SEQ ID NO: 15
  REGION 1..622: note = Cedar virus G

SEQ ID NO: 19 - protein, 568 aa, synthetic construct
  REGION 1..568: note = (E501 A, W504A, Q530A, E533A) NiV G protein (Gc delta 34)
MKKINEGLLDSKILSAFNTVIALLGSIVIIVMNIMIIQNYTRSTDNQAVIKDALQGIQQQIKGLADKIGTEIGPKVSLIDTSSTITIPANIGLLGSKISQSTASINENVNEKCKFTLPPLKIHECNISCPNPLPFREYRPQTEGVSNLVGLPNNICLQKTSNQILKPKLISYTLPVVGQSGTCITDPLLAMDEGYFAYSHLERIGSCSRGVSKQRIIGVGEVLDRGDEVPSLFMTNVWTPPNPNTVYHCSAVYNNEFYYVLCAVSTVGDPILNSTYWSGSLMMTRLAVKPKSNGGGYNQHQLALRSIEKGRYDKVMPYGPSGIKQGDTLYFPAVGFLVRTEFKYNDSNCPITKCQYSKPENCRLSMGIRPNSHYILRSGLLKYNLSDGENPKVVFIEISDQRLSIGSPSKIYDSLGQPVFYQASFSWDTMIKFGDVLTVNPLVVNWRNNTVISRPGQSQCPRFNTCPAICAEGVYNDAFLIDRINWISAGVFLDSNATAANPVFTVFKDNEILYRAQLASEDTNAQKTITNCFLLKNKIWCISLVEIYDTGDNVIRPKLFAVKIPEQC

SEQ ID NO: 20 - protein, 5 aa, synthetic construct
  REGION 1..5: note = Linker
GGGGS

SEQ ID NO: 21 - protein, 6 aa, synthetic construct
  REGION 1..6: note = Linker
GGGGGS

SEQ ID NO: 22 - protein, 5 aa, synthetic construct
  REGION 1..5: note = Linker
  REPEAT 1..5: note = repeat any number of times
GGGGS

SEQ ID NO: 23 - protein, 6 aa, synthetic construct
  REGION 1..6: note = Linker
  REPEAT 1..6: note = repeat any number of times
GGGGGS

SEQ ID NO: 24 - protein, 21 aa, synthetic construct
  REGION 1..21: note = CD8aplha signal peptide
MALPVTALLLPLALLLHAARP

SEQ ID NO: 25 - protein, 20 aa, synthetic construct
  REGION 1..20: note = IgK signal peptide
METDTLLLWVLLLWVPGSTG

SEQ ID NO: 26 - protein, 22 aa, synthetic construct
  REGION 1..22: note = GMCSFR-aplha (CSF2RA) signal peptide
MLLLVTSLLLCELPHPAFLLIP

SEQ ID NO: 27 - protein, 45 aa, synthetic construct
  REGION 1..45: note = CD8aplha hinge domain
TTTPAPRPPTPAPTIASQPLSLRPEACRPAAGGAVHTRGLDFACD

SEQ ID NO: 28 - protein, 39 aa, synthetic construct
  REGION 1..39: note = CD28 hinge domain
IEVMYPPPYLDNEKSNGTIIHVKGKHLCPSPLFPGPSKP

SEQ ID NO: 29 - protein, 42 aa, synthetic construct
  REGION 1..42: note = CD28 hinge domain
AAAIEVMYPPPYLDNEKSNGTIIHVKGKHLCPSPLFPGPSKP

SEQ ID NO: 30 - protein, 12 aa, synthetic construct
  REGION 1..12: note = IgG4 hinge domain
ESKYGPPCPPCP

SEQ ID NO: 31 - protein, 12 aa, synthetic construct
  REGION 1..12: note = IgG4 hinge domain
ESKYGPPCPSCP

SEQ ID NO: 32 - protein, 229 aa, synthetic construct
  REGION 1..229: note = IgG4 hinge-CH2-CH3 domain
ESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 33 - protein, 24 aa, synthetic construct
  REGION 1..24: note = CD8alpha transmembrane domain
IYIWAPLAGTCGVLLLSLVITLYC

SEQ ID NO: 34 - protein, 27 aa, synthetic construct
  REGION 1..27: note = CD28 transmembrane domain
FWVLVVVGGVLACYSLLVTVAFIIFWV

SEQ ID NO: 35 - protein, 28 aa, synthetic construct
  REGION 1..28: note = CD28 transmembrane domain
MFWVLVVVGGVLACYSLLVTVAFIIFWV

SEQ ID NO: 36 - protein, 42 aa, synthetic construct
  REGION 1..42: note = 4-1BB costimulatory domain
KRGRKKLLYIFKQPFMRPVQTTQEEDGCSCRFPEEEEGGCEL

SEQ ID NO: 37 - protein, 41 aa, synthetic construct
  REGION 1..41: note = CD28 costimulatory domain
RSKRSRLLHSDYMNMTPRRPGPTRKHYQPYAPPRDFAAYRS

SEQ ID NO: 38 - protein, 112 aa, synthetic construct
  REGION 1..112: note = CD3zeta signaling domain
RVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 39 - protein, 112 aa, synthetic construct
  REGION 1..112: note = CD3zeta signaling domain (with Q to K mutation at position 14)
RVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 40 - protein, 245 aa, synthetic construct
  REGION 1..245: note = Anti-CD19 FMC63 scFv entire sequence, with Whitlow linker
DIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEITGSTSGSGKPGSGEGSTKGEVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSS

SEQ ID NO: 41 - protein, 107 aa, synthetic construct
  REGION 1..107: note = Anti-CD19 FMC63 scFv light chain variable region
DIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEIT

SEQ ID NO: 42 - protein, 6 aa, synthetic construct
  REGION 1..6: note = Anti-CD19 FMC63 scFv light chain CDR1
QDISKY

SEQ ID NO: 43 - skipped

SEQ ID NO: 44 - protein, 9 aa, synthetic construct
  REGION 1..9: note = Anti-CD19 FMC63 scFv light chain CDR3
QQGNTLPYT

SEQ ID NO: 45 - protein, 18 aa, synthetic construct
  REGION 1..18: note = Whitlow linker
GSTSGSGKPGSGEGSTKG

SEQ ID NO: 46 - protein, 120 aa, synthetic construct
  REGION 1..120: note = Anti-CD19 FMC63 scFv heavy chain variable region
EVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSS

SEQ ID NO: 47 - protein, 8 aa, synthetic construct
  REGION 1..8: note = Anti-CD19 FMC63 scFv heavy chain CDR1
GVSLPDYG

SEQ ID NO: 48 - protein, 7 aa, synthetic construct
  REGION 1..7: note = Anti-CD19 FMC63 scFv heavy chain CDR2
IWGSETT

SEQ ID NO: 49 - protein, 14 aa, synthetic construct
  REGION 1..14: note = Anti-CD19 FMC63 scFv heavy chain CDR3
AKHYYYGGSYAMDY

SEQ ID NO: 50 - protein, 242 aa, synthetic construct
  REGION 1..242: note = Anti-CD19 FMC63 scFv entire sequence, with 3xG4S linker
DIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEITGGGGSGGGGSGGGGSEVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSS

SEQ ID NO: 51 - protein, 15 aa, synthetic construct
  REGION 1..15: note = 3xG4S linker
GGGGSGGGGSGGGGS

SEQ ID NO: 52 - DNA, 1467 bp, unidentified
  source 1..1467: mol_type = other DNA
  misc_feature 1..1467: note = Exemplary CD19 CAR nucleotide sequence
atggccttaccagtgaccgccttgctcctgccgctggccttgctgctccacgccgccaggccggacatccagatgacacagactacatcctccctgtctgcctctctgggagacagagtcaccatcagttgcagggcaagtcaggacattagtaaatatttaaattggtatcagcagaaaccagatggaactgttaaactcctgatctaccatacatcaagattacactcaggagtcccatcaaggttcagtggcagtgggtctggaacagattattctctcaccattagcaacctggagcaagaagatattgccacttacttttgccaacagggtaatacgcttccgtacacgttcggaggggggaccaagctggagatcacaggctccacctctggatccggcaagcccggatctggcgagggatccaccaagggcgaggtgaaactgcaggagtcaggacctggcctggtggcgccctcacagagcctgtccgtcacatgcactgtctcaggggtctcattacccgactatggtgtaagctggattcgccagcctccacgaaagggtctggagtggctgggagtaatatggggtagtgaaaccacatactataattcagctctcaaatccagactgaccatcatcaaggacaactccaagagccaagttttcttaaaaatgaacagtctgcaaactgatgacacagccatttactactgtgccaaacattattactacggtggtagctatgctatggactactggggccaaggaacctcagtcaccgtctcctcaaccacgacgccagcgccgcgaccaccaacaccggcgcccaccatcgcgtcgcagcccctgtccctgcgcccagaggcgtgccggccagcggcggggggcgcagtgcacacgagggggctggacttcgcctgtgatatctacatctgggcgcccttggccgggacttgtggggtccttctcctgtcactggttatcaccctttactgcaaacggggcagaaagaaactcctgtatatattcaaacaaccatttatgagaccagtacaaactactcaagaggaagatggctgtagctgccgatttccagaagaagaagaaggaggatgtgaactgagagtgaagttcagcaggagcgcagacgcccccgcgtaccagcagggccagaaccagctctataacgagctcaatctaggacgaagagaggagtacgatgttttggacaagagacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgc

SEQ ID NO: 53 - protein, 489 aa, synthetic construct
  REGION 1..489: note = Exemplary CD19 CAR amino acid sequence
MALPVTALLLPLALLLHAARPDIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEITGSTSGSGKPGSGEGSTKGEVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSSTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 54 - DNA, 1458 bp, unidentified
  source 1..1458: mol_type = other DNA
  misc_feature 1..1458: note = Tisagenlecleucel CD19 CAR nucleotide sequence
atggccttaccagtgaccgccttgctcctgccgctggccttgctgctccacgccgccaggccggacatccagatgacacagactacatcctccctgtctgcctctctgggagacagagtcaccatcagttgcagggcaagtcaggacattagtaaatatttaaattggtatcagcagaaaccagatggaactgttaaactcctgatctaccatacatcaagattacactcaggagtcccatcaaggttcagtggcagtgggtctggaacagattattctctcaccattagcaacctggagcaagaagatattgccacttacttttgccaacagggtaatacgcttccgtacacgttcggaggggggaccaagctggagatcacaggtggcggtggctcgggcggtggtgggtcgggtggcggcggatctgaggtgaaactgcaggagtcaggacctggcctggtggcgccctcacagagcctgtccgtcacatgcactgtctcaggggtctcattacccgactatggtgtaagctggattcgccagcctccacgaaagggtctggagtggctgggagtaatatggggtagtgaaaccacatactataattcagctctcaaatccagactgaccatcatcaaggacaactccaagagccaagttttcttaaaaatgaacagtctgcaaactgatgacacagccatttactactgtgccaaacattattactacggtggtagctatgctatggactactggggccaaggaacctcagtcaccgtctcctcaaccacgacgccagcgccgcgaccaccaacaccggcgcccaccatcgcgtcgcagcccctgtccctgcgcccagaggcgtgccggccagcggcggggggcgcagtgcacacgagggggctggacttcgcctgtgatatctacatctgggcgcccttggccgggacttgtggggtccttctcctgtcactggttatcaccctttactgcaaacggggcagaaagaaactcctgtatatattcaaacaaccatttatgagaccagtacaaactactcaagaggaagatggctgtagctgccgatttccagaagaagaagaaggaggatgtgaactgagagtgaagttcagcaggagcgcagacgcccccgcgtacaagcagggccagaaccagctctataacgagctcaatctaggacgaagagaggagtacgatgttttggacaagagacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgc

SEQ ID NO: 55 - protein, 486 aa, synthetic construct
  REGION 1..486: note = Tisagenlecleucel CD19 CAR amino acid sequence
MALPVTALLLPLALLLHAARPDIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEITGGGGSGGGGSGGGGSEVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSSTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 56 - DNA, 1383 bp, unidentified
  source 1..1383: mol_type = other DNA
  misc_feature 1..1383: note = Lisocabtagene maraleucel CD19 CAR nucleotide sequence
atgctgctgctggtgaccagcctgctgctgtgcgagctgccccaccccgcctttctgctgatccccgacatccagatgacccagaccacctccagcctgagcgccagcctgggcgaccgggtgaccatcagctgccgggccagccaggacatcagcaagtacctgaactggtatcagcagaagcccgacggcaccgtcaagctgctgatctaccacaccagccggctgcacagcggcgtgcccagccggtttagcggcagcggctccggcaccgactacagcctgaccatctccaacctggaacaggaagatatcgccacctacttttgccagcagggcaacacactgccctacacctttggcggcggaacaaagctggaaatcaccggcagcacctccggcagcggcaagcctggcagcggcgagggcagcaccaagggcgaggtgaagctgcaggaaagcggccctggcctggtggcccccagccagagcctgagcgtgacctgcaccgtgagcggcgtgagcctgcccgactacggcgtgagctggatccggcagccccccaggaagggcctggaatggctgggcgtgatctggggcagcgagaccacctactacaacagcgccctgaagagccggctgaccatcatcaaggacaacagcaagagccaggtgttcctgaagatgaacagcctgcagaccgacgacaccgccatctactactgcgccaagcactactactacggcggcagctacgccatggactactggggccagggcaccagcgtgaccgtgagcagcgaatctaagtacggaccgccctgccccccttgccctatgttctgggtgctggtggtggtcggaggcgtgctggcctgctacagcctgctggtcaccgtggccttcatcatcttttgggtgaaacggggcagaaagaaactcctgtatatattcaaacaaccatttatgagaccagtacaaactactcaagaggaagatggctgtagctgccgatttccagaagaagaagaaggaggatgtgaactgcgggtgaagttcagcagaagcgccgacgcccctgcctaccagcagggccagaatcagctgtacaacgagctgaacctgggcagaagggaagagtacgacgtcctggataagcggagaggccgggaccctgagatgggcggcaagcctcggcggaagaacccccaggaaggcctgtataacgaactgcagaaagacaagatggccgaggcctacagcgagatcggcatgaagggcgagcggaggcggggcaagggccacgacggcctgtatcagggcctgtccaccgccaccaaggatacctacgacgccctgcacatgcaggccctgcccccaagg

SEQ ID NO: 57 - protein, 461 aa, synthetic construct
  REGION 1..461: note = Lisocabtagene maraleucel CD19 CAR amino acid sequence
MLLLVTSLLLCELPHPAFLLIPDIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEITGSTSGSGKPGSGEGSTKGEVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSSESKYGPPCPPCPMFWVLVVVGGVLACYSLLVTVAFIIFWVKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 58 - DNA, 1467 bp, unidentified
  source 1..1467: mol_type = other DNA
  misc_feature 1..1467: note = Axicabtagene ciloleucel CD19 CAR nucleotide sequence
atgcttctcctggtgacaagccttctgctctgtgagttaccacacccagcattcctcctgatcccagacatccagatgacacagactacatcctccctgtctgcctctctgggagacagagtcaccatcagttgcagggcaagtcaggacattagtaaatatttaaattggtatcagcagaaaccagatggaactgttaaactcctgatctaccatacatcaagattacactcaggagtcccatcaaggttcagtggcagtgggtctggaacagattattctctcaccattagcaacctggagcaagaagatattgccacttacttttgccaacagggtaatacgcttccgtacacgttcggaggggggactaagttggaaataacaggctccacctctggatccggcaagcccggatctggcgagggatccaccaagggcgaggtgaaactgcaggagtcaggacctggcctggtggcgccctcacagagcctgtccgtcacatgcactgtctcaggggtctcattacccgactatggtgtaagctggattcgccagcctccacgaaagggtctggagtggctgggagtaatatggggtagtgaaaccacatactataattcagctctcaaatccagactgaccatcatcaaggacaactccaagagccaagttttcttaaaaatgaacagtctgcaaactgatgacacagccatttactactgtgccaaacattattactacggtggtagctatgctatggactactggggtcaaggaacctcagtcaccgtctcctcagcggccgcaattgaagttatgtatcctcctccttacctagacaatgagaagagcaatggaaccattatccatgtgaaagggaaacacctttgtccaagtcccctatttcccggaccttctaagcccttttgggtgctggtggtggttgggggagtcctggcttgctatagcttgctagtaacagtggcctttattattttctgggtgaggagtaagaggagcaggctcctgcacagtgactacatgaacatgactccccgccgccccgggcccacccgcaagcattaccagccctatgccccaccacgcgacttcgcagcctatcgctccagagtgaagttcagcaggagcgcagacgcccccgcgtaccagcagggccagaaccagctctataacgagctcaatctaggacgaagagaggagtacgatgttttggacaagagacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgc

SEQ ID NO: 59 - protein, 489 aa, synthetic construct
  REGION 1..489: note = Axicabtagene ciloleucel CD19 CAR amino acid sequence
MLLLVTSLLLCELPHPAFLLIPDIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEITGSTSGSGKPGSGEGSTKGEVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSSAAAIEVMYPPPYLDNEKSNGTIIHVKGKHLCPSPLFPGPSKPFWVLVVVGGVLACYSLLVTVAFIIFWVRSKRSRLLHSDYMNMTPRRPGPTRKHYQPYAPPRDFAAYRSRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 60 - protein, 246 aa, synthetic construct
  REGION 1..246: note = Anti-CD20 Leu16 scFv entire sequence, with Whitlow linker
DIVLTQSPAILSASPGEKVTMTCRASSSVNYMDWYQKKPGSSPKPWIYATSNLASGVPARFSGSGSGTSYSLTISRVEAEDAATYYCQQWSFNPPTFGGGTKLEIKGSTSGSGKPGSGEGSTKGEVQLQQSGAELVKPGASVKMSCKASGYTFTSYNMHWVKQTPGQGLEWIGAIYPGNGDTSYNQKFKGKATLTADKSSSTAYMQLSSLTSEDSADYYCARSNYYGSSYWFFDVWGAGTTVTVSS

SEQ ID NO: 61 - protein, 106 aa, synthetic construct
  REGION 1..106: note = Anti-CD20 Leu16 scFv light chain variable region
DIVLTQSPAILSASPGEKVTMTCRASSSVNYMDWYQKKPGSSPKPWIYATSNLASGVPARFSGSGSGTSYSLTISRVEAEDAATYYCQQWSFNPPTFGGGTKLEIK

SEQ ID NO: 62 - protein, 10 aa, synthetic construct
  REGION 1..10: note = Anti-CD20 Leu16 scFv light chain CDR1
RASSSVNYMD

SEQ ID NO: 63 - protein, 7 aa, synthetic construct
  REGION 1..7: note = Anti-CD20 Leu16 scFv light chain CDR2
ATSNLAS

SEQ ID NO: 64 - protein, 9 aa, synthetic construct
  REGION 1..9: note = Anti-CD20 Leu16 scFv light chain CDR3
QQWSFNPPT

SEQ ID NO: 65 - protein, 122 aa, synthetic construct
  REGION 1..122: note = Anti-CD20 Leu16 scFv heavy chain
EVQLQQSGAELVKPGASVKMSCKASGYTFTSYNMHWVKQTPGQGLEWIGAIYPGNGDTSYNQKFKGKATLTADKSSSTAYMQLSSLTSEDSADYYCARSNYYGSSYWFFDVWGAGTTVTVSS

SEQ ID NO: 66 - protein, 5 aa, synthetic construct
  REGION 1..5: note = Anti-CD20 Leu16 scFv heavy chain CDR1
SYNMH

SEQ ID NO: 67 - protein, 17 aa, synthetic construct
  REGION 1..17: note = Anti-CD20 Leu16 scFv heavy chain CDR2
AIYPGNGDTSYNQKFKG

SEQ ID NO: 68 - protein, 13 aa, synthetic construct
  REGION 1..13: note = Anti-CD20 Leu16 scFv heavy chain CDR3
SNYYGSSYWFFDV

SEQ ID NO: 69 - protein, 246 aa, synthetic construct
  REGION 1..246: note = Anti-CD22 m971 scFv entire sequence, with 3xG4S linker
QVQLQQSGPGLVKPSQTLSLTCAISGDSVSSNSAAWNWIRQSPSRGLEWLGRTYYRSKWYNDYAVSVKSRITINPDTSKNQFSLQLNSVTPEDTAVYYCAREVTGDLEDAFDIWGQGTMVTVSSGGGGSGGGGSGGGGSDIQMTQSPSSLSASVGDRVTITCRASQTIWSYLNWYQQRPGKAPNLLIYAASSLQSGVPSRFSGRGSGTDFTLTISSLQAEDFATYYCQQSYSIPQTFGQGTKLEIK

SEQ ID NO: 70 - protein, 124 aa, synthetic construct
  REGION 1..124: note = Anti-CD22 m971 scFv heavy chain variable region
QVQLQQSGPGLVKPSQTLSLTCAISGDSVSSNSAAWNWIRQSPSRGLEWLGRTYYRSKWYNDYAVSVKSRITINPDTSKNQFSLQLNSVTPEDTAVYYCAREVTGDLEDAFDIWGQGTMVTVSS

SEQ ID NO: 71 - protein, 10 aa, synthetic construct
  REGION 1..10: note = Anti-CD22 m971 scFv heavy chain CDR1
GDSVSSNSAA

SEQ ID NO: 72 - protein, 9 aa, synthetic construct
  REGION 1..9: note = Anti-CD22 m971 scFv heavy chain CDR2
TYYRSKWYN

SEQ ID NO: 73 - protein, 14 aa, synthetic construct
  REGION 1..14: note = Anti-CD22 m971 scFv heavy chain CDR3
AREVTGDLEDAFDI

SEQ ID NO: 74 - protein, 107 aa, synthetic construct
  REGION 1..107: note = Anti-CD22 m971 scFv light chain
DIQMTQSPSSLSASVGDRVTITCRASQTIWSYLNWYQQRPGKAPNLLIYAASSLQSGVPSRFSGRGSGTDFTLTISSLQAEDFATYYCQQSYSIPQTFGQGTKLEIK

SEQ ID NO: 75 - protein, 6 aa, synthetic construct
  REGION 1..6: note = Anti-CD22 m971 scFv light chain CDR1
QTIWSY

SEQ ID NO: 76 - skipped

SEQ ID NO: 77 - protein, 9 aa, synthetic construct
  REGION 1..9: note = Anti-CD22 m971 scFv light chain CDR3
QQSYSIPQT

SEQ ID NO: 78 - protein, 246 aa, synthetic construct
  REGION 1..246: note = Anti-CD22 m971-L7 scFv entire sequence, with 3xG4S linker
QVQLQQSGPGMVKPSQTLSLTCAISGDSVSSNSVAWNWIRQSPSRGLEWLGRTYYRSTWYNDYAVSMKSRITINPDTNKNQFSLQLNSVTPEDTAVYYCAREVTGDLEDAFDIWGQGTMVTVSSGGGGSGGGGSGGGGSDIQMIQSPSSLSASVGDRVTITCRASQTIWSYLNWYRQRPGEAPNLLIYAASSLQSGVPSRFSGRGSGTDFTLTISSLQAEDFATYYCQQSYSIPQTFGQGTKLEIK

SEQ ID NO: 79 - protein, 124 aa, synthetic construct
  REGION 1..124: note = Anti-CD22 m971-L7 scFv heavy chain variable region
QVQLQQSGPGMVKPSQTLSLTCAISGDSVSSNSVAWNWIRQSPSRGLEWLGRTYYRSTWYNDYAVSMKSRITINPDTNKNQFSLQLNSVTPEDTAVYYCAREVTGDLEDAFDIWGQGTMVTVSS

SEQ ID NO: 80 - protein, 10 aa, synthetic construct
  REGION 1..10: note = Anti-CD22 m971-L7 scFv heavy chain CDR1
GDSVSSNSVA

SEQ ID NO: 81 - protein, 9 aa, synthetic construct
  REGION 1..9: note = Anti-CD22 m971-L7 scFv heavy chain CDR2
TYYRSTWYN

SEQ ID NO: 82 - protein, 14 aa, synthetic construct
  REGION 1..14: note = Anti-CD22 m971-L7 scFv heavy chain CDR3
AREVTGDLEDAFDI

SEQ ID NO: 83 - protein, 107 aa, synthetic construct
  REGION 1..107: note = Anti-CD22 m971-L7 scFv light chain variable region
DIQMIQSPSSLSASVGDRVTITCRASQTIWSYLNWYRQRPGEAPNLLIYAASSLQSGVPSRFSGRGSGTDFTLTISSLQAEDFATYYCQQSYSIPQTFGQGTKLEIK

SEQ ID NO: 84 - protein, 6 aa, synthetic construct
  REGION 1..6: note = Anti-CD22 m971-L7 scFv light chain CDR1
QTIWSY

SEQ ID NO: 85 - skipped

SEQ ID NO: 86 - protein, 9 aa, synthetic construct
  REGION 1..9: note = Anti-CD22 m971-L7 scFv light chain CDR3
QQSYSIPQT

SEQ ID NO: 87 - protein, 246 aa, synthetic construct
  REGION 1..246: note = Anti-BCMA C11D5.3 scFv entire sequence, with Whitlow linker
DIVLTQSPASLAMSLGKRATISCRASESVSVIGAHLIHWYQQKPGQPPKLLIYLASNLETGVPARFSGSGSGTDFTLTIDPVEEDDVAIYSCLQSRIFPRTFGGGTKLEIKGSTSGSGKPGSGEGSTKGQIQLVQSGPELKKPGETVKISCKASGYTFTDYSINWVKRAPGKGLKWMGWINTETREPAYAYDFRGRFAFSLETSASTAYLQINNLKYEDTATYFCALDYSYAMDYWGQGTSVTVSS

SEQ ID NO: 88 - protein, 111 aa, synthetic construct
  REGION 1..111: note = Anti-BCMA C11D5.3 scFv light chain variable region
DIVLTQSPASLAMSLGKRATISCRASESVSVIGAHLIHWYQQKPGQPPKLLIYLASNLETGVPARFSGSGSGTDFTLTIDPVEEDDVAIYSCLQSRIFPRTFGGGTKLEIK

SEQ ID NO: 89 - protein, 15 aa, synthetic construct
  REGION 1..15: note = Anti-BCMA C11D5.3 scFv light chain CDR1
RASESVSVIGAHLIH

SEQ ID NO: 90 - protein, 7 aa, synthetic construct
  REGION 1..7: note = Anti-BCMA C11D5.3 scFv light chain CDR2
LASNLET

SEQ ID NO: 91 - protein, 9 aa, synthetic construct
  REGION 1..9: note = Anti-BCMA C11D5.3 scFv light chain CDR3
LQSRIFPRT

SEQ ID NO: 92 - protein, 117 aa, synthetic construct
  REGION 1..117: note = Anti-BCMA C11D5.3 scFv heavy chain variable region
QIQLVQSGPELKKPGETVKISCKASGYTFTDYSINWVKRAPGKGLKWMGWINTETREPAYAYDFRGRFAFSLETSASTAYLQINNLKYEDTATYFCALDYSYAMDYWGQGTSVTVSS

SEQ ID NO: 93 - protein, 5 aa, synthetic construct
  REGION 1..5: note = Anti-BCMA C11D5.3 scFv heavy chain CDR1
DYSIN

SEQ ID NO: 94 - protein, 17 aa, synthetic construct
  REGION 1..17: note = Anti-BCMA C11D5.3 scFv heavy chain CDR2
WINTETREPAYAYDFRG

SEQ ID NO: 95 - protein, 8 aa, synthetic construct
  REGION 1..8: note = Anti-BCMA C11D5.3 scFv heavy chain CDR3
DYSYAMDY

SEQ ID NO: 96 - protein, 246 aa, synthetic construct
  REGION 1..246: note = Anti-BCMA C12A3.2 scFv entire sequence, with Whitlow linker
DIVLTQSPPSLAMSLGKRATISCRASESVTILGSHLIYWYQQKPGQPPTLLIQLASNVQTGVPARFSGSGSRTDFTLTIDPVEEDDVAVYYCLQSRTIPRTFGGGTKLEIKGSTSGSGKPGSGEGSTKGQIQLVQSGPELKKPGETVKISCKASGYTFRHYSMNWVKQAPGKGLKWMGRINTESGVPIYADDFKGRFAFSVETSASTAYLVINNLKDEDTASYFCSNDYLYSLDFWGQGTALTVSS

SEQ ID NO: 97 - protein, 111 aa, synthetic construct
  REGION 1..111: note = Anti-BCMA C12A3.2 scFv light chain variable region
DIVLTQSPPSLAMSLGKRATISCRASESVTILGSHLIYWYQQKPGQPPTLLIQLASNVQTGVPARFSGSGSRTDFTLTIDPVEEDDVAVYYCLQSRTIPRTFGGGTKLEIK

SEQ ID NO: 98 - protein, 15 aa, synthetic construct
  REGION 1..15: note = Anti-BCMA C12A3.2 scFv light chain CDR1
RASESVTILGSHLIY

SEQ ID NO: 99 - protein, 7 aa, synthetic construct
  REGION 1..7: note = Anti-BCMA C12A3.2 scFv light chain CDR2
LASNVQT

SEQ ID NO: 100 - protein, 9 aa, synthetic construct
  REGION 1..9: note = Anti-BCMA C12A3.2 scFv light chain CDR3
LQSRTIPRT

SEQ ID NO: 101 - protein, 117 aa, synthetic construct
  REGION 1..117: note = Anti-BCMA C12A3.2 scFv heavy chain variable region
QIQLVQSGPELKKPGETVKISCKASGYTFRHYSMNWVKQAPGKGLKWMGRINTESGVPIYADDFKGRFAFSVETSASTAYLVINNLKDEDTASYFCSNDYLYSLDFWGQGTALTVSS

SEQ ID NO: 102 - protein, 5 aa, synthetic construct
  REGION 1..5: note = Anti-BCMA C12A3.2 scFv heavy chain CDR1
HYSMN

SEQ ID NO: 103 - protein, 17 aa, synthetic construct
  REGION 1..17: note = Anti-BCMA C12A3.2 scFv heavy chain CDR2
RINTESGVPIYADDFKG

SEQ ID NO: 104 - protein, 8 aa, synthetic construct
  REGION 1..8: note = Anti-BCMA C12A3.2 scFv heavy chain CDR3
DYLYSLDF

SEQ ID NO: 105 - protein, 121 aa, synthetic construct
  REGION 1..121: note = Anti-BCMA FHVH33 entire sequence
EVQLLESGGGLVQPGGSLRLSCAASGFTFSSYAMSWVRQAPGKGLEWVSSISGSGDYIYYADSVKGRFTISRDISKNTLYLQMNSLRAEDTAVYYCAKEGTGANSSLADYRGQGTLVTVSS

SEQ ID NO: 106 - protein, 8 aa, synthetic construct
  REGION 1..8: note = Anti-BCMA FHVH33 CDR1
GFTFSSYA

SEQ ID NO: 107 - protein, 8 aa, synthetic construct
  REGION 1..8: note = Anti-BCMA FHVH33 CDR2
ISGSGDYI

SEQ ID NO: 108 - protein, 14 aa, synthetic construct
  REGION 1..14: note = Anti-BCMA FHVH33 CDR3
AKEGTGANSSLADY

SEQ ID NO: 109 - protein, 243 aa, synthetic construct
  REGION 1..243: note = Anti-BCMA CT103A scFv entire sequence, with Whitlow linker
DIQMTQSPSSLSASVGDRVTITCRASQSISSYLNWYQQKPGKAPKLLIYAASSLQSGVPSRFSGSGSGTDFTLTISSLQPEDFATYYCQQKYDLLTFGGGTKVEIKGSTSGSGKPGSGEGSTKGQLQLQESGPGLVKPSETLSLTCTVSGGSISSSSYYWGWIRQPPGKGLEWIGSISYSGSTYYNPSLKSRVTISVDTSKNQFSLKLSSVTAADTAVYYCARDRGDTILDVWGQGTMVTVSS

SEQ ID NO: 110 - protein, 106 aa, synthetic construct
  REGION 1..106: note = Anti-BCMA CT103A scFv light chain variable region
DIQMTQSPSSLSASVGDRVTITCRASQSISSYLNWYQQKPGKAPKLLIYAASSLQSGVPSRFSGSGSGTDFTLTISSLQPEDFATYYCQQKYDLLTFGGGTKVEIK

SEQ ID NO: 111 - protein, 6 aa, synthetic construct
  REGION 1..6: note = Anti-BCMA CT103A scFv light chain CDR1
QSISSY

SEQ ID NO: 112 - skipped

SEQ ID NO: 113 - protein, 8 aa, synthetic construct
  REGION 1..8: note = Anti-BCMA CT103A scFv light chain CDR3
QQKYDLLT

SEQ ID NO: 114 - protein, 119 aa, synthetic construct
  REGION 1..119: note = Anti-BCMA CT103A scFv heavy chain variable region
QLQLQESGPGLVKPSETLSLTCTVSGGSISSSSYYWGWIRQPPGKGLEWIGSISYSGSTYYNPSLKSRVTISVDTSKNQFSLKLSSVTAADTAVYYCARDRGDTILDVWGQGTMVTVSS

SEQ ID NO: 115 - protein, 10 aa, synthetic construct
  REGION 1..10: note = Anti-BCMA CT103A scFv heavy chain CDR1
GGSISSSSYY

SEQ ID NO: 116 - protein, 7 aa, synthetic construct
  REGION 1..7: note = Anti-BCMA CT103A scFv heavy chain CDR2
ISYSGST

SEQ ID NO: 117 - protein, 11 aa, synthetic construct
  REGION 1..11: note = Anti-BCMA CT103A scFv heavy chain CDR3
ARDRGDTILDV

SEQ ID NO: 118 - DNA, 1503 bp, unidentified
  source 1..1503: mol_type = other DNA
  misc_feature 1..1503: note = Exemplary BCMA CAR nucleotide sequence
atggccttaccagtgaccgccttgctcctgccgctggccttgctgctccacgccgccaggccggacatccagatgacccagtctccatcctccctgtctgcatctgtaggagacagagtcaccatcacttgccgggcaagtcagagcattagcagctatttaaattggtatcagcagaaaccagggaaagcccctaagctcctgatctatgctgcatccagtttgcaaagtggggtcccatcaaggttcagtggcagtggatctgggacagatttcactctcaccatcagcagtctgcaacctgaagattttgcaacttactactgtcagcaaaaatacgacctcctcacttttggcggagggaccaaggttgagatcaaaggcagcaccagcggctccggcaagcctggctctggcgagggcagcacaaagggacagctgcagctgcaggagtcgggcccaggactggtgaagccttcggagaccctgtccctcacctgcactgtctctggtggctccatcagcagtagtagttactactggggctggatccgccagcccccagggaaggggctggagtggattgggagtatctcctatagtgggagcacctactacaacccgtccctcaagagtcgagtcaccatatccgtagacacgtccaagaaccagttctccctgaagctgagttctgtgaccgccgcagacacggcggtgtactactgcgccagagatcgtggagacaccatactagacgtatggggtcagggtacaatggtcaccgtcagctcattcgtgcccgtgttcctgcccgccaaacctaccaccacccctgcccctagacctcccaccccagccccaacaatcgccagccagcctctgtctctgcggcccgaagcctgtagacctgctgccggcggagccgtgcacaccagaggcctggacttcgcctgcgacatctacatctgggcccctctggccggcacctgtggcgtgctgctgctgagcctggtgatcaccctgtactgcaaccaccggaacaaacggggcagaaagaaactcctgtatatattcaaacaaccatttatgagaccagtacaaactactcaagaggaagatggctgtagctgccgatttccagaagaagaagaaggaggatgtgaactgagagtgaagttcagcagatccgccgacgcccctgcctaccagcagggacagaaccagctgtacaacgagctgaacctgggcagacgggaagagtacgacgtgctggacaagcggagaggccgggaccccgagatgggcggaaagcccagacggaagaacccccaggaaggcctgtataacgaactgcagaaagacaagatggccgaggcctacagcgagatcggcatgaagggcgagcggaggcgcggcaagggccacgatggcctgtaccagggcctgagcaccgccaccaaggacacctacgacgccctgcacatgcaggccctgccccccaga

SEQ ID NO: 119 - protein, 501 aa, synthetic construct
  REGION 1..501: note = Exemplary BCMA CAR amino acid sequence
MALPVTALLLPLALLLHAARPDIQMTQSPSSLSASVGDRVTITCRASQSISSYLNWYQQKPGKAPKLLIYAASSLQSGVPSRFSGSGSGTDFTLTISSLQPEDFATYYCQQKYDLLTFGGGTKVEIKGSTSGSGKPGSGEGSTKGQLQLQESGPGLVKPSETLSLTCTVSGGSISSSSYYWGWIRQPPGKGLEWIGSISYSGSTYYNPSLKSRVTISVDTSKNQFSLKLSSVTAADTAVYYCARDRGDTILDVWGQGTMVTVSSFVPVFLPAKPTTTPAPRPPTPAPTIASQPLSLRPEACRPAAGGAVHTRGLDFACDIYIWAPLAGTCGVLLLSLVITLYCNHRNKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 120 - protein, 122 aa, synthetic construct
  REGION 1..122: note = CD8_1 VH
QVQLVQSGAEVKKPGASVKVSCKASGGTFSSYAISWVRQAPGQGLEWMGIIDPSDGNTNYAQNFQGRVTMTRDTSTSTVYMELSSLRSEDTAVYYCAKERAAAGYYYYMDVWGQGTTVTVSS

SEQ ID NO: 121 - protein, 108 aa, synthetic construct
  REGION 1..108: note = CD8_1 VL
DIQMTQSPSSLSASVGDRVTITCRASQSISSYLNWYQQKPGKAPKLLIYAASSLQSGVPSRFSGSGSGTDFTLTISSLQPEDFATYYCQQSYSTPLTFGGGTKVEIKR

SEQ ID NO: 122 - protein, 119 aa, synthetic construct
  REGION 1..119: note = CD8_2 VH
QVQLVQSGAEVKKPGASVKVSCKASGYTFTDYYIQWVRQAPGQGLEWMGWINPNSGGTSYAQKFQGRVTMTRDTSTSTVYMELSSLRSEDTAVYYCAKEGDYYYGMDAWGQGTMVTVSS

SEQ ID NO: 123 - protein, 113 aa, synthetic construct
  REGION 1..113: note = CD8_2 VL
DIVMTQSPLSLPVTPGEPASISCRSSQSLLHSNGYNYLDWYLQKPGQSPQLLIYLGSNRASGVPDRFSGSGSGTDFTLKISRVEAEDVGVYYCMQGLQTPHTFGQGTKVEIKR

SEQ ID NO: 124 - protein, 117 aa, synthetic construct
  REGION 1..117: note = CD8_3 VH
QVQLVQSGAEVKKPGASVKVSCKASGYTFTSYYMHWVRQAPGQGLEWMGGFDPEDGETIYAQKFQGRVTMTRDTSTSTVYMELSSLRSEDTAVYYCARDQGWGMDVWGQGTTVTVSS

SEQ ID NO: 125 - protein, 108 aa, synthetic construct
  REGION 1..108: note = CD8_3 VL
DIQMTQSPSSLSASVGDRVTITCRASQSISSYLNWYQQKPGKAPKLLIYAASSLQSGVPSRFSGSGSGTDFTLTISSLQPEDFATYYCQQTYSTPYTFGQGTKLEIKR

SEQ ID NO: 126 - protein, 118 aa, synthetic construct
  REGION 1..118: note = CD8_4 VH
QVQLVQSGAEVKKPGASVKVSCKASGYTFTNHYMHWVRQAPGQGLEWMGWMNPNSGNTGYAQKFQGRVTMTRDTSTSTVYMELSSLRSEDTAVYYCASSESGSDLDYWGQGTLVTVSS

SEQ ID NO: 127 - protein, 108 aa, synthetic construct
  REGION 1..108: note = CD8_4 VL
DIQMTQSPSSLSASVGDRVTITCRASQTIGNYVNWYQQKPGKAPKLLIYGASNLHTGVPSRFSGSGSGTDFTLTISSLQPEDFATYYCQQTYSAPLTFGGGTKVEIKR

SEQ ID NO: 128 - protein, 125 aa, synthetic construct
  REGION 1..125: note = CD8_VHH
QVQLVESGGGLVQAGGSLRLSCAASGRTFSGYVMGWFRQAPGKQRKFVAAISRGGLSTSYADSVKGRFTISRDNAKNTVFLQMNTLKPEDTAVYYCAADRSDLYEITAASNIDSWGQGTLVTVSS

(19) United States
(12) Patent Application Publication
(10) Pub. No.: US 2025/0144235 A1
(43) Pub. Date: May 8, 2025

(54) METHODS OF REPEAT DOSING AND ADMINISTRATION OF LIPID PARTICLES OR VIRAL VECTORS AND RELATED SYSTEMS AND USES

(71) Applicant: Sana Biotechnology, Inc., Seattle, WA (US)

(72) Inventor: Suvi JAIN, Waltham, MA (US)

(73) Assignee: Sana Biotechnology, Inc., Seattle, WA (US)

(21) Appl. No.: 18/835,218

(22) PCT Filed: Feb. 2, 2023
(86) PCT No.: PCT/US2023/061888

§ 371 Date: Aug. 1, 2024

Related U.S. Application Data
(60) Provisional application No. 63/341,968, filed on May 13, 2022.
(60) Provisional application No. 63/306,071, filed on Feb. 2, 2022.

(51) Int. Cl. A61K 48/00; C12N 15/86

(52) U.S. Cl. CPC A61K 48/0041; C12N 15/86; C12N 2740/10045; C12N 2740/15043; C12N 2760/18022; C12N 2810/6072

(57) ABSTRACT

Provided herein are methods of repeated administration of a lipid particle or viral vector, such as for delivery of a payload gene, to a subject.

### CROSS-REFERENCE TO RELATED APPLICATIONS

This application claims priority to U.S. provisional application nos. 63/306,071, filed Feb. 2, 2022, entitled “METHODS OF REPEAT DOSING AND ADMINISTRATION OF LIPID PARTICLES OR VIRAL VECTORS AND RELATED SYSTEMS AND USES,” and 63/341,968, filed May 13, 2022, entitled, “METHODS OF REPEAT DOSING AND ADMINISTRATION OF LIPID PARTICLES OR VIRAL VECTORS AND RELATED SYSTEMS AND USES,” the contents of each of which are incorporated by reference in their entirety for all purposes.

### INCORPORATION BY REFERENCE OF SEQUENCE LISTING

The present application is being filed along with a Sequence Listing in electronic format. The Sequence Listing is provided as a file entitled 18615_2006340_Seq.XML, created Jan. 30, 2023, which is 184,521 bytes in size. The information in the electronic format of the Sequence Listing is incorporated by reference in its entirety.

### FIELD

The present disclosure provides methods of repeated administration of a lipid particle or viral vector, such as for delivery of a payload gene, to a subject.

### SUMMARY

Provided herein is a method of delivering an exogenous agent to a subject, the method comprising: administering to a subject a first dose of a targeted lipid particle comprising an exogenous agent, and administering to the subject a second dose of the targeted lipid particle, wherein the targeted lipid particle comprises one or more fusogen proteins, and wherein the first and second doses are administered within one month of each other. In some embodiments, the first and second doses are administered within four weeks, within three weeks, within two weeks, within seven days, within six days, within five days, within four days, within three days, within two days, or within one day of each other. In some embodiments, the fusogen includes one or more Paramyxovirus envelope proteins.

Also provided herein is a method of delivering an exogenous agent to a subject, the method comprising: administering to a subject a first dose of a targeted lipid particle comprising an exogenous agent, and administering to the subject a second dose of the targeted lipid particle, wherein the targeted lipid particle comprises one or more fusogens, and wherein the second dose is administered at time between the first day and the twenty-eighth day, inclusive, following the first dose. In some embodiments, the fusogen includes one or more Paramyxovirus envelope proteins.

Also provided herein is a method of delivering an exogenous agent to a subject, the method comprising: administering to a subject a first dose of a targeted lipid particle comprising an exogenous agent, and administering to the subject a second dose of the targeted lipid particle, wherein the targeted lipid particle comprises one or more fusogens, and wherein the second dose is administered (i) on the first day following the first dose or (ii) on the second, third, fourth, fifth, sixth, seventh, fourteenth, twenty-first, or twenty-eighth day following the first dose. In some embodiments, the fusogen includes one or more Paramyxovirus envelope proteins.

Provided here in is a method of delivering an exogenous agent to a subject, the method comprising: administering to a subject a first dose of a targeted lipid particle comprising an exogenous agent, and administering to the subject a second dose of the targeted lipid particle, wherein the targeted lipid particle comprises one or more Paramyxovirus envelope proteins, and wherein the first and second doses are administered within one month of each other. In some embodiments, the first and second doses are administered within four weeks, within three weeks, within two weeks, within seven days, within six days, within five days, within four days, within three days, within two days, or within one day of each other.

Also provided herein is a method of delivering an exogenous agent to a subject, the method comprising: administering to a subject a first dose of a targeted lipid particle comprising an exogenous agent, and administering to the subject a second dose of the targeted lipid particle, wherein the targeted lipid particle comprises one or more Paramyxovirus envelope proteins, and wherein the second dose is administered at time between the first day and the twenty-eighth day, inclusive, following the first dose.

Also provided herein is a method of delivering an exogenous agent to a subject, the method comprising: administering to a subject a first dose of a targeted lipid particle comprising an exogenous agent, and administering to the subject a second dose of the targeted lipid particle, wherein the targeted lipid particle comprises one or more Paramyxovirus envelope proteins, and wherein the second dose is administered (i) on the first day following the first dose or (ii) on the second, third, fourth, fifth, sixth, seventh, fourteenth, twenty-first, or twenty-eighth day following the first dose.

In some embodiments, the second dose is administered on the first day following the first dose. In some embodiments, the second dose is administered on the second day following the first dose. In some embodiments, the second dose is administered on the third day following the first dose. In some embodiments, the second dose is administered on the fourth day following the first dose. In some embodiments, the second dose is administered on the fifth day following the first dose. In some embodiments, the second dose is administered on the sixth day following the first dose. In some embodiments, the second dose is administered on the seventh day following the first dose. In some embodiments, the second dose is administered on the fourteenth day following the first dose. In some embodiments, the second dose is administered on the twenty-first day following the first dose. In some embodiments, the second dose is administered on the twenty-eighth day following the first dose.

In some of any of the provided embodiments, the one or more Paramyxovirus envelope proteins have fusogenic activity. In some of any of the provided embodiments, the native binding tropism of the one or more of the Paramyxovirus envelope proteins is reduced. In some of any of the provided embodiments, the one or more Paramyxovirus envelope proteins is derived from an H protein molecule or a biologically active portion thereof from a Paramyxovirus and/or an HN protein molecule or a biologically active portion thereof from a Paramyxovirus. In some of any of the provided embodiments, the one or more Paramyxovirus envelope proteins comprises an F protein molecule or a biologically active portion thereof and/or a glycoprotein G (G protein) or a biologically active portion thereof. In some of any of the provided embodiments, the one or more Paramyxovirus envelope proteins comprises an F protein molecule or a biologically active portion thereof from a Paramyxovirus and a glycoprotein G (G protein) or a biologically active portion thereof from a Paramyxovirus.

In some of any of the provided embodiments, the paramyxovirus is a henipavirus. In some of any of the provided embodiments, the paramyxovirus is Measles morbillivirus. In some of any of the provided embodiments, the paramyxovirus is a Hendra virus. In some of any of the provided embodiments, the paramyxovirus is Nipah virus. In some of any of the provided embodiments, the F protein or the biologically active portion thereof is a wild-type Nipah virus F (NiV-F) protein or is a functionally active variant or biologically active portion thereof.

In some of any of the provided embodiments, the F protein molecule or a biologically active portion thereof is a NiV-F protein that has the amino acid sequence set forth in SEQ ID NO: 7 or an amino acid sequence having at or about 80%, at least at or about 81%, at least at or about 82%, at least at or about 83%, at or about 84%, at least at or about 85%, at least at or about 86%, or at least at or about 87%, at least at or about 88%, or at least at or about 89%, at least at or about 90%, at least at or about 91%, at least at or about 92%, at least at or about 93%, at least at or about 94%, at least at or about 95%, at or about 96%, at least at or about 97%, at least at or about 98%, or at least at or about 99% sequence identity to SEQ ID NO:7. In some of any of the provided embodiments, the NiV-F protein is a biologically active portion that is truncated and lacks up to 40 contiguous amino acid residues at or near the C-terminus of the wild-type NiV-F protein (SEQ ID NO:7). In some of any of the provided embodiments, the NiV-F protein is a biologically active portion that is truncated at the C-terminus of wild-type NiV-F and has the sequence set forth in any of SEQ ID NOS: 1-10 or an amino acid sequence having at least at or about 80%, at least at or about 81%, at least at or about 82%, at least at or about 83%, at least at or about 84%, at least at or about 85%, at least at or about 86%, at least at or about 87%, at least at or about 88%, at least at or about 89%, at least at or about 90%, at least at or about 91%, at least at or about 92%, at least at or about 93%, at least at or about 94%, at least at or about 95%, at or about 96%, at least at or about 97%, at least at or about 98%, or at least at or about 99% sequence identity to SEQ ID NOs: 1-10. In some of any of the provided embodiments, the NiV-F protein is a biologically active portion that has a truncation at or near the C-terminus of the wild-type NiV-F selected from the group consisting of a 5 amino acid truncation at or near the C-terminus of the wild-type NiV-F protein, a 10 amino acid truncation at or near the C-terminus of the wild-type NiV-F protein, a 15 amino acid truncation at or near the C-terminus, a 20 amino acid truncation at or near the C-terminus of the wild-type NiV-F protein, a 22 amino acid truncation at or near the C-terminus of the wild-type NiV-F protein, or a 25 amino acid truncation at or near the C-terminus of the wild-type NiV-F protein, optionally wherein the wild-type NiV-F protein is set forth in SEQ ID NO:7. In some of any of the provided embodiments, the F protein is a NiV-F protein that is a biologically active portion that has a 20 amino acid truncation at or near the C-terminus of the wild-type NiV-F protein (SEQ ID NO:7).

In some of any of the provided embodiments, the NiV-F protein has an amino acid sequence set forth in SEQ ID NO:2 or an amino acid sequence having at or about 80%, at least at or about 81%, at least at or about 82%, at least at or about 83%, at or about 84%, at least at or about 85%, at least at or about 86%, or at least at or about 87%, at least at or about 88%, or at least at or about 89%, at least at or about 90%, at least at or about 91%, at least at or about 92%, at least at or about 93%, at least at or about 94%, at least at or about 95%, at or about 96%, at least at or about 97%, at least at or about 98%, or at least at or about 99% sequence identity to SEQ ID NO: 2. In some of any of the provided embodiments, the F protein is a NiV-F protein that is a biologically active portion thereof that has a 22 amino acid truncation at or near the C-terminus of the wild-type NiV-F protein (SEQ ID NO:7). In some of any of the provided embodiments, the NiV-F protein has an amino acid sequence set forth in SEQ ID NO:11 or a sequence having at or about 80%, at least at or about 81%, at least at or about 82%, at least at or about 83%, at or about 84%, at least at or about 85%, at least at or about 86%, or at least at or about 87%, at least at or about 88%, or at least at or about 89%, at least at or about 90%, at least at or about 91%, at least at or about 92%, at least at or about 93%, at least at or about 94%, at least at or about 95%, at or about 96%, at least at or about 97%, at least at or about 98%, or at least at or about 99% sequence identity to SEQ ID NO: 11.

In some of any of the provided embodiments, the NiV-F protein is a biologically active portion that has a truncation at or near the C-terminus of the wild-type NiV-F selected from the group consisting of a 5 amino acid truncation at or near the C-terminus of the wild-type NiV-F protein, a 10 amino acid truncation at or near the C-terminus of the wild-type NiV-F protein, a 15 amino acid truncation at or near the C-terminus, a 20 amino acid truncation at or near the C-terminus of the wild-type NiV-F protein, a 22 amino acid truncation at or near the C-terminus of the wild-type NiV-F protein, or a 25 amino acid truncation at or near the C-terminus of the wild-type NiV-F protein, optionally wherein the wild-type NiV-F protein is set forth in SEQ ID NO:7. In some of any of the provided embodiments, the F protein is a NiV-F protein that is a biologically active portion that has a 20 amino acid truncation at or near the C-terminus of the wild-type NiV-F protein (SEQ ID NO:7).

In some of any of the provided embodiments, the NiV-F protein has an amino acid sequence set forth in SEQ ID NO:2 or an amino acid sequence having at or about 80%, at least at or about 81%, at least at or about 82%, at least at or about 83%, at or about 84%, at least at or about 85%, at least at or about 86%, or at least at or about 87%, at least at or about 88%, or at least at or about 89%, at least at or about 90%, at least at or about 91%, at least at or about 92%, at least at or about 93%, at least at or about 94%, at least at or about 95%, at or about 96%, at least at or about 97%, at least at or about 98%, or at least at or about 99% sequence identity to SEQ ID NO: 2.

In some of any of the provided embodiments, the F protein is a NiV-F protein that is a biologically active portion thereof that has a 22 amino acid truncation at or near the C-terminus of the wild-type NiV-F protein (SEQ ID NO:7). In some of any of the provided embodiments, the NiV-F protein has an amino acid sequence set forth in SEQ ID NO:11 or a sequence having at or about 80%, at least at or about 81%, at least at or about 82%, at least at or about 83%, at or about 84%, at least at or about 85%, at least at or about 86%, or at least at or about 87%, at least at or about 88%, or at least at or about 89%, at least at or about 90%, at least at or about 91%, at least at or about 92%, at least at or about 93%, at least at or about 94%, at least at or about 95%, at or about 96%, at least at or about 97%, at least at or about 98%, or at least at or about 99% sequence identity to SEQ ID NO: 11. In some of any of the provided embodiments, the NiV-F protein has an amino acid sequence set forth in SEQ ID NO:12 or a sequence having at or about 80%, at least at or about 81%, at least at or about 82%, at least at or about 83%, at or about 84%, at least at or about 85%, at least at or about 86%, or at least at or about 87%, at least at or about 88%, or at least at or about 89%, at least at or about 90%, at least at or about 91%, at least at or about 92%, at least at or about 93%, at least at or about 94%, at least at or about 95%, at or about 96%, at least at or about 97%, at least at or about 98%, or at least at or about 99% sequence identity to SEQ ID NO: 12.

In some of any of the provided embodiments, the F protein or the biologically active portion thereof is a NiV-F protein that is a functionally active variant that comprises a point mutation on an N-linked glycosylation site of the wild-type NiV-F protein (SEQ ID NO:7) or a biologically active portion thereof. In some of any of the provided embodiments, the F protein or the biologically active portion thereof is a NiV-F protein that is a functionally active variant that comprises: i) a 20 amino acid truncation at or near the C-terminus of the wild-type NiV-F protein (SEQ ID NO:7); and/or ii) a point mutation on an N-linked glycosylation site.

In some of any of the provided embodiments, the NiV-F protein has an amino acid sequence set forth in SEQ ID NO:11 or an amino acid sequence having at or about 80%, at least at or about 81%, at least at or about 82%, at least at or about 83%, at or about 84%, at least at or about 85%, at least at or about 86%, or at least at or about 87%, at least at or about 88%, or at least at or about 89%, at least at or about 90%, at least at or about 91%, at least at or about 92%, at least at or about 93%, at least at or about 94%, at least at or about 95%, at or about 96%, at least at or about 97%, at least at or about 98%, or at least at or about 99% sequence identity to SEQ ID NO: 11.

In some of any of the provided embodiments, the G protein or the biologically active portion thereof is a wild-type Nipah virus G (NiV-G) protein or a Hendra virus G protein or is a functionally active variant or biologically active portion thereof. In some of any of the provided embodiments, the G protein or the biologically active portion thereof is a wild-type NiV-G protein or a functionally active variant or biologically active portion thereof. In some of any of the provided embodiments, the G protein or the biologically active portion thereof is a mutant NiV-G protein that is modified to exhibit reduced native binding tropism. In some of any of the provided embodiments, the G protein or the biologically active portion thereof is a mutant NiV-G protein that exhibits reduced binding to Ephrin B2 or Ephrin B3.

In some of any of the provided embodiments, the G protein or the biologically active portion thereof is a mutant NiV-G protein comprising one or more amino acid substitutions corresponding to amino acid substitutions selected from the group consisting of E501A, W504A, Q530A and E533A with reference to numbering set forth in SEQ ID NO:14. In some of any of the provided embodiments, the NiV-G protein is a biologically active portion that is truncated and lacks up to 40 contiguous amino acid residues at or near the N-terminus of the wild-type NiV-G protein (SEQ ID NO:14).

In some of any of the provided embodiments, the NiV-G protein is a biologically active portion that has a truncation at or near the N-terminus of the wild-type NiV-G selected from the group consisting of a 5 amino acid truncation at or near the N-terminus of the wild-type NiV-G protein, a 10 amino acid truncation at or near the N-terminus of the wild-type NiV-G protein, a 15 amino acid truncation at or near the N-terminus of the wild-type NiV-G protein, a 20 amino acid truncation at or near the N-terminus of the wild-type NiV-G protein, a 25 amino acid truncation at or near the N-terminus of the wild-type NiV-G protein, a 30 amino acid truncation at or near the N-terminus of the wild-type NiV-G protein, or a 34 amino acid truncation at or near the N-terminus of the wild-type NiV-G protein, optionally wherein the wild-type NiV-G protein is set forth in SEQ ID NO:13, SEQ ID NO:14, or SEQ ID NO: 19.

In some of any of the provided embodiments, the NiV-G protein is a biologically active portion that is truncated at the N-terminus of wild-type NiV-G and has the sequence set forth in any of SEQ ID NOS: 13, 14, or 19 or an amino acid sequence having at least at or about 80%, at least at or about 81%, at least at or about 82%, at least at or about 83%, at least at or about 84%, at least at or about 85%, at least at or about 86%, at least at or about 87%, at least at or about 88%, at least at or about 89%, at least at or about 90%, at least at or about 91%, at least at or about 92%, at least at or about 93%, at least at or about 94%, at least at or about 95%, at or about 96%, at least at or about 97%, at least at or about 98%, or at least at or about 99% sequence identity to SEQ ID NOs 13, 14, or 19.

In some of any of the provided embodiments, the G protein molecule or a biologically active portion thereof NiV-G protein has the amino acid sequence set forth in SEQ ID NO: 13 or an amino acid sequence having at or about 80%, at least at or about 81%, at least at or about 82%, at least at or about 83%, at or about 84%, at least at or about 85%, at least at or about 86%, or at least at or about 87%, at least at or about 88%, or at least at or about 89%, at least at or about 90%, at least at or about 91%, at least at or about 92%, at least at or about 93%, at least at or about 94%, at least at or about 95%, at or about 96%, at least at or about 97%, at least at or about 98%, or at least at or about 99% sequence identity to SEQ ID NO:13. In some of any of the provided embodiments, the G protein or the biologically active portion thereof is a mutant NiV-G protein having the amino acid sequence set forth in SEQ ID NO: 14 or an amino acid sequence having at or about 80%, at least at or about 81%, at least at or about 82%, at least at or about 83%, at or about 84%, at least at or about 85%, at least at or about 86%, or at least at or about 87%, at least at or about 88%, or at least at or about 89%, at least at or about 90%, at least at or about 91%, at least at or about 92%, at least at or about 93%, at least at or about 94%, at least at or about 95%, at or about 96%, at least at or about 97%, at least at or about 98%, or at least at or about 99% sequence identity to SEQ ID NO:14. In some of any of the provided embodiments, the G protein or the biologically active portion thereof is a mutant NiV-G protein having the amino acid sequence set forth in SEQ ID NO:19 or an amino acid sequence having at or about 80%, at least at or about 81%, at least at or about 82%, at least at or about 83%, at or about 84%, at least at or about 85%, at least at or about 86%, or at least at or about 87%, at least at or about 88%, or at least at or about 89%, at least at or about 90%, at least at or about 91%, at least at or about 92%, at least at or about 93%, at least at or about 94%, at least at or about 95%, at or about 96%, at least at or about 97%, at least at or about 98%, or at least at or about 99% sequence identity to SEQ ID NO:19.

In some of any of the provided embodiments the F protein comprises the sequence set forth in SEQ ID NO. 12 and the G protein comprises the sequence set forth in SEQ ID NO. 19.

In some of any of the provided embodiments, at least one of the one or more Paramyxovirus envelope proteins are linked to a secondary moiety that is a targeting domain or a functional domain. In some of any of the provided embodiments, the at least one of the one or more Paramyxovirus is a glycoprotein G (G protein) or a biologically active portion thereof and the G protein or biologically active portion thereof is linked to the secondary moiety. In some of any of the provided embodiments, the secondary moiety is a functional domain and the functional domain is selected from a cytokine, growth factor, hormone, neurotransmitter, receptor, or apoptosis ligand. In some of any of the provided embodiments, the secondary moiety is a targeting domain and the targeting domain is specific for a cell surface receptor on a target cell. In some of any of the provided embodiments, the targeting domain is a Design ankyrin repeat proteins (DARPin), a single domain antibody (sdAb), a single chain variable fragment (scFv), or an antigen-binding fibronectin type III (Fn3) scaffold.

In some of any of the provided embodiments, the at least one of the one or more Paramyxovirus envelope proteins and the secondary moiety are directly linked. In some of any of the provided embodiments, the at least one of the one or more Paramyxovirus envelope proteins and secondary moiety are indirectly linked via a linker. In some of any of the provided embodiments, the linker is a peptide linker. In some of any of the provided embodiments, the peptide linker is (GmS)n (SEQ ID NO: 11), wherein each of m and n is an integer between 1 to 4, inclusive.

In some of any of the provided embodiments, the exogenous agent is a nucleic acid or a polypeptide. In some of any of the provided embodiments, the exogenous agent is a nucleic acid encoding a payload gene, optionally wherein the nucleic acid encodes a chimeric antigen receptor (CAR).

In some of any of the provided embodiments, the target cell is one or more of a monocyte, macrophage, neutrophil, dendritic cell, eosinophil, mast cell, platelet, large granular lymphocyte, Langerhans' cell, natural killer (NK) cell, T lymphocyte (e.g., T cell), a Gamma delta T cell, B lymphocyte (e.g., B cell), CD3+ T cell, a CD4+ T cell, a CD8+ T cell, a hepatocyte, a hematopoietic stem cell, a CD34+ hematopoietic stem cell, a CD105+ hematopoietic stem cell, a CD117+ hematopoietic stem cell, a CD105+ endothelial cell, a B cell, a CD20+ B cell, a CD19+ B cell, a cancer cell, a CD133+ cancer cell, an EpCAM+ cancer cell, a CD19+ cancer cell, a Her2/Neu+ cancer cell, a GluA2+ neuron, a GluA4+ neuron, a NKG2D+ natural killer cell, a SLC1A3+ astrocyte, a SLC7A10+ adipocyte, or a CD30+ lung epithelial cell.

In some of any of the provided embodiments, the first and second dose are administered ex vivo to the subject. In some of any of the provided embodiments, the first and second dose are administered to the subject intravenously. In some of any of the provided embodiments, the time period between the first and second dose is no more than one month. In some of any of the provided embodiments, the time period between the first and second dose is no more than one week. In some of any of the provided embodiments, the time period between the first and second dose is no more than three days.

In some of any of the provided embodiments, the method further comprises administration of a third dose of the targeted lipid particle. In some of any of the provided embodiments, the third dose is administered within one month (e.g., within four weeks, within three weeks, within two weeks, within seven days, within six days, within five days, within four days, within three days, within two days, or within one day) of the second dose. In some of any of the provided embodiments, the third dose is administered (i) on the first day following the second dose or (ii) on the second, third, fourth, fifth, sixth, seventh, fourteenth, twenty-first, or twenty-eighth day following the second dose. In some of any of the provided embodiments, the third dose is administered ex vivo to the subject. In some of any of the provided embodiments, the third dose is administered to the subject intravenously. In some of any of the provided embodiments, the time period between the first and third dose is no more than one month. In some of any of the provided embodiments, the time period between the first and third dose is no more than one week. In some of any of the provided embodiments, the time period between the first and third dose is no more than three days.

Provided herein is a targeted lipid particle comprising one or more Paramyxovirus envelope proteins for use in any of the provided methods. In some of any of the provided embodiments, the lipid particle is a viral vector and/or viral-like particle. In some of any of the provided embodiments, the lipid particle is a viral vector, optionally wherein the lipid particle is a lentiviral vector.

### BRIEF DESCRIPTION OF THE DRAWINGS

FIG. 1A depicts percent of hepatocytes positive for transgene expression (GFP reporter) in mice administered a single dose of an exemplary pseudotyped lentiviral vector retargeted for delivery to hepatocytes.

FIG. 1B depicts percent of hepatocytes positive for transgene expression (GFP reporter) after successive repeat doses (dose 1, *1; dose 2, *2; and dose 3, *3) of mice administered with an exemplary pseudotyped lentiviral vector retargeted for delivery to hepatocytes at different vector potency.

FIG. 1C depicts vector copy number (VCN) in hepatocytes after successive repeat doses (dose 1, *1; dose 2, *2; and dose 3, *3) of mice administered with an exemplary pseudotyped lentiviral vector retargeted for delivery to hepatocytes at different vector potency.

FIG. 2A depicts expression of an exemplary therapeutic gene (hOTC) following administration of an exemplary pseudotyped lentiviral vector retargeted for delivery to hepatocytes at different vector potency. FIG. 2B depicts the correlation between this expression and transgene activity. Vector copy number following administration of the exemplary pseudotyped lentiviral vector is shown in FIG. 2C.

FIG. 3 depicts an exemplary flow diagram of one embodiment of the provided method of administering a lipid nanoparticle (e.g. viral vector) to a subject.

FIG. 4 depicts an exemplary flow diagram outlining an alternative embodiment of the method in FIG. 3 in which one or more various optional features can be additionally incorporated into the method.

FIG. 5A depicts assessment of transduction and transgene expression in vivo after repeat administration with an exemplary lentiviral vector (LV) comprising a nucleic acid encoding a red fluorescent protein (RFP) transgene. An increase in RFP positive cells was seen in hcCD34 and CD34+ cells (FIG. 5B), as well as CD19+ and CD13+/CD33+ cells (FIG. 5C).

### DETAILED DESCRIPTION

Provided herein are methods for dosing a lipid particle or viral vector in repeated doses in two or more successive administrations, such as two, three or four successive administrations. The provided embodiments thus relate to methods in which the overall dose of a lipid particle or viral vector is provided by repeated dosing. In particular embodiments, the lipid particles or viral vectors are targeted for delivery to a target cell. In some embodiments, the lipid particle or viral vector contains one or more Paramyxovirus envelope proteins (e.g. Nipah virus G and F proteins or truncated variants thereof) that facilitate targeting and/or fusion of the lipid particle or viral vector to the target cell. In some embodiments, the lipid particle or viral vector contains a Nipah virus G (NiV-G) and F (NiV-F) protein or truncated variant thereof that lacks a portion of the cytoplasmic domain, in which the NiV-G is retargeted for delivery to the target cell by fusion with a targeting moiety (e.g. scFv or VHH binding domain). Exemplary features of the provided lipid particles and viral vectors are described in following sections.

In some embodiments, provided herein is a method for administration of a lipid particle or viral vector to a subject, the method comprising administering to a subject a first dose of a targeted lipid particle, and administering to the subject a second dose of the targeted lipid particle, wherein the targeted lipid particle comprises one or more Paramyxovirus envelope proteins. Also provided herein is a method for administration of a payload gene to subject, the method comprising administering to a subject a first dose of a targeted lipid particle comprising a payload gene encoding an exogenous agent, and administering to the subject a second dose of the targeted lipid particle, wherein the targeted lipid particle comprises one or more Paramyxovirus envelope proteins. In some embodiments, the methods may further include administration of one or more successive doses. In some embodiments, the time between administration of any successive dose (e.g. second dose) compared to a prior dose (e.g. first dose) is no more than one month, such as within three weeks, two weeks, one week or less. In some embodiments, the time between administration of any successive dose (e.g. second dose) compared to a prior dose (e.g. first dose) is less than one week, such that the time between any two doses is within at or about 96 hours, within at or about 72 hours, within at or about 48 hours or within at or about 24 hours. In some embodiments, the time of administration of each successive dose is between 12 and 36 hours, such as at or about 24 hours.

In some embodiments, the methods provide for a strategy for administration of lipid particles or viral vectors, as carriers for therapeutic payloads. In some embodiments, the provided methods provide for repeat dosing of a lipid particle or viral vector including for delivery of a payload gene contained therein to a subject. In some embodiments, the viral vector may be a viral vector, such as a viral vector that is pseudotyped for targeting to a desired target cell. Thus, in some embodiments, the provided methods provide for transduction for delivery of a viral vector or payload gene to target cells of interest for therapy. In some embodiments, delivery of the payload gene to target cells may provide a therapeutic intervention or treatment for a disease or condition, such as cancer or a genetic deficiency.

The provided methods can in some aspects increase efficiency of on-target cell delivery and reduce total amount of lipid particle or viral vector needed for any given dose. For instance, repeated administration as provided allows for surprisingly high total efficiency transfection and/or transduction even where the effective dose of each administered dose of the lipid particle or viral vector is reduced and administered over successive days. For instance, the provided methods offer advantages of dosing viral vectors with overall lower vector potency. In some aspects, repeat administration also allows for administration of smaller volumes, reducing the total viral particles needed for therapeutic composition manufacturing, transport, and delivery. In some cases, the methods permit delivery of a lipid particle (viral vector) at a defined, small volume, which can increase the certain of transduction events even at lower doses. In some embodiments, methods of repeat dosing in accord with the provided methods also can minimize off target toxicity, such as to organs, compared to methods involving systemic (e.g. intravenous) delivery of the lipid particles at only one time point.

All publications, including patent documents, scientific articles and databases, referred to in this application are incorporated by reference in their entirety for all purposes to the same extent as if each individual publication were individually incorporated by reference. If a definition set forth herein is contrary to or otherwise inconsistent with a definition set forth in the patents, applications, published applications and other publications that are herein incorporated by reference, the definition set forth herein prevails over the definition that is incorporated herein by reference.

The section headings used herein are for organizational purposes only and are not to be construed as limiting the subject matter described. Those skilled in the art will recognize that several embodiments are possible within the scope and spirit of the present disclosure. The following description illustrates the disclosure and, of course, should not be construed in any way as limiting the scope of the inventions described herein.

### I. DEFINITIONS

Unless defined otherwise, all terms of art, notations and other technical and scientific terms or terminology used herein are intended to have the same meaning as is commonly understood by one of ordinary skill in the art to which the claimed subject matter pertains. In some cases, terms with commonly understood meanings are defined herein for clarity and/or for ready reference, and the inclusion of such definitions herein should not necessarily be construed to represent a substantial difference over what is generally understood in the art.

Unless defined otherwise, all technical and scientific terms, acronyms, and abbreviations used herein have the same meaning as commonly understood by one of ordinary skill in the art to which the invention pertains. Unless indicated otherwise, abbreviations and symbols for chemical and biochemical names is per IUPAC-IUB nomenclature. Unless indicated otherwise, all numerical ranges are inclusive of the values defining the range as well as all integer values in-between.

As used herein, the articles “a” and “an” refer to one or to more than one (i.e. to at least one) of the grammatical object of the article. By way of example, “an element” means one element or more than one element.

As used herein, the term “about” will be understood by persons of ordinary skill in the art and will vary to some extent on the context in which it is used. As used herein, “about” when referring to a measurable value such as an amount, a temporal duration, and the like, is meant to encompass variations of ±20% or ±10%, more preferably ±5%, even more preferably ±1%, and still more preferably ±0.1% from the specified value, as such variations are appropriate to perform the disclosed methods.

As used herein, “lipid particle” refers to any biological or synthetic particle that contains a bilayer of amphipathic lipids enclosing a lumen or cavity. Typically a lipid particle does not contain a nucleus. Such lipid particles include, but are not limited to, viral particles (e.g. lentiviral particles), virus-like particles, viral vectors (e.g., lentiviral vectors) exosomes, enucleated cells, various vesicles, such as a microvesicle, a membrane vesicle, an extracellular membrane vesicle, a plasma membrane vesicle, a giant plasma membrane vesicle, an apoptotic body, a mitoparticle, a pyrenocyte, or a lysosome. In some embodiments, a lipid particle can be a fusosome. In some embodiments, the lipid particle is not a platelet. In some embodiments, the fusosome is derived from a source cell. A lipid particle also may include an exogenous agent or a nucleic acid encoding an exogenous agent, which may be present in the lumen of the lipid particle.

The terms “viral vector particle” and “viral vector” are used interchangeably herein and refer to a vector for transfer of an exogenous agent (e.g. non-viral or exogenous nucleic acid) into a recipient or target cell and that contains one or more viral structural proteins in addition to at least one non-structural viral genomic component or functional fragment thereof (i.e., a polymerase, an integrase, a protease or other non-structural component). The viral vector thus contains the exogenous agent, such as heterologous nucleic acid that includes non-viral coding sequences, to be transferred into a cell. Examples of viral vectors are retroviral vectors, such as lentiviral vectors.

The term “retroviral vector” refers to a viral vector that contains retroviral nucleic acid or is derived from a retrovirus. A retroviral vector particle includes the following components: a vector genome (retrovirus nucleic acid), a nucleocapsid encapsidating the nucleic acid, and a membrane envelope surrounding the nucleocapsid. Typically, a retroviral vector contains sufficient retroviral genetic information to allow packaging of an RNA genome, in the presence of packaging components, into a viral particle capable of infecting a target cell, Infection of the target cell may include reverse transcription and integration into the target cell genome. A retroviral vector may be a recombinant retroviral vector that is replication defective and lacks genes essential for replication, such as a functional gag-pol and/or env gene and/or other genes essential for replication. A retroviral vector also may be a self-inactivating (SIN) vector.

As used herein, a “lentiviral vector” or LV refers to a viral vector that contains lentiviral nucleic acid or is derived from a lentivirus. A lentiviral vector particle includes the following components: a vector genome (lentivirus nucleic acid), a nucleocapsid encapsidating the nucleic acid, and a membrane surrounding the nucleocapsid, Typically, a lentiviral vector contains sufficient lentiviral genetic information to allow packaging of an RNA genome, in the presence of packaging components, into a viral particle capable of infecting a target cell. Infection of the target cell may include reverse transcription and integration into the target cell genome. A lentiviral vector may be a recombinant lentiviral vector that is replication defective and lacks genes essential for replication, such as a functional gag-pol and/or env gene and/or other genes essential for replication. A lentiviral vector also may be a self-inactivating (SIN) vector.

As used herein, a “retroviral nucleic acid,” refers to a nucleic acid containing at least the minimal sequence requirements for packaging into a retroviral vector, alone or in combination with a helper cell, helper virus, or helper plasmid. In the case of “lentiviral nucleic acid” the nucleic acid refers to at least the minimal sequence requirements for packaging into a lentiviral vector, alone or in combination with a helper cell, helper virus, or helper plasmid. In some embodiments, the viral nucleic acid comprises one or more of (e.g., all of) a 5′ LTR (e.g., to promote integration), U3 (e.g., to activate viral genomic RNA transcription), R (e.g., a Tat-binding region), U5, a 3′ LTR (e.g., to promote integration), a packaging site (e.g., psi (Ψ)), RRE (e.g., to bind to Rev and promote nuclear export). The viral nucleic acid can comprise RNA (e.g., when part of a virion) or DNA (e.g., when being introduced into a source cell or after reverse transcription in a recipient cell). In some embodiments, the viral nucleic acid is packaged using a helper cell, helper virus, or helper plasmid which comprises one or more of (e.g., all of) gag, pol, and env.

As used herein, “fusosome” refers to a lipid particle containing a bilayer of amphipathic lipids enclosing a lumen or cavity and a fusogen that interacts with the amphipathic lipid bilayer. In some embodiments, the fusosome is a membrane enclosed preparation. In some embodiments, the fusosome is derived from a source cell. A fusosome also may include an exogenous agent or a nucleic acid encoding an exogenous agent, which may be present in the lumen of the fusosome.

As used herein, “fusosome composition” refers to a composition comprising one or more fusosomes.

As used herein, “fusogen” refers to an agent or molecule that creates an interaction between two membrane enclosed lumens. In embodiments, the fusogen facilitates fusion of the membranes. In other embodiments, the fusogen creates a connection, e.g., a pore, between two lumens (e.g., a lumen of a retroviral vector and a cytoplasm of a target cell). In some embodiments, the fusogen comprises a complex of two or more proteins, e.g., wherein neither protein has fusogenic activity alone. In some embodiments, the fusogen comprises a targeting domain. Examples of fusogens include paramyxovirus F and G proteins such as those from Nipah Virus (NiV) and biologically active portions or variants thereof including any as described.

As used herein, a “re-targeted fusogen,” such as a re-targeted G protein, refers to a fusogen that comprises a targeting moiety having a sequence that is not part of the naturally-occurring form of the fusogen in which the targeting moiety targets or binds a molecule on a desired cell type. In embodiments, the fusogen comprises a different targeting moiety relative to the targeting moiety in the naturally-occurring form of the fusogen. In embodiments, the naturally-occurring form of the fusogen lacks a targeting domain, and the re-targeted fusogen comprises a targeting moiety that is absent from the naturally-occurring form of the fusogen. In embodiments, the fusogen is modified to comprise a targeting moiety. In some such embodiments, the attachment of the targeting moiety to a fusogen (e.g. G protein) may be directly or indirectly via a linker, such as a peptide linker. In embodiments, the fusogen comprises one or more sequence alterations outside of the targeting moiety relative to the naturally-occurring form of the fusogen, e.g., in a transmembrane domain, fusogenically active domain, or cytoplasmic domain.

As used herein, a “target cell” refers to a cell of a type to which it is desired that a targeted lipid particle or viral vector delivers an exogenous agent. In embodiments, a target cell is a cell of a specific tissue type or class, e.g., an immune effector cell, e.g., a T cell. In some embodiments, a target cell is a diseased cell, e.g., a cancer cell. In some embodiments, the fusogen, e.g., re-targeted fusogen leads to preferential delivery of the exogenous agent to a target cell compared to a non-target cell.

As used herein a “non-target cell” refers to a cell of a type to which it is not desired that a targeted lipid particle or viral vector delivers an exogenous agent. In some embodiments, a non-target cell is a cell of a specific tissue type or class. In some embodiments, a non-target cell is a non-diseased cell, e.g., a non-cancerous cell. In some embodiments, the fusogen, e.g., re-targeted fusogen leads to lower delivery of the exogenous agent to a non-target cell compared to a target cell.

As used herein a “biologically active portion,” such as with reference to a protein such as a G protein or an F protein, refers to a portion of the protein that exhibits or retains an activity or property of the full-length of the protein. For example, a biologically active portion of an F protein retains fusogenic activity in conjunction with the G protein when each are embedded in a lipid bilayer. A biologically active portion of the G protein retains fusogenic activity in conjunction with an F protein when each is embedded in a lipid bilayer. The retained activity can include 10%-150% or more of the activity of a full-length or wild-type F protein or G protein. Examples of biologically active portions of F and G proteins include proteins with truncations of the cytoplasmic domain, such as any of the described NiV-F with a truncated cytoplasmic tail.

As used herein, “percent (%) amino acid sequence identity” and “homology” with respect to a peptide, polypeptide or antibody sequence are defined as the percentage of amino acid residues in a candidate sequence that are identical with the amino acid residues in the specific peptide or polypeptide sequence, after aligning the sequences and introducing gaps, if necessary, to achieve the maximum percent sequence identity, and not considering any conservative substitutions as part of the sequence identity. Alignment for purposes of determining percent amino acid sequence identity can be achieved in various ways that are within the skill in the art, for instance, using publicly available computer software such as BLAST, BLAST-2, ALIGN or MEGALIGN (DNASTAR) software. Those skilled in the art can determine appropriate parameters for measuring alignment, including any algorithms needed to achieve maximal alignment over the full length of the sequences being compared.

An amino acid substitution may include but are not limited to the replacement of one amino acid in a polypeptide with another amino acid. Exemplary substitutions are shown in Table 1. Amino acid substitutions may be introduced into an antibody of interest and the products screened for a desired activity, for example, retained/improved binding.

TABLE 1

| Original Residue | Exemplary Substitutions |
|---|---|
| Ala (A) | Val; Leu; Ile |
| Arg (R) | Lys; Gln; Asn |
| Asn (N) | Gln; His; Asp, Lys; Arg |
| Asp (D) | Glu; Asn |
| Cys (C) | Ser; Ala |
| Gln (Q) | Asn; Glu |
| Glu (E) | Asp; Gln |
| Gly (G) | Ala |
| His (H) | Asn; Gln; Lys; Arg |
| Ile (I) | Leu; Val; Met; Ala; Phe; Norleucine |
| Leu (L) | Norleucine; Ile; Val; Met; Ala; Phe |
| Lys (K) | Arg; Gln; Asn |
| Met (M) | Leu; Phe; Ile |
| Phe (F) | Trp; Leu; Val; Ile; Ala; Tyr |
| Pro (P) | Ala |
| Ser (S) | Thr |
| Thr (T) | Val; Ser |
| Trp (W) | Tyr; Phe |
| Tyr (Y) | Trp; Phe; Thr; Ser |
| Val (V) | Ile; Leu; Met; Phe; Ala; Norleucine |

Amino acids may be grouped according to common side-chain properties:
(1) hydrophobic: Norleucine, Met, Ala, Val, Leu, Ile;
(2) neutral hydrophilic: Cys, Ser, Thr, Asn, Gln;
(3) acidic: Asp, Glu;
(4) basic: His, Lys, Arg;
(5) residues that influence chain orientation: Gly, Pro;
(6) aromatic: Trp, Tyr, Phe.

Non-conservative substitutions will entail exchanging a member of one of these classes for another class.

The term, “corresponding to” with reference to positions of a protein, such as recitation that nucleotides or amino acid positions “correspond to” nucleotides or amino acid positions in a disclosed sequence, such as set forth in the Sequence listing, refers to nucleotides or amino acid positions identified upon alignment with the disclosed sequence based on structural sequence alignment or using a standard alignment algorithm, such as the GAP algorithm. For example, corresponding residues of a similar sequence (e.g. fragment or species variant) can be determined by alignment to a reference sequence by structural alignment methods. By aligning the sequences, one skilled in the art can identify corresponding residues, for example, using conserved and identical amino acid residues as guides.

The term “isolated” as used herein refers to a molecule that has been separated from at least some of the components with which it is typically found in nature or produced. For example, a polypeptide is referred to as “isolated” when it is separated from at least some of the components of the cell in which it was produced. Where a polypeptide is secreted by a cell after expression, physically separating the supernatant containing the polypeptide from the cell that produced it is considered to be “isolating” the polypeptide. Similarly, a polynucleotide is referred to as “isolated” when it is not part of the larger polynucleotide (such as, for example, genomic DNA or mitochondrial DNA, in the case of a DNA polynucleotide) in which it is typically found in nature, or is separated from at least some of the components of the cell in which it was produced, for example, in the case of an RNA polynucleotide. Thus, a DNA polynucleotide that is contained in a vector inside a host cell may be referred to as “isolated”.

The term “effective amount” as used herein means an amount of a pharmaceutical composition which is sufficient to significantly and positively modify the symptoms and/or conditions to be treated (e.g., provide a positive clinical response). The effective amount of an active ingredient for use in a pharmaceutical composition will vary with the particular condition being treated, the severity of the condition, the duration of treatment, the nature of concurrent therapy, the particular active ingredient(s) being employed, the particular pharmaceutically-acceptable excipient(s) and/or carrier(s) utilized, and like factors with the knowledge and expertise of the attending physician.

An “exogenous agent” as used herein with reference to a lipid particle or viral vector refers to an agent that is neither comprised by nor encoded in the corresponding wild-type virus or fusosome made from a corresponding wild-type source cell. In some embodiments, the exogenous agent does not naturally exist, such as a protein or nucleic acid that has a sequence that is altered (e.g., by insertion, deletion, or substitution) relative to a naturally occurring protein. In some embodiments, the exogenous agent does not naturally exist in the source cell. In some embodiments, the exogenous agent exists naturally in the source cell but is exogenous to the virus. In some embodiments, the exogenous agent does not naturally exist in the recipient cell. In some embodiments, the exogenous agent exists naturally in the recipient cell, but is not present at a desired level or at a desired time. In some embodiments, the exogenous agent comprises RNA or protein.

As used herein, a “promoter” refers to a cis-regulatory DNA sequence that, when operably linked to a gene coding sequence, drives transcription of the gene. The promoter may comprise a transcription factor binding sites. In some embodiments, a promoter works in concert with one or more enhancers which are distal to the gene.

As used herein, a composition refers to any mixture of two or more products, substances, or compounds, including cells. It may be a solution, a suspension, liquid, powder, a paste, aqueous, non-aqueous or any combination thereof.

As used herein, the term “pharmaceutically acceptable” refers to a material, such as a carrier or diluent, which does not abrogate the biological activity or properties of the compound, and is relatively nontoxic, i.e., the material may be administered to an individual without causing undesirable biological effects or interacting in a deleterious manner with any of the components of the composition in which it is contained.

As used herein, the term “pharmaceutical composition” refers to a mixture of at least one compound of the invention with other chemical components, such as carriers, stabilizers, diluents, dispersing agents, suspending agents, thickening agents, and/or excipients. The pharmaceutical composition facilitates administration of the compound to an organism. Multiple techniques of administering a compound exist in the art including, but not limited to, intravenous, oral, aerosol, parenteral, ophthalmic, pulmonary and topical administration.

A “disease” or “disorder” as used herein refers to a condition where treatment is needed and/or desired.

As used herein, the terms “treat,” “treating,” or “treatment” refer to ameliorating a disease or disorder, e.g., slowing or arresting or reducing the development of the disease or disorder or reducing at least one of the clinical symptoms thereof. For purposes of this disclosure, ameliorating a disease or disorder can include obtaining a beneficial or desired clinical result that includes, but is not limited to, any one or more of: alleviation of one or more symptoms, diminishment of extent of disease, preventing or delaying spread (for example, metastasis, for example metastasis to the lung or to the lymph node) of disease, preventing or delaying recurrence of disease, delay or slowing of disease progression, amelioration of the disease state, inhibiting the disease or progression of the disease, inhibiting or slowing the disease or its progression, arresting its development, and remission (whether partial or total).

The terms “individual” and “subject” are used interchangeably herein to refer to an animal; for example a mammal. The term patient includes human and veterinary subjects. In some embodiments, methods of treating mammals, including, but not limited to, humans, rodents, simians, felines, canines, equines, bovines, porcines, ovines, caprines, mammalian laboratory animals, mammalian farm animals, mammalian sport animals, and mammalian pets, are provided. The subject can be male or female and can be any suitable age, including infant, juvenile, adolescent, adult, and geriatric subjects. In some examples, an “individual” or “subject” refers to an individual or subject in need of treatment for a disease or disorder. In some embodiments, the subject to receive the treatment can be a patient, designating the fact that the subject has been identified as having a disorder of relevance to the treatment, or being at adequate risk of contracting the disorder. In particular embodiments, the subject is a human, such as a human patient.

### II. METHODS OF DOSING A LIPID PARTICLE OR VIRAL VECTOR INCLUDING IN METHODS OF TREATMENT

Provided herein are methods for administration of a lipid particle or viral vector to a subject. In some embodiments, the lipid particle or viral vector may contain a payload gene encoding an exogenous agent, and the methods are for delivering the payload gene to a target cell in the subject. In provided embodiments, the methods include repeat administration of the lipid particles or viral vector in which the overall dose is divided into two or more doses given successively.

Thus, in provided embodiments, administration of a total dose of a lipid particle or viral vector includes administration of a total desired dose that includes at least two repeated doses that are each separately administered resulting in multiple administrations over a specified time period. In some embodiments, each repeated dose may be administered from a separate composition containing the lipid particle or viral vector so that the total dose is provided as a plurality of compositions that are administered separately over a specified time period. In some embodiments, the plurality of compositions (e.g. providing a first dose and a second dose, and optionally one or more successive doses) are administered over a time period that is no more than one month. In some embodiments, a first dose and second dose, and in some cases one or more additional doses, are administered over more than one day. In some embodiments, the plurality of compositions (e.g. providing a first dose and a second dose, and optionally one or more successive doses) are administered over a time period that is no more than one week. In some embodiments, the repeated doses are administered over a period of no more than three days, such as once a day for two days (e.g. a first dose and a second dose) or once a day for three days (e.g. a first dose, a second dose, and a third dose).

In some embodiments, the methods of dosing provided herein include treatment methods for treating a disease or condition in a subject. In some embodiments, the administration of the repeated doses delivers the viral vectors to a target cell (e.g., hepatocytes, T cells or other target cell) in the subject. In such embodiments, the subject may be at risk of, may have a symptom of, or may be diagnosed with or identified as having, a particular disease or condition. In some embodiments, the methods thereby treat the disease or condition or disorder in the subject. In one embodiment, the subject has cancer. In one embodiment, the subject has an infectious disease. In some embodiments, the lipid particle or viral vector, e.g. retroviral particles other viral vectors, contains nucleic acid sequences encoding an exogenous agent for treating the disease or condition in the subject. For example, the exogenous agent is one that targets or is specific for a protein of a neoplastic cells and the lipid particle or viral vector, e.g. retroviral particles such as lentiviral vector, is administered to a subject for treating a tumor or cancer in the subject. In another example, the exogenous agent is an inflammatory mediator or immune molecule, such as a cytokine, and the lipid particle or viral vector, e.g. retroviral particles such as a lentiviral vector, is administered to a subject for treating any condition in which it is desired to modulate (e.g. increase) the immune response, such as a cancer or infectious disease. In some embodiments, the repeated doses of the lipid particles or viral vector, e.g. retroviral particles such as lentiviral vector, is administered to provide an effective amount or total dose to effect treatment of the disease, condition or disorder.

In such embodiments, the subject may be at risk of, may have a symptom of, or may be diagnosed with or identified as having, a particular disease or condition. In one embodiment, the subject has cancer. In one embodiment, the subject has an infectious disease. In some embodiments, the lipid particle, such as a targeted lipid particle, contains nucleic acid sequences encoding the payload agent (also interchangeably called an exogenous agent or in some cases “cargo”) for treating the disease or condition in the subject. Thus, in some embodiments, the disease or condition that is treated is any that may be treatable by the encoded payload agent. For instance, in some embodiments the payload agent encodes a chimeric antigen receptor (CAR) that specifically binds to an antigen, and the disease or condition to be treated can be any in which expression of the antigen is associated with and/or involved in the etiology of a disease condition or disorder, e.g. causes, exacerbates or otherwise is involved in such disease, condition, or disorder. Exemplary diseases and conditions can include diseases or conditions associated with malignancy or transformation of cells (e.g. cancer), autoimmune or inflammatory disease, or an infectious disease, e.g. caused by bacterial, viral or other pathogens. Exemplary antigens, which include antigens associated with various diseases and conditions that can be treated, include any of antigens described herein.

Also among embodiments provided herein are uses of any of the provided lipid particles or viral vectors, e.g. retroviral particles such as lentiviral vector, in such methods and treatments, and in the preparation of a medicament in order to carry out such therapeutic methods. In some embodiments, the uses or medicaments are for use in methods that are carried out by administering the lipid particles or viral vector, e.g. retroviral particles other viral vectors or fusosomes thereof, or compositions comprising the same, as a repeated dose to a subject having, having had, or suspected of having the disease or condition or disorder. In some embodiments, the uses or medicaments are for use in methods that thereby treat the disease or condition or disorder in the subject. Also provided herein are uses of any of the compositions, such as pharmaceutical compositions provided herein, for repeated dosing to a subject for the treatment of a disease, condition or disorder associated with a particular gene or protein targeted by or provided by the exogenous agent.

This disclosure provides a method of administering a viral vector or lipid particle to a subject (e.g., a human subject), a target tissue, or a cell, comprising administering to the subject, a plurality of compositions comprising a viral vector or lipid particles described herein, thereby administering the viral vector or lipid particle to the subject. In some embodiments, the lipid particle or viral vector contains a payload gene encoding an exogenous agent. Thus, in some embodiments, the viral vector or lipid particle is capable of delivering (e.g., delivers) an exogenous agent (i.e., a payload gene) to a target cell.

Among provided methods herein are methods that comprise delivering an agent (i.e., a payload gene) to a target cell. In some embodiments, the exogenous agent is an agent that is entirely heterologous or not produced or normally expressed by the target cell (i.e., a gene that is not produced or normally expressed by the target cell). In some embodiments, delivery of the exogenous agent to the target cell can provide a therapeutic effect to treat a disease or condition in the subject. The therapeutic effect may be by targeting, modulating or altering an antigen or protein present or expressed by the target cell that is associated with or involved in a disease or condition. The therapeutic effect may be by providing an exogenous agent in which the exogenous agent is a protein (or a nucleic acid encoding the protein, e.g., an mRNA encoding the protein) which is absent, mutant, or at a lower level than wild-type in the target cell. In some embodiments, the target cell is from a subject having a genetic disease, e.g., a monogenic disease, e.g., a monogenic intracellular protein disease.

In some embodiments, the provided methods or uses involve administration of the dose as a pharmaceutical composition by oral, inhaled, transdermal or parenteral (including intravenous, intratumoral, intraperitoneal, intramuscular, intracavity, and subcutaneous) administration. In some embodiments, the lipid particles or viral vectors may be administered alone or formulated as a pharmaceutical composition. In some embodiments, the lipid particles or viral vectors or pharmaceutical compositions described herein can be administered to a subject, e.g., a mammal, e.g., a human. In some of any embodiments, the subject may be at risk of, may have a symptom of, or may be diagnosed with or identified as having, a particular disease or condition (e.g., a disease or condition described herein). In some embodiments, the disease is a disease or disorder.

The viral vectors described herein can be administered to a subject, e.g., a mammal, e.g., a human. In such embodiments, the subject may be at risk of, may have a symptom of, or may be diagnosed with or identified as having, a particular disease or condition (e.g., a disease or condition described herein). In some embodiments, the disease or condition may be one that is treated by delivery of the exogenous agent (i.e., payload gene) contained in the administered viral vector to a target cell in the subject.

A medical doctor, e.g., physician or veterinarian, having ordinary skill in the art may readily determine and prescribe the effective amount of the pharmaceutical composition required for administration in several divided repeat doses.

In some embodiments, it is especially advantageous to formulate the composition in dosage unit form for ease of administration and uniformity of dosage. In some embodiments, dosage unit form as used herein refers to physically discrete units suited as unitary dosages for administering as a dose, such as repeat dose, for the subjects to be treated; each unit containing a predetermined quantity of lipid particle or viral vector calculated to produce the desired therapeutic effect in association with the required pharmaceutical vehicle.

In some embodiments, the compositions provided herein containing a provided viral vector such as any of the viral vectors or virus-based particles described herein, can be formulated in dosage units of genome copies (GC). Suitable method for determining GC have been described and include, e.g., qPCR or digital droplet PCR (ddPCR) as described in, e.g., M. Lock et al, Hu Gene Therapy Methods, Hum Gene Ther Methods 25(2):115-25. 2014, which is incorporated herein by reference. In some embodiments, the dosage, such as total dose administered as repeat doses, for administration of a viral vector or virus-like particle is from about 10⁴ to about 10¹⁰ GC units, inclusive. In some embodiments, the dosage, such as total dose administered as repeat doses, for administration of a viral vector or virus-like particle is from about 10⁹ to about 10¹⁵ GC units, inclusive. In some embodiments, the dosage, such as total dose administered as repeat doses, for administration of a viral vector or virus-like particle is from about 10⁵ to about 10⁹ GC units, inclusive. In some embodiments, the dosage, such as total dose administered as repeat doses, for administration of a viral vector or virus-like particle is from about 10⁶ to about 10⁹ GC units, inclusive. In some embodiments, the dosage, such as total dose administered as repeat doses, for administration of a viral vector or virus-like particle is from about 10⁶ to about 10¹² GC units, inclusive. In some embodiments, the dosage, such as total dose administered as repeat doses, for administration of a viral vector or virus-like particle is from about 10¹² to about 10¹⁴ GC units, inclusive. In some embodiments, the dosage, such as total dose administered as repeat doses, for administration is 1.0×10⁹ GC units, 5.0×10⁹ GC units, 1.0×10¹⁰ GC units, 5.0×10¹⁰ GC units, 1.0×10¹¹ GC units, 5.0×10¹¹ GC units, 1.0×10¹² GC units, 5.0×10¹² GC units, or 1.0×10¹¹ GC units, 5.0×10¹¹ GC units, 1.0×10¹⁴ GC units, 5.0×10¹⁴ GC units, or 1.0×10¹⁵ GC units.

In some embodiments, the compositions provided herein containing a provided viral vector such as any of the viral vectors or virus-based particles described herein, can be formulated in dosage units of vector genomes (VG). Suitable method for determining VG have been described and include, e.g., qPCR or digital droplet PCR (ddPCR). In some embodiments, the dosage, such as total dose administered as repeat doses, for administration of a viral vector or virus-like particle is from about 10⁴ to about 10¹⁰ VG units, inclusive. In some embodiments, the dosage, such as total dose administered as repeat doses, for administration of a viral vector or virus-like particle is from about 10⁹ to about 10¹⁵ VG units, inclusive. In some embodiments, the dosage, such as total dose administered as repeat doses, for administration of a viral vector or virus-like particle is from about 10⁵ to about 10⁹ VG units, inclusive. In some embodiments, the dosage, such as total dose administered as repeat doses, for administration of a viral vector or virus-like particle is from about 10⁶ to about 10⁹ VG units, inclusive. In some embodiments, the dosage, such as total dose administered as repeat doses, for administration of a viral vector or virus-like particle is from about 10⁹ to about 10¹² VG units, inclusive. In some embodiments, the dosage, such as total dose administered as repeat doses, for administration of a viral vector or virus-like particle is from about 10¹² to about 10¹⁴ VG units, inclusive. In some embodiments, the dosage, such as total dose administered as repeat doses, for administration is 1.0×10⁹ VG units, 5.0×10⁹ VG units, 1.0×10¹⁰ VG units, 5.0×10¹⁰ VG units, 1.0×10¹¹ VG units, 5.0×10¹¹ VG units, 1.0×10¹² VG units, 5.0×10¹² VG units, or 1.0×10¹³ VG units, 5.0×10¹³ VG units, 1.0×10¹⁴ VG units, 5.0×10¹⁴ VG units, or 1.0×10¹⁵ VG units.

In some embodiments, the dosage, such as total dose administered as repeat doses, for administration of a viral vector or virus-like particle is from about 10⁴ to about 10¹⁰ infectious units, inclusive. In some embodiments, the dosage, such as total dose administered as repeat doses, for administration of a viral vector or virus-like particle is from about 10⁹ to about 10¹⁵ infectious units, inclusive. In some embodiments, the dosage, such as total dose administered as repeat doses, for administration of a viral vector or virus-like particle is from about 10⁵ to about 10⁹ infectious units. In some embodiments, the dosage, such as total dose administered as repeat doses, for administration of a viral vector or virus-like particle is from about 10⁶ to about 10⁹ infectious units. In some embodiments, the dosage, such as total dose administered as repeat doses, for administration of a viral vector or virus-like particle is from about 10⁹ to about 10¹² infectious units, inclusive. In some embodiments, the dosage, such as total dose administered as repeat doses, for administration of a viral vector or virus-like particle is from about 10¹² to about 10¹⁴ infectious units, inclusive. In some embodiments, the dosage, such as total dose administered as repeat doses, for administration is 1.0×10⁹ infectious units, 5.0×10⁹ infectious units, 1.0×10¹⁰ infectious units, 5.0×10¹⁰ infectious units, 1.0×10¹¹ infectious units, 5.0×10¹¹ infectious units, 1.0×10¹² infectious units, 5.0×10¹² infectious units, or 1.0×10¹³ infectious units, 5.0×10¹³ infectious units, 1.0×10¹⁴ infectious units, 5.0×10¹⁴ infectious units, or 1.0×10¹⁵ infectious units. The techniques available for quantifying infectious units are routine in the art and include viral particle number determination, fluorescence microscopy, and titer by plaque assay. For example, the number of adenovirus particles can be determined by measuring the absorbance at A260. Similarly, infectious units can also be determined by quantitative immunofluorescence of vector specific proteins using monoclonal antibodies or by plaque assay.

In some embodiments, methods that calculate the infectious units include the plaque assay, in which titrations of the virus are grown on cell monolayers and the number of plaques is counted after several days to several weeks. For example, the infectious titer is determined, such as by plaque assay, for example an assay to assess cytopathic effects (CPE). In some embodiments, a CPE assay is performed by serially diluting virus on monolayers of cells, such as HFF cells, that are overlaid with agarose. After incubation for a time period to achieve a cytopathic effect, such as for about 3 to 28 days, generally 7 to 10 days, the cells can be fixed and foci of absent cells visualized as plaques are determined. In some embodiments, infectious units can be determined using an endpoint dilution (TCID₅₀) method, which determines the dilution of virus at which 50% of the cell cultures are infected and hence, generally, can determine the titer within a certain range, such as one log.

In some embodiments, the dosage, such as total dose administered as repeat doses, for administration of a viral vector or virus-like particle is from about 10⁴ to about 10¹⁰ plaque forming units (pfu), inclusive. In some embodiments, the dosage, such as total dose administered as repeat doses, for administration of a viral vector or virus-like particle is from about 10⁹ to about 10¹⁵ pfu, inclusive. In some embodiments, the dosage, such as total dose administered as repeat doses, for administration of a viral vector or virus-like particle is from about 10⁵ to about 10⁹ pfu. In some embodiments, the dosage, such as total dose administered as repeat doses, for administration of a viral vector or virus-like particle is from about 10⁶ to about 10⁹ pfu. In some embodiments, the dosage, such as total dose administered as repeat doses, for administration of a viral vector or virus-like particle is from about 10⁹ to about 10¹² pfu, inclusive. In some embodiments, the dosage, such as total dose administered as repeat doses, for administration of a viral vector or virus-like particle is from about 10¹² to about 10¹⁴ pfu, inclusive. In some embodiments, the dosage of administration is 1.0×10⁹ pfu, 5.0×10⁹ pfu, 1.0×10¹⁰ pfu, 5.0×10¹⁰ pfu, 1.0×10¹¹ pfu, 5.0×10¹¹ pfu, 1.0×10¹² pfu, 5.0×10¹² pfu, or 1.0×10¹³ pfu, 5.0×10¹³ pfu, 1.0×10¹⁴ pfu, 5.0×10¹⁴ pfu, or 1.0×10¹⁵ pfu.

In some embodiments, one TU produces one integration event in target cells. In some aspects, if the percentage of infected cells is at or below 20% of the total cells, the number of integrations is approximately equal to the number of transduced cells whereby the TU and number of transduced cells have a linear relationship. In some aspects, at higher transduction levels, the fraction of transduced cells with multiple integrations increases. Therefore in some aspects, the percentage of transduced cells relative to integration events per cell (TU) is no longer linear.

In some aspects, the dosage of administration of a lipid particle or viral vector within the pharmaceutical compositions provided herein varies depending on a subject's body weight. For example, a composition may be formulated as GC/kg, VG/kg infectious units/kg, pfu/kg, TU/kg, etc. In some aspects, the dosage at which a therapeutic effect is obtained is from at or about 10⁸ TU/kg to at or about 10¹⁴ TU/kg of the subject's body weight, inclusive. In some aspects, the dosage is from at or about 10⁸ infectious units/kg to at or about 10¹⁴ infectious units/kg of the subject's body weight, inclusive.

In some embodiments, the compositions provided herein containing a provided viral vector such as any of the viral vectors or virus-based particles described herein, can be formulated in dosage units of transduction units (TU), including in TU/kg of the patient bodyweight. In some embodiments, the dosage, such as total dose administered as repeat doses, for administration of a viral vector or virus-like particle is from about 10⁴ to about 10¹⁰ TU/kg, inclusive. In some embodiments, the dosage, such as total dose administered as repeat doses, for administration of a viral vector or virus-like particle is from about 10⁹ to about 10¹⁵ TU/kg, inclusive In some embodiments, the dosage, such as total dose administered as repeat doses, for administration of a viral vector or virus-like particle is from about 10⁵ to about 10⁹ TU/kg. In some embodiments, the dosage, such as total dose administered as repeat doses, for administration of a viral vector or virus-like particle is from about 10⁶ to about 10⁹ TU/kg. In some embodiments, the dosage, such as total dose administered as repeat doses, for administration of a viral vector or virus-like particle is from about 10⁹ to about 10¹² TU/kg, inclusive. In some embodiments, the dosage, such as total dose administered as repeat doses, for administration of a viral vector or virus-like particle is from about 10¹² to about 10¹⁴ TU/kg, inclusive. In some embodiments, the dosage, such as total dose administered as repeat doses, for administration is 1.0×10⁹ TU/kg, 5.0×10⁹ TU/kg, 1.0×10¹⁰ TU/kg, 5.0×10¹⁰ TU/kg, 1.0×10¹¹ TU/kg, 5.0×10¹¹ TU/kg, 1.0×10¹² TU/kg, 5.0×10¹² TU/kg, or 1.0×10¹³ TU/kg, 5.0×10¹³ TU/kg, 1.0×10¹⁴ TU/kg, 5.0×10¹⁴ TU/kg, or 1.0×10¹⁵ TU/kg. The techniques available for quantifying TU are routine in the art. These methods include calculating TU based on the following equation: TU/ml=(# of cells at Transduction)×[MOI/(ml of vector used at Transduction)].

In some embodiments, the compositions provided herein containing a provided viral vector such as any of the viral vectors or virus-based particles described herein, can be formulated in dosage units of VG/kg of the patient bodyweight. In some embodiments, the dosage, such as total dose administered as repeat doses, for administration of a viral vector or virus-like particle is from about 10⁴ to about 101 VG/kg, inclusive. In some embodiments, the dosage, such as total dose administered as repeat doses, for administration of a viral vector or virus-like particle is from about 10′ to about 10¹⁵ VG/kg, inclusive In some embodiments, the dosage, such as total dose administered as repeat doses, for administration of a viral vector or virus-like particle is from about 105 to about 10′ VG/kg. In some embodiments, the dosage, such as total dose administered as repeat doses, for administration of a viral vector or virus-like particle is from about 10⁶ to about 10′ VG/kg. In some embodiments, the dosage, such as total dose administered as repeat doses, for administration of a viral vector or virus-like particle is from about 10′ to about 10¹² VG/kg, inclusive. In some embodiments, the dosage, such as total dose administered as repeat doses, for administration of a viral vector or virus-like particle is from about 10¹² to about 10¹⁴ VG/kg, inclusive. In some embodiments, the dosage, such as total dose administered as repeat doses, for administration is 1.0×10⁹ VG/kg, 5.0×10⁹ VG/kg, 1.0×10¹⁰ VG/kg, 5.0×10¹⁰ VG/kg, 1.0×10¹¹ VG/kg, 5.0×10¹¹ VG/kg, 1.0×10¹² VG/kg, 5.0×10¹² VG/kg, or 1.0×10¹¹ VG/kg, 5.0×10¹³ VG/kg, 1.0×10¹⁴ VG/kg, 5.0×10¹⁴ VG/kg, or 1.0×10¹⁵ VG/kg.

In some embodiments, the total dose is administered as two repeated doses, i.e. a first dose and a second dose. In some embodiments, the second dose is administered within one month after the first dose. In some embodiments, the second dose is administered within 4 weeks, three weeks, two weeks or one week after the first dose. In some embodiments, the second dose is administered within one week of the first dose. In some embodiments, the second dose is administered within 1 day, 2 days, 3 days, 4 days, 5 days, 6 days or 7 days of the first dose. In some embodiments, the time between administering the first and second dose is no more than 3 days. In some embodiments, the second dose is administered within 6 hours, 12 hours, 24 hours, 48 hours, or 72 hours of the first dose.

In some embodiments, the amount administered in the first and second dose is about the same.

In some embodiments, the total dose administered may also include a third repeated dose. In some embodiments, the total dose is administered as three repeated doses, i.e. a first dose, a second dose and a third dose. In some embodiments, the third dose is administered within one month after the second dose. In some embodiments, the third dose is administered within 4 weeks, three weeks, two weeks or one week after the second dose. In some embodiments, the third dose is administered within one week of the second dose. In some embodiments, the third dose is administered within 1 day, 2 days, 3 days, 4 days, 5 days, 6 days or 7 days of the second dose. In some embodiments, the time between administering the second dose and third dose is no more than 3 days. In some embodiments, the third dose is administered within 6 hours, 12 hours, 24 hours, 48 hours, or 72 hours of the second dose.

In some embodiments, the time between administering the third dose and the first dose is no more than one month. In some embodiments, the time between administering the third dose and the first dose is no more than three weeks. In some embodiments, the time between administering the third dose and the first dose is no more than two weeks. In some embodiments, the time between administering the third dose and the first dose is no more than one week. In some embodiments, the time between administering the third dose and the first dose is not more than 3 days.

In some embodiments, the amount administered in the first dose, second dose and third dose is about the same.

In some embodiments, each repeated dose is from the same composition or preparation of lipid particle or viral vector. In some embodiments, at least one of the repeated dose is from a different composition or preparation of the lipid particle or viral vector. It is understood that the compositions to be administered as the dose contains the same particle (e.g. same targeted lipid particle or targeted viral vector, including encoding the same exogenous agent) but may, in some cases, differ in that the particular preparation or composition may be from a different batch or lot. Hence, it is understood that the exact amount of the repeated doses may not be identical such as due to variation in the potency of the different batch or lot. In other cases, the dose may be administered as a unit dosage that takes into account potency such as based on transduction units (TU).

In some embodiments, each repeated dose is administered directly in vivo to the subject. In some embodiments, the dose is administered intravenously, such as by infusion to the subject.

In some embodiments, each repeated dose is administered to the subject by ex vivo dosing. In some embodiments, the repeated dosing according to the present disclosure is administered using an ex vivo system. For instance, in embodiments in which a PBMC cell subset (e.g. T cells) are targeted for delivery, PBMCs may be obtained from a subject, contacted with the targeted lipid particle or viral vector and reinfused to the subject. In some embodiments, an ex vivo dose is administered by a method that includes a) obtaining whole blood from the subject; b) collecting the fraction of blood containing peripheral blood mononuclear cells (PBMCs) or a subset thereof (e.g. a leukocyte component such as T cells); c) contacting the collected PBMCs or subset thereof with a composition comprising the lipid particle or viral vector to create a transfection mixture; and d) reinfusing the contacted PBMCs or subset thereof and/or the transfection mixture to the subject, thereby administering the lipid particle and/or payload gene to the subject.

In some embodiments, the ex vivo system for dosing is by a method that may include the use of a combination of various apheresis machine hardware components, a software control module, and a sensor module to measure citrate or other solute levels in-line to ensure the maximum accuracy and safety of treatment prescriptions, and the use of replacement fluids designed to fully exploit the design of the system according to the present methods. It is understood that components described for one system according to the present invention can be implemented within other systems according to the present invention as well.

In some embodiments, the method for administration of an ex vivo dose of viral vector and/or lipid particle to the subject comprises the use of a blood processing set for obtaining the whole blood from the subject, a separation chamber for collecting the fraction of blood containing leukocyte components, a contacting container for the contacting the cells with the composition comprising the viral vector and/or lipid particle, and a further fluid circuit for reinfusion of cells to the patient. In some embodiments, the method further comprises any of i) a washing component for concentrating cells, and ii) a sensor and/or module for monitoring cell density and/or concentration. In some embodiments, the methods allow processing of blood directly from the patient, transduction with the viral vector and/or lipid particle, and reinfusion directly to the patient without any steps of selection. Further the methods also can be carried out without cryopreserving or freezing any cells before or between any one or more of the steps, such that there is no step of formulating cells with a cryoprotectant, e.g. DMSO. In some embodiments, the ex vivo methods of dosing does not include a lymphodepletion regimen. In some embodiments, the each repeated dose administered by ex vivo methods including steps (a)-(d) can be carried out for a time of no more than 24 hours, such as between 2 hours and 12 hours, for example 3 hours to 6 hours.

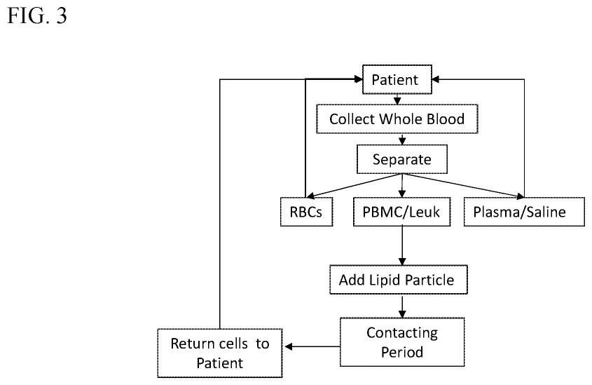

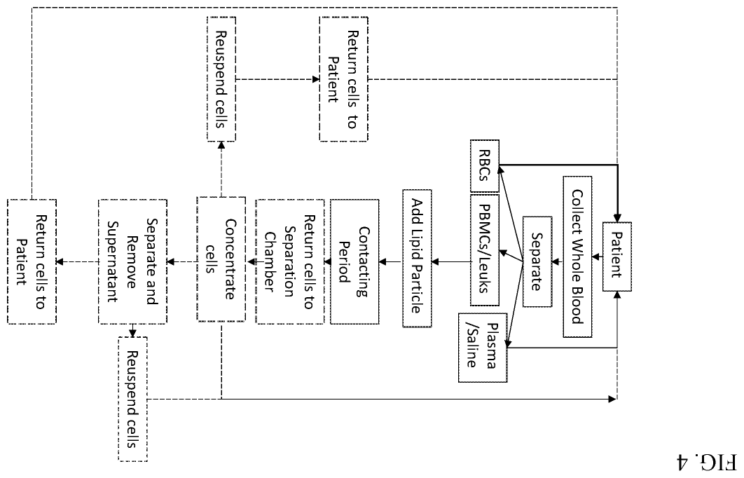

In some embodiments, the method is performed in-line. In some embodiments, the method is performed in a closed fluid circuit, or a functionally closed fluid circuit. In some embodiments, each of steps (a)-(d) are performed in-line in a closed fluid circuit in which all parts of the system are operably connected, such as via at least one tubing line. In some embodiments, the system is sterile. In some embodiments, the closed fluid circuit is sterile. An exemplary system for ex vivo administration of a dose is shown in FIG. 3 or 4.

In some embodiments, the lipid particle or viral vector is targeted for delivery to a particular target or tissue, such as one that is desired for delivering the payload gene or for treating the disease or conditions. Exemplary retargeted vectors and methods are described in Section III.F. In some embodiments, the target cell or tissue is any such listed in any of WO 2020/102499, WO 2020/102485, WO 2019/222403, WO 2020/014209, and WO 2020/102503, each of which is hereby incorporated by reference in its entirety. In some embodiments the target cell is an effector cell, e.g., a cell of the immune system that expresses one or more Fc receptors and mediates one or more effector functions. In some embodiments, a target cell may include, but may not be limited to, one or more of a monocyte, macrophage, neutrophil, dendritic cell, eosinophil, mast cell, platelet, large granular lymphocyte, Langerhans' cell, natural killer (NK) cell, T lymphocyte (e.g., T cell), a Gamma delta T cell, B lymphocyte (e.g., B cell) and may be from any organism including but not limited to humans, mice, rats, rabbits, and monkeys. In some embodiments, the target cell is selected from the group consisting of a CD3+ T cell, a CD4+ T cell, a CD8+ T cell, a hepatocyte, a hematopoietic stem cell, a CD34+ hematopoietic stem cell, a CD105+ hematopoietic stem cell, a CD117+ hematopoietic stem cell, a CD105+ endothelial cell, a B cell, a CD20+ B cell, a CD19+ B cell, a cancer cell, a CD133+ cancer cell, an EpCAM+ cancer cell, a CD19+ cancer cell, a Her2/Neu+ cancer cell, a GluA2+ neuron, a GluA4+ neuron, a NKG2D+ natural killer cell, a SLC1A3+ astrocyte, a SLC7A10+ adipocyte, or a CD30+ lung epithelial cell.

In some embodiments, the target cell is a T cell. In some embodiments, the target cell is any of a CD4+ T cell, a CD8+ T cell, an alpha beta T cell, a gamma delta T cell, a naive T cell, an effector T cell, a cytotoxic T cell (e.g., a CD8+ cytotoxic T cell), a regulatory T cell (e.g., a thymus-derived regulatory T cell, a peripherally derived regulatory T cell, a CD4+Foxp3+regulatory T cell, or a CD4+FoxP3− type 1 regulatory T (Trl) cell), a helper T cell (e.g., a CD4+ helper T cell, a Th1 cell, a Th2 cell, a Th3 cell, a Th9 cell, a Th17 cell, a Th22 cell, or a T follicular helper (Tfh) cell), a memory T cell (e.g., a stem cell memory T cell, a central memory T cell, or an effector memory T cell), a NKT cell, and a Mucosal associated invariant T (MAIT) cell.

In some embodiments, the payload agent is a chimeric antigen receptor, including any as described in Section III.G and/or that specifically binds an antigen described in Section III.G. Exemplary target antigens include, but are not limited to, CD5, CD19, CD20, CD22, CD23, CD30, CD70, Kappa, Lambda, and B cell maturation agent (BCMA), G-protein coupled receptor family C group 5 member D (GPRC5D) (associated with leukemias); CS1/SLAMF7, CD38, CD138, GPRC5D, TACI, and BCMA (associated with myelomas); GD2, HER2, EGFR, EGFRvIII, B7H3, PSMA, PSCA, CAIX, CD171, CEA, CSPG4, EPHA2, FAP, FRα, IL-13Rα, Mesothelin, MUC1, MUC16, and ROR1 (associated with solid tumors).

In some embodiments, the disease or condition is a B cell malignancy and the antigen targeted by the CAR is expressed by cells associated with the B cell malignancy. In some embodiments, the antigen is CD19. In some embodiments, the antigen is CD20. In some embodiments, the antigen is CD22. In some embodiments, the antigen is BCMA. In some embodiments, the B cell malignancy is a Large B-cell Lymphoma (LBCL). In some embodiments, the disease or condition has relapsed in or the subject is refractory to treatment of the disease or condition. For instance, in some embodiments, the disease or condition is relapsed and/or refractory Large B-cell Lymphoma (LBCL). In some embodiments, LBCL include Non-Hodgkin's lymphoma, including diffuse large B-cell lymphoma (DLBCL) not otherwise specified (including DLBCL arising from indolent lymphoma), primary mediastinal large B-cell lymphoma, high grade B-cell lymphoma, follicular lymphoma, and marginal zone lymphoma. In some embodiments, the subject has received or is receiving prior to the ex vivo dosing provided herein prior therapies, such as two or more lines of systemic therapy for treating the disease or condition. In some embodiments, the subject has relapsed and/or is refractory to the prior therapies. In some embodiments, the prior therapies include two or more prior therapies from a chemotherapy containing regimen, such as with anthracycline, or an anti-CD20 mAb (unless CD20 negative), or after autologous stem cell transplant (ASCT). In some embodiments, the subject has one or more measurable PET-positive lesion, such as measured per Lugano classification. In some embodiments, the subject as an ECOG performance status of 0 or 1. In some embodiments, the subject has adequate organ function.

In some embodiments, the disease or condition is a multiple myeloma and the antigen targeted by the CAR is expressed by cells associated with the multiple myeloma. In some embodiments, the antigen is BCMA. In some embodiments, the subject has or is suspected of having a multiple myeloma that is associated with expression of B cell maturation antigen (BCMA). In some embodiments, the multiple myeloma is a relapsed and/or refractory multiple myeloma.

In some aspects, response rates in subjects, such as subjects with LBCL, are based on the Lugano criteria. (Cheson et al., (2014) JCO., 32(27):3059-3067; Johnson et al., (2015) Radiology 2:323-338; Cheson, B. D. (2015) Chin. Clin. Oncol. 4(1):5). In some aspects, response assessment utilizes any of clinical, hematologic, and/or molecular methods. In some aspects, response assessed using the Lugano criteria involves the use of positron emission tomography (PET)-computed tomography (CT) and/or CT as appropriate. PET-CT evaluations may further comprise the use of fluorodeoxyglucose (FDG) for FDG-avid lymphomas. In some aspects, where PET-CT will be used to assess response in FDG-avid histologies, a 5-point scale may be used. In some respects, the 5-point scale comprises the following criteria: 1, no uptake above background; 2, uptake≤mediastinum; 3, uptake>mediastinum but≤liver; 4, uptake moderately>liver; 5, uptake markedly higher than liver and/or new lesions; X, new areas of uptake unlikely to be related to lymphoma.

In some embodiments, response is based on lack of detectable minimal residual disease (MRD negativity) which means that no disease is detected. Methods for assessing MRD include, but are not limited to flow cytometry, polymerase chain reaction (PCR) and next-generation sequencing. In some embodiments, a sample of bone marrow cells and/or peripheral blood cells is assessed for disease. For instance, certain mutations or genetic abnormalities can be assessed that are known to be associated with the cancer. A skilled artisan is familiar with methods to assess MRD.

In some cases, the pharmacokinetics of cells expressing the payload agent (e.g. CAR) are determined to assess the bioavailability of the engineered cells in vivo. Methods for determining the pharmacokinetics of engineered cells in vivo may include drawing peripheral blood from subjects that have received the ex vivo dosing and determining the number of engineered cells in the blood based on detection of the engineered payload agent (CAR) expressed by the cells. For example, an anti-idiotypic antibody against the CAR may be used for detection of the CAR-expressing cells.

In some embodiments, the provided embodiments do not involve a lymphodepletion therapy prior to the ex vivo administration of the lipid particle (e.g. viral vector). Thus, the provided methods do not involve administration of lymphopleting regimens, such as those including cyclophosphamide and/or fludarabine and/or bendamustine, or other lymphodepleting regimens or protocols, prior to receiving administration of the lipid particles. It is understood that the exclusion of lymphodepleting therapies in accord to the provided methods does not exclude that the subject may have previous in time (e.g. months to years earlier) may have received a lymphodepleting therapy. Rather, the provided embodiments include those in which the subject has not in accord with the present dosing methods received a lymphodepleting therapy, such as within 60 days or 30 days, prior to the ex vivo administration of the lipid particles (e.g. viral vector) of the present methods.

In some embodiments, the lipid particle composition comprising an exogenous agent or cargo, may be used to deliver such exogenous agent or cargo to a cell tissue or subject. In some embodiments, delivery of a cargo by administration of a lipid particle composition described herein may modify cellular protein expression levels. In certain embodiments, the administered composition directs upregulation of (via expression in the cell, delivery in the cell, or induction within the cell) of one or more cargo (e.g., a polypeptide or mRNA) that provide a functional activity which is substantially absent or reduced in the cell in which the polypeptide is delivered. In some embodiments, the missing functional activity may be enzymatic, structural, or regulatory in nature. In some embodiments, the administered composition directs up-regulation of one or more polypeptides that increases (e.g., synergistically) a functional activity which is present but substantially deficient in the cell in which the polypeptide is upregulated. In some of any embodiments, the administered composition directs downregulation of (via expression in the cell, delivery in the cell, or induction within the cell) of one or more cargo (e.g., a polypeptide, siRNA, or miRNA) that repress a functional activity which is present or upregulated in the cell in which the polypeptide, siRNA, or miRNA is delivered. In some of any embodiments, the upregulated functional activity may be enzymatic, structural, or regulatory in nature. In some embodiments, the administered composition directs down-regulation of one or more polypeptides that decreases (e.g., synergistically) a functional activity which is present or upregulated in the cell in which the polypeptide is downregulated. In some embodiments, the administered composition directs upregulation of certain functional activities and downregulation of other functional activities.

In some of any embodiments, the lipid particle composition (e.g., one comprising mitochondria or DNA) mediates an effect on a target cell, and the effect lasts for at least 1, 2, 3, 4, 5, 6, or 7 days, 2, 3, or 4 weeks, or 1, 2, 3, 6, or 12 months. In some embodiments (e.g., wherein the lipid particle composition comprises an exogenous protein), the effect lasts for less than 1, 2, 3, 4, 5, 6, or 7 days, 2, 3, or 4 weeks, or 1, 2, 3, 6, or 12 months.

In some embodiments, the viral vector delivers the exogenous agent to at least 40%, 45%, 50%, 55%, 60%, 65%, 70%, 75%, 80%, 85%, 90%, 95%, 96%, 97%, 98%, or 99% of the number of cells in the target cell population. In some embodiments, the viral vector delivers at least 40%, 45%, 50%, 55%, 60%, 65%, 70%, 75%, 80%, 85%, 90%, 95%, 96%, 97%, 98%, or 99% of the exogenous agent to the target cell.

In some embodiments, the viral vector and/or lipid particle delivers at least 40%, 45%, 50%, 55%, 60%, 65%, 70%, 75%, 80%, 85%, 90%, 95%, 96%, 97%, 98%, or 99% more of the exogenous agent to the target cell population compared to a non-target cell population. In some embodiments, the viral vector and/or lipid particle delivers more exogenous agent to the target cell population based on the viral vector and/or lipid particle comprising a fusogen or re-target fusogen that facilitates binding to the target cell population, but not the non-target cell population. The viral vector and/or lipid particle can comprise any of the exemplary fusogens and re-targeted fusogens described herein. In some embodiments, when the plurality of viral vectors and/or lipid particles are contacted with a cell population comprising target cells and non-target cells, the exogenous agent is present in at least 10-fold more target cells than non-target cells. In some embodiments, when the plurality of viral vectors and/or lipid particles are contacted with a cell population comprising target cells) and non-target cells, the exogenous agent is present at least 2-fold, 5-fold, 10-fold, 20-fold, or 50-fold higher in target cells than non-target cells and/or the exogenous agent is present at least 2-fold, 5-fold, 10-fold, 20-fold, or 50-fold higher in target cells than non-target cells. In some embodiments, the viral vectors and/or lipid particles of the plurality fuse at a higher rate with a target cell than with a non-target cell by at least 50%.

In some embodiments, the viral vector and/or lipid particle is capable of delivering (e.g., delivers) a nucleic acid to a target cell, e.g., to stably modify the genome of the target cell, e.g., for gene therapy. Similarly, in some embodiments, a method herein comprises delivering a nucleic acid to a target cell.

In some embodiments, a method herein comprises causing ligand presentation on the surface of a target cell by presenting cell surface ligands on the viral vector. In some embodiments, the viral vector is capable of causing cell death of the target cell. In some embodiments, the viral vector is from a NK source cell.

In some embodiments, a viral vector and/or lipid particle or target cell is capable of phagocytosis (e.g., of a pathogen). Similarly, in some embodiments, a method herein comprises causing phagocytosis.

In some embodiments, the viral vector and/or lipid particle comprises (e.g., is capable of delivering to the target cell) a membrane protein or a nucleic acid encoding the membrane protein.

In some embodiments, the viral vector and/or lipid particle, e.g., fusosome, fuses at a higher rate with a target cell than with a non-target cell based on the viral vector and/or lipid particle comprising a fusogen or re-target fusogen that facilitates binding to the target cell, but not the non-target cell. The viral vector and/or lipid particle can comprise any of the exemplary fusogens and re-targeted fusogens described herein. In some embodiments, the viral vector and/or lipid particle, e.g., fusosome, fuses at a higher rate with a target cell than with a non-target cell, e.g., by at least at least 1%, 2%, 3%, 4%, 5%, 10%, 20%, 30%, 40%, 50%, 60%, 70%, 80%, 90%, 2-fold, 3-fold, 4-fold, 5-fold, 10-fold, 20-fold, 50-fold, or 100-fold. In some embodiments, the viral vector and/or lipid particle, e.g., fusosome, fuses at a higher rate with a target cell than with other viral vectors and/or lipid particle, e.g., by at least 10%, 20%, 30%, 40%, 50%, 60%, 70%, 80%, or 90%. In some embodiments, the viral vector and/or lipid particle, e.g., fusosome, fuses with target cells at a rate such that an exogenous agent or nucleic acid encoding an exogenous agent in the viral vector and/or lipid particle is delivered to at least 10%, 20%, 30%, 40%, 50%, 60%, 70%, 80%, or 90%, of target cells after 24, 48, or 72 hours. In embodiments, the amount of targeted fusion is about 30%-70%, 35%-65%, 40%-60%, 45%-55%, or 45%-50%. In embodiments, the amount of targeted fusion is about 20%-40%, 25%-35%, or 30%-35%.

In some embodiments, the fusogen is present at a copy number of at least, or no more than, 10, 50, 100, 500, 1,000, 2,000, 5,000, 10,000, 20,000, 50,000, 100,000, 200,000, 500,000, 1,000,000, 5,000,000, 10,000,000, 50,000,000, 100,000,000, 500,000,000, or 1,000,000,000 copies. In some embodiments, at least 10%, 20%, 30%, 40%, 50%, 60%, 70%, 80%, 90%, 95%, 96%, 97%, 98%, or 99% of the fusogen comprised by the viral vector and/or lipid particle is disposed in the cell membrane. In embodiments, the viral vector and/or lipid particle also comprises fusogen internally, e.g., in the cytoplasm or an organelle. In some embodiments, the fusogen comprises (or is identified as comprising) about 0.1%, 0.2%, 0.3%, 0.4%, 0.5%, 0.6%, 0.7%, 0.8%, 0.9%, 1%, 5%, 10%, 11%, 12%, 13%, 14%, 15%, 20%, or more, or about 1-30%, 5-20%, 10-15%, 12-15%, 13-14%, or 13.6% of the total protein in a viral vector, e.g., as determined by a mass spectrometry assay. In embodiments, the fusogen comprises (or is identified as comprising) about 13.6% of the total protein in the viral vector and/or lipid particle. In some embodiments, the fusogen is (or is identified as being) more or less abundant than one or more additional proteins of interest. In an embodiment, the fusogen has (or is identified as having) a ratio to EGFP of about 140, 145, 150, 151, 152, 153, 154, 155, 156, 157 (e.g., 156.9), 158, 159, 160, 165, or 170. In another embodiment, the fusogen has (or is identified as having) a ratio to CD63 of about 2700, 2800, 2900, 2910 (e.g., 2912), 2920, 2930, 2940, 2950, 2960, 2970, 2980, 2990, or 3000, or about 1000-5000, 2000-4000, 2500-3500, 2900-2930, 2910-2915, or 2912.0, e.g., by a mass spectrometry assay. In an embodiment, the fusogen has (or is identified as having) a ratio to ARRDC1 of about 600, 610, 620, 630, 640, 650, 660 (e.g., 664.9), 670, 680, 690, or 700. In another embodiment, the fusogen has (or is identified as having) a ratio to GAPDH of about 50, 55, 60, 65, 70 (e.g., 69), 75, 80, or 85, or about 1-30%, 5-20%, 10-15%, 12-15%, 13-14%, or 13.6%. In another embodiment, the fusogen has (or is identified as having) a ratio to CNX of about 500, 510, 520, 530, 540, 550, 560 (e.g., 558.4), 570, 580, 590, or 600, or about 300-800, 400-700, 500-600, 520-590, 530-580, 540-570, 550-560, or 558.4, e.g., by a mass spectrometry assay.

In some of any embodiments, the lipid particle composition described herein is delivered for ex-vivo administration to a cell or tissue, e.g., a human cell or tissue. In embodiments, the composition improves function of a cell or tissue ex-vivo, e.g., improves cell viability, respiration, or other function (e.g., another function described herein).

In some embodiments, the composition is delivered for ex vivo administration to a tissue that is in an injured state (e.g., from trauma, disease, hypoxia, ischemia or other damage).

In some embodiments, the composition is delivered for ex-vivo transplant (e.g., a tissue explant or tissue for transplantation, e.g., a human vein, a musculoskeletal graft such as bone or tendon, cornea, skin, heart valves, nerves; or an isolated or cultured organ, e.g., an organ to be transplanted into a human, e.g., a human heart, liver, lung, kidney, pancreas, intestine, thymus, eye). In some embodiments, the composition is delivered to the tissue or organ before, during and/or after transplantation.

In some embodiments, the lipid particle compositions described herein can be administered to a subject, e.g., a mammal, e.g., a human. In such embodiments, the subject may be at risk of, may have a symptom of, or may be diagnosed with or identified as having, a particular disease or condition (e.g., a disease or condition described herein).

In some embodiments, the source of lipid particles are from the same subject that is administered a lipid particle composition. In other embodiments, they are different. In some embodiments, the source of lipid particles and recipient tissue may be autologous (from the same subject) or heterologous (from different subjects). In some embodiments, the donor tissue for lipid particle compositions described herein may be a different tissue type than the recipient tissue. In some embodiments, the donor tissue may be muscular tissue and the recipient tissue may be connective tissue (e.g., adipose tissue). In other embodiments, the donor tissue and recipient tissue may be of the same or different type, but from different organ systems.

In some embodiments, the lipid particle composition described herein may be administered to a subject having a cancer, an autoimmune disease, an infectious disease, a metabolic disease, a neurodegenerative disease, or a genetic disease (e.g., enzyme deficiency). In some embodiments, the subject is in need of regeneration.

In some embodiments, the lipid particle is co-administered with an inhibitor of a protein that inhibits membrane fusion. For example, Suppressyn is a human protein that inhibits cell-cell fusion (Sugimoto et al., “A novel human endogenous retroviral protein inhibits cell-cell fusion” Scientific Reports 3: 1462 (DOI: 10.1038/srep01462)). In some embodiments, the lipid particle particles is co-administered with an inhibitor of sypressyn, e.g., a siRNA or inhibitory antibody.

### III. LIPID PARTICLES AND VIRAL VECTORS FOR ADMINISTRATION

The provided methods and embodiments can be used to deliver of lipid particles or viral vectors for administration to a subject

In some embodiments, vectors that package a polynucleotide encoding a payload agent may be used to deliver the payload agent according to the provided methods. These vectors may be of any kind, including DNA vectors, RNA vectors, plasmids, viral vectors and particles. Viral vector technology is well known and described in Sambrook et al. (2001, Molecular Cloning: A Laboratory Manual, Cold Spring Harbor Laboratory, New York). Viruses, which are useful as vectors include, but are not limited to lentiviral vectors, adenoviral vectors, adeno-associated viral (AAV) vectors, herpes simplex viral vectors, retroviral vectors, oncolytic viruses, and the like.

In some embodiments, the vector may be a viral vector such as a lentiviral vector, a gamma-retroviral vector, a recombinant AAV, an adenoviral vector or an oncolytic viral vector. In other aspects, non-viral vectors for example, nanoparticles and liposomes may also be used for introducing and delivery of a polynucleotide encoding the payload agent.

In some embodiments, the lipid particle is a viral vector or is derived from a viral vector. In other embodiments, the vehicle is a non-viral vector, such as a cellular particle, liposome, nanoparticle, or other synthetic particle. Non-viral vectors and methods employing the use of polymers, surfactants, and/or excipients have been employed to introduce polynucleotides and polypeptides into cells including conjugation with a targeting moiety, conjugation with a cell penetrating peptide, derivatization with a lipid and incorporation into liposomes, lipid nanoparticles, and cationic liposomes. The majority of non-viral vectors are composed of plasmid DNA complexed with lipids or polycations. Many different lipids with ability to deliver plasmid DNA to cells in vitro and in vivo have been reported (Gao, et al., Gene Therapy 2:710-722 (1995)).

In any of the provided embodiments, the lipid particle or viral encodes a payload gene for delivery to a cell or a cell in a subject.

In particular embodiments, the payload gene is encapsulated within the lumen of a lipid particle in which the lipid particle contains a lipid bilayer, a lumen surrounded by the lipid bilayer. In some embodiments, the lipid particle can be a viral particle, a virus-like particle, a nanoparticle, a vesicle, an exosome, a dendrimer, a lentivirus, a viral vector, an enucleated cell, a microvesicle, a membrane vesicle, an extracellular membrane vesicle, a plasma membrane vesicle, a giant plasma membrane vesicle, an apoptotic body, a mitoparticle, a pyrenocyte, a lysosome, another membrane enclosed vesicle, or a lentiviral vector, a viral based particle, a virus like particle (VLP) or a cell derived particle.

In some embodiments, the lipid bilayer includes membrane components of the host cell from which the lipid bilayer is derived, e.g., phospholipids, membrane proteins, etc. In some embodiments, the lipid bilayer includes a cytosol that includes components found in the cell from which the vehicle is derived, e.g., solutes, proteins, nucleic acids, etc., but not all of the components of a cell, e.g., lacking a nucleus. In some embodiments, the lipid bilayer is considered to be exosome-like. The lipid bilayer may vary in size, and in some instances have a diameter ranging from 30 and 300 nm, such as from 30 and 150 nm, and including from 40 to 100 nm.

In some embodiments, the lipid bilayer is a viral envelope. In some embodiments, the viral envelope is obtained from a host cell. In some embodiments, the viral envelope is obtained by the viral capsid from the source cell plasma membrane. In some embodiments, the lipid bilayer is obtained from a membrane other than the plasma membrane of a host cell. In some embodiments, the viral envelope lipid bilayer is embedded with viral proteins, including viral glycoproteins.

In other aspects, the lipid bilayer includes synthetic lipid complex. In some embodiments, the synthetic lipid complex is a liposome. In some embodiments, the lipid bilayer is a vesicular structure characterized by a phospholipid bilayer membrane and an inner aqueous medium. In some embodiments, the lipid bilayer has multiple lipid layers separated by aqueous medium. In some embodiments, the lipid bilayer forms spontaneously when phospholipids are suspended in an excess of aqueous solution. In some examples, the lipid components undergo self-rearrangement before the formation of closed structures and entrap water and dissolved solutes between the lipid bilayers.

In some embodiments, the lipid particle comprises several different types of lipids. In some embodiments, the lipids are amphipathic lipids. In some embodiments, the amphipathic lipids are phospholipids. In some embodiments, the phospholipids comprise phosphatidylcholine, phosphatidylethanolamine, phosphatidylinositol, and phosphatidylserine. In some embodiments, the lipids comprise phospholipids such as phosphocholines and phosphoinositols. In some embodiments, the lipids comprise DMPC, DOPC, and DSPC.

### A. Viral Vectors

In some embodiments, the lipid particles include viral vector particles. In some embodiment the viral particles include those derived from retroviruses or lentiviruses. In some embodiments, the viral particle's bilayer of amphipathic lipids is or comprises the viral envelope. In some embodiments, the viral particle's bilayer of amphipathic lipids is or comprises lipids derived from an infected host cell.

Biological methods for introducing an exogenous agent to a host cell include the use of DNA and RNA vectors. DNA and RNA vectors can also be used to house and deliver polynucleotides and polypeptides. Viral vectors, and especially retroviral vectors, have become the most widely used method for inserting genes into mammalian, e.g., human cells. Other viral vectors can be derived from lentivirus, poxviruses, herpes simplex virus I, adenoviruses and adeno-associated viruses, and the like. See, for example, U.S. Pat. Nos. 5,350,674 and 5,585,362. Methods for producing cells comprising vectors and/or exogenous acids are well-known in the art. See, for example, Sambrook et al., 2001, Molecular Cloning: A Laboratory Manual, Cold Spring Harbor Laboratory, New York.

In some embodiments, the polynucleotides (e.g. encoding a payload gene) are comprised within a viral vector. In some embodiments, the polynucleotides (e.g. encoding a payload gene) comprised within a recombinant virus particles.

In some embodiments, the viral vector is a vectors derived from adenoviruses and adeno-associated virus (AAV). Such vectors or viral particles may be designed to utilize any of the known serotype capsids or combinations of serotype capsids. The serotype capsids may include capsids from any identified AAV serotypes and variants thereof, for example, AAV1, AAV2, AAV2G9, AAV3, AAV4, AAV4-4, AAV5, AAV6, AAV7, AAV8, AAV9, AAV10, AAV11, AAV12 and AAVrh10. In some embodiments, the AAV serotype may be or have a sequence as described in United States Publication No. US20030138772; Pulicherla et al. Molecular Therapy, 2011, 19(6): 1070-1078; U.S. Pat. Nos. 6,156,303; 7,198,951; U.S. Patent Publication Nos.: US2015/0159173 and US2014/0359799; and International Patent Publication NOs.: WO1998/011244, WO2005/033321 and WO2014/14422.

In some embodiments, the AAV vector is of serotype 1, 2, 6, 8 or 9. In some embodiments, the AAV vector is of serotype 6.2. In some embodiments, the AAV vector includes a capsid that is a chimera between AAV2 (aa 1-128) and AAV5 (aa 129-725) with one point mutation (A581T) (AAV2.5T, Excoffon et al. Proc Natl Acad Sci. 106(10):3875-70, 2009). In some embodiments, the AAV is a single-stranded DNA parvovirus which is capable of host genome integration during the latent phase of infectivity. For example, AAV of serotype 2 is largely endemic to the human and primate populations and frequently integrates site-specifically into human chromosome 19 q13.3. In some aspects, AAV is considered a dependent virus because it requires helper functions from either adenovirus or herpes-virus in order to replicate. In the absence of either of these helper viruses, AAV has been observed to integrate its genome into the host cell chromosome. However, these virions are not capable of propagating infection to new cells. AAV vectors include not only single stranded vectors but self-complementary AAV vectors (scAAVs). scAAV vectors contain DNA which anneals together to form double stranded vector genome. By skipping second strand synthesis, scAAVs allow for rapid expression in the cell. The rAAV vectors may be manufactured by standard methods in the art such as by triple transfection, in sf9 insect cells or in suspension cell cultures of human cells such as HEK293 cells.

In some embodiment, suitable host cells for producing AAV derived vehicles include microorganisms, yeast cells, insect cells, and mammalian cells. In some embodiments, the term host cell includes the progeny of the original cell which has been transfected. Thus, as indicated above, a “host cell,” or “producer cell,” as used herein, generally refers to a cell which has been transfected with a vector vehicle as described herein. For example, cells from the stable human cell line, 293 (ATCC Accession No. CRL1573) are familiar to those in the art as a producer cell for AAV vectors. The 293 cell line is a human embryonic kidney cell line that has been transformed with adenovirus type-5 DNA fragments (Graham et al., J. Gen. Virol., 36:59 (1977)), and expresses the adenoviral Ela and E1b genes (Aiello et al., Virol., 94:460 (1979)). The 293 cell line is readily transfected, and thus provides a particularly useful system in which to produce AAV virions.

Producer cells as described above containing the AAV vehicles provided herein must be rendered capable of providing AAV helper functions. In some embodiments, producer cells allow AAV vectors to replicate and encapsulate polynucleotide sequences. In some embodiments, producer cells yield AAV virions. AAV helper functions are generally AAV-derived coding sequences that may be expressed to provide AAV gene products that, in turn, function for productive AAV replication. In some embodiments, AAV helper functions are used to complement necessary AAV functions that are missing from the AAV vectors. In some embodiments, AAV helper functions include at least one of the major AAV ORFs. In some embodiments, the helper functions include at least the rep coding region, or a functional homolog thereof. In some embodiments, the helper function includes at least the cap coding region, or a functional homolog thereof.

In some embodiments, the AAV helper functions are introduced into the host cell by transfecting the host cell with a mixture of AAV helper constructs either prior to, or concurrently with, the transfection of the AAV vector. In some embodiments, the AAV helper constructs are used to provide transient expression of AAV rep and/or cap genes. In some embodiments, the AAV helper constructs lack AAV packaging sequences and can neither replicate nor package themselves.

In some embodiments, an AAV genome can be cross-packaged with a heterologous virus. Cross-genera packing of the rAAV2 genome into the human bocavirus type 1 (HBoV1) capsid (rAAV2/HBoV1 hybrid vector), for example, results in a hybrid vector that is highly tropic for airway epithelium (Yan et al., 2013, Mol. Ther., 21:2181-94).

In some embodiments, the virus particles are lentivirus. In some embodiments, the lentiviral vector particle is Human Immunodeficiency Virus-1 (HIV-1).

In some embodiments, the retroviral vector has a long terminal repeat sequence (LTR), e.g., a retroviral vector derived from the Moloney murine leukemia virus (MoMLV), myeloproliferative sarcoma virus (MPSV), murine embryonic stem cell virus (MESV), murine stem cell virus (MSCV), spleen focus forming virus (SFFV), or adeno-associated virus (AAV). Most retroviral vectors are derived from murine retroviruses. In some embodiments, the retroviruses include those derived from any avian or mammalian cell source. The retroviruses typically are amphotropic, meaning that they are capable of infecting host cells of several species, including humans. In one embodiment, the gene to be expressed replaces the retroviral gag, pol and/or env sequences. A number of illustrative retroviral systems have been described (e.g., U.S. Pat. Nos. 5,219,740; 6,207,453; 5,219,740).

Methods of lentiviral transduction are known. Exemplary methods are described in, e.g., Wang et al., J. Immunother. 35(9): 689-701, 2012; Cooper et al., Blood. 101:1637-1644, 2003; Verhoeyen et al., Methods Mol Biol. 506: 97-114, 2009; and Cavalieri et al., Blood. 102(2): 497-505, 2003. Exemplary methods for generating viral vectors including lentiviral vectors are described further below.

In some embodiments, the viral vector is a lentiviral vector. Lentiviral vectors are particularly useful means for successful viral transduction as they permit stable expression of the gene contained within the delivered nucleic acid transcript. Lentiviral vectors express reverse transcriptase and integrase, two enzymes required for stable expression of the gene contained within the delivered nucleic acid transcript. Reverse transcriptase converts an RNA transcript into DNA, while integrase inserts and integrates the DNA into the genome of the target cell. Once the DNA has been integrated stably into the genome, it divides along with the host. The gene of interest contained within the integrated DNA may be expressed constitutively or it may be inducible. As part of the host cell genome, it may be subject to cellular regulation, including activation or repression, depending on a host of factors in the target cell.

Lentiviruses are subgroup of the Retroviridae family of viruses, named because reverse transcription of viral RNA genomes to DNA is required before integration into the host genome. As such, the most important features of lentiviral vehicles/particles are the integration of their genetic material into the genome of a target/host cell. Some examples of lentivirus include the Human Immunodeficiency Viruses: HIV-1 and HIV-2, the Simian Immunodeficiency Virus (SIV), feline immunodeficiency virus (FIV), bovine immunodeficiency virus (BIV), Jembrana Disease Virus (JDV), equine infectious anemia virus (EIAV), equine infectious anemia, virus, visna-maedi and caprine arthritis encephalitis virus (CAEV).

Typically, lentiviral particles making up the gene delivery vehicle are replication defective on their own (also referred to as “self-inactivating”). Lentiviruses are able to infect both dividing and non-dividing cells by virtue of the entry mechanism through the intact host nuclear envelope (Naldini L et al., Curr. Opin. Bioiecknol, 1998, 9: 457-463). Recombinant lentiviral vehicles/particles have been generated by multiply attenuating the HIV virulence genes, for example, the genes Env, Vif, Vpr, Vpu, Nef and Tat are deleted making the vector biologically safe. Correspondingly, lentiviral vehicles, for example, derived from HIV-1/HIV-2 can mediate the efficient delivery, integration and long-term expression of transgenes into non-dividing cells.

Lentiviral particles may be generated by co-expressing the virus packaging elements and the vector genome itself in a producer cell such as human HEK293T cells. These elements are usually provided in three (in second generation lentiviral systems) or four separate plasmids (in third generation lentiviral systems). The producer cells are co-transfected with plasmids that encode lentiviral components including the core (i.e. structural proteins) and enzymatic components of the virus, and the envelope protein(s) (referred to as the packaging systems), and a plasmid that encodes the genome including a foreign transgene, to be transferred to the target cell, the vehicle itself (also referred to as the transfer vector). In general, the plasmids or vectors are included in a producer cell line. The plasmids/vectors are introduced via transfection, transduction or infection into the producer cell line. Methods for transfection, transduction or infection are well known by those of skill in the art. As non-limiting example, the packaging and transfer constructs can be introduced into producer cell lines by calcium phosphate transfection, lipofection or electroporation, generally together with a dominant selectable marker, such as neomycin (neo), dihydrofolate reductase (DHFR), glutamine synthetase or adenosine deaminase (ADA), followed by selection in the presence of the appropriate drug and isolation of clones.

The producer cell produces recombinant viral particles that contain the foreign gene, for example, the payload gene. The recombinant viral particles are recovered from the culture media and titrated by standard methods used by those of skill in the art. The recombinant lentiviral vehicles can be used to infect target cells.

Cells that can be used to produce high-titer lentiviral particles may include, but are not limited to, HEK293T cells, 293G cells, STAR cells (Relander et al., Mol Ther. 2005, 11: 452-459), FreeStyle™ 293 Expression System (ThermoFisher, Waltham, MA), and other HEK293T-based producer cell lines (e.g., Stewart et al., Hum Gene Ther. 2011, 2, 2.(3):357˜369; Lee et al, Biotechnol Bioeng, 2012, 10996): 1551-1560; Throm et al., Blood. 2009, 113(21): 5104-5110).

In some aspects, the envelope proteins may be heterologous envelope protein from other viruses, such as the G protein of vesicular stomatitis virus (VSV G) or baculoviral gp64 envelop proteins. The VSV-G glycoprotein may especially be chosen among species classified in the vesiculovirus genus: Carajas virus (CJSV), Chandipura virus (CHPV), Cocal virus (COCV), Isfahan virus (ISFV), Maraba virus (MARAV), Piry virus (PIRYV), Vesicular stomatitis Aiagoas virus (VSAV), Vesicular stomatitis Indiana virus (VSTV) and Vesicular stomatitis New Jersey virus (VSNJV) and/or stains provisionally classified in the vesiculovims genus as Grass carp rhabdovirus, BeAn 157575 virus (BeAn 157575), Boteke virus (BTKV), Calchaqui virus (CQFV), Eel virus American (EVA), Gray Lodge virus (GLOV), Jurona virus (JURY), Klamath virus (KLAVj. Kwatta virus (KWAV), La Joya virus (LJV), Malpais Spring virus (MSPV), Mount Elgon bat virus (MEB V), Ferine t virus (PERV), Pike fry rhabdovirus (PFRV), Porton virus (PORV), Radi virus (RADIV), Spring viremia of carp virus (SVCV), Tupaia virus (TUPV), Ulcerative disease rhabdovirus (UDRV) and Yug Bogdanovac virus (YBV). The gp64 or other baculoviral env protein can be derived from Autographa californica nucleopolyhedroviras (AcMNPV), Anagrapha falcifera nuclear polyhedrosis virus, Bombyx mori nuclear polyhedrosis virus, Choristoneura fiimiferana nucleopolyhedroviras, Orgyia pseudotsugata single capsid nuclear polyhedrosis virus, Epiphyas postvittana nucleopolyhedroviras, Hypharitria cunea nucleopolyhedroviras, Galleria mellonella nuclear polyhedrosis virus, Dhori virus, Thogoto virus, Antheraea pemyi nucleopolyhedroviras or Batken virus.

In some embodiments, the envelope protein may be a fusogen. Exemplary fusogens include paramyxovirus fusogens such as described below.

Additional elements provided in lentiviral particles may comprise retroviral LTR (long-terminal repeat) at either 5′ or 3′ terminus, a retroviral export element, optionally a lentiviral reverse response element (RRE), a promoter or active portion thereof, and a locus control region (LCR) or active portion thereof. Other elements include central polypurine tract (cPPT) sequence to improve transduction efficiency in non-dividing cells, Woodchuck Hepatitis Virus (WHP) Posttranscriptional Regulatory Element (WPRE) which enhances the expression of the transgene, and increases titer.

Methods for generating recombinant lentiviral particles are known to a skilled artisan, for example, U.S. Pat. Nos. 8,846,385; 7,745,179; 7,629,153; 7,575,924; 7,179,903; and 6,808,905. Lentivirus vectors used may be selected from, but are not limited to pLVX, pLenti, pLenti6, pLJMI, FUGW, pWPXL, pWPI, pLenti CMV puro DEST, pLJMI-EGFP, pULTRA, pInducer2Q, pHIV-EGFP, pCW57.1, pTRPE, pELPS, pRRL, and pLionII, Any known lentiviral vehicles may also be used (See, U.S. Pat. Nos. 9,260,725; 9,068,199; 9,023,646; 8,900,858; 8,748,169; 8,709,799; 8,420,104; 8,329,462; 8,076,106; 6,013,516; and 5,994,136; International Patent Publication NO.: WO2012079000).

Other retroviral vectors also may be used to package a payload agent for delivery to a target cell. Retroviral vectors (RVs) allow the permanent integration of a transgene in target cells. In addition to lentiviral vectors based on complex HIV-1/2, retroviral vectors based on simple gamma-retroviruses have been widely used to deliver therapeutic genes and demonstrated clinically as one of the most efficient and powerful gene delivery systems capable of transducing a broad range of cell types. Example species of Gamma retroviruses include the murine leukemia viruses (MLVs) and the feline leukemia viruses (FeLV).

In some embodiments, gamma-retro viral vectors derived from a mammalian gamma-retrovirus such as murine leukemia viruses (MLVs), are recombinant. The MLV families of gamma retroviruses include the ecotropic, amphotropic, xenotropic and polytropic subfamilies. Ecotropic viruses are able to infect only murine cells using mCAT-1 receptor. Examples of ecotropic viruses are Moloney MLV and AKV. Amphotropic viruses infect murine, human and other species through the Pit-2 receptor. One example of an amphotropic virus is the 4070A virus. Xenotropic and polytropic viruses utilize the same (XprI) receptor, but differ in their species tropism. Xenotropic viruses such as NZB-9-1 infect human and other species but not murine species, whereas polytropic viruses such as focus-forming viruses (MCF) infect murine, human and other species.

Gamma-retroviral vectors may be produced in packaging cells by co-transfecting the cells with several plasmids including one encoding the retroviral structural and enzymatic (gag-pol) polyprotein, one encoding the envelope (env) protein, and one encoding the vector mRNA comprising polynucleotide encoding the payload agent that is to be packaged in newly formed viral particles.

In some aspects, the recombinant gamma-retroviral vectors are pseudotyped with envelope proteins from other viruses. Envelope glycoproteins are incorporated in the outer lipid layer of the viral particles which can increase/alter the cell tropism. Exemplary envelope proteins include the gibbon ape leukemia vims envelope protein (GALV) or vesicular stomatitis virus G protein (VSV-G), or Simian endogenous retrovirus envelope protein, or Measles Virus H and F proteins, or Human immunodeficiency virus gp120 envelope protein, or cocal vesiculovirus envelope protein (See, e.g., U.S. application publication NO.: 2012/164118). In other aspects, envelope glycoproteins may be genetically modified to incorporate targeting/binding ligands into gamma-retroviral vectors, binding ligands including, but not limited to, peptide ligands, single chain antibodies and growth factors (Waehier et aL, Nat. Rev. Genet. 2007, 8(8):573-587). These engineered glycoproteins can retarget vectors to cells expressing their corresponding target moieties. In other aspects, a “molecular bridge” may be introduced to direct vectors to specific cells. The molecular bridge has dual specificities: one end can recognize viral glycoproteins, and the other end can bind to the molecular determinant on the target cell. Such molecular bridges, for example ligand-receptor, avidin-biotin, and chemical conjugations, monoclonal antibodies and engineered fusogenic proteins, can direct the attachment of viral vectors to target cells for transduction (Yang et al, Biotechnol Bioeng., 2008, 101(2): 357-368; and Maetzig et al, Viruses, 2011, 3, 677-713).

Exemplary envelope proteins including fusogens retargeted with a target moiety for binding to a target cell are described below.

In some embodiments, the recombinant gamma-retroviral vectors are self-inactivating (SIN) gammaretroviral vectors. The vectors may be replication incompetent. SIN vectors may harbor a deletion within the 3′ U3 region initially comprising enhancer/promoter activity. Furthermore, the 5′ U3 region may be replaced with strong promoters (needed in the packaging cell line) derived from Cytomegalovirus or RSV, or an internal promoter of choice, and/or an enhancer element. The choice of the internal promoters may be made according to specific requirements of gene expression needed for a particular purpose.

In some embodiments, polynucleotides encoding the payload agent are inserted within the recombinant viral genome. The other components of the viral mRNA of a recombinant gamma-retroviral vector may be modified by insertion or removal of naturally occurring sequences (e.g., insertion of an IRES, insertion of a heterologous polynucleotide encoding a polypeptide, shuffling of a more effective promoter from a different retrovirus or virus in place of the wild-type promoter and the like). In some examples, the recombinant gamma-retroviral vectors may comprise modified packaging signal, and/or primer binding site (PBS), and/or 5′-enhancer/promoter elements in the U3-region of the 5′-long terminal repeat (LTR), and/or 3′-SIN elements modified in the US-region of the 3-LTR. These modifications may increase the titers and the ability of infection. Gamma retroviral vectors suitable for delivering the heterologous agent(s) (e.g. CAR and/or immunomodulator, such as a cytokine) may be selected from those disclosed in U.S. Pat. Nos. 8,828,718; 7,585,676; 7,351,585; U.S. application publication NO.: US2007/048285; PCT application publication NOs.: WO2010/113037; WO2014/121005; WO2015/056014; and EP Pat, NOs.: EP1757702; EP1757703).

### B. Virus-Like Particles

In some embodiments, the lipid particle is a virus-like particle. The VLPs include those derived from retroviruses or lentiviruses. While VLPs mimic native virion structure, they lack the viral genomic information necessary for independent replication within a host cell. Therefore, in some aspects, VLPs are non-infectious. In some embodiments, the VLP's bilayer of amphipathic lipids is or comprises the viral envelope. In some embodiments, the lipid particle's bilayer of amphipathic lipids is or comprises lipids derived from a cell. A VLP typically comprises at least one type of structural protein from a virus. In most cases this protein will form a proteinaceous capsid (e.g. VLPs comprising a lentivirus, adenovirus or paramyxovirus structural protein). In some cases the capsid will also be enveloped in a lipid bilayer originating from the cell from which the assembled VLP has been released (e.g. VLPs comprising a human immunodeficiency virus structural protein such as GAG). In some embodiments, the VLP is pseudotyped and/or further comprises a targeting moiety as an envelope protein within the lipid bilayer.

In some embodiments, the VLP comprises supramolecular complexes formed by viral proteins that self-assemble into capsids. In some embodiments, the VLP is derived from viral capsids. In some embodiments, the VLP is derived from viral nucleocapsids. In some embodiments, the VLP is nucleocapsid-derived and retains the property of packaging nucleic acids. In some embodiments, the VLP includes only viral structural glycoproteins. In some embodiments, the VLP does not contain a viral genome.

Among VLPs are that are derived from virus, such as those derived from retroviruses or lentiviruses. In some embodiments, the viral particles are derived from paramyxoviruses. Thus, in some examples, the viral-like particle is derived from Nipah, Hendra, or Rubeola viruses.

Exemplary methods of producing paramyxovirus-based VLPs are disclosed in US2017/0175086.

### C. Non-Viral Vectors

In some embodiments, the payload gene is not comprised in a viral or virally derived vector. In some embodiments, synthetic or natural biodegradable agents may be used for delivery of a payload agent such as cationic lipids, lipid nano emulsions, nanoparticles, peptide based vectors, or polymer based vectors.

In some embodiments, the lipid particle is a non-viral vector. In some embodiments, the lipid particle comprises a naturally derived bilayer of amphipathic lipids. In some embodiments, the bilayer may be comprised of one or more lipids of the same or different type. In some embodiments, the lipids comprise phospholipids such as phosphocholines and phosphoinositols. In some embodiments, the lipids comprise DMPC, DOPC, and DSPC.

In some embodiments, the lipid particles contain a cationic lipid. Cationic lipids are amphiphilic molecules that have a cationic head group and a hydrophobic tail group connected by either stable or degradable linkages. Felgner and colleagues were the first to demonstrate the use of cationic lipids for DNA delivery in 1987 (Felgner et al. PNAS (84) 21:7413-7417, 1987). Many cationic lipids since then have been synthesized and evaluated for nucleic acid delivery, including for example GL67A.

In some embodiments, the payload agent is incorporated in lipid nanoparticles. In some embodiments, the lipid particle is a lipid nanoparticle. In some embodiments, the formulation is a nanoparticle which may comprise at least one lipid. The lipid may be selected from, but is not limited to, DLin-DMA, DLin-K-DMA, 98N12-5, C12-200, DLin-MC3-DMA, DLin-KC2-DMA, DODMA, PLGA, PEG, PEG-DMG and PEGylated lipids. In another aspect, the lipid may be a cationic lipid such as, but not limited to, DLin-DMA, DLin-D-DMA, DLin-MC 3-DMA, DLin-KC2-DMA and DODMA

LNPs particularly useful for in the present methods comprise a cationic lipid selected from DLin-DMA (1,2-dilinoleyloxy-3-dimethylaminopropane), DLin-MC3-DM A (dilinoleylmethyl-4-dimethylaminobutyrate), DLin-KC2-DMA (2,2-dilinoleyl-4-(2-dimethylaminoethyl)-[1,3]-dioxolane), DODMA (1,2-dioleyloxy-N,N-dimethyl-3-aminopropane), SS-OP (Bis[2-(4-{2-[4-(cis-9 octadecenoyloxy)phenylacetoxy]ethyl}piperidinyl)ethyl]disulfide), and derivatives thereof. DLin-MC3-DMA and derivatives thereof are described, for example, in WO 2010144740. DODMA and derivatives thereof are described, for example, in U.S. Pat. No. 7,745,651 and Mok et al. (1999), Biochimica et Biophysica Acta, 1419(2): 137-150. DLin-DMA and derivatives thereof are described, for example, in U.S. Pat. No. 7,799,565. DLin-KC2-DMA and derivatives thereof are described, for example, in U.S. Pat. No. 9,139,554. SS-OP (NOF America Corporation, White Plains, NY) is described, for example, at www.nofamerica.com/store/index.php?dispatch=products. view &product_id=962. Additional and non-limiting examples of cationic lipids include methylpyridiyl-dialkyl acid (MPDACA), palmitoyl-oleoyl-nor-arginine (PONA), guanidino-dialkyl acid (GUADACA), 1,2-di-O-octadecenyl-3-trimethylammonium propane (DOTMA), 1,2-dioleoyl-3-trimethylammonium-propane (DOTAP), Bis{2-[N-methyl-N-(a-D-tocopherolhemisuccinatepropyl)amino]ethyl}disulfide (SS-33/3AP05), Bis{2-[4-(a-D-tocopherolhemisuccinateethyl)piperidyl]ethyl}disulfide (SS33/4PE15), Bis{2-[4-(cis-9-octadecenoateethyl)-1-piperidinyl]ethyl}disulfide (SS18/4PE16), and Bis{2-[4-(cis,cis-9,12-octadecadienoateethyl)-1-piperidinyl]ethyl}disulfide (SS18/4PE13). In further embodiments, the lipid nanoparticles also comprise one or more non-cationic lipids and a lipid conjugate.

In some embodiments, the molar concentration of the cationic lipid is from about 20% to about 80%, from about 30% to about 70%, from about 40% to about 60%, from about 45% to about 55%, or about 20%, about 25%, about 30%, about 35%, about 40%, about 45%, about 50%, about 55%, about 60%, about 65%, about 70%, about 75%, or about 80% of the total lipid molar concentration, wherein the total lipid molar concentration is the sum of the cationic lipid, the non-cationic lipid, and the lipid conjugate molar concentrations. In certain embodiments, the lipid nanoparticles comprise a molar ratio of cationic lipid to mRNA of from about 1 to about 20, from about 2 to about 16, from about 4 to about 12, from about 6 to about 10, or about 1, about 2, about 3, about 4, about 5, about 6, about 7, about 8, about 9, about 10, about 11, about 12, about 13, about 14, about 15, about 16, about 17, about 18, about 19, or about 20.

In some embodiments, the lipid nanoparticles utilized in the presently disclosed methods can comprise at least one non-cationic lipid. In particular embodiments, the molar concentration of the non-cationic lipids is from about 20% to about 80%, from about 30% to about 70%, from about 40% to about 70%, from about 40% to about 60%, from about 46% to about 50%, or about 20%, about 25%, about 30%, about 35%, about 40%, about 45%, about 48.5%, about 50%, about 55%, about 60%, about 65%, about 70%, about 75%, or about 80% of the total lipid molar concentration. Non-cationic lipids include, in some embodiments, phospholipids and steroids.

In some embodiments, phospholipids useful for the lipid nanoparticles described herein include, but are not limited to, 1,2-Distearoyl-sn-glycero-3-phosphocholine (DSPC), 1,2-Didecanoyl-sn-glycero-3-phosphocholine (DDPC), 1,2-Dierucoyl-sn-glycero-3-phosphate(Sodium Salt) (DEPA-NA), 1,2-Dierucoyl-sn-glycero-3-phosphocholine (DEPC), 1,2-Dierucoyl-sn-glycero-3-phosphoethanolamine (DEPE), 1,2-Dierucoyl-sn-glycero-3[Phospho-rac-(1-glycerol)(Sodium Salt) (DEPG-NA), 1,2-Dilinoleoyl-sn-glycero-3-phosphocholine (DLOPC), 1,2-Dilauroyl-sn-glycero-3-phosphate(Sodium Salt) (DLPA-NA), 1,2-Dilauroyl-sn-glycero-3-phosphocholine (DLPC), 1,2-Dilauroyl-sn-glycero-3-phosphoethanolamine (DLPE), 1,2-Dilauroyl-sn-glycero-3[Phospho-rac-(1-glycerol . . . )(Sodium Salt) (DLPG-NA), 1,2-Dilauroyl-sn-glycero-3[Phospho-rac-(1-glycerol)(Ammonium Salt) (DLPG-NH4), 1,2-Dilauroyl-sn-glycero-3-phosphoserine(Sodium Salt) (DLPS-NA), 1,2-Dimyristoyl-sn-glycero-3-phosphate(SodiumSalt) (DMPA-NA), 1,2-Dimyristoyl-sn-glycero-3-phosphocholine (DMPC), 1,2-Dimyristoyl-sn-glycero-3-phosphoethanolamine (DMPE), 1,2-Dimyristoyl-sn-glycero-3[Phospho-rac-(1-glycerol)(Sodium Salt) (DMPG-NA), 1,2-Dimyristoyl-sn-glycero-3[Phospho-rac-(1-glycerol)(Ammonium Salt) (DMPG-NH4), 1,2-Dimyristoyl-sn-glycero-3[Phospho-rac-(1-glycerol)(Sodium/Ammonium Salt) (DMPG-NH4/NA), 1,2-Dimyristoyl-sn-glycero-3-phosphoserine(Sodium Salt) (DMPS-NA), 1,2-Dioleoyl-sn-glycero-3-phosphate(Sodium Salt) (DOPA-NA), 1,2-Dioleoyl-sn-glycero-3-phosphocholine (DOPC), 1,2-Dioleoyl-sn-glycero-3-phosphoethanolamine (DOPE), 1,2-Dioleoyl-sn-glycero-3[Phospho-rac-(1-glycerol)(Sodium Salt) (DOPG-NA), 1,2-Dioleoyl-sn-glycero-3-phosphoserine(Sodium Salt) (DOPS-NA), 1,2-Dipalmitoyl-sn-glycero-3-phosphate(Sodium Salt) (DPPA-NA), 1,2-Dipalmitoyl-sn-glycero-3-phosphocholine (DPPC), 1,2-Dipalmitoyl-sn-glycero-3-phosphoethanolamine (DPPE), 1,2-Dipalmitoyl-sn-glycero-3[Phospho-rac-(1-glycerol)(Sodium Salt) (DPPG-NA), 1,2-Dipalmitoyl-sn-glycero-3[Phospho-rac-(1-glycerol)(Ammonium Salt) (DPPG-NH4), 1,2-Dipalmitoyl-sn-glycero-3-phosphoserine(Sodium Salt) (DPPS-NA), 1,2-Distearoyl-sn-glycero-3-phosphate(Sodium Salt) (DSPA-NA), 1,2-Distearoyl-sn-glycero-3-phosphoethanolamine (DSPE), 1,2-Distearoyl-sn-glycero-3[Phospho-rac-(1-glycerol)(Sodium Salt) (DSPG-NA), 1,2-Distearoyl-sn-glycero-3[Phospho-rac-(1-glycerol)(Ammonium Salt) (DSPG-NH4), 1,2-Distearoyl-sn-glycero-3-phosphoserine(Sodium Salt) (DSPS-NA), Egg-PC (EPC), Hydrogenated Egg PC (HEPC), Hydrogenated Soy PC (HSPC), 1-Myristoyl-sn-glycero-3-phosphocholine (LY S OPCM YRIS TIC), 1-Palmitoyl-sn-glycero-3-phosphocholine (LYSOPCPALMITIC), 1-Stearoyl-sn-glycero-3-phosphocholine (LYSOPC STEARIC), 1-Myristoyl-2-palmitoyl-sn-glycero3-phosphocholine (MPPC), 1-Myristoyl-2-stearoyl-sn-glycero-3-phosphocholine (MSPC), 1-Palmitoyl-2-myristoyl-sn-glycero-3-phosphocholine (PMPC), 1-Palmitoyl-2-oleoyl-sn-glycero-3-phosphocholine (POPC), 1-Palmitoyl-2-oleoyl-sn-glycero-3-phosphoethanolamine (POPE), 1-Palmitoyl-2-oleoyl-sn-glycero-3[Phospho-rac-(1-glycerol)](Sodium Salt) (POPG-NA), 1-Palmitoyl-2-stearoyl-sn-glycero-3-phosphocholine (PS PC), 1-Stearoyl-2-myristoyl-sn-glycero-3-phosphocholine (SMPC), 1-Stearoyl-2-oleoyl-sn-glycero-3-phosphocholine (SOPC), and 1-Stearoyl-2-palmitoyl-sn-glycero-3-phosphocholine (SPPC). In particular embodiments, the phospholipid is DSPC. In particular embodiments, the phospholipid is DOPE. In particular embodiments, the phospholipid is DOPC.

In some embodiments, the non-cationic lipids comprised by the lipid nanoparticles include one or more steroids. Steroids useful for the lipid nanoparticles described herein include, but are not limited to, cholestanes such as cholesterol, cholanes such as cholic acid, pregnanes such as progesterone, androstanes such as testosterone, and estranes such as estradiol. Further steroids include, but are not limited to, cholesterol (ovine), cholesterol sulfate, desmosterol-d6, cholesterol-d7, lathosterol-d7, desmosterol, stigmasterol, lanosterol, dehydrocholesterol, dihydrolanosterol, zymosterol, lathosterol, zymosterol-d5, 14-demethyl-lanosterol, 14-demethyl-lanosterol-d6, 8(9)-dehydrocholesterol, 8(14)-dehydrocholesterol, diosgenin, DHEA sulfate, DHEA, lanosterol-d6, dihydrolanosterol-d7, campesterol-d6, sitosterol, lanosterol-95, Dihydro FF-MAS-d6, zymostenol-d7, zymostenol, sitostanol, campestanol, campesterol, 7-dehydrodesmosterol, pregnenolone, sitosterol-d7, Dihydro T-MAS, Delta 5-avenasterol, Brassicasterol, Dihydro FF-MAS, 24-methylene cholesterol, cholic acid derivatives, cholesteryl esters, and glycosylated sterols. In particular embodiments, the lipid nanoparticles comprise cholesterol.

In some embodiments, the lipid nanoparticles comprise a lipid conjugate. Such lipid conjugates include, but are not limited to, ceramide PEG derivatives such as C8 PEG2000 ceramide, C16 PEG2000 ceramide, C8 PEG5000 ceramide, C16 PEG5000 ceramide, C8 PEG750 ceramide, and C16 PEG750 ceramide, phosphoethanolamine PEG derivatives such as 16:0 PEG5000PE, 14:0 PEG5000 PE, 18:0 PEG5000 PE, 18:1 PEG5000 PE, 16:0 PEG3000 PE, 14:0 PEG3000 PE, 18:0 PEG3000 PE, 18:1 PEG3000 PE, 16:0 PEG2000 PE, 14:0 PEG2000 PE, 18:0 PEG2000 PE, 18:1 PEG2000 PE 16:0 PEG1000 PE, 14:0 PEG1000 PE, 18:0 PEG1000 PE, 18:1 PEG1000 PE, 16:0 PEG750 PE, 14:0 PEG750 PE, 18:0 PEG750 PE, 18:1 PEG750 PE, 16:0 PEG550 PE, 14:0 PEG550 PE, 18:0 PEG550 PE, 18:1 PEG550 PE, 16:0 PEG350 PE, 14:0 PEG350 PE, 18:0 PEG350 PE, and 18:1 PEG350, sterol PEG derivatives such as Chol-PEG600, and glycerol PEG derivatives such as DMG-PEG5000, DSG-PEG5000, DPG-PEG5000, DMG-PEG3000, DSG-PEG3000, DPG-PEG3000, DMG-PEG2000, DSG-PEG2000, DPG-PEG2000, DMG-PEG1000, DSG-PEG1000, DPG-PEG1000, DMG-PEG750, DSG-PEG750, DPG-PEG750, DMG-PEG550, DSG-PEG550, DPG-PEG550, DMG-PEG350, DSG-PEG350, and DPG-PEG350. In some embodiments, the lipid conjugate is a DMG-PEG. In some particular embodiments, the lipid conjugate is DMG-PEG2000. In some particular embodiments, the lipid conjugate is DMG-PEG5000.

It is within the level of a skilled artisan to select the cationic lipids, non-cationic lipids and/or lipid conjugates which comprise the lipid nanoparticle, as well as the relative molar ratio of such lipids to each other, such as based upon the characteristics of the selected lipid(s), the nature of the delivery to the intended target cells, and the characteristics of the mRNA to be delivered. Additional considerations include, for example, the saturation of the alkyl chain, as well as the size, charge, pH, pKa, fusogenicity and toxicity of the selected lipid(s). Thus, the molar ratios of each individual component may be adjusted accordingly.

The lipid nanoparticles for use in the method can be prepared by various techniques which are known to a skilled artisan. Nucleic acid-lipid particles and their method of preparation are disclosed in, for example, U.S. Patent Publication Nos. 20040142025 and 20070042031.

In some embodiments, the lipid nanoparticles will have a size within the range of about 25 to about 500 nm. In some embodiments, the lipid nanoparticles have a size from about 50 nm to about 300 nm, or from about 60 nm to about 120 nm. The size of the lipid nanoparticles may be determined by quasi-electric light scattering (QELS) as described in Bloomfield, Ann. Rev. Biophys. Bioeng., 10:421A150 (1981). A variety of methods are known in the art for producing a population of lipid nanoparticles of particular size ranges, for example, sonication or homogenization. One such method is described in U.S. Pat. No. 4,737,323.

In some embodiments, the lipid nanoparticles comprise an immune cell targeting molecule such as, for example, a targeting ligand (e.g., antibodies, scFv proteins, DART molecules, peptides, aptamers, and the like) anchored on the surface of the lipid nanoparticle that selectively binds the lipid nanoparticles to target cells.

### D. Methods of Generating Viral-Based Particles

The provided viral-based particles include particles derived from a virus, such as viral particles or virus-like particles, including those derived from retroviruses or lentiviruses.

In some embodiments, the viral particle or virus-like particle is produced from virus family members comprising Parvoviridae (e.g. adeno-associated virus), Retroviridae (e.g. HIV), Flaviviridae (e.g. Hepatitis C virus), Paramyxoviridae (e.g. Nipah) and bacteriophages.

In some embodiments, the viral particle or virus-like particle is produced utilizing proteins (e.g., envelope proteins) from a virus within the Paramyxoviridae family. In some embodiments, the Paramyxoviridae family comprises members within the Henipavirus genus. In some embodiments, the Henipavirus is or comprises a Hendra (HeV) or a Nipah (NiV) virus. In particular embodiments, the viral particles or virus-like particles incorporate a fusogen, such as a targeted envelope protein and fusogen as described in Section IV.

In some embodiments, viral particles or virus-like particles may be produced in multiple cell culture systems including bacteria, mammalian cell lines, insect cell lines, yeast and plant cells.

In some embodiments, the assembly of a viral particle or virus-like particle is initiated by binding of the core protein to a unique encapsidation sequence within the viral genome (e.g. UTR with stem-loop structure). In some embodiments, the interaction of the core with the encapsidation sequence facilitates oligomerization.

Any of a variety of known methods can be used to produce retroviral particles whose genome contains an RNA copy of the viral vector genome. In some embodiments, at least two components are involved in making a virus-based gene delivery system: first, packaging plasmids, encompassing the structural proteins as well as the enzymes necessary to generate a viral vector particle, and second, the viral vector itself, i.e., the genetic material to be transferred. Biosafety safeguards can be introduced in the design of one or both of these components.

In some embodiments, the packaging plasmid can contain all retroviral, such as HIV-1, proteins other than envelope proteins (Naldini et al., 1998). In other embodiments, viral vectors can lack additional viral genes, such as those that are associated with virulence, e.g. vpr, vif, vpu and nef, and/or Tat, a primary transactivator of HIV. In some embodiments, lentiviral vectors, such as HIV-based lentiviral vectors, comprise only three genes of the parental virus: gag, pol and rev, which reduces or eliminates the possibility of reconstitution of a wild-type virus through recombination.

In some embodiments, a viral vectors and transfer plasmids comprise structural and/or functional genetic elements that are primarily derived from a virus. A retroviral vector can comprise a viral vector or plasmid containing structural and functional genetic elements, or portions thereof, that are primarily derived from a retrovirus. A lentiviral vector can comprise a viral vector or plasmid containing structural and functional genetic elements, or portions thereof, including LTRs that are primarily derived from a lentivirus.

In embodiments, a lentiviral vector (e.g., lentiviral expression vector) may comprise a lentiviral transfer plasmid (e.g., as naked DNA) or an infectious lentiviral particle. With respect to elements such as cloning sites, promoters, regulatory elements, heterologous nucleic acids, etc., it is to be understood that the sequences of these elements can be present in RNA form in lentiviral particles and can be present in DNA form in DNA plasmids.

In some embodiments, the virus particle of viral-like particle, such as a retrovirus or retroviral-like particle (e.g. a lentivirus or lentiviral-like particle) is pseudotyped. In some examples, a pseudotyped virus of viral-like particle has a modification to one or more of its envelope proteins, e.g., an envelope protein is substituted with an envelope protein from another virus. For example, HIV can be pseudotyped with vesicular stomatitis virus G-protein (VSV-G) envelope proteins, which allows HIV to infect a wider range of cells because HIV envelope proteins (encoded by the env gene) normally target the virus to CD4+ presenting cells.

In some embodiments, retroviral envelope proteins, e.g. lentiviral envelope proteins, are pseudotyped with VSV-G. In one embodiment, source cells produce recombinant retrovirus or retrovirus-like particles, e.g., lentivirus or lentiviral-like particles, pseudotyped with the VSV-G envelope glycoprotein.

In one embodiment, source cells produce recombinant retrovirus or retrovirus-like particles, e.g., lentivirus or lentiviral-like particles, pseudotyped with the VSV-G envelope glycoprotein. In some embodiments, retroviral envelope proteins, e.g. lentiviral envelope proteins, are pseudotyped with an envelope glycoprotein G or H of a virus of the Paramyxoviridae family. In some embodiments, the virus of the Paramyxovirus family is a Henipavirus or is a Morbillivirus. In some aspects, the envelope glycoprotein a Nipah virus G (Niv-G) protein. In other aspects, the envelope glycoprotein is a Hendra virus G protein. In some embodiment, source cells produce recombinant retrovirus or retrovirus-like particles, e.g., lentivirus or lentiviral-like particles, pseudotyped with the envelope glycoprotein G or H of a virus of the Paramyxoviridae family.

In some embodiments, in the vectors described herein at least part of one or more protein coding regions that contribute to or are essential for replication may be absent compared to the corresponding wild-type virus. In some embodiments, the viral vector replication-defective. In some embodiments, the vector is capable of transducing a target non-dividing host cell and/or integrating its genome into a host genome.

In some embodiments, the structure of a wild-type retrovirus genome often comprises a 5′ long terminal repeat (LTR) and a 3′ LTR, between or within which are located a packaging signal to enable the genome to be packaged, a primer binding site, integration sites to enable integration into a host cell genome and gag, pol and env genes encoding the packaging components which promote the assembly of viral particles. More complex retroviruses have additional features, such as rev and RRE sequences in HIV, which enable the efficient export of RNA transcripts of the integrated provirus from the nucleus to the cytoplasm of an infected target cell. In the provirus, the viral genes are flanked at both ends by regions called long terminal repeats (LTRs). In some embodiments, the LTRs are involved in proviral integration and transcription. In some embodiments, LTRs serve as enhancer-promoter sequences and can control the expression of the viral genes. In some embodiments, encapsidation of the retroviral RNAs occurs by virtue of a psi sequence located at the 5′ end of the viral genome.

In some embodiments, LTRs are similar sequences that can be divided into three elements, which are called U3, R and U5. U3 is derived from the sequence unique to the 3′ end of the RNA. R is derived from a sequence repeated at both ends of the RNA and U5 is derived from the sequence unique to the 5′ end of the RNA. The sizes of the three elements can vary considerably among different retroviruses.

In some embodiments, for the viral genome, the site of transcription initiation is typically at the boundary between U3 and R in one LTR and the site of poly (A) addition (termination) is at the boundary between R and U5 in the other LTR. U3 contains most of the transcriptional control elements of the provirus, which include the promoter and multiple enhancer sequences responsive to cellular and in some cases, viral transcriptional activator proteins. In some embodiments, retroviruses comprise any one or more of the following genes that code for proteins that are involved in the regulation of gene expression: tat, rev, tax and rex.

In some embodiments, the structural genes gag, pol and env, gag encodes the internal structural protein of the virus. In some embodiments, Gag protein is proteolytically processed into the mature proteins MA (matrix), CA (capsid) and NC (nucleocapsid). In some embodiments, the pol gene encodes the reverse transcriptase (RT), which contains DNA polymerase, associated RNase H and integrase (IN), which mediate replication of the genome. In some embodiments, the env gene encodes the surface (SU) glycoprotein and the transmembrane (TM) protein of the virion, which form a complex that interacts specifically with cellular receptor proteins. In some embodiments, the interaction promotes infection by fusion of the viral membrane with the cell membrane.

In some embodiments, a replication-defective retroviral vector genome gag, pol and env may be absent or not functional. In some embodiments, the R regions at both ends of the RNA are typically repeated sequences. In some embodiments, U5 and U3 represent unique sequences at the 5′ and 3′ ends of the RNA genome respectively.

In some embodiments, retroviruses may also contain additional genes which code for proteins other than gag, pol and env. Examples of additional genes include (in HIV), one or more of vif, vpr, vpx, vpu, tat, rev and nef. EIAV has (amongst others) the additional gene S2. In some embodiments, proteins encoded by additional genes serve various functions, some of which may be duplicative of a function provided by a cellular protein. In EIAV, for example, tat acts as a transcriptional activator of the viral LTR (Derse and Newbold 1993 Virology 194:530-6; Maury et al. 1994 Virology 200:632-42). It binds to a stable, stem-loop RNA secondary structure referred to as TAR. Rev regulates and co-ordinates the expression of viral genes through rev-response elements (RRE) (Martarano et al. 1994 J. Virol. 68:3102-11).

In some embodiments, in addition to protease, reverse transcriptase and integrase, non-primate lentiviruses contain a fourth pol gene product which codes for a dUTPase. In some embodiments, this a role in the ability of these lentiviruses to infect certain non-dividing or slowly dividing cell types.

In embodiments, a recombinant lentiviral vector (RLV) is a vector with sufficient retroviral genetic information to allow packaging of an RNA genome, in the presence of packaging components, into a viral particle capable of infecting a target cell. In some embodiments, infection of the target cell can comprise reverse transcription and integration into the target cell genome. In some embodiments, the RLV typically carries non-viral coding sequences which are to be delivered by the vector to the target cell. In some embodiments, an RLV is incapable of independent replication to produce infectious retroviral particles within the target cell. In some embodiments, the RLV lacks a functional gag-pol and/or env gene and/or other genes involved in replication. In some embodiments, the vector may be configured as a split-intron vector, e.g., as described in PCT patent application WO 99/15683, which is herein incorporated by reference in its entirety.

In some embodiments, the lentiviral vector comprises a minimal viral genome, e.g., the viral vector has been manipulated so as to remove the non-essential elements and to retain the essential elements in order to provide the required functionality to infect, transduce and deliver a nucleotide sequence of interest to a target host cell, e.g., as described in WO 98/17815, which is herein incorporated by reference in its entirety.

In some embodiments, a minimal lentiviral genome may comprise, e.g., (5′)R-U5-one or more first nucleotide sequences-U3-R(3′). In some embodiments, the plasmid vector used to produce the lentiviral genome within a source cell can also include transcriptional regulatory control sequences operably linked to the lentiviral genome to direct transcription of the genome in a source cell. In some embodiments, the regulatory sequences may comprise the natural sequences associated with the transcribed retroviral sequence, e.g., the 5′ U3 region, or they may comprise a heterologous promoter such as another viral promoter, for example the CMV promoter. In some embodiments, lentiviral genomes comprise additional sequences to promote efficient virus production. In some embodiments, in the case of HIV, rev and RRE sequences may be included. In some embodiments, alternatively or combination, codon optimization may be used, e.g., the gene encoding the exogenous agent may be codon optimized, e.g., as described in WO 01/79518, which is herein incorporated by reference in its entirety. In some embodiments, alternative sequences which perform a similar or the same function as the rev/RRE system may also be used. In some embodiments, a functional analogue of the rev/RRE system is found in the Mason Pfizer monkey virus. In some embodiments, this is known as CTE and comprises an RRE-type sequence in the genome which is believed to interact with a factor in the infected cell. The cellular factor can be thought of as a rev analogue. In some embodiments, CTE may be used as an alternative to the rev/RRE system. In some embodiments, the Rex protein of HTLV-I can functionally replace the Rev protein of HIV-I. Rev and Rex have similar effects to IRE-BP.

In some embodiments, a retroviral nucleic acid (e.g., a lentiviral nucleic acid, e.g., a primate or non-primate lentiviral nucleic acid) (1) comprises a deleted gag gene wherein the deletion in gag removes one or more nucleotides downstream of about nucleotide 350 or 354 of the gag coding sequence; (2) has one or more accessory genes absent from the retroviral nucleic acid; (3) lacks the tat gene but includes the leader sequence between the end of the 5′ LTR and the ATG of gag; and (4) combinations of (1), (2) and (3). In an embodiment the lentiviral vector comprises all of features (1) and (2) and (3). This strategy is described in more detail in WO 99/32646, which is herein incorporated by reference in its entirety.

In some embodiments, a primate lentivirus minimal system requires none of the HIV/SIV additional genes vif, vpr, vpx, vpu, tat, rev and nef for either vector production or for transduction of dividing and non-dividing cells. In some embodiments, an EIAV minimal vector system does not require S2 for either vector production or for transduction of dividing and non-dividing cells.

In some embodiments, the deletion of additional genes may permit vectors to be produced without the genes associated with disease in lentiviral (e.g. HIV) infections. In some embodiments, tat is associated with disease. In some embodiments, the deletion of additional genes permits the vector to package more heterologous DNA. In some embodiments, genes whose function is unknown, such as S2, may be omitted, thus reducing the risk of causing undesired effects. Examples of minimal lentiviral vectors are disclosed in WO 99/32646 and in WO 98/17815.

In some embodiments, the retroviral nucleic acid is devoid of at least tat and S2 (if it is an EIAV vector system), and possibly also vif, vpr, vpx, vpu and nef. In some embodiments, the retroviral nucleic acid is also devoid of rev, RRE, or both.

In some embodiments the retroviral nucleic acid comprises vpx. The Vpx polypeptide binds to and induces the degradation of the SAMHD1 restriction factor, which degrades free dNTPs in the cytoplasm. In some embodiments, the concentration of free dNTPs in the cytoplasm increases as Vpx degrades SAMHD1 and reverse transcription activity is increased, thus facilitating reverse transcription of the retroviral genome and integration into the target cell genome.

In some embodiments, different cells differ in their usage of particular codons. In some embodiments, this codon bias corresponds to a bias in the relative abundance of particular tRNAs in the cell type. In some embodiments, by altering the codons in the sequence so that they are tailored to match with the relative abundance of corresponding tRNAs, it is possible to increase expression. In some embodiments, it is possible to decrease expression by deliberately choosing codons for which the corresponding tRNAs are known to be rare in the particular cell type. In some embodiments, an additional degree of translational control is available. An additional description of codon optimization is found, e.g., in WO 99/41397, which is herein incorporated by reference in its entirety.

In some embodiments viruses, including HIV and other lentiviruses, use a large number of rare codons and by changing these to correspond to commonly used mammalian codons, increased expression of the packaging components in mammalian producer cells can be achieved.

In some embodiments, codon optimization has a number of other advantages. In some embodiments, by virtue of alterations in their sequences, the nucleotide sequences encoding the packaging components may have RNA instability sequences (INS) reduced or eliminated from them. At the same time, the amino acid sequence coding sequence for the packaging components is retained so that the viral components encoded by the sequences remain the same, or at least sufficiently similar that the function of the packaging components is not compromised. In some embodiments, codon optimization also overcomes the Rev/RRE requirement for export, rendering optimized sequences Rev independent. In some embodiments, codon optimization also reduces homologous recombination between different constructs within the vector system (for example between the regions of overlap in the gag-pol and env open reading frames). In some embodiments, codon optimization leads to an increase in viral titer and/or improved safety.

In some embodiments, only codons relating to INS are codon optimized. In other embodiments, the sequences are codon optimized in their entirety, with the exception of the sequence encompassing the frameshift site of gag-pol.

The gag-pol gene comprises two overlapping reading frames encoding the gag-pol proteins. The expression of both proteins depends on a frameshift during translation. This frameshift occurs as a result of ribosome “slippage” during translation. This slippage is thought to be caused at least in part by ribosome-stalling RNA secondary structures. Such secondary structures exist downstream of the frameshift site in the gag-pol gene. For HIV, the region of overlap extends from nucleotide 1222 downstream of the beginning of gag (wherein nucleotide 1 is the A of the gag ATG) to the end of gag (nt 1503). Consequently, a 281 bp fragment spanning the frameshift site and the overlapping region of the two reading frames is preferably not codon optimized. In some embodiments, retaining this fragment will enable more efficient expression of the gag-pol proteins. For EIAV, the beginning of the overlap is at nt 1262 (where nucleotide 1 is the A of the gag ATG). The end of the overlap is at nt 1461. In order to ensure that the frameshift site and the gag-pol overlap are preserved, the wild type sequence may be retained from nt 1156 to 1465.

In some embodiments, derivations from optimal codon usage may be made, for example, in order to accommodate convenient restriction sites, and conservative amino acid changes may be introduced into the gag-pol proteins.

In some embodiments, codon optimization is based on codons with poor codon usage in mammalian systems. The third and sometimes the second and third base may be changed.

In some embodiments, due to the degenerate nature of the genetic code, it will be appreciated that numerous gag-pol sequences can be achieved by a skilled worker. Also, there are many retroviral variants described which can be used as a starting point for generating a codon optimized gag-pol sequence. Lentiviral genomes can be quite variable. For example there are many quasi-species of HIV-I which are still functional. This is also the case for EIAV. These variants may be used to enhance particular parts of the transduction process. Examples of HIV-I variants may be found in the HIV databases maintained by Los Alamos National Laboratory. Details of EIAV clones may be found at the NCBI database maintained by the National Institutes of Health.

In some embodiments, the strategy for codon optimized gag-pol sequences can be used in relation to any retrovirus, e.g., EIAV, FIV, BIV, CAEV, VMR, SIV, HIV-I and HIV-2. In addition this method could be used to increase expression of genes from HTLV-I, HTLV-2, HFV, HSRV and human endogenous retroviruses (HERV), MLV and other retroviruses.

In embodiments, the retroviral vector comprises a packaging signal that comprises from 255 to 360 nucleotides of gag in vectors that still retain env sequences, or about 40 nucleotides of gag in a particular combination of splice donor mutation, gag and env deletions. In some embodiments, the retroviral vector includes a gag sequence which comprises one or more deletions, e.g., the gag sequence comprises about 360 nucleotides derivable from the N-terminus.

In some embodiments, the retroviral vector, helper cell, helper virus, or helper plasmid may comprise retroviral structural and accessory proteins, for example gag, pol, env, tat, rev, vif, vpr, vpu, vpx, or nef proteins or other retroviral proteins. In some embodiments the retroviral proteins are derived from the same retrovirus. In some embodiments the retroviral proteins are derived from more than one retrovirus, e.g. 2, 3, 4, or more retroviruses.

In some embodiments, the gag and pol coding sequences are generally organized as the Gag-Pol Precursor in native lentivirus. The gag sequence codes for a 55-kD Gag precursor protein, also called p55. The p55 is cleaved by the virally encoded protease4 (a product of the pol gene) during the process of maturation into four smaller proteins designated MA (matrix [p17]), CA (capsid [p24]), NC (nucleocapsid [p9]), and p6. The pol precursor protein is cleaved away from Gag by a virally encoded protease, and further digested to separate the protease (p10), RT (p50), RNase H (p15), and integrase (p31) activities.

In some embodiments, the lentiviral vector is integration-deficient. In some embodiments, the pol is integrase deficient, such as by encoding due to mutations in the integrase gene. For example, the pol coding sequence can contain an inactivating mutation in the integrase, such as by mutation of one or more of amino acids involved in catalytic activity, i.e. mutation of one or more of aspartic 64, aspartic acid 116 and/or glutamic acid 152. In some embodiments, the integrase mutation is a D64V mutation. In some embodiments, the mutation in the integrase allows for packaging of viral RNA into a lentivirus. In some embodiments, the mutation in the integrase allows for packaging of viral proteins into a lentivirus. In some embodiments, the mutation in the integrase reduces the possibility of insertional mutagenesis. In some embodiments, the mutation in the integrase decreases the possibility of generating replication-competent recombinants (RCRs) (Wanisch et al. 2009. Mol Ther. 1798):1316-1332).

In some embodiments, native Gag-Pol sequences can be utilized in a helper vector (e.g., helper plasmid or helper virus), or modifications can be made. These modifications include, chimeric Gag-Pol, where the Gag and Pol sequences are obtained from different viruses (e.g., different species, subspecies, strains, clades, etc.), and/or where the sequences have been modified to improve transcription and/or translation, and/or reduce recombination.

In some embodiments, the retroviral nucleic acid includes a polynucleotide encoding a 150-250 (e.g., 168) nucleotide portion of a gag protein that (i) includes a mutated INS1 inhibitory sequence that reduces restriction of nuclear export of RNA relative to wild-type INS1, (ii) contains two nucleotide insertion that results in frame shift and premature termination, and/or (iii) does not include INS2, INS3, and INS4 inhibitory sequences of gag.

In some embodiments, a vector described herein is a hybrid vector that comprises both retroviral (e.g., lentiviral) sequences and non-lentiviral viral sequences. In some embodiments, a hybrid vector comprises retroviral e.g., lentiviral, sequences for reverse transcription, replication, integration and/or packaging.

In some embodiments, most or all of the viral vector backbone sequences are derived from a lentivirus, e.g., HIV-1. However, it is to be understood that many different sources of retroviral and/or lentiviral sequences can be used or combined and numerous substitutions and alterations in certain of the lentiviral sequences may be accommodated without impairing the ability of a transfer vector to perform the functions described herein. A variety of lentiviral vectors are described in Naldini et al., (1996a, 1996b, and 1998); Zufferey et al., (1997); Dull et al., 1998, U.S. Pat. Nos. 6,013,516; and 5,994,136, many of which may be adapted to produce a retroviral nucleic acid.

In some embodiments, at each end of the provirus, long terminal repeats (LTRs) are typically found. An LTR typically comprises a domain located at the ends of retroviral nucleic acid which, in their natural sequence context, are direct repeats and contain U3, R and U5 regions. LTRs generally promote the expression of retroviral genes (e.g., promotion, initiation and polyadenylation of gene transcripts) and viral replication. The LTR can comprise numerous regulatory signals including transcriptional control elements, polyadenylation signals and sequences for replication and integration of the viral genome. The viral LTR is typically divided into three regions called U3, R and U5. The U3 region typically contains the enhancer and promoter elements. The U5 region is typically the sequence between the primer binding site and the R region and can contain the polyadenylation sequence. The R (repeat) region can be flanked by the U3 and U5 regions. The LTR is typically composed of U3, R and U5 regions and can appear at both the 5′ and 3′ ends of the viral genome. In some embodiments, adjacent to the 5′ LTR are sequences for reverse transcription of the genome (the tRNA primer binding site) and for efficient packaging of viral RNA into particles (the Psi site).

In some embodiments, a packaging signal can comprise a sequence located within the retroviral genome which mediate insertion of the viral RNA into the viral capsid or particle, see e.g., Clever et al., 1995. J. of Virology, Vol. 69, No. 4; pp. 2101-2109. Several retroviral vectors use a minimal packaging signal (a psi [Ψ] sequence) for encapsidation of the viral genome.

In various embodiments, retroviral nucleic acids comprise modified 5′ LTR and/or 3′ LTRs. Either or both of the LTR may comprise one or more modifications including, but not limited to, one or more deletions, insertions, or substitutions. Modifications of the 3′ LTR are often made to improve the safety of lentiviral or retroviral systems by rendering viruses replication-defective, e.g., virus that is not capable of complete, effective replication such that infective virions are not produced (e.g., replication-defective lentiviral progeny).

In some embodiments, a vector is a self-inactivating (SIN) vector, e.g., replication-defective vector, e.g., retroviral or lentiviral vector, in which the right (3′) LTR enhancer-promoter region, known as the U3 region, has been modified (e.g., by deletion or substitution) to prevent viral transcription beyond the first round of viral replication. This is because the right (3′) LTR U3 region can be used as a template for the left (5′) LTR U3 region during viral replication and, thus, absence of the U3 enhancer-promoter inhibits viral replication. In embodiments, the 3′ LTR is modified such that the U5 region is removed, altered, or replaced, for example, with an exogenous poly(A) sequence The 3′ LTR, the 5′ LTR, or both 3′ and 5′ LTRs, may be modified LTRs.

In some embodiments, the U3 region of the 5′ LTR is replaced with a heterologous promoter to drive transcription of the viral genome during production of viral particles. Examples of heterologous promoters which can be used include, for example, viral simian virus 40 (SV40) (e.g., early or late), cytomegalovirus (CMV) (e.g., immediate early), Moloney murine leukemia virus (MoMLV), Rous sarcoma virus (RSV), and herpes simplex virus (HSV) (thymidine kinase) promoters. In some embodiments, promoters are able to drive high levels of transcription in a Tat-independent manner. In certain embodiments, the heterologous promoter has additional advantages in controlling the manner in which the viral genome is transcribed. For example, the heterologous promoter can be inducible, such that transcription of all or part of the viral genome will occur only when the induction factors are present. Induction factors include, but are not limited to, one or more chemical compounds or the physiological conditions such as temperature or pH, in which the host cells are cultured.

In some embodiments, viral vectors comprise a TAR (trans-activation response) element, e.g., located in the R region of lentiviral (e.g., HIV) LTRs. This element interacts with the lentiviral trans-activator (tat) genetic element to enhance viral replication. However, this element is not required, e.g., in embodiments wherein the U3 region of the 5′ LTR is replaced by a heterologous promoter.

In some embodiments, the R region, e.g., the region within retroviral LTRs beginning at the start of the capping group (i.e., the start of transcription) and ending immediately prior to the start of the poly A tract can be flanked by the U3 and U5 regions. The R region plays a role during reverse transcription in the transfer of nascent DNA from one end of the genome to the other.

In some embodiments, the retroviral nucleic acid can also comprise a FLAP element, e.g., a nucleic acid whose sequence includes the central polypurine tract and central termination sequences (cPPT and CTS) of a retrovirus, e.g., HIV-1 or HIV-2. Suitable FLAP elements are described in U.S. Pat. No. 6,682,907 and in Zennou, et al., 2000, Cell, 101:173, which are herein incorporated by reference in their entireties. During HIV-1 reverse transcription, central initiation of the plus-strand DNA at the central polypurine tract (cPPT) and central termination at the central termination sequence (CTS) can lead to the formation of a three-stranded DNA structure: the HIV-1 central DNA flap. In some embodiments, the retroviral or lentiviral vector backbones comprise one or more FLAP elements upstream or downstream of the gene encoding the exogenous agent. For example, in some embodiments a transfer plasmid includes a FLAP element, e.g., a FLAP element derived or isolated from HIV-1.

In embodiments, a retroviral or lentiviral nucleic acid comprises one or more export elements, e.g., a cis-acting post-transcriptional regulatory element which regulates the transport of an RNA transcript from the nucleus to the cytoplasm of a cell. Examples of RNA export elements include, but are not limited to, the human immunodeficiency virus (HIV) rev response element (RRE) (see e.g., Cullen et al., 1991. J. Virol. 65: 1053; and Cullen et al., 1991. Cell 58: 423), and the hepatitis B virus post-transcriptional regulatory element (HPRE), which are herein incorporated by reference in their entireties. Generally, the RNA export element is placed within the 3′ UTR of a gene, and can be inserted as one or multiple copies.

In some embodiments, expression of heterologous sequences in viral vectors is increased by incorporating one or more of, e.g., all of, posttranscriptional regulatory elements, polyadenylation sites, and transcription termination signals into the vectors. A variety of posttranscriptional regulatory elements can increase expression of a heterologous nucleic acid at the protein, e.g., woodchuck hepatitis virus posttranscriptional regulatory element (WPRE; Zufferey et al., 1999, J. Virol., 73:2886); the posttranscriptional regulatory element present in hepatitis B virus (HPRE) (Huang et al., Mol. Cell. Biol., 5:3864); and the like (Liu et al., 1995, Genes Dev., 9:1766), each of which is herein incorporated by reference in its entirety. In some embodiments, a retroviral nucleic acid described herein comprises a posttranscriptional regulatory element such as a WPRE or HPRE

In some embodiments, a retroviral nucleic acid described herein lacks or does not comprise a posttranscriptional regulatory element such as a WPRE or HPRE.

In some embodiments, elements directing the termination and polyadenylation of the heterologous nucleic acid transcripts may be included, e.g., to increases expression of the exogenous agent. Transcription termination signals may be found downstream of the polyadenylation signal. In some embodiments, vectors comprise a polyadenylation sequence 3′ of a polynucleotide encoding the exogenous agent. A polyA site may comprise a DNA sequence which directs both the termination and polyadenylation of the nascent RNA transcript by RNA polymerase II. Polyadenylation sequences can promote mRNA stability by addition of a polyA tail to the 3′ end of the coding sequence and thus, contribute to increased translational efficiency. Illustrative examples of polyA signals that can be used in a retroviral nucleic acid, include AATAAA, ATTAAA, AGTAAA, a bovine growth hormone polyA sequence (BGHpA), a rabbit β-globin polyA sequence (rpgpA), or another suitable heterologous or endogenous polyA sequence.

In some embodiments, a retroviral or lentiviral vector further comprises one or more insulator elements, e.g., an insulator element described herein.

In various embodiments, the vectors comprise a promoter operably linked to a polynucleotide encoding an exogenous agent. The vectors may have one or more LTRs, wherein either LTR comprises one or more modifications, such as one or more nucleotide substitutions, additions, or deletions. The vectors may further comprise one of more accessory elements to increase transduction efficiency (e.g., a cPPT/FLAP), viral packaging (e.g., a Psi (Ψ) packaging signal, RRE), and/or other elements that increase exogenous gene expression (e.g., poly (A) sequences), and may optionally comprise a WPRE or HPRE.

In some embodiments, a lentiviral nucleic acid comprises one or more of, e.g., all of, e.g., from 5′ to 3′, a promoter (e.g., CMV), an R sequence (e.g., comprising TAR), a U5 sequence (e.g., for integration), a PBS sequence (e.g., for reverse transcription), a DIS sequence (e.g., for genome dimerization), a psi packaging signal, a partial gag sequence, an RRE sequence (e.g., for nuclear export), a cPPT sequence (e.g., for nuclear import), a promoter to drive expression of the exogenous agent, a gene encoding the exogenous agent, a WPRE sequence (e.g., for efficient transgene expression), a PPT sequence (e.g., for reverse transcription), an R sequence (e.g., for polyadenylation and termination), and a U5 signal (e.g., for integration).

Some lentiviral vectors integrate inside active genes and possess strong splicing and polyadenylation signals that could lead to the formation of aberrant and possibly truncated transcripts.

Mechanisms of proto-oncogene activation may involve the generation of chimeric transcripts originating from the interaction of promoter elements or splice sites contained in the genome of the insertional mutagen with the cellular transcriptional unit targeted by integration (Gabriel et al. 2009. Nat Med 15: 1431-1436; Bokhoven, et al. J Virol 83:283-29). Chimeric fusion transcripts comprising vector sequences and cellular mRNAs can be generated either by read-through transcription starting from vector sequences and proceeding into the flanking cellular genes, or vice versa.

In some embodiments, a lentiviral nucleic acid described herein comprises a lentiviral backbone in which at least two of the splice sites have been eliminated, e.g., to improve the safety profile of the lentiviral vector. Species of such splice sites and methods of identification are described in WO2012156839A2, all of which is included by reference.

Large scale viral particle production is often useful to achieve a desired viral titer. Viral particles can be produced by transfecting a transfer vector into a packaging cell line that comprises viral structural and/or accessory genes, e.g., gag, pol, env, tat, rev, vif, vpr, vpu, vpx, or nef genes or other retroviral genes.

In some embodiments, the packaging vector is an expression vector or viral vector that lacks a packaging signal and comprises a polynucleotide encoding one, two, three, four or more viral structural and/or accessory genes. Typically, the packaging vectors are included in a packaging cell, and are introduced into the cell via transfection, transduction or infection. A retroviral, e.g., lentiviral, transfer vector can be introduced into a packaging cell line, via transfection, transduction or infection, to generate a source cell or cell line. The packaging vectors can be introduced into human cells or cell lines by standard methods including, e.g., calcium phosphate transfection, lipofection or electroporation. In some embodiments, the packaging vectors are introduced into the cells together with a dominant selectable marker, such as neomycin, hygromycin, puromycin, blastocidin, zeocin, thymidine kinase, DHFR, Gln synthetase or ADA, followed by selection in the presence of the appropriate drug and isolation of clones. A selectable marker gene can be linked physically to genes encoding by the packaging vector, e.g., by IRES or self-cleaving viral peptides.

In some embodiments, producer cell lines include cell lines that do not contain a packaging signal, but do stably or transiently express viral structural proteins and replication enzymes (e.g., gag, pol and env) which can package viral particles. Any suitable cell line can be employed, e.g., mammalian cells, e.g., human cells. Suitable cell lines which can be used include, for example, CHO cells, BHK cells, MDCK cells, C3H 10T1/2 cells, FLY cells, Psi-2 cells, BOSC 23 cells, PA317 cells, WEHI cells, COS cells, BSC 1 cells, BSC 40 cells, BMT 10 cells, VERO cells, W138 cells, MRC5 cells, A549 cells, HT1080 cells, 293 cells, 293T cells, B-50 cells, 3T3 cells, NIH3T3 cells, HepG2 cells, Saos-2 cells, Huh7 cells, HeLa cells, W163 cells, 211 cells, and 211A cells. In embodiments, the packaging cells are 293 cells, 293T cells, or A549 cells.

In some embodiments, a source cell line includes a cell line which is capable of producing recombinant retroviral particles, comprising a producer cell line and a transfer vector construct comprising a packaging signal. Methods of preparing viral stock solutions are illustrated by, e.g., Y. Soneoka et al. (1995) Nucl. Acids Res. 23:628-633, and N. R. Landau et al. (1992) J. Virol. 66:5110-5113, which are incorporated herein by reference. Infectious virus particles may be collected from the producer cells, e.g., by cell lysis, or collection of the supernatant of the cell culture. The collected virus particles may be enriched or purified.

In some embodiments, the source cell comprises one or more plasmids coding for viral structural proteins and replication enzymes (e.g., gag, pol and env) which can package viral particles. In some embodiments, the sequences coding for at least two of the gag, pol, and env precursors are on the same plasmid. In some embodiments, the sequences coding for the gag, pol, and env precursors are on different plasmids. In some embodiments, the sequences coding for the gag, pol, and env precursors have the same expression signal, e.g., promoter. In some embodiments, the sequences coding for the gag, pol, and env precursors have a different expression signal, e.g., different promoters. In some embodiments, expression of the gag, pol, and env precursors is inducible. In some embodiments, the plasmids coding for viral structural proteins and replication enzymes are transfected at the same time or at different times. In some embodiments, the plasmids coding for viral structural proteins and replication enzymes are transfected at the same time or at a different time from the packaging vector.

In some embodiments, the source cell line comprises one or more stably integrated viral structural genes. In some embodiments expression of the stably integrated viral structural genes is inducible.

In some embodiments, expression of the viral structural genes is regulated at the transcriptional level. In some embodiments, expression of the viral structural genes is regulated at the translational level. In some embodiments, expression of the viral structural genes is regulated at the post-translational level.

In some embodiments, expression of the viral structural genes is regulated by a tetracycline (Tet)-dependent system, in which a Tet-regulated transcriptional repressor (Tet-R) binds to DNA sequences included in a promoter and represses transcription by steric hindrance (Yao et al, 1998; Jones et al, 2005). Upon addition of doxycycline (dox), Tet-R is released, allowing transcription. Multiple other suitable transcriptional regulatory promoters, transcription factors, and small molecule inducers are suitable to regulate transcription of viral structural genes.

In some embodiments, the third-generation lentivirus components, human immunodeficiency virus type 1 (HIV) Rev, Gag/Pol, and an envelope under the control of Tet-regulated promoters and coupled with antibiotic resistance cassettes are separately integrated into the source cell genome. In some embodiments the source cell only has one copy of each of Rev, Gag/Pol, and an envelope protein integrated into the genome.

In some embodiments, a retroviral nucleic acid described herein is unable to undergo reverse transcription. Such a nucleic acid, in embodiments, is able to transiently express an exogenous agent. The retrovirus or VLP, may comprise a disabled reverse transcriptase protein, or may not comprise a reverse transcriptase protein. In embodiments, the retroviral nucleic acid comprises a disabled primer binding site (PBS) and/or att site. In embodiments, one or more viral accessory genes, including rev, tat, vif, nef, vpr, vpu, vpx and S2 or functional equivalents thereof, are disabled or absent from the retroviral nucleic acid. In embodiments, one or more accessory genes selected from S2, rev and tat are disabled or absent from the retroviral nucleic acid

In some embodiments, the retroviral vector systems described herein comprise viral genomes bearing cis-acting vector sequences for transcription, reverse-transcription, integration, translation and packaging of viral RNA into the viral particles, and (2) producer cells lines which express the trans-acting retroviral gene sequences (e.g., gag, pol and env) needed for production of virus particles. In some embodiments, by separating the cis- and trans-acting vector sequences completely, the virus is unable to maintain replication for more than one cycle of infection. Generation of live virus can be avoided by a number of strategies, e.g., by minimizing the overlap between the cis- and trans-acting sequences to avoid recombination.

In some embodiments, a viral vector particle which comprises a sequence that is devoid of or lacking viral RNA may be the result of removing or eliminating the viral RNA from the sequence. In one embodiment this may be achieved by using an endogenous packaging signal binding site on gag. In some embodiments, the endogenous packaging signal binding site is on pol. In this embodiment, the RNA which is to be delivered will contain a cognate packaging signal. In another embodiment, a heterologous binding domain (which is heterologous to gag) located on the RNA to be delivered, and a cognate binding site located on gag or pol, can be used to ensure packaging of the RNA to be delivered. In some embodiments, the heterologous sequence could be non-viral or it could be viral, in which case it may be derived from a different virus. In some embodiments, the vector particles are used to deliver therapeutic RNA, in which case functional integrase and/or reverse transcriptase is not required. In some embodiments, the vector particles could also be used to deliver a therapeutic gene of interest, in which case pol is typically included.

In some embodiments, gag-pol are altered, and the packaging signal is replaced with a corresponding packaging signal. In this embodiment, the particle can package the RNA with the new packaging signal. The advantage of this approach is that it is possible to package an RNA sequence which is devoid of viral sequence for example, RNAi.

In some embodiments, an alternative approach is to rely on over-expression of the RNA to be packaged. In one embodiment the RNA to be packaged is over-expressed in the absence of any RNA containing a packaging signal. This may result in a significant level of therapeutic RNA being packaged, and that this amount is sufficient to transduce a cell and have a biological effect.

In some embodiments, a polynucleotide comprises a nucleotide sequence encoding a viral gag protein or retroviral gag and pol proteins, wherein the gag protein or pol protein comprises a heterologous RNA binding domain capable of recognizing a corresponding sequence in an RNA sequence to facilitate packaging of the RNA sequence into a viral vector particle.

In some embodiments, the heterologous RNA binding domain comprises an RNA binding domain derived from a bacteriophage coat protein, a Rev protein, a protein of the U1 small nuclear ribonucleoprotein particle, a Nova protein, a TF111A protein, a TIS11 protein, a trp RNA-binding attenuation protein (TRAP) or a pseudouridine synthase.

When a recombinant plasmid and the retroviral LTR and packaging sequences are introduced into a producer cell line (e.g., by calcium phosphate precipitation for example), the packaging sequences may permit the RNA transcript of the recombinant plasmid to be packaged into viral particles, which then may be secreted into the culture media. The media containing the recombinant retroviruses in some embodiments is then collected, optionally concentrated, and used for gene transfer. For example, in some aspects, after cotransfection of the packaging plasmids and the transfer vector to the packaging cell line, the viral vector particles are recovered from the culture media and titered by standard methods used by those of skill in the art.

In some embodiments, a retroviral vector, such as a lentiviral vector, can be produced in a producer cell line, such as an exemplary HEK 293T cell line, by introduction of plasmids to allow generation of lentiviral particles. In some embodiments, a producer cell is transfected and/or contains a polynucleotide encoding gag and pol, and, in some cases, a polynucleotide encoding an exogenous agent. In some embodiments, the producer cell line is optionally and/or additionally transfected with and/or contains a polynucleotide encoding a rev protein. In some embodiments, the producer cell line is optionally and/or additionally transfected with and/or contains a polynucleotide encoding a non-native envelope glycoprotein, such as VSV-G. In some such embodiments, approximately two days after transfection of cells, e.g. HEK 293T cells, the cell supernatant contains recombinant lentiviral vectors, which can be recovered and titered.

In some embodiments, a method herein comprises detecting or confirming the absence of replication competent retrovirus. The methods may include assessing RNA levels of one or more target genes, such as viral genes, e.g. structural or packaging genes, from which gene products are expressed in certain cells infected with a replication-competent retrovirus, such as a gammaretrovirus or lentivirus, but not present in a viral vector used to transduce cells with a heterologous nucleic acid and not, or not expected to be, present and/or expressed in cells not containing replication-competent retrovirus. Replication competent retrovirus may be determined to be present if RNA levels of the one or more target genes is higher than a reference value, which can be measured directly or indirectly, e.g. from a positive control sample containing the target gene. For further disclosure, see WO2018023094A1

### E. Targeting and Retargeting of Lipid Particles

In some embodiments, the lipid particle further comprises a vector-surface targeting moiety which specifically binds to a target ligand on a target cell. It will be recognized by those skilled in the art that, the lipid particles provided herein harbor the attachment and/or fusion glycoproteins and are capable of binding to target cells and delivering the vehicle contents to the cytoplasm of the target cells. It will also be recognized by those skilled in the art that this is due to the natural viral entry mechanism that involves fusion of the viral membrane directly with the target cell plasma membrane.

It will further be recognized by those skilled in the art that many viruses such as paramyxoviruses bind to sialic acid receptors, and hence the corresponding derivative vehicles can deliver their contents generically to nearly any kind of cell that expresses sialic acid receptors. Other viruses such as Nipah virus and HIV bind to protein receptors, and hence the corresponding vehicles have a specificity that matches the natural tropisms for each virus and its surface proteins.

Furthermore, it will be recognized that technology exists to “re-target” attachment proteins, making it so that the vehicles only interact with particular cells or cell types that express a marker protein of interest (Msaouel et al., Meths Mol Biol 797: 141-162, 2012). Thus, vehicle surface glycoproteins proteins can be supplemented with or replaced by other targeting proteins, including but not necessarily limited to antibodies and antigen binding fragments thereof, receptor ligands, and other approaches that will be apparent to those skilled in the art given the benefit of the present disclosure. In some embodiments, the vector-surface targeting moiety is a polypeptide. In some embodiments, the polypeptide is a fusogen.

In some embodiments, the lipid particle comprises one or more fusogens. In some embodiments, the fusogen facilitates the fusion of the lipid particle to a cell membrane to delivery the payload gene into the cell. In some embodiments, the membrane is a plasma cell membrane. In some embodiments, the fusogen targets the lipid particle to a target cell of interest. In some embodiments, the fusogen contains a targeting moiety that provides retargeting (compared to the natural tropism of the fusogen) to the target cell of interest.

Provided herein are methods of administration of lipid particles (e.g. viral vectors) containing a fusogen disposed or embedded in the lipid bilayer. Exemplary fusogens are described in subsections below. In some embodiments, the fusogen is composed of one or more Paramyxovirus envelope protein or a biologically active portion thereof. In some embodiments, the Paramyxovirus envelope protein or a biologically active portion thereof harbors the attachment and/or fusion glycoproteins and are capable of binding to target cells and delivering the vehicle contents to the cytoplasm of the target cells.

### F. Fusogens

In some embodiments, the lipid particles, such as viral vectors or viral-like particles, contain one or more fusogens. In some embodiments, the lipid particle, e.g. viral vector or viral-like particle, contains an exogenous or overexpressed fusogen. In some embodiments, the fusogen is disposed in the lipid bilayer. In some embodiments, the fusogen facilitates the fusion of the lipid particle to a membrane. In some embodiments, the membrane is a plasma cell membrane of a target cell. In some embodiments, the lipid particle, such as a viral or non-viral vector, comprising the fusogen integrates into the membrane into a lipid bilayer of a target cell. In some embodiments, the fusogen results in mixing between lipids in the lipid particle and lipids in the target cell. In some embodiments, the fusogen results in formation of one or more pores between the interior of the non-cell particle and the cytosol of the target cell.

In some embodiments, fusogens are protein based, lipid based, and chemical based fusogens. In some embodiments, the lipid particle, e.g. viral vector or viral-like particle, contain a first fusogen that is a protein fusogen and a second fusogen that is a lipid fusogen or chemical fusogen. In some embodiments, the fusogen binds a fusogen binding partner on a target cell surface. In some embodiments, the lipid particle is a viral vector or viral-like particle that is pseudotyped with the fusogen. In some examples, a virus of viral-like particle has a modification to one or more of its envelope proteins, e.g., an envelope protein is substituted with an envelope protein from another virus. In some embodiments, retroviral envelope proteins, e.g. lentiviral envelope proteins, are pseudotyped with a fusogen.

In some embodiments, the fusogen is a protein fusogen, e.g., a mammalian protein or a homologue of a mammalian protein (e.g., having 50%, 60%, 70%, 80%, 85%, 90%, 95%, 96%, 97%, 98%, 99%, or greater identity), a non-mammalian protein such as a viral protein or a homologue of a viral protein (e.g., having 50%, 60%, 70%, 80%, 85%, 90%, 95%, 96%, 97%, 98%, 99%, or greater identity), a native protein or a derivative of a native protein, a synthetic protein, a fragment thereof, a variant thereof, a protein fusion comprising one or more of the fusogens or fragments, and any combination thereof.

In some embodiments, the fusogen may include a mammalian protein. Examples of mammalian fusogens may include, but are not limited to, a SNARE family protein such as vSNAREs and tSNAREs, a syncytin protein such as Syncytin-1 (DOI: 10.1128/JVI.76.13.6442-6452.2002), and Syncytin-2, myomaker (biorxiv.org/content/early/2017/04/02/123158, doi.org/10.1101/123158, doi: 10.1096/fj.201600945R, doi:10.1038/nature12343), myomixer (www.nature.com/nature/journal/v499/n7458/full/nature12343.html, doi:10.1038/nature12343), myomerger (science.sciencemag.org/content/early/2017/04/05/science.aam9361, DOI: 10.1126/science.aam9361), FGFRL1 (fibroblast growth factor receptor-like 1), Minion (doi.org/10.1101/122697), an isoform of glyceraldehyde-3-phosphate dehydrogenase (GAPDH) (e.g., as disclosed in U.S. Pat. No. 6,099,857A), a gap junction protein such as connexin 43, connexin 40, connexin 45, connexin 32 or connexin 37 (e.g., as disclosed in US 2007/0224176, Hap2, any protein capable of inducing syncytium formation between heterologous cells, any protein with fusogen properties, a homologue thereof, a fragment thereof, a variant thereof, and a protein fusion comprising one or more proteins or fragments thereof. In some embodiments, the fusogen is encoded by a human endogenous retroviral element (hERV) found in the human genome. Additional exemplary fusogens are disclosed in U.S. Pat. No. 6,099,857A and US 2007/0224176, the entire contents of which are hereby incorporated by reference.

In some embodiments, the fusogen may include a non-mammalian protein, e.g., a viral protein. In some embodiments, a viral fusogen is a Class I viral membrane fusion protein, a Class II viral membrane protein, a Class III viral membrane fusion protein, a viral membrane glycoprotein, or other viral fusion proteins, or a homologue thereof, a fragment thereof, a variant thereof, or a protein fusion comprising one or more proteins or fragments thereof.

In some embodiments, Class I viral membrane fusion proteins include, but are not limited to, Baculovirus F protein, e.g., F proteins of the nucleopolyhedrovirus (NPV) genera, e.g., Spodoptera exigua MNPV (SeMNPV) F protein and Lymantria dispar MNPV (LdMNPV), and paramyxovirus F proteins.

In some embodiments, Class II viral membrane proteins include, but are not limited to, tick bone encephalitis E (TBEV E), Semliki Forest Virus E1/E2.

In some embodiments, Class III viral membrane fusion proteins include, but are not limited to, rhabdovirus G (e.g., fusogenic protein G of the Vesicular Stomatitis Virus (VSV-G)), herpesvirus glycoprotein B (e.g., Herpes Simplex virus 1 (HSV-1) gB)), Epstein Barr Virus glycoprotein B (EBV gB), thogotovirus G, baculovirus gp64 (e.g., Autographa California multiple NPV (AcMNPV) gp64), Baboon endogenous retrovirus envelope glycoprotein (BaEV), and Borna disease virus (BDV) glycoprotein (BDV G).

In some embodiments, the fusogen is a Baboon Endogenous Retrovirus (BaEV) envelope glycoprotein. Exemplary BaEV envelope glycoproteins and variants thereof are described in PCT/US2022/031459; U.S. Pat. No. 9,249,426; Aguila et al. Journal of Virology 2003 77(2):1281-1291; Bernadin et al. Blood Advances 2019 3(3):461-475; Colamartino et al. Frontiers in Immunology 2019 10:2873; Girard-Gagnepain et al. Blood 2014 124(8):1221-1231; and Levy et al. Journal of Thrombosis and Haemostasis 2016 14:2478-2492. Further exemplary BaEV envelope glycoproteins and variants thereof are also described in Application PCT/US2022/031459, the contents of which are hereby incorporated in their entirety.

In some embodiments, the truncated BaEV envelope glycoprotein comprises a cytoplasmic tail with a partial fusion inhibitory R peptide relative to a wild-type BaEV envelope glycoprotein, wherein the R peptide contains a contiguous portion of the inhibitory R peptide but lacks the full length R peptide of wild-type BaEV envelope glycoprotein. In some embodiments, the truncated BaEV envelope glycoprotein has a cytoplasmic tail that is composed of a partial inhibitory R peptide with at least one, at least two, or at least three contiguous amino-terminal amino acids of the inhibitory R peptide but less than the full-length R peptide relative to wild-type BaEV envelope glycoprotein. In some embodiments, the truncated BaEV envelope glycoprotein has a cytoplasmic tail that has a partial inhibitory R peptide composed of 1 to 16 contiguous amino-terminal amino acids of the inhibitory R peptide of the wild-type BaEV envelope glycoprotein, such as is composed of 1, 2, 3, 4, 5, 6, 7, 8, 9, 10, 11, 12, 12, 14, 15 or 16 amino-terminal amino acids of the inhibitory R peptide of the wild-type BaEV envelope glycoprotein.

In some embodiments, the fusogen is a modified BaEV envelope glycoprotein. In some embodiments, the cytoplasmic tail domain of the BaEV envelope glycoprotein is devoid of the fusion inhibitory R peptide. The expression “fusion inhibitory R peptide” refers to the C-terminal portion of the cytoplasmic tail domain of the envelope glycoprotein which harbors a tyrosine endocytosis signal—YXXL—and which is cleaved by viral protease during virion maturation, thus enhancing membrane fusion of the envelope glycoprotein. The fusion inhibitory R peptide of the BaEV envelope glycoprotein is typically located between amino acids 547 and 564 of the wild-type BaEV envelope glycoprotein

Examples of other viral fusogens, e.g., membrane glycoproteins and viral fusion proteins, include, but are not limited to: viral syncytia proteins such as influenza hemagglutinin (HA) or mutants, or fusion proteins thereof; human immunodeficiency virus type 1 envelope protein (HIV-1 ENV), gp120 from HIV binding LFA-1 to form lymphocyte syncytium, HIV gp41, HIV gp160, or HIV Trans-Activator of Transcription (TAT); viral glycoprotein VSV-G, viral glycoprotein from vesicular stomatitis virus of the Rhabdoviridae family; glycoproteins gB and gH-gL of the varicella-zoster virus (VZV); murine leukemia virus (MLV)-10A1; Gibbon Ape Leukemia Virus glycoprotein (GaLV); type G glycoproteins in Rabies, Mokola, vesicular stomatitis virus and Togaviruses; murine hepatitis virus JHM surface projection protein; porcine respiratory coronavirus spike- and membrane glycoproteins; avian infectious bronchitis spike glycoprotein and its precursor; bovine enteric coronavirus spike protein; the F and H, HN or G genes of Measles virus; canine distemper virus, Newcastle disease virus, human parainfluenza virus 3, simian virus 41, Sendai virus and human respiratory syncytial virus; gH of human herpesvirus 1 and simian varicella virus, with the chaperone protein gL; human, bovine and cercopithicine herpesvirus gB; envelope glycoproteins of Friend murine leukemia virus and Mason Pfizer monkey virus; mumps virus hemagglutinin neuraminidase, and glycoproteins F1 and F2; membrane glycoproteins from Venezuelan equine encephalomyelitis; paramyxovirus F protein; SIV gp160 protein; Ebola virus G protein; or Sendai virus fusion protein, or a homologue thereof, a fragment thereof, a variant thereof, and a protein fusion comprising one or more proteins or fragments thereof.

Non-mammalian fusogens include viral fusogens, homologues thereof, fragments thereof, and fusion proteins comprising one or more proteins or fragments thereof. Viral fusogens include class I fusogens, class II fusogens, class III fusogens, and class IV fusogens. In embodiments, class I fusogens such as human immunodeficiency virus (HIV) gp41, have a characteristic post fusion conformation with a signature trimer of α-helical hairpins with a central coiled-coil structure. Class I viral fusion proteins include proteins having a central post fusion six-helix bundle. Class I viral fusion proteins include influenza HA, parainfluenza F, HIV Env, Ebola GP, hemagglutinins from orthomyxoviruses, F proteins from paramyxoviruses (e.g. Measles, (Katoh et al. BMC Biotechnology 2010, 10:37)), ENV proteins from retroviruses, and fusogens of filoviruses and coronaviruses. In embodiments, class II viral fusogens such as dengue E glycoprotein, have a structural signature of (3-sheets forming an elongated ectodomain that refolds to result in a trimer of hairpins. In embodiments, the class II viral fusogen lacks the central coiled coil. Class II viral fusogen can be found in alphaviruses (e.g., E1 protein) and flaviviruses (e.g., E glycoproteins). Class II viral fusogens include fusogens from Semliki Forest virus, Sinbis, rubella virus, and dengue virus. In embodiments, class III viral fusogens such as the vesicular stomatitis virus G glycoprotein, combine structural signatures found in classes I and II. In embodiments, a class III viral fusogen comprises α helices (e.g., forming a six-helix bundle to fold back the protein as with class I viral fusogens), and β sheets with an amphiphilic fusion peptide at its end, reminiscent of class II viral fusogens. Class III viral fusogens can be found in rhabdoviruses and herpesviruses. In embodiments, class IV viral fusogens are fusion-associated small transmembrane (FAST) proteins (doi:10.1038/sj.emboj.7600767, Nesbitt, Rae L., “Targeted Intracellular Therapeutic Delivery Using Liposomes Formulated with Multifunctional FAST proteins” (2012). Electronic Thesis and Dissertation Repository. Paper 388), which are encoded by nonenveloped reoviruses. In embodiments, the class IV viral fusogens are sufficiently small that they do not form hairpins (doi: 10.1146/annurev-cellbio-101512-122422, doi:10.1016/j.devcel.2007.12.008).

Additional exemplary fusogens are disclosed in U.S. Pat. No. 9,695,446, US 2004/0028687, U.S. Pat. Nos. 6,416,997, 7,329,807, US 2017/0112773, US 2009/0202622, WO 2006/027202, and US 2004/0009604, the entire contents of all of which are hereby incorporated by reference.

In some embodiments, the fusogen is a poxviridae fusogen.

In some embodiments the fusogen is a paramyxovirus fusogen. In some embodiments, the fusogen may be an envelope glycoprotein G, H HN and/or an F protein of the Paramyxoviridae family. In some embodiments the fusogen contains a Nipah virus protein F, a measles virus F protein, a tupaia paramyxovirus F protein, a paramyxovirus F protein, a Hendra virus F protein, a Henipavirus F protein, a Morbilivirus F protein, a respirovirus F protein, a Sendai virus F protein, a rubulavirus F protein, or an avulavirus F protein. In some embodiments, the lipid particle includes contains a henipavirus envelope attachment glycoprotein G (G protein) or a biologically active portion thereof and/or a henipavirus envelope fusion glycoprotein F (F protein) or a biologically active portion thereof.

In particular embodiments, the fusogen is glycoprotein GP64 of baculovirus, glycoprotein GP64 variant E45K/T259A.

In some embodiments, the fusogen is a hemagglutinin-neuraminidase (HN) and fusion (F) proteins (F/HN) from a respiratory paramyxovirus. In some embodiments, the respiratory paramyxovirus is a Sendai virus. The HN and F glycoproteins of Sendai viruses function to attach to sialic acids via the HN protein, and to mediate cell fusion for entry to cells via the F protein. In some embodiments, the fusogen is a F and/or HN protein from the murine parainfluenza virus type 1 (See e.g., U.S. patent Ser. No. 10/704,061).

In some embodiments the fusogen is a paramyxovirus fusogen. In some embodiments, the fusogen may be or an envelope glycoprotein G, H and/or an F protein of the Paramyxoviridae family. In some embodiments the fusogen contains a Nipah virus protein F, a measles virus F protein, a canine distemper virus F protein, a tupaia paramyxovirus F protein, a paramyxovirus F protein, a Hendra virus F protein, a Henipavirus F protein, a Morbilivirus F protein, a respirovirus F protein, a Sendai virus F protein, a rubulavirus F protein, or an avulavirus F protein. In some embodiments, the lipid particle includes contains a henipavirus envelope attachment glycoprotein G (G protein) or a biologically active portion thereof and/or a henipavirus envelope fusion glycoprotein F (F protein) or a biologically active portion thereof.

In some embodiments, the lipid particle (e.g. viral vector) is psedutoptyed with viral glycoproteins as described herein such as a NiV-F and/or NiV-G protein.

In some embodiments, the viral vector further comprises a vector-surface targeting moiety which specifically binds to a target ligand. In some embodiments, the vector-surface targeting moiety is a polypeptide. In some embodiments, the nucleic acid encoding the one of the Paramyxovirus envelope protein (e.g. G protein) is modified with a targeting moiety to specifically bind to a target molecule on a target cells. In some embodiments, the targeting moiety can be any targeting protein, including but not necessarily limited to antibodies and antigen binding fragments thereof.

It has been reported that the henipavirus F proteins from various species exhibit compatibility with G proteins from other species to trigger fusion (Brandel-Tretheway et al. Journal of Virology. 2019. 93(13):e00577-19). In some aspects of the provided lipid particles (e.g. lentiviral vector), the F protein is heterologous to the G protein, i.e. the F and G protein or biologically active portions are from different henipavirus species. For example, the G protein is from Hendra virus and the F protein is a NiV-F as described. In other aspects, the F and/or G protein can be a chimeric F and/or G protein containing regions of F and/or G proteins from different species of Henipavirus. In some embodiments, switching a region of amino acid residues of the F protein from one species of Henipavirus to another can result in fusion to the G protein of the species comprising the amino acid insertion. (Brandel-Tretheway et al. 2019). In some cases, the chimeric F and/or G protein contains an extracellular domain from one henipavirus species and a transmembrane and/or cytoplasmic domain from a different henipavirus species. For example, the F protein contains an extracellular domain of Hendra virus and a transmembrane/cytoplasmic domain of Nipah virus.

### 1. F Proteins

In some embodiments, the lipid particles (e.g. lentiviral vectors) comprises a protein with a hydrophobic fusion peptide domain. In some embodiments, the protein with a hydrophobic fusion peptide domain may be an envelope glycoprotein F protein of the Paramyxoviridae family (i.e., a paramyxovirus F protein). In some embodiments, the envelope glycoprotein F protein comprises a henipavirus F protein molecule or biologically active portion thereof. In some embodiments, the Henipavirus F protein is a Hendra (HeV) virus F protein, a Nipah (NiV) virus F-protein, a Cedar (CedPV) virus F protein, a Mojiang virus F protein or a bat Paramyxovirus F protein or a biologically active portion thereof. Exemplary F proteins or biologically active variants include any as described herein. Such envelope proteins, as well as variants thereof, also include any as disclosed in U.S. Patent Applications 63/291,316 and 63/291,323, each of whose disclosure is hereby incorporated by reference.

In some embodiments, the fusogen comprises a protein with a hydrophobic fusion peptide domain. In some embodiments, the fusogen comprises a henipavirus F protein molecule or biologically active portion thereof. In some embodiments, the Henipavirus F protein is a Hendra (HeV) virus F protein, a Nipah (NiV) virus F-protein, a Cedar (CedPV) virus F protein, a Mojiang virus F protein or a bat Paramyxovirus F protein or a biologically active portion thereof.

F proteins of henipaviruses are encoded as F₀ precursors containing a signal peptide. Following cleavage of the signal peptide, the mature F₀ is transported to the cell surface, then endocytosed and cleaved by cathepsin L into the mature fusogenic subunits F1 and F2. For instance, with reference to NiV-F the NiV-F is encoded as F0 precursors containing a signal peptide (e.g. corresponding to amino acid residues 1-26 of the below). Following cleavage of the signal peptide, the mature F0 (SEQ ID NO:2 lacking the signal peptide, i.e. set forth in SEQ ID NO:7) is transported to the cell surface, then endocytosed and cleaved by cathepsin L (e.g. between amino acids 109-110 of NiV-F corresponding to amino acids set forth in SEQ ID NO:2) into the mature fusogenic subunits F1 (e.g. corresponding to amino acids 110-546 of NiV-F set forth in SEQ ID NO:2) and F2 (e.g. corresponding to amino acid residues 27-109 of NiV-F set forth in SEQ ID NO:2). The F1 and F2 subunits are associated by a disulfide bond and recycled back to the cell surface. The F1 subunit contains the fusion peptide domain located at the N terminus of the F1 subunit (e.g. corresponding to amino acids 110-129 of the below e.g. NiV-F set forth in SEQ ID NO:2), where it is able to insert into a cell membrane to drive fusion. Without wishing to be bound by theory, in some aspects, fusion is blocked by association of the F protein with G protein, until the G protein engages with a target molecule resulting in its disassociation from F and exposure of the fusion peptide to mediate membrane fusion.

In some embodiments, the F protein (e.g. NiV-F protein) of the lipid particle, such as lentiviral vector, exhibits fusogenic activity. In some embodiments, the F protein (e.g. NiV-F) facilitates the fusion of the lipid particle (e.g. lentiviral vector) to a membrane. In particular embodiments, the F protein or the functionally active variant or biologically active portion thereof retains fusogenic activity in conjunction with a Henipavirus G protein, such as a G protein set forth below. Fusogenic activity includes the activity of the F protein in conjunction with a G protein to promote or facilitate fusion of two membrane lumens, such as the lumen of the targeted lipid particle having embedded in its lipid bilayer a henipavirus F and G protein, and a cytoplasm of a target cell, e.g. a cell that contains a surface receptor or molecule that is recognized or bound by the targeted envelope protein. In some embodiments, the F protein and G protein are from the same Henipavirus species (e.g. NiV-G and NiV-F). In some embodiments, the F protein and G protein are from different Henipavirus species (e.g. NiV-G and HeV-F). In particular embodiments, the F protein of the functionally active variant or biologically active portion retains the cleavage site cleaved by cathepsin L (e.g. corresponding to the cleavage site between amino acids 109-110 of SEQ ID NO:2).

In some embodiments, the F protein or the functionally active variant or biologically active portion thereof comprises an F1 subunit or a fusogenic portion thereof. In some embodiments, the F1 subunit is a proteolytically cleaved portion of the F0 precursor. In some embodiments, the F0 precursor is inactive. In some embodiments, the cleavage of the F0 precursor forms a disulfide-linked F1+F2 heterodimer. In some embodiments, the cleavage exposes the fusion peptide and produces a mature F protein. In some embodiments, the cleavage occurs at or around a single basic residue. In some embodiments, the cleavage occurs at Arginine 10⁹ of NiV-F protein. In some embodiments, cleavage occurs at Lysine 10⁹ of the Hendra virus F protein.

Table 1A provides non-limiting examples of F proteins. In some embodiments, the N-terminal hydrophobic fusion peptide domain of the F protein molecule or biologically active portion thereof is exposed on the outside of lipid bilayer.

Among different henipavirus species, the sequence and activity of the F protein is highly conserved. For examples, the F protein of NiV and HeV viruses share 89% amino acid sequence identity. Further, in some cases, the henipavirus F proteins exhibit compatibility with G proteins from other species to trigger fusion (Brandel-Tretheway et al. Journal of Virology. 2019. 93(13):e00577-19). In some aspects of the provided lipid particles, the F protein is heterologous to the G protein, i.e. the F and G protein or biologically active portions are from different henipavirus species. For example, the F protein is from Hendra virus and the G protein is from Nipah virus. In other aspects, the F protein can be a chimeric F protein containing regions of F proteins from different species of Henipavirus. In some embodiments, switching a region of amino acid residues of the F protein from one species of Henipavirus to another can result in fusion to the G protein of the species comprising the amino acid insertion. (Brandel-Tretheway et al. 2019). In some cases, the chimeric F protein contains an extracellular domain from one henipavirus species and a transmembrane and/or cytoplasmic domain from a different henipavirus species. For example, the F protein contains an extracellular domain of Hendra virus and a transmembrane/cytoplasmic domain of Nipah virus. F protein sequences disclosed herein are predominantly disclosed as expressed sequences including an N-terminal signal sequence. As such N-terminal signal sequences are commonly cleaved co- or post-translationally, the mature protein sequences for all F protein sequences disclosed herein are also contemplated as lacking the N-terminal signal sequence.

TABLE 1A
Non-limiting Examples of F Proteins

| | | | | | SEQ ID |
|---|---|---|---|---|---|
| | | | | | NO |
| | Nucleo- | | | | (without |
| Genbank | tides | Full | | | signal |
| ID | of CDS | Gene Name | Sequence | SEQ ID | sequence) |
| AF017149 | 6618-8258 | gb: | MATQEVRLKCLLCGIIVLVLSLEGLGIL | 1 | 6 |
| | | 1AF07149\| | HYEKLSKIGLVKGITRKYKIKSNPLTKDI | | |
| | | Organism: | VIKMIPNVSNVSKCTGTVMENYKSRLT | | |
| | | Hendra virus\| | GILSPIKGAIELYNNNTHDLVGDVKLAG | | |
| | | Strain | VVMAGIAIGIATAAQITAGVALYEAMK | | |
| | | Name: | NADNINKLKSSIESTNEAVVKLQETAEK | | |
| | | UNKNOWN- | TVYVLTALQDYINTNLVPTIDQISCKQT | | |
| | | AF017149\| | ELALDLALSKYLSDLLFVFGPNLQDPVS | | |
| | | Protein | NSMTIQAISQAFGGNYETLLRTLGYATE | | |
| | | Name: | DFDDLLESDSIAGQIVYVDLSSYYIIVRV | | |
| | | fusion\| | YFPILTEIQQAYVQELLPVSFNNDNSEWI | | |
| | | Gene | SIVPNFVLIRNTLISNIEVKYCLITKKSVI | | |
| | | Symbol: | CNQDYATPMTASVRECLTGSTDKCPRE | | |
| | | F | LVVSSHVPRFALSGGVLFANCISVTCQC | | |
| | | | QTTGRAISQSGEQTLLMIDNTTCTTVVL | | |
| | | | GNIIISLGKYLGSINYNSESIAVGPPVYTD | | |
| | | | KVDISSQISSMNQSLQQSKDYIKEAQKIL | | |
| | | | DTVNPSLISMLSMIILYVLSIAALCIGLIT | | |
| | | | FISFVIVEKKRGNYSRLDDRQVRPVSNG | | |
| | | | DLYYIGT | | |
| Q9IH63 | | Additional in | MVVILDKRCYCNLLILILMISECSVGILH | 2 | 7 |
| | | cluster: | YEKLSKIGLVKGVTRKYKIKSNPLTKDI | | |
| | | sp\| | VIKMIPNVSNMSQCTGSVMENYKTRLN | | |
| | | Q9IH63\| | GILTPIKGALEIYKNNTHDLVGDVRLAG | | |
| | | FUS_NIPAV | VIMAGVAIGIATAAQITAGVALYEAMK | | |
| | | Fusion | NADNINKLKSSIESTNEAVVKLQETAEK | | |
| | | glycoprotein | TVYVLTALQDYINTNLVPTIDKISCKQT | | |
| | | F0 | ELSLDLALSKYLSDLLFVFGPNLQDPVS | | |
| | | OS = Nipah | NSMTIQAISQAFGGNYETLLRTLGYATE | | |
| | | virus | DFDDLLESDSITGQIIYVDLSSYYIIVRVY | | |
| | | | FPILTEIQQAYIQELLPVSFNNDNSEWISI | | |
| | | | VPNFILVRNTLISNIEIGFCLITKRSVICN | | |
| | | | QDYATPMTNNMRECLTGSTEKCPRELV | | |
| | | | VSSHVPRFALSNGVLFANCISVTCQCQT | | |
| | | | TGRAISQSGEQTLLMIDNTTCPTAVLGN | | |
| | | | VIISLGKYLGSVNYNSEGIAIGPPVFTDK | | |
| | | | VDISSQISSMNQSLQQSKDYIKEAQRLL | | |
| | | | DTVNPSLISMLSMIILYVLSIASLCIGLITF | | |
| | | | ISFIIVEKKRNTYSRLEDRRVRPTSSGDL | | |
| | | | YYIGT | | |
| JQ001776 | 6129-8166 | gb: | MSNKRTTVLIIISYTLFYLNNAAIVGFDF | 3 | 8 |
| | | JQ001776: | DKLNKIGVVQGRVLNYKIKGDPMTKDL | | |
| | | 6129-8166\| | VLKFIPNIVNITECVREPLSRYNETVRRL | | |
| | | Organism: | LLPIHNMLGLYLNNTNAKMTGLMIAGV | | |
| | | Cedar | IMGGIAIGIATAAQITAGFALYEAKKNT | | |
| | | virus\| | ENIQKLTDSIMKTQDSIDKLTDSVGTSIL | | |
| | | Strain | ILNKLQTYINNQLVPNLELLSCRQNKIEF | | |
| | | Name: | DLMLTKYLVDLMTVIGPNINNPVNKDM | | |
| | | CG1a\| | TIQSLSLLFDGNYDIMMSELGYTPQDFL | | |
| | | Protein | DLIESKSITGQIIYVDMENLYVVIRTYLP | | |
| | | Name: | TLIEVPDAQIYEFNKITMSSNGGEYLSTI | | |
| | | fusion | PNFILIRGNYMSNIDVATCYMTKASVIC | | |
| | | glycoprotein\| | NQDYSLPMSQNLRSCYQGETEYCPVEA | | |
| | | Gene | VIASHSPRFALTNGVIFANCINTICRCQD | | |
| | | Symbol: | NGKTITQNINQFVSMIDNSTCNDVMVD | | |
| | | F | KFTIKVGKYMGRKDINNINIQIGPQIIIDK | | |
| | | | VDLSNEINKMNQSLKDSIFYLREAKRIL | | |
| | | | DSVNISLISPSVQLFLIIISVLSFIILLIIIVY | | |
| | | | LYCKSKHSYKYNKFIDDPDYYNDYKRE | | |
| | | | RINGKASKSNNIYYVGD | | |
| NC_025352 | 5950-8712 | gb: | MALNKNMFSSLFLGYLLVYATTVQSSI | 4 | 9 |
| | | NC025352: | HYDSLSKVGVIKGLTYNYKIKGSPSTKL | | |
| | | 5950-8712\| | MVVKLIPNIDSVKNCTQKQYDEYKNLV | | |
| | | Organism: | RKALEPVKMAIDTMLNNVKSGNNKYR | | |
| | | Mojiang | FAGAIMAGVALGVATAATVTAGIALHR | | |
| | | virus\| | SNENAQAIANMKSAIQNTNEAVKQLQL | | |
| | | Strain | ANKQTLAVIDTIRGEINNNIIPVINQLSC | | |
| | | Name: | DTIGLSVGIRLTQYYSEIITAFGPALQNP | | |
| | | Tongguan1\| | VNTRITIQAISSVFNGNFDELLKIMGYTS | | |
| | | Protein | GDLYEILHSELIRGNIIDVDVDAGYIALE | | |
| | | Name: | IEFPNLTLVPNAVVQELMPISYNIDGDE | | |
| | | fusion | WVTLVPRFVLTRTTLLSNIDTSRCTITDS | | |
| | | protein\| | SVICDNDYALPMSHELIGCLQGDTSKCA | | |
| | | Gene | REKVVSSYVPKFALSDGLVYANCLNTIC | | |
| | | Symbol: | RCMDTDTPISQSLGATVSLLDNKRCSVY | | |
| | | F | QVGDVLISVGSYLGDGEYNADNVELGP | | |
| | | | PIVIDKIDIGNQLAGINQTLQEAEDYIEK | | |
| | | | SEEFLKGVNPSIITLGSMVVLYIFMILIAI | | |
| | | | VSVIALVLSIKLTVKGNVVRQQFTYTQH | | |
| | | | VPSMENINYVSH | | |
| NC_025256 | 6865-8853 | gb: | MKKKTDNPTISKRGHNHSRGIKSRALLR | 5 | 10 |
| | | NC_025256: | ETDNYSNGLIVENLVRNCHHPSKNNLN | | |
| | | 6865-8853\| | YTKTQKRDSTIPYRVEERKGHYPKIKHL | | |
| | | Organism: | IDKSYKHIKRGKRRNGHNGNIITIILLLIL | | |
| | | Bat | ILKTQMSEGAIHYETLSKIGLIKGITREY | | |
| | | Paramyxovirus | KVKGTPSSKDIVIKLIPNVTGLNKCTNIS | | |
| | | Eid_hel/ | MENYKEQLDKILIPINNIIELYANSTKSA | | |
| | | GH- | PGNARFAGVIIAGVALGVAAAAQITAGI | | |
| | | M74a/GHA/ | ALHEARQNAERINLLKDSISATNNAVAE | | |
| | | 2009\| | LQEATGGIVNVITGMQDYINTNLVPQID | | |
| | | Strain | KLQCSQIKTALDISLSQYYSEILTVFGPN | | |
| | | Name: | LQNPVTTSMSIQAISQSFGGNIDLLLNLL | | |
| | | BatPV/Eid_ | GYTANDLLDLLESKSITGQITYINLEHYF | | |
| | | hel/GH- | MVIRVYYPIMTTISNAYVQELIKISFNVD | | |
| | | M74a/GHA/ | GSEWVSLVPSYILIRNSYLSNIDISECLIT | | |
| | | 2009\| | KNSVICRHDFAMPMSYTLKECLTGDTE | | |
| | | Protein | KCPREAVVTSYVPRFAISGGVIYANCLS | | |
| | | Name: | TTCQCYQTGKVIAQDGSQTLMMIDNQT | | |
| | | fusion | CSIVRIEEILISTGKYLGSQEYNTMHVSV | | |
| | | protein\| | GNPVFTDKLDITSQISNINQSIEQSKFYL | | |
| | | Gene | DKSKAILDKINLNLIGSVPISILFIIAILSLI | | |
| | | Symbol: | LSIITFVIVMIIVRRYNKYTPLINSDPSSR | | |
| | | F | RSTIQDVYIIPNPGEHSIRSAARSIDRDRD | | |

In some embodiments, the F protein or the biologically active portion thereof is a wild-type Nipah virus F (NiV-F) protein or a Hendra virus F protein or is a functionally active variant or biologically active portion thereof. For instance, in some embodiments, the F protein or the biologically active portion thereof is a wild-type NiV-F protein or a functionally active variant or a biologically active portion thereof.

In some embodiments, the F protein has the sequence of amino acids set forth in SEQ ID NO:1, SEQ ID NO:2, SEQ ID NO:3, SEQ ID NO:4, or SEQ ID NO:5, or is a functionally active variant thereof or a biologically active portion thereof that retains fusogenic activity. In some embodiments, the functionally active variant comprises an amino acid sequence having at least at or about 80%, at least at or about 85%, at least at or about 90%, at least at or about 91%, at least at or about 92%, at least at or about 93%, at least at or about 94%, at least at or about 95%, at or about 96%, at least at or about 97%, at least at or about 98%, or at least at or about 99% sequence identity to SEQ ID NO:1, SEQ ID NO:2, SEQ ID NO:3, SEQ ID NO:4, or SEQ ID NO:5, and retains fusogenic activity in conjunction with a G protein, such as a variant NiV-G as provided herein. In some embodiments, the biologically active portion has an amino acid sequence having at least at or about 80%, at least at or about 85%, at least at or about 90%, at least at or about 91%, at least at or about 92%, at least at or about 93%, at least at or about 94%, at least at or about 95%, at or about 96%, at least at or about 97%, at least at or about 98%, or at least at or about 99% sequence identity to SEQ ID NO:1, SEQ ID NO:2, SEQ ID NO:3, SEQ ID NO:4, or SEQ ID NO:5.

In particular embodiments, the F protein has the sequence of amino acids set forth in SEQ ID NO:6, SEQ ID NO:7, SEQ ID NO:8, SEQ ID NO:9, or SEQ ID NO:10, or is a functionally active variant thereof or a biologically active portion thereof that retains fusogenic activity. In some embodiments, the functionally active variant comprises an amino acid sequence having at least at or about 80%, at least at or about 85%, at least at or about 90%, at least at or about 91%, at least at or about 92%, at least at or about 93%, at least at or about 94%, at least at or about 95%, at or about 96%, at least at or about 97%, at least at or about 98%, or at least at or about 99% sequence identity to SEQ ID NO:6, SEQ ID NO:7, SEQ ID NO:8, SEQ ID NO:9, or SEQ ID NO:1, and retains fusogenic activity in conjunction with a G protein, such as a variant NiV-G as provided herein. In some embodiments, the biologically active portion has an amino acid sequence having at least at or about 80%, at least at or about 85%, at least at or about 90%, at least at or about 91%, at least at or about 92%, at least at or about 93%, at least at or about 94%, at least at or about 95%, at or about 96%, at least at or about 97%, at least at or about 98%, or at least at or about 99% sequence identity to SEQ ID NO:6, SEQ ID NO:7, SEQ ID NO:8, SEQ ID NO:9, or SEQ ID NO:1.

Fusogenic activity includes the activity of the F protein in conjunction with a G protein to promote or facilitate fusion of two membrane lumens, such as the lumen of the targeted lipid particle having embedded in its lipid bilayer a henipavirus F and G protein, and a cytoplasm of a target cell, e.g. a cell that contains a surface receptor or molecule that is recognized or bound by the targeted envelope protein. In some embodiments, the F protein and G protein are from the same Henipavirus species (e.g. NiV-G and NiV-F). In some embodiments, the F protein and G protein are from different Henipavirus species (e.g. NiV-G and HeV-F). In particular embodiments, the F protein of the functionally active variant or biologically active portion retains the cleavage site cleaved by cathepsin L (e.g. corresponding to the cleavage site between amino acids 109-110 of SEQ ID NO:2).

Reference to retaining fusogenic activity includes activity (in conjunction with a G protein, such as a variant G protein provided herein) that is between at or about 10% and at or about 150% or more of the level or degree of binding of the corresponding wild-type F protein, such as set forth in SEQ ID NO:1, SEQ ID NO:2, SEQ ID NO:3, SEQ ID NO:4, or SEQ ID NO:5, SEQ ID NO:6, SEQ ID NO:7, SEQ ID NO:8, SEQ ID NO:9, or SEQ ID NO:10 or a cathepsin L cleaved from thereof containing an F1 and F2 subunit. In some embodiments, the fusogenic activity is at least or at least about 10% of the level or degree of fusogenic activity of the corresponding wild-type F protein, such as at least or at least about 15% of the level or degree of fusogenic activity of the corresponding wild-type F protein, such as at least or at least about 20% of the level or degree of fusogenic activity of the corresponding wild-type F protein, such as at least or at least about 25% of the level or degree of fusogenic activity of the corresponding wild-type F protein, such as at least or at least about 30% of the level or degree of fusogenic activity of the corresponding wild-type F protein, such as at least or at least about 35% of the level or degree of fusogenic activity of the corresponding wild-type F protein, such as at least or at least about 40% of the level or degree of fusogenic activity of the corresponding wild-type F protein, such as at least or at least about 45% of the level or degree of fusogenic activity of the corresponding wild-type F protein, such as at least or at least about 50% of the level or degree of fusogenic activity of the corresponding wild-type F protein, such as at least or at least about 55% of the level or degree of fusogenic activity of the corresponding wild-type F protein, such as at least or at least about 60% of the level or degree of fusogenic activity of the corresponding wild-type F protein, such as at least or at least about 65% of the level or degree of fusogenic activity of the corresponding wild-type F protein, such as at least or at least about 70% of the level or degree of fusogenic activity of the corresponding wild-type F protein, such as at least or at least about 75% of the level or degree of fusogenic activity of the corresponding wild-type F protein, such as at least or at least about 80% of the level or degree of fusogenic activity of the corresponding wild-type F protein, such as at least or at least about 85% of the level or degree of fusogenic activity of the corresponding wild-type F protein, such as at least or at least about 90% of the level or degree of fusogenic activity of the corresponding wild-type F protein, such as at least or at least about 95% of the level or degree of fusogenic activity of the corresponding wild-type F protein, such as at least or at least about 100% of the level or degree of fusogenic activity of the corresponding wild-type F protein, or such as at least or at least about 120% of the level or degree of fusogenic activity of the corresponding wild-type F protein.

In some embodiments, the F protein is a mutant F protein that is a functionally active fragment or a biologically active portion containing one or more amino acid mutations, such as one or more amino acid insertions, deletions, substitutions or truncations. In some embodiments, the mutations described herein relate to amino acid insertions, deletions, substitutions or truncations of amino acids compared to a reference F protein sequence. In some embodiments, the reference F protein sequence is the wild-type sequence of an F protein or a biologically active portion thereof. In some embodiments, the mutant F protein or the biologically active portion thereof is a mutant of a wild-type Hendra (Hev) virus F protein, a Nipah (NiV) virus F-protein, a Cedar (CedPV) virus F protein, a Mojiang virus F protein or a bat Paramyxovirus F protein. In some embodiments, the wild-type F protein is encoded by a sequence of nucleotides that encodes any one of SEQ ID NO:1, SEQ ID NO:2, SEQ ID NO:3, SEQ ID NO:4, or SEQ ID NO:5, SEQ ID NO:6, SEQ ID NO:7, SEQ ID NO:8, SEQ ID NO:9, or SEQ ID NO:10 or a cathepsin L cleaved from thereof containing an F1 and F2 subunit.

In some embodiments, the mutant F protein is a biologically active portion that is truncated and lacks up to 22 contiguous amino acid residues at or near the C-terminus of the wild-type F protein, such as a wild-type F protein set forth in any one of SEQ ID NO:1, SEQ ID NO:2, SEQ ID NO:3, SEQ ID NO:4, or SEQ ID NO:5, SEQ ID NO:6, SEQ ID NO:7, SEQ ID NO:8, SEQ ID NO:9, or SEQ ID NO:10. In some embodiments, the mutant F protein is truncated and lacks up to 22 contiguous amino acids, such as up to 21, 20, 19, 18, 17, 16, 15, 14, 13, 12, 11, 10, 9, 8, 7, 6, 5, 4, 3, 2, or 1 contiguous amino acids at the C-terminus of the wild-type F protein.

In some embodiments, the NiV-F, of a provided lipid particle includes the F0 precursor or a proteolytically cleaved form thereof containing the F1 and F2 subunits, such as resulting following proteolytic cleavage at the cleavage site (e.g. between amino acids corresponding to amino acids between amino acids 109-110 of SEQ ID NO:2) to produce two chains that can be linked by disulfide bond. In some embodiments, the NiV-F, is produced or encoded as an F0 precursor which then is able to be proteolytically cleaved to result in an F protein containing the F1 and F2 subunit linked by a disulfide bond. Hence, it is understood that reference to a particular sequence (SEQ ID NO) of a NiV-F herein is typically with reference to the F0 precursor sequence but also is understood to include the proteolytically cleaved form or sequence thereof containing the two cleaved chains, F1 and F2. For instance, the NiV-F, such as a mutant or truncated NiV-F, contains an F1 subunit corresponding to amino acids 110-546 of NiV-F set forth in SEQ ID NO:2 or truncated or mutant sequence thereof, and an F2 corresponding to amino acid residues 27-109 of NiV-F set forth in SEQ ID NO:2.

In some embodiments, the mutant F protein is a biologically active portion that is truncated and lacks up to 22 contiguous amino acid residues at or near the C-terminus of the wild-type NiV-F protein, such as a wild-type NiV-F protein set forth in SEQ ID NO:2 or SEQ ID NO:7. In some embodiments, the mutant F protein is truncated and lacks up to 22 contiguous amino acids, such as up to 21, 20, 19, 18, 17, 16, 15, 14, 13, 12, 11, 10, 9, 8, 7, 6, 5, 4, 3, 2, or 1 contiguous amino acids at the C-terminus of the wild-type NiV-F protein, such as a wild-type NiV-F protein set forth in SEQ ID NO:2 or SEQ ID NO:7. In some embodiments, the mutant F protein contains an F1 subunit and an F2 subunit in which (1) the F1 subunit is truncated and lacks up to 22 contiguous amino acids at or near the C-terminus of the wild-type F1 subunit, such as lacks up to 22 contiguous amino acids at or near the C-terminus of the wild-type F1 subunit corresponding to amino acids 110-546 of NiV-F set forth in SEQ ID NO:2, and (2) the F2 subunit has the sequence corresponding to amino acid residues 27-109 of NiV-F set forth in SEQ ID NO:2.

In some embodiments, the F protein is a mutant NiV-F protein that is a biologically active portion thereof that comprises a 22 amino acid truncation at or near the C-terminus of the wild-type NiV-F protein (SEQ ID NO:2 or SEQ ID NO:7). In some embodiments, the NiV-F protein is encoded by a nucleotide sequence that encodes the sequence set forth in SEQ ID NO: 11. In some embodiments, the NiV-F protein has at least at or about 90%, at least at or about 91%, at least at or about 92%, at least at or about 93%, at least at or about 94%, at least at or about 95%, at or about 96%, at least at or about 97%, at least at or about 98%, or at least at or about 99% sequence identity to SEQ ID NO: 11. In particular embodiments, the F protein is a mutant NiV-F protein that has the sequence of amino acids set forth in SEQ ID NO:12. In some embodiments, the NiV-F protein has a sequence having at least at or about 90%, at least at or about 91%, at least at or about 92%, at least at or about 93%, at least at or about 94%, at least at or about 95%, at or about 96%, at least at or about 97%, at least at or about 98%, or at least at or about 99% sequence identity to SEQ ID NO: 12. In some embodiments, the F protein molecule or biologically active portion thereof comprises the sequence set forth in SEQ ID NO: 12.

In some embodiments, the mutant F protein contains an F1 subunit and an F2 subunit in which (1) the F1 subunit is set forth as amino acids 110-524 of SEQ ID NO:11, and (2) the F2 subunit is set forth as amino acids 27-109 of SEQ ID NO:11.

In some embodiments, the mutant F protein contains an F1 subunit and an F2 subunit in which (1) the F1 subunit is set forth as amino acids 84-498 of SEQ ID NO:12, and (2) the F2 subunit is set forth as amino acids 1-83 of SEQ ID NO:12.

### 2. Attachment (G/H/HN) Proteins

In some embodiments, the one or more paramyxovirus fusogen includes a paramyxovirus attachment glycoprotein (e.g. G protein). In some embodiments, the G protein is a Nipah (NiV) virus G-protein or a biologically active portion thereof. Exemplary G proteins or biologically active variants include any as described herein. Such G proteins, as well as variants thereof, also include any as disclosed in U.S. Patent Applications 63/291,316 and 63/291,323, each of whose disclosure is hereby incorporated by reference.

Paramyxoviral attachment proteins are type II transmembrane glycoproteins that are designated as hemagglutinin-neuraminidase (HN), hemagglutinin (H), or glycoproteins (G), depending on two characteristics; the ability to agglutinate erythrocytes (hemagglutination) and the presence or absence of neuraminidase activity (cleavage of sialic acid). Specifically, the HN attachment glycoprotein is characteristic of the Avulavirus, Respirovirus, and Rubulavirus genera, the H attachment glycoproteins are found in members of the Morbillivirus genus, while the G attachment glycoproteins are utilized by the viruses of the genus Henipavirus and the Pneumovirinae subfamily. The geometries of HN, H, or G glycoproteins possess high structural similarity, however although H and G glycoproteins are capable of recognizing protein receptors, they lack neuraminidase activity.

Paramyxoviral attachment glycoproteins contain a short N-terminal cytoplasmic tail, a transmembrane domain, and an extracellular domain containing an extracellular stalk and a globular head. The N-terminal cytoplasmic domain is within the inner lumen of the lipid bilayer and the C-terminal portion is the extracellular domain that is exposed on the outside of the lipid bilayer. The receptor binding and antigenic sites reside on the extracellular domain. Regions of the stalk in the C-terminal region have been shown to be involved in interactions with the F protein and triggering of fusion with a target cell membrane (Liu et al. 2015 J of Virology 89:1838). The F protein undergoes significant conformational change that facilitates the insertion of the fusion peptide into target membranes, bringing the two HR regions together in the formation of a six-helix bundle structure or trimer-of-hairpins during or immediately following fusion of virus and cell membranes (Bishop et al. 2008. J of Virology 82(22): 11398-11409). The cytoplasmic tails play a role in particle formation, incorporation into packaged particles, and serves as a signal peptide to modulate protein maturation and surface transport (Sawatsky et al. 2016. J of Virology 97:1066-1076).

In some embodiments, any of the provided lipid particles (lentiviral vectors) that contains a paramyxovirus attachment glycoprotein (e.g. G protein, such as NiV-G) or a biologically active portion thereof may also contain an F protein, such as a NiV-F protein, such as a full-length NiV-F protein or a biologically active portion thereof or a variant thereof.

In some embodiments, the envelope protein contains a henipavirus envelope attachment glycoprotein G (G protein) or a biologically active portion thereof. In some embodiments, the G protein may be retargeted by linkage to a targeting moiety, such as a binding molecule (e.g. antibody or antigen-binding fragment, e.g. sdAb or scFv) that binds to a target cell. In some embodiments, the G protein and the NiV-F protein provided herein together exhibit fusogenic activity to a target cell, such as to deliver an exogenous agent.

The attachment G proteins are type II transmembrane glycoproteins containing an N-terminal cytoplasmic tail (e.g. corresponding to amino acids 1-49 of SEQ ID NO:14), a transmembrane domain (e.g. corresponding to amino acids 50-70 of SEQ ID NO:14), and an extracellular domain containing an extracellular stalk (e.g. corresponding to amino acids 71-187 of SEQ ID NO:14), and a globular head (corresponding to amino acids 188-602 of SEQ ID NO:14). The N-terminal cytoplasmic domain is within the inner lumen of the lipid bilayer and the C-terminal portion is the extracellular domain that is exposed on the outside of the lipid bilayer. Regions of the stalk in the C-terminal region (e.g. corresponding to amino acids 71-187 of SEQ ID NO: 14) have been shown to be involved in interactions with F protein and triggering of F protein fusion (Liu et al. 2015 J of Virology 89:1838). In wild-type G protein, the globular head mediates receptor binding to henipavirus entry receptors Ephrin B2 and Ephrin B3, but is dispensable for membrane fusion (Brandel-Tretheway et al. Journal of Virology. 2019. 93(13)e00577-19). In some embodiments herein, tropism of the G protein is altered by linkage of the G protein or biologically active fragment thereof (e.g. cytoplasmic truncation) to a sdAb variable domain. Binding of the G protein to a binding partner can trigger fusion mediated by a compatible F protein or biologically active portion thereof. G protein sequences disclosed herein are predominantly disclosed as expressed sequences including an N-terminal methionine required for start of translation. As such N-terminal methionines are commonly cleaved co- or post-translationally, the mature protein sequences for all G protein sequences disclosed herein are also contemplated as lacking the N-terminal methionine.

G glycoproteins are highly conserved between henipavirus species. For example, the G protein of NiV and HeV viruses share 79% amino acids identity. Studies have shown a high degree of compatibility among G proteins with F proteins of different species as demonstrated by heterotypic fusion activation (Brandel-Tretheway et al. Journal of Virology. 2019). As described, a lipid particle can contain heterologous G and F proteins from different species. In particular embodiments, the F protein or the functionally active variant or biologically active portion thereof retains fusogenic activity in conjunction with a G protein as provided, such as any set forth below. Fusogenic activity includes the activity of the F protein in conjunction with a G protein to promote or facilitate fusion of two membrane lumens, such as the lumen of the lipid particle provided herein (e.g. having embedded in its lipid bilayer, such as exposed on its surface, a G protein and a F protein), and a cytoplasm of a target cell, e.g. a cell that contains a surface receptor or molecule that is recognized or bound by the G protein.

Exemplary Henipavirus protein G sequences are provided in Table 2

TABLE 2
Henipavirus protein G sequence clusters. Column 1, Genbank ID includes the Genbank
ID of the whole genome sequence of the virus that is the centroid sequence of the cluster.
Column 2, nucleotides of CDS provides the nucleotides corresponding to the CDS of the gene
in the whole genome. Column 3, Full Gene Name, provides the full name of the gene including
Genbank ID, virus species, strain, and protein name. Column 4, Sequence, provides the amino
acid sequence of the gene. Column 5, #Sequences/Cluster, provides the number of sequences
that cluster with this centroid sequence. Column 6 provides the SEQ ID numbers for the
described sequences.

| | Nucleo- | | | SEQ |
|---|---|---|---|---|
| Genbank | tides | Full sequence | | ID |
| ID | of CDS | ID | Sequence | NO |
| AF017149 | 8913-10727 | gb: AF017149\| | MMADSKLVSLNNNLSGKIKDQGKVIKN | 13 |
| | | Organism: | YYGTMDIKKINDGLLDSKILGAFNTVIA | |
| | | Hendra | LLGSIIIIVMNIMIIQNYTRTTDNQALIKE | |
| | | virus\|Strain | SLQSVQQQIKALTDKIGTEIGPKVSLIDT | |
| | | Name: | SSTITIPANIGLLGSKISQSTSSINENVND | |
| | | UNKNOWN- | KCKFTLPPLKIHECNISCPNPLPFREYRPI | |
| | | AF017149\| | SQGVSDLVGLPNQICLQKTTSTILKPRLI | |
| | | Protein | SYTLPINTREGVCITDPLLAVDNGFFAYS | |
| | | Name: | HLEKIGSCTRGIAKQRIIGVGEVLDRGD | |
| | | glycoprotein\| | KVPSMFMTNVWTPPNPSTIHHCSSTYHE | |
| | | Gene | DFYYTLCAVSHVGDPILNSTSWTESLSLI | |
| | | Symbol: | RLAVRPKSDSGDYNQKYIAITKVERGK | |
| | | G | YDKVMPYGPSGIKQGDTLYFPAVGFLP | |
| | | | RTEFQYNDSNCPIIHCKYSKAENCRLSM | |
| | | | GVNSKSHYILRSGLLKYNLSLGGDIILQF | |
| | | | IEIADNRLTIGSPSKIYNSLGQPVFYQAS | |
| | | | YSWDTMIKLGDVDTVDPLRVQWRNNS | |
| | | | VISRPGQSQCPRFNVCPEVCWEGTYND | |
| | | | AFLIDRLNWVSAGVYLNSNQTAENPVF | |
| | | | AVFKDNEILYQVPLAEDDTNAQKTITDC | |
| | | | FLLENVIWCISLVEIYDTGDSVIRPKLFA | |
| | | | VKIPAQCSES | |
| AF212302 | 8943-10751 | gb: AF212302\| | MPAENKKVRFENTTSDKGKIPSKVIKSY | 14 |
| | | Organism: | YGTMDIKKINEGLLDSKILSAFNTVIALL | |
| | | Nipah virus\|Strain | GSIVIIVMNIMIIQNYTRSTDNQAVIKDA | |
| | | Name: | LQGIQQQIKGLADKIGTEIGPKVSLIDTSS | |
| | | UNKNOWN- | TITIPANIGLLGSKISQSTASINENVNEKC | |
| | | AF212302\| | KFTLPPLKIHECNISCPNPLPFREYRPQTE | |
| | | Protein | GVSNLVGLPNNICLQKTSNQILKPKLISY | |
| | | Name: | TLPVVGQSGTCITDPLLAMDEGYFAYSH | |
| | | attachment | LERIGSCSRGVSKQRIIGVGEVLDRGDE | |
| | | glycoprotein\| | VPSLFMTNVWTPPNPNTVYHCSAVYNN | |
| | | Gene | EFYYVLCAVSTVGDPILNSTYWSGSLM | |
| | | Symbol: | MTRLAVKPKSNGGGYNQHQLALRSIEK | |
| | | G | GRYDKVMPYGPSGIKQGDTLYFPAVGF | |
| | | | LVRTEFKYNDSNCPITKCQYSKPENCRL | |
| | | | SMGIRPNSHYILRSGLLKYNLSDGENPK | |
| | | | VVFIEISDQRLSIGSPSKIYDSLGQPVFYQ | |
| | | | ASFSWDTMIKFGDVLTVNPLVVNWRNN | |
| | | | TVISRPGQSQCPRFNTCPEICWEGVYND | |
| | | | AFLIDRINWISAGVFLDSNQTAENPVFTV | |
| | | | FKDNEILYRAQLASEDTNAQKTITNCFL | |
| | | | LKNKIWCISLVEIYDTGDNVIRPKLFAV | |
| | | | KIPEQCT | |
| JQ001776 | 8170-10275 | gb: JQ001776: | MLSQLQKNYLDNSNQQGDKMNNPDKK | 15 |
| | | 8170-10275\| | LSVNFNPLELDKGQKDLNKSYYVKNKN | |
| | | Organism: | YNVSNLLNESLHDIKFCIYCIFSLLIIITIIN | |
| | | Cedar | IITISIVITRLKVHEENNGMESPNLQSIQD | |
| | | virus\|Strain | SLSSLTNMINTEITPRIGILVTATSVTLSSS | |
| | | Name: | INYVGTKTNQLVNELKDYITKSCGFKVP | |
| | | CG1a\|Protein | ELKLHECNISCADPKISKSAMYSTNAYA | |
| | | Name: | ELAGPPKIFCKSVSKDPDFRLKQIDYVIP | |
| | | attachment | VQQDRSICMNNPLLDISDGFFTYIHYEGI | |
| | | glycoprotein\| | NSCKKSDSFKVLLSHGEIVDRGDYRPSL | |
| | | Gene | YLLSSHYHPYSMQVINCVPVTCNQSSFV | |
| | | Symbol: | FCHISNNTKTLDNSDYSSDEYYITYFNGI | |
| | | G | DRPKTKKIPINNMTADNRYIHFTFSGGG | |
| | | | GVCLGEEFIIPVTTVINTDVFTHDYCESF | |
| | | | NCSVQTGKSLKEICSESLRSPINSSRYNL | |
| | | | NGIMIISQNNMTDFKIQLNGITYNKLSFG | |
| | | | SPGRLSKTLGQVLYYQSSMSWDTYLKA | |
| | | | GFVEKWKPFTPNWMNNTVISRPNQGNC | |
| | | | PRYHKCPEICYGGTYNDIAPLDLGKDM | |
| | | | YVSVILDSDQLAENPEITVENSTTILYKE | |
| | | | RVSKDELNTRSTTTSCFLFLDEPWCISVL | |
| | | | ETNRFNGKSIRPEIYSYKIPKYC | |
| NC_025256 | 9117-11015 | gb: NC_025256: | MPQKTVEFINMNSPLERGVSTLSDKKTL | 16 |
| | | 9117-11015\| | NQSKITKQGYFGLGSHSERNWKKQKNQ | |
| | | Organism: | NDHYMTVSTMILEILVVLGIMENLIVLT | |
| | | Bat | MVYYQNDNINQRMAELTSNITVLNLNL | |
| | | Paramyxovirus | NQLTNKIQREIIPRITLIDTATTITIPSAITY | |
| | | Eid_hel/GH- | ILATLTTRISELLPSINQKCEFKTPTLVLN | |
| | | M74a/GHA/2009\| | DCRINCTPPLNPSDGVKMSSLATNLVAH | |
| | | Strain | GPSPCRNFSSVPTIYYYRIPGLYNRTALD | |
| | | Name: | ERCILNPRLTISSTKFAYVHSEYDKNCTR | |
| | | BatPV/ | GFKYYELMTFGEILEGPEKEPRMFSRSF | |
| | | Eid_hel/GH- | YSPTNAVNYHSCTPIVTVNEGYFLCLEC | |
| | | M74a/GHA/2009\| | TSSDPLYKANLSNSTFHLVILRHNKDEKI | |
| | | Protein | VSMPSFNLSTDQEYVQIIPAEGGGTAES | |
| | | Name: | GNLYFPCIGRLLHKRVTHPLCKKSNCSR | |
| | | glycoprotein\| | TDDESCLKSYYNQGSPQHQVVNCLIRIR | |
| | | Gene | NAQRDNPTWDVITVDLTNTYPGSRSRIF | |
| | | Symbol: | GSFSKPMLYQSSVSWHTLLQVAEITDLD | |
| | | G | KYQLDWLDTPYISRPGGSECPFGNYCPT | |
| | | | VCWEGTYNDVYSLTPNNDLFVTVYLKS | |
| | | | EQVAENPYFAIFSRDQILKEFPLDAWISS | |
| | | | ARTTTISCFMFNNEIWCIAALEITRLNDD | |
| | | | IIRPIYYSFWLPTDCRTPYPHTGKMTRVP | |
| | | | LRSTYNY | |
| NC_025352 | 8716-11257 | gb: NC_025352: | MATNRDNTITSAEVSQEDKVKKYYGVE | 17 |
| | | 8716-11257\| | TAEKVADSISGNKVFILMNTLLILTGAIIT | |
| | | Organism: | ITLNITNLTAAKSQQNMLKIIQDDVNAK | |
| | | Mojiang | LEMFVNLDQLVKGEIKPKVSLINTAVSV | |
| | | virus\|Strain | SIPGQISNLQTKFLQKYVYLEESITKQCT | |
| | | Name: | CNPLSGIFPTSGPTYPPTDKPDDDTTDDD | |
| | | Tongguan1\| | KVDTTIKPIEYPKPDGCNRTGDHFTMEP | |
| | | Protein | GANFYTVPNLGPASSNSDECYTNPSFSIG | |
| | | Name: | SSIYMFSQEIRKTDCTAGEILSIQIVLGRI | |
| | | attachment | VDKGQQGPQASPLLVWAVPNPKIINSCA | |
| | | glycoprotein\| | VAAGDEMGWVLCSVTLTAASGEPIPHM | |
| | | Gene | FDGFWLYKLEPDTEVVSYRITGYAYLL | |
| | | Symbol: | DKQYDSVFIGKGGGIQKGNDLYFQMYG | |
| | | G | LSRNRQSFKALCEHGSCLGTGGGGYQV | |
| | | | LCDRAVMSFGSEESLITNAYLKVNDLAS | |
| | | | GKPVIIGQTFPPSDSYKGSNGRMYTIGD | |
| | | | KYGLYLAPSSWNRYLRFGITPDISVRSTT | |
| | | | WLKSQDPIMKILSTCTNTDRDMCPEICN | |
| | | | TRGYQDIFPLSEDSEYYTYIGITPNNGGT | |
| | | | KNFVAVRDSDGHIASIDILQNYYSITSAT | |
| | | | ISCFMYKDEIWCIAITEGKKQKDNPQRIY | |
| | | | AHSYKIRQMCYNMKSATVTVGNAKNIT | |
| | | | IRRY | |

In some embodiments, the G protein has a sequence set forth in any of SEQ ID NOS: 14, 13, 15, 16 or 17 or is a functionally active variant or biologically active portion thereof that has a sequence that is at least at or about 80%, at least at or about 81%, at least at or about 82%, at least at or about 83%, at least at or about 84%, at least at or about 85%, at least at or about 86%, at least at or about 87%, at least at or about 88%, at least at or about 89%, at least at or about 90%, at least at or about 91%, at least at or about 92%, at least at or about 93%, at least at or about 94%, at least at or about 95%, at least at or about 96%, at least at or about 97%, at least at or about 98%, or at least at or about 99% identical to any one of SEQ ID NOS: 14, 13, 15, 16 or 17.

In particular embodiments, the G protein or functionally active variant or biologically active portion is a protein that retains fusogenic activity in conjunction with a Henipavirus F protein, such as a NiV-F protein described herein. Fusogenic activity includes the activity of the G protein in conjunction with a Henipavirus F protein to promote or facilitate fusion of two membrane lumens, such as the lumen of the targeted lipid particle having embedded in its lipid bilayer a henipavirus F and G protein, and a cytoplasm of a target cell, e.g. a cell that contains a surface receptor or molecule that is recognized or bound by the targeted envelope protein. In some embodiments, the F protein and G protein are from the same Henipavirus species (e.g. NiV-G and NiV-F).

In some embodiments the G protein is a mutant G protein that is a functionally active variant or biologically active portion containing one or more amino acid mutations, such as one or more amino acid insertions, deletions, substitutions or truncations. In some embodiments, the mutations described herein relate to amino acid insertions, deletions, substitutions or truncations of amino acids compared to a reference G protein sequence. In some embodiments, the reference G protein sequence is the wild-type sequence of a G protein or a biologically active portion thereof. In some embodiments, the functionally active variant or the biologically active portion thereof is a mutant of a wild-type Hendra (HeV) virus G protein, a wild-type Nipah (NiV) virus G-protein (NiV-G), a wild-type Cedar (CedPV) virus G-protein, a wild-type Mojiang virus G-protein, a wild-type bat Paramyxovirus G-protein or biologically active portion thereof. In some embodiments, the wild-type G protein has the sequence set forth in any one of SEQ ID NOS: 14, 13, 15, 16 or 17.

In some embodiments, the G protein is a mutant G protein that is a biologically active portion that is an N-terminally and/or C-terminally truncated fragment of a wild-type Hendra (HeV) virus G protein, a wild-type Nipah (NiV) virus G-protein (NiV-G), a wild-type Cedar (CedPV) virus G-protein, a wild-type Mojiang virus G-protein, a wild-type bat Paramyxovirus G-protein. In particular embodiments, the truncation is an N-terminal truncation of all or a portion of the cytoplasmic domain. In some embodiments, the mutant G protein is a biologically active portion that is truncated and lacks up to 49 contiguous amino acid residues at or near the N-terminus of the wild-type G protein, such as a wild-type G protein set forth in any one of SEQ ID NOS: 14, 13, 15, 16 or 17. In some embodiments, the mutant G protein is truncated and lacks up to 49 contiguous amino acids, such as up to 49, 48, 47, 46, 45, 44, 43, 42, 41, 40, 30, 38, 37, 36, 35, 34, 33, 32, 31, 30, 29, 28, 27, 26, 25, 24, 23, 22, 21, 20, 19, 18, 17, 16, 15, 14, 13, 12, 11, 10, 9, 8, 7, 6, 5, 4, 3, 2 or 1 contiguous amino acids at the N-terminus of the wild-type G protein.

In some embodiments, the G protein is a wild-type Nipah virus G (NiV-G) protein or a Hendra virus G protein, or is a functionally active variant or biologically active portion thereof. In some embodiments, the G protein is a NiV-G protein that has the sequence set forth in SEQ ID NO:14, or is a functional variant or a biologically active portion thereof that has an amino acid sequence having at least at or about 80%, at least at or about 81%, at least at or about 82%, at least at or about 83%, at least at or about 84%, at least at or about 85%, at least at or about 86%, at least at or about 87%, at least at or about 88%, at least at or about 89%, at least at or about 90%, at least at or about 91%, at least at or about 92%, at least at or about 93%, at least at or about 94%, at least at or about 95%, at or about 96%, at least at or about 97%, at least at or about 98%, at least at or about 99% sequence identity to SEQ ID NO:14.

In some embodiments, the G protein is a mutant NiV-G protein that is a biologically active portion of a wild-type NiV-G. In some embodiments, the biologically active portion is an N-terminally truncated fragment. In some embodiments, the mutant NiV-G protein is truncated and lacks up to 5 contiguous amino acid residues at or near the N-terminus of the wild-type NiV-G protein, such as compared to wild-type NiV-G set forth in SEQ ID NO: 14. In some embodiments, the mutant NiV-G protein is truncated and lacks up to 10 contiguous amino acid residues at or near the N-terminus of the wild-type NiV-G protein, such as compared to wild-type NiV-G set forth in SEQ ID NO: 14. In some embodiments, the mutant NiV-G protein is truncated and lacks up to 15 contiguous amino acid residues at or near the N-terminus of the wild-type NiV-G protein, such as compared to wild-type NiV-G set forth in SEQ ID NO: 14. In some embodiments, the mutant NiV-G protein is truncated and lacks up to 20 contiguous amino acid residues at or near the N-terminus of the wild-type NiV-G protein, such as compared to wild-type NiV-G set forth in SEQ ID NO: 14. In some embodiments, the mutant NiV-G protein is truncated and lacks up to 25 contiguous amino acid residues at or near the N-terminus of the wild-type NiV-G protein, such as compared to wild-type NiV-G set forth in SEQ ID NO: 14. In some embodiments, the mutant NiV-G protein is truncated and lacks up to 30 contiguous amino acid residues at or near the N-terminus of the wild-type NiV-G protein, such as compared to wild-type NiV-G set forth in SEQ ID NO: 14. In some embodiments, the mutant NiV-G protein is truncated and lacks up to 35 contiguous amino acid residues at or near the N-terminus of the wild-type NiV-G protein, such as compared to wild-type NiV-G set forth in SEQ ID NO: 14. In some embodiments, the mutant NiV-G protein (also called variant NiV-G) contains an N-terminal methionine.

In some embodiments, the mutant NiV-G protein is truncated and lacks up to amino acid 34 at or near the N-terminus of the wild-type NiV-G protein, such as compared to wild-type NiV-G set forth in SEQ ID NO: 14. In some embodiments, the mutant NiV-G protein (also called variant NiV-G) contains an N-terminal methionine. In some embodiments, the mutant NiV-G protein lacks amino acids 2-34 as compared to wild-type NiV-G set forth in SEQ ID NO: 14.

In some embodiments, the G protein or the functionally active variant or biologically active portion thereof binds to Ephrin B2 or Ephrin B3. In some embodiments, the G protein is a mutant G protein, such as a truncated G protein as described and retains binding to Ephrin B2 or B3. Reference to retaining binding to Ephrin B2 or B3 includes binding that is similar to the level or degree of binding of the corresponding wild-type G protein, such as set forth in SEQ ID NO: 14, 13, 15, 16 or 17, such as at least 50%, at least 60%, at least 70%, at least 80% or at least 90% of the binding of the wild-type G protein.

In some embodiments, the G protein or the biologically thereof is a mutant G protein that exhibits reduced binding for the native binding partner of a wild-type G protein. In some embodiments, the mutant G protein or the biologically active portion thereof is a mutant of wild-type Niv-G and exhibits reduced binding to one or both of the native binding partners Ephrin B2 or Ephrin B3. In some embodiments, the mutant G-protein or the biologically active portion, such as a mutant NiV-G protein, exhibits reduced binding to the native binding partner. In some embodiments, the reduced binding to Ephrin B2 or Ephrin B3 is reduced by greater than at or about 5%, at or about 10%, at or about 15%, at or about 20%, at or about 25%, at or about 30%, at or about 40%, at or about 50%, at or about 60%, at or about 70%, at or about 80%, at or about 90%, or at or about 100%.

In some embodiments, the mutations can improve transduction efficiency. In some embodiments, the mutations allow for specific targeting of other desired cell types that are not Ephrin B2 or Ephrin B3. In some embodiments, the mutations result in at least the partial inability to bind at least one natural receptor, such as reduce the binding to at least one of Ephrin B2 or Ephrin B3. In some embodiments, the mutations described herein interfere with natural receptor recognition.

In some embodiments, the G protein contains one or more amino acid substitutions in a residue that is involved in the interaction with one or both of Ephrin B2 and Ephrin B3. In some embodiments, the amino acid substitutions correspond to mutations E501A, W504A, Q530A and E533A with reference to numbering set forth in SEQ ID NO:14. In some embodiments, the G protein is a mutant G protein containing one or more amino acid substitutions selected from the group consisting of E501A, W504A, Q530A and E533A with reference to numbering set forth in SEQ ID NO:14. In some embodiments, the G protein is a mutant G protein that contains one or more amino acid substitutions elected from the group consisting of E501A, W504A, Q530A and E533A with reference to SEQ ID NO:14 and is a biologically active portion thereof containing an N-terminal truncation.

In particular embodiments, the G protein has the sequence of amino acids set forth in SEQ ID NO: 19, or is a functionally active variant thereof or a biologically active portion thereof that retains binding and/or fusogenic activity. In some embodiments, the functionally active variant comprises an amino acid sequence having at least at or about 80%, at least at or about 85%, at least at or about 90%, at least at or about 91%, at least at or about 92%, at least at or about 93%, at least at or about 94%, at least at or about 95%, at or about 96%, at least at or about 97%, at least at or about 98%, or at least at or about 99% sequence identity to SEQ ID NO: 19 and retains fusogenic activity in conjunction with a NiV-F protein as described.

Reference to retaining fusogenic activity includes activity of a lipid particle (e.g. lentiviral vector) containing a variant NiV-F protein as described or biologically active portion or functionally active variant of the F protein (in conjunction with a G protein, such as a NiV-G protein as described) that is between at or about 10% and at or about 150% or more of the level or degree of binding of a reference lipid particle (e.g. lentiviral vector) that is similar, such as contains the same variant NiV-F, but that contains the corresponding wild-type G protein, such as set forth in SEQ ID NO: 14. For instance, a lipid particle (e.g. lentiviral vector) that retains fusogenic activity has at least or at least about 10% of the level or degree of fusogenic activity of the reference lipid particle that is similar (such as contains the same variant NiV-F) but that contains the corresponding wild-type G protein, such as at least or at least about 15% of the level or degree of fusogenic activity, at least or at least about 20% of the level or degree of fusogenic activity, at least or at least about 25% of the level or degree of fusogenic activity, at least or at least about 30% of the level or degree of fusogenic activity, at least or at least about 35% of the level or degree of fusogenic activity, at least or at least about 40% of the level or degree of fusogenic activity, at least or at least about 45% of the level or degree of fusogenic activity, at least or at least about 50% of the level or degree of fusogenic activity, at least or at least about 55% of the level or degree of fusogenic activity, at least or at least about 60% of the level or degree of fusogenic activity, at least or at least about 65% of the level or degree of fusogenic activity, at least or at least about 70% of the level or degree of fusogenic activity, at least or at least about 75% of the level or degree of fusogenic activity, at least or at least about 80% of the level or degree of fusogenic activity, at least or at least about 85% of the level or degree of fusogenic activity, at least or at least about 90% of the level or degree of fusogenic activity, at least or at least about 95% of the level or degree of fusogenic activity, at least or at least about 100% of the level or degree of fusogenic activity, or at least or at least about 120% of the level or degree of fusogenic activity.

3. Re-Targeted Fusogens (e.g. Re-Targeted G Proteins)

In some embodiments, the fusogen (e.g. F or G protein) is a targeted envelope protein that contains a vector-surface targeting moiety. In some embodiments, the vector-surface targeting moiety binds a target ligand. In some embodiments, the target ligand can be expressed on a target cell of interest, such as a target cell present as a leukocyte component. In some aspects, a fusogen can be retargeted to display altered tropism. In some embodiments, the binding confers re-targeted binding compared to the binding of a wild-type surface glycoprotein protein in which a new or different binding activity is conferred.

In some embodiments, a G protein (such as NiV-G) is further attached or linked to a binding domain that binds to a target molecule, such as a cell surface marker. For instance, provided in some aspects is a targeted lipid particle (e.g. targeted lentiviral vector) that includes a re-targeted G protein containing any of the provided G proteins attached to a binding domain, in which the re-targeted G protein is exposed on the surface of the targeted lipid particle (e.g. targeted lentiviral vector). In particular embodiments, the fusogen (e.g. G protein) is mutated to reduce binding for the native binding partner of the fusogen. In some embodiments, the fusogen is or contains a mutant G protein or a biologically active portion thereof that is a mutant of wild-type NiV-G and exhibits reduced binding to one or both of the native binding partners Ephrin B2 or Ephrin B3, including any as described above. In particular embodiments, the binding confers re-targeted binding compared to the binding of a wild-type G protein in which a new or different binding activity is conferred.

In some embodiments, the targeted envelope protein contains a G protein provided herein.

In some embodiments the G protein is any as described above, including NiV-G proteins with cytoplasmic domain modifications, truncated NiV-G cytoplasmic tails, or modified NiV-G cytoplasmic tails.

In some embodiments, the binding domain can be any agent that binds to a cell surface molecule on a target cells. In some embodiments, protein fusogens may be re-targeted by covalently conjugating a targeting-moiety to the fusion protein. In some embodiments, the fusogen and targeting moiety are covalently conjugated by expression of a chimeric protein comprising the fusogen linked to the targeting moiety. In some embodiments, a target includes any peptide (e.g. a receptor) that is displayed on a target cell. In some embodiments, the target is expressed at higher levels on a target cell than non-target cells. In some embodiments, a single-chain variable fragment (scFv) can be conjugated to fusogens to redirect fusion activity towards cells that display the scFv binding target (doi:10.1038/nbt1060, DOI 10.1182/blood-2012-11-468579, doi:10.1038/nmeth.1514, doi:10.1006/mthe.2002.0550, HUMAN GENE THERAPY 11:817-826, doi:10.1038/nbt942, doi:10.1371/journal.pone.0026381, DOI 10.1186/s12896-015-0142-z). In some embodiments, designed ankyrin repeat proteins (DARPin) can be conjugated to fusogens to redirect fusion activity towards cells that display the DARPin binding target (doi:10.1038/mt.2013.16, doi:10.1038/mt.2010.298, doi: 10.4049/jimmunol.1500956), as well as combinations of different DARPins (doi:10.1038/mto.2016.3). In some embodiments, receptor ligands and antigens can be conjugated to fusogens to redirect fusion activity towards cells that display the target receptor (DOI: 10.1089/hgtb.2012.054, DOI: 10.1128/JVI.76.7.3558-3563.2002). In some embodiments, a targeting protein can also include an antibody or an antigen-binding fragment thereof (e.g., Fab, Fab′, F(ab′)2, Fv fragments, scFv antibody fragments, disulfide-linked Fvs (sdFv), a Fd fragment consisting of the VH and CH1 domains, linear antibodies, single domain antibodies such as sdAb (either VL or VH), nanobodies, or camelid VHH domains), an antigen-binding fibronectin type III (Fn3) scaffold such as a fibronectin polypeptide minibody, a ligand, a cytokine, a chemokine, or a T cell receptor (TCRs). In some embodiments, protein fusogens may be re-targeted by non-covalently conjugating a targeting moiety to the fusion protein or targeting protein (e.g. the hemagglutinin protein). In some embodiments, the fusion protein can be engineered to bind the Fc region of an antibody that targets an antigen on a target cell, redirecting the fusion activity towards cells that display the antibody's target (DOI: 10.1128/JVI.75.17.8016-8020.2001, doi:10.1038/nml192). In some embodiments, altered and non-altered fusogens may be displayed on the same retroviral vector or VLP (doi: 10.1016/j.biomaterials.2014.01.051).

In some embodiments, a targeting moiety comprises a humanized antibody molecule, intact IgA, IgG, IgE or IgM antibody; bi- or multi-specific antibody (e.g., Zybodies®, etc); antibody fragments such as Fab fragments, Fab′ fragments, F(ab′)2 fragments, Fd′ fragments, Fd fragments, and isolated CDRs or sets thereof; single chain Fvs; polypeptide-Fc fusions; single domain antibodies (e.g., shark single domain antibodies such as IgNAR or fragments thereof); cameloid antibodies; masked antibodies (e.g., Probodies®); Small Modular ImmunoPharmaceuticals (“SMIPsTM”); single chain or Tandem diabodies (TandAb®); VHHs; Anticalins®; Nanobodies®; minibodies; BiTE®s; ankyrin repeat proteins or DARPINs®; Avimers®; DARTs; TCR-like antibodies; Adnectins®; Affilins®; Trans-bodies®; Affibodies®; TrimerX®; MicroProteins; Fynomers®, Centyrins®; and KALBITOR®s.

In some embodiments, the targeting moiety is a binding domain that can be an antibody or an antibody portion or fragment. In some embodiments, the binding domain is a single domain antibody (sdAb). In some embodiments, the binding domain is a single chain variable fragment (scFv). In some examples, the binding domain can be linked directly or indirectly to the G protein (e.g. NiV-G or a biologically active portion). In particular embodiments, the binding domain is linked to the C-terminus (C-terminal amino acid) of the G protein or the biologically active portion thereof. The linkage can be via a peptide linker, such as a flexible peptide linker.

The binding domain may be modulated to have different binding strengths. For example, scFvs and antibodies with various binding strengths may be used to alter the fusion activity of the chimeric attachment proteins towards cells that display high or low amounts of the target antigen. For example DARPins with different affinities may be used to alter the fusion activity towards cells that display high or low amounts of the target antigen. Binding domains may also be modulated to target different regions on the target ligand, which will affect the fusion rate with cells displaying the target.

The binding domain may comprise a humanized antibody molecule, intact IgA, IgG, IgE or IgM antibody; bi- or multi-specific antibody (e.g., Zybodies®, etc); antibody fragments such as Fab fragments, Fab′ fragments, F(ab′)2 fragments, Fd′ fragments, Fd fragments, and isolated CDRs or sets thereof; single chain Fvs; polypeptide-Fc fusions; single domain antibodies (e.g., shark single domain antibodies such as IgNAR or fragments thereof); cameloid antibodies; masked antibodies (e.g., Probodies®); Small Modular ImmunoPharmaceuticals (“SMIPsTM”); single chain or Tandem diabodies (TandAb®); VHHs; Anticalins®; Nanobodies®; minibodies; BiTE®s; ankyrin repeat proteins or DARPINs®; Avimers®; DARTs; TCR-like antibodies; Adnectins®; Affilins®; Trans-bodies®; Affibodies®; TrimerX®; MicroProteins; Fynomers®, Centyrins®; and KALBITOR®s. A targeting moiety can also include an antibody or an antigen-binding fragment thereof (e.g., Fab, Fab′, F(ab′)2, Fv fragments, scFv antibody fragments, disulfide-linked Fvs (sdFv), a Fd fragment consisting of the VH and CH1 domains, linear antibodies, single domain antibodies such as sdAb (either VL or VH), nanobodies, or camelid VHH domains), an antigen-binding fibronectin type III (Fn3) scaffold such as a fibronectin polypeptide minibody, a ligand, a cytokine, a chemokine, or a T cell receptor (TCRs).

In some embodiments, the binding domain is a single chain molecule. In some embodiments, the binding domain is a single domain antibody. In some embodiments, the binding domain is a single chain variable fragment. In particular embodiments, the binding domain contains an antibody variable sequence (s) that is human or humanized.

In some embodiments, the binding domain is a single domain antibody. In some embodiments, the single domain antibody can be human or humanized. In some embodiments, the single domain antibody or portion thereof is naturally occurring. In some embodiments, the single domain antibody or portion thereof is synthetic.

In some embodiments, the single domain antibodies are antibodies whose complementary determining regions are part of a single domain polypeptide. In some embodiments, the single domain antibody is a heavy chain only antibody variable domain. In some embodiments, the single domain antibody does not include light chains.

In some embodiments, the heavy chain antibody devoid of light chains is referred to as VHH. In some embodiments, the single domain antibody antibodies have a molecular weight of 12-15 kDa. In some embodiments, the single domain antibody antibodies include camelid antibodies or shark antibodies. In some embodiments, the single domain antibody molecule is derived from antibodies raised in Camelidae species, for example in camel, llama, dromedary, alpaca, vicuna and guanaco. In some embodiments, the single domain antibody is referred to as immunoglobulin new antigen receptors (IgNARs) and is derived from cartilaginous fishes. In some embodiments, the single domain antibody is generated by splitting dimeric variable domains of human or mouse IgG into monomers and camelizing critical residues.

In some embodiments, the single domain antibody can be generated from phage display libraries. In some embodiments, the phage display libraries are generated from a VHH repertoire of camelids immunized with various antigens, as described in Arbabi et al., FEBS Letters, 414, 521-526 (1997); Lauwereys et al., EMBO J., 17, 3512-3520 (1998); Decanniere et al., Structure, 7, 361-370 (1999). In some embodiments, the phage display library is generated comprising antibody fragments of a non-immunized camelid. In some embodiments, single domain antibodies a library of human single domain antibodies is synthetically generated by introducing diversity into one or more scaffolds.

In some embodiments, the C-terminus of the binding domain is attached to the C-terminus of the G protein or biologically active portion thereof. In some embodiments, the N-terminus of the binding domain is exposed on the exterior surface of the lipid bilayer. In some embodiments, the N-terminus of the binding domain binds to a cell surface molecule of a target cell. In some embodiments, the binding domain specifically binds to a cell surface molecule present on a target cell. In some embodiments, the cell surface molecule is a protein, glycan, lipid or low molecular weight molecule. In some embodiments, the binding domain is one of any binding domains as described above.

In embodiments, the re-targeted fusogen binds a cell surface marker on the target cell, e.g., a protein, glycoprotein, receptor, cell surface ligand, agonist, lipid, sugar, class I transmembrane protein, class II transmembrane protein, or class III transmembrane protein. In some embodiments, a binding domain (e.g. sdAb or one of any binding domains as described herein) binds to a cell surface antigen of a cell. In some embodiments, a cell surface antigen is characteristic of one type of cell. In some embodiments, a cell surface antigen is characteristic of more than one type of cell.

In some embodiments, the cell surface molecule of a target cell is an antigen or portion thereof. In some embodiments, the single domain antibody or portion thereof is an antibody having a single monomeric domain antigen binding/recognition domain that is able to bind selectively to a specific antigen. In some embodiments, the single domain antibody binds an antigen present on a target cell.

Exemplary target cells include cells present in a blood sample from a subject. In some embodiments, the cells include a leukocyte component. In some embodiments, the target cells include polymorphonuclear cells (also known as PMN, PML, PMNL, or granulocytes), In some embodiments, the target cells include lymphocytes, monocytes, macrophages, dendritic cells, natural killer cells, T cells (e.g. CD4 or CD8 T cells including cytotoxic T lymphocytes) or B cells. In some embodiments, the target cells include hematopoietic stem cells (HSCs).

In some embodiments, the target cell is a CD3+ T cell, a CD4+ T cell, a CD8+ T cell.

In some embodiments, the target cell is an antigen presenting cell, an MHC class II+ cell, a professional antigen presenting cell, an atypical antigen presenting cell, a macrophage, a dendritic cell, a myeloid dendritic cell, a plasmacyteoid dendritic cell, a CD11c+ cell, a CD11b+ cell, or a B cell.

In some embodiments, the binding domain (e.g. sdAb) variable domain binds a cell surface molecule or antigen. In some embodiments, the cell surface molecule is ASGR1, ASGR2, TM4SF5, CD3, CD8, CD4, or low density lipoprotein receptor (LDL-R). In some embodiments, the cell surface molecule is ASGR1. In some embodiments, the cell surface molecule is ASGR2. In some embodiments, the cell surface molecule is TM4SF5. In some embodiments, the cell surface molecule is CD3. In some embodiments, the cell surface molecule is CD8. In some embodiments, the cell surface molecule is CD4. In some embodiments, the cell surface molecule is LDL-R.

The viral vectors disclosed herein include one or more CD4 binding agents. For example, a CD4 binding agent may be fused to or incorporated in a protein fusogen or viral envelope protein. In another embodiment, a CD4 binding agent may be incorporated into the viral envelope via fusion with a transmembrane domain.

Exemplary CD4 binding agents include antibodies and fragments thereof (e.g., scFv, VHH) that bind to CD4. Such antibodies may be derived from any species, and may be for example, mouse, rabbit, human, humanized, or camelid antibodies. Exemplary antibodies include ibalizumab, zanolimumab, tregalizumab, priliximab, cedelizumab, clenoliximab, keliximab, and anti-CD4 antibodies disclosed in WO2002102853, WO2004083247, WO2004067554, WO2007109052, WO2008134046, WO2010074266, WO2012113348, WO2013188870, WO2017104735, WO2018035001, WO2018170096, WO2019203497, WO2019236684, WO2020228824, U.S. Pat. Nos. 5,871,732, 7,338,658, 7,722,873, 8,399,621, 8,911,728, 9,587,022, 9,745,552; as well as antibodies B486A1, RPA-T4, CE9.1 (Novus Biologicals); GK1.5, RM4-5, RPA-T4, OKT4, 4SM95, S3.5, N1UG0 (ThermoFisher); GTX50984, ST0488, 10BS, EP204 (GeneTex); GK1.3, 5A8, 10C12, W3/25, 8A5, 13B8.2, 6G5 (Absolute Antibody); VIT4, M-T466, M-T321, REA623, (Miltenyi); MEM115, MT310 (Enzo Life Sciences); H129.19, 5B4, 6A17, 18-46, A-1, C-1, OX68 (Santa Cruz); EP204, D2E6M (Cell Signaling Technology). Other exemplary binding agents include designed ankyrin repeat proteins (DARPins) (e.g., the anti-CD4 DARPin disclosed in WO2017182585) and binding agents based on fibronectin type III (Fn3) scaffolds.

In some embodiments, protein fusogens or viral envelope proteins may be re-targeted by mutating amino acid residues in a fusion protein or a targeting protein (e.g. the hemagglutinin (H) protein or G protein). In particular embodiments, the fusogen (e.g. G protein) is mutated to reduce binding for the native binding partner of the fusogen. In some embodiments, the fusogen is or contains a mutant G protein or a biologically active portion thereof that is a mutant of wild-type Niv-G and exhibits reduced binding to one or both of the native binding partners Ephrin B2 or Ephrin B3, including any as described above. Thus, in some aspects, a fusogen can be retargeted to display altered tropism. In some embodiments, the binding confers re-targeted binding compared to the binding of a wild-type surface glycoprotein protein in which a new or different binding activity is conferred. In particular embodiments, the binding confers re-targeted binding compared to the binding of a wild-type G protein in which a new or different binding activity is conferred. In some embodiments the fusogen is randomly mutated. In some embodiments the fusogen is rationally mutated. In some embodiments the fusogen is subjected to directed evolution. In some embodiments the fusogen is truncated and only a subset of the peptide is used in the viral vector. In some embodiments, amino acid residues in the measles hemagglutinin protein may be mutated to alter the binding properties of the protein, redirecting fusion (doi:10.1038/nbt942, Molecular Therapy vol. 16 no. 8, 1427-1436 August 2008, doi:10.1038/nbt1060, DOI: 10.1128/JVI.76.7.3558-3563.2002, DOI: 10.1128/JVI.75.17.8016-8020.2001, doi: 10.1073pnas.0604993103).

In some embodiments, protein fusogens may be re-targeted by covalently conjugating a CD4 binding agent to the fusion protein or targeting protein (e.g. the hemagglutinin protein). In some embodiments, the fusogen and CD4 binding agent are covalently conjugated by expression of a chimeric protein comprising the fusogen linked to the CD4 binding agent. In some embodiments, a single-chain variable fragment (scFv) can be conjugated to fusogens to redirect fusion activity towards cells that display the scFv binding target (doi:10.1038/nbt1060, DOI 10.1182/blood-2012-11-468579, doi:10.1038/nmeth.1514, doi:10.1006/mthe.2002.0550, HUMAN GENE THERAPY 11:817-826, doi:10.1038/nbt942, doi:10.1371/journal.pone.0026381, DOI 10.1186/s12896-015-0142-z). In some embodiments, designed ankyrin repeat proteins (DARPin) can be conjugated to fusogens to redirect fusion activity towards cells that display the DARPin binding target (doi:10.1038/mt.2013.16, doi:10.1038/mt.2010.298, doi: 10.4049/jimmunol.1500956), as well as combinations of different DARPins (doi:10.1038/mto.2016.3). In some embodiments, receptor ligands and antigens can be conjugated to fusogens to redirect fusion activity towards cells that display the target receptor (DOI: 10.1089/hgtb.2012.054, DOI: 10.1128/JVI.76.7.3558-3563.2002). In some embodiments, a targeting protein can also include an antibody or an antigen-binding fragment thereof (e.g., Fab, Fab′, F(ab′)2, Fv fragments, scFv antibody fragments, disulfide-linked Fvs (sdFv), a Fd fragment consisting of the VH and CH1 domains, linear antibodies, single domain antibodies such as sdAb (either VL or VH), nanobodies, or camelid VHH domains), an antigen-binding fibronectin type III (Fn3) scaffold such as a fibronectin polypeptide minibody, a ligand, a cytokine, a chemokine, or a T cell receptor (TCRs). In some embodiments, protein fusogens may be re-targeted by non-covalently conjugating a CD4 binding agent to the fusion protein or targeting protein (e.g. the hemagglutinin protein). In some embodiments, the fusion protein can be engineered to bind the Fc region of an antibody that targets an antigen on a target cell, redirecting the fusion activity towards cells that display the antibody's target (DOI: 10.1128/JVI.75.17.8016-8020.2001, doi:10.1038/nml192). In some embodiments, altered and non-altered fusogens may be displayed on the same retroviral vector or VLP (doi: 10.1016/j.biomaterials.2014.01.051).

In some embodiments, a CD4 binding agent comprises a humanized antibody molecule, intact IgA, IgG, IgE or IgM antibody; bi- or multi-specific antibody (e.g., Zybodies®, etc); antibody fragments such as Fab fragments, Fab′ fragments, F(ab′)₂ fragments, Fd′ fragments, Fd fragments, and isolated CDRs or sets thereof; single chain Fvs; polypeptide-Fc fusions; single domain antibodies (e.g., shark single domain antibodies such as IgNAR or fragments thereof); camelid antibodies; masked antibodies (e.g., Probodies®); Small Modular ImmunoPharmaceuticals (“SMIPsTM”); single chain or Tandem diabodies (TandAb®); VHHs; Anticalins®; Nanobodies®; minibodies; BiTE®s; ankyrin repeat proteins or DARPINs®; Avimers®; DARTs; TCR-like antibodies; Adnectins®; Affilins®; Trans-bodies®; Affibodies®; TrimerX®; MicroProteins; Fynomers®, Centyrins®; and KALBITOR®s.

In some embodiments, the CD4 binding agent is a peptide. In some embodiments, the CD4 binding agent is an antibody, such as a single-chain variable fragment (scFv). In some embodiments, the CD4 binding agent is an antibody, such as a single domain antibody. In some embodiments, the antibody can be human or humanized. In some embodiments, the CD4 binding agent is a VHH. In some embodiments, the antibody or portion thereof is naturally occurring. In some embodiments, the antibody or portion thereof is synthetic.

In some embodiments, the antibody can be generated from phage display libraries to have specificity for a desired target ligand. In some embodiments, the phage display libraries are generated from a VHH repertoire of camelids immunized with various antigens, as described in Arbabi et al., FEBS Letters, 414, 521-526 (1997); Lauwereys et al., EMBO J., 17, 3512-3520 (1998); Decanniere et al., Structure, 7, 361-370 (1999). In some embodiments, the phage display library is generated comprising antibody fragments of a non-immunized camelid. In some embodiments, a library of human single domain antibodies is synthetically generated by introducing diversity into one or more scaffolds.

In some embodiments, the C-terminus of the CD4 binding agent is attached to the C-terminus of the G protein (e.g., fusogen) or biologically active portion thereof. In some embodiments, the N-terminus of the CD4 binding agent is exposed on the exterior surface of the lipid bilayer.

In some embodiments, the CD4 binding agent is the only surface displayed non-viral sequence of the viral vector. In some embodiments, the CD4 binding agent is the only membrane bound non-viral sequence of the viral vector. In some embodiments, the viral vector does not contain a molecule that engages or stimulates T cells other than the CD4 binding agent.

In some embodiments, viral vectors may display CD4 binding agents that are not conjugated to protein fusogens in order to redirect the fusion activity towards a cell that is bound by the targeting moiety, or to affect homing.

In some embodiments, a protein fusogen derived from a virus or organism that do not infect humans does not have a natural fusion targets in patients, and thus has high specificity.

The viral vectors disclosed herein include one or more CD8 binding agents. For example, a CD8 binding agent may be fused to or incorporated in a protein fusogen or viral envelope protein. In another embodiment, a CD8 binding agent may be incorporated into the viral envelope via fusion with a transmembrane domain.

Exemplary CD8 binding agents include antibodies and fragments thereof (e.g., scFv, VHH) that bind to one or more of CD8 alpha and CD8 beta. Such antibodies may be derived from any species, and may be for example, mouse, rabbit, human, humanized, or camelid antibodies.

Exemplary antibodies include those disclosed in WO2014025828, WO2014164553, WO2020069433, WO2015184203, US20160176969, WO2017134306, WO2019032661, WO2020257412, WO2018170096, WO2020060924, U.S. Ser. No. 10/730,944, US20200172620, and the non-human antibodies OKT8; RPA-T8, 12.C7 (Novus); 17D8, 3B5, LT8, RIV11, SP16, YTC182.20, MEM-31, MEM-87, RAVB3, C8/144B (Thermo Fisher); 2ST8.5H7, Bu88, 3C39, Hit8a, SPM548, CA-8, SK1, RPA-T8 (GeneTex); UCHT4 (Absolute Antibody); BW135/80 (Miltenyi); G42-8 (BD Biosciences); C8/1779R, mAB 104 (Enzo Life Sciences); B-Z31 (Sapphire North America); 32-M4, 5F10, MCD8, UCH-T4, 5F2 (Santa Cruz); D8A8Y, RPA-T8 (Cell Signaling Technology). Further exemplary anti-CD8 binding agents and G proteins are described in U.S. provisional application No. 63/172,518, which is incorporated by reference herein. Other exemplary binding agents include designed ankyrin repeat proteins (DARPins) and binding agents based on fibronectin type III (Fn3) scaffolds.

In some embodiments, the CD8 binding agent is an scFv that contains a VH and VL set forth from any as below, in which the VH and VL are separated by linker. In some embodiments, the CD8 binding agent is a VHH having the sequence set forth below. In some embodiments, the CD8 binding agent is linked to the C-terminus of a truncated NiV-G set forth in SEQ ID NO: 19 to provide a re-targeted NiV-G. In some embodiments, the retargeted NiV-G is pseudotyped on a lentiviral vector with the a NiV-F (e.g. set forth in SEQ ID NO:12). In some embodiments, the lentiviral vector further contains a payload gene encoding an anti-CD19 CAR. In some embodiments, the anti-CD19 CAR contains an anti-CD19 FMC63 scFv binding domain set forth in SEQ ID NO:40, a CD8 hinge set forth in SEQ ID NO:27, a CD8 transmembrane domain set forth in SEQ ID NO: 33, a 4-1bb signaling domain set forth in SEQ ID NO:36. a CD3zeta signaling domain set forth in SEQ ID NO: 38.

CD8_1
VH (SEQ ID NO.: 120):
QVQLVQSGAEVKKPGASVKVSCKASGGTFSSYAISWVRQAPGQGLEWMG
IIDPSDGNTNYAQNFQGRVTMTRDTSTSTVYMELSSLRSEDTAVYYCAK
ERAAAGYYYYMDVWGQGTTVTVSS
VL (SEQ ID NO.: 121:
DIQMTQSPSSLSASVGDRVTITCRASQSISSYLNWYQQKPGKAPKLLIY
AASSLQSGVPSRFSGSGSGTDFTLTISSLQPEDFATYYCQQSYSTPLTF
GGGTKVEIKR
CD8_2
VH (SEQ ID NO.: 122):
QVQLVQSGAEVKKPGASVKVSCKASGYTFTDYYIQWVRQAPGQGLEWMG
WINPNSGGTSYAQKFQGRVTMTRDTSTSTVYMELSSLRSEDTAVYYCAK
EGDYYYGMDAWGQGTMVTVSS
VL (SEQ ID NO.: 123):
DIVMTQSPLSLPVTPGEPASISCRSSQSLLHSNGYNYLDWYLQKPGQSP
QLLIYLGSNRASGVPDRFSGSGSGTDFTLKISRVEAEDVGVYYCMQGLQ
TPHTFGQGTKVEIKR
CD8_3
VH (SEQ ID NO.: 124):
QVQLVQSGAEVKKPGASVKVSCKASGYTFTSYYMHWVRQAPGQGLEWMG
GFDPEDGETIYAQKFQGRVTMTRDTSTSTVYMELSSLRSEDTAVYYCAR
DQGWGMDVWGQGTTVTVSS
VL (SEQ ID NO.: 125):
DIQMTQSPSSLSASVGDRVTITCRASQSISSYLNWYQQKPGKAPKLLIY
AASSLQSGVPSRFSGSGSGTDFTLTISSLQPEDFATYYCQQTYSTPYTF
GQGTKLEIKR
CD8_4
VH (SEQ ID NO.: 126):
QVQLVQSGAEVKKPGASVKVSCKASGYTFTNHYMHWVRQAPGQGLEWMG
WMNPNSGNTGYAQKFQGRVTMTRDTSTSTVYMELSSLRSEDTAVYYCAS
SESGSDLDYWGQGTLVTVSS
VL (SEQ ID NO.: 127):
DIQMTQSPSSLSASVGDRVTITCRASQTIGNYVNWYQQKPGKAPKLLIY
GASNLHTGVPSRFSGSGSGTDFTLTISSLQPEDFATYYCQQTYSAPLTF
GGGTKVEIKR

In some embodiments, the CD8 binding agent is VHH set forth as:

VHH (SEQ ID NO.: 128):
QVQLVESGGGLVQAGGSLRLSCAASGRTFSGYVMGWFRQAPGKQRKFVA
AISRGGLSTSYADSVKGRFTISRDNAKNTVFLQMNTLKPEDTAVYYCAA
DRSDLYEITAASNIDSWGQGTLVTVSS

In some embodiments, protein fusogens or viral envelope proteins may be re-targeted by mutating amino acid residues in a fusion protein or a targeting protein (e.g. the hemagglutinin protein). In particular embodiments, the fusogen (e.g. G protein) is mutated to reduce binding for the native binding partner of the fusogen. In some embodiments, the fusogen is or contains a mutant G protein or a biologically active portion thereof that is a mutant of wild-type Niv-G and exhibits reduced binding to one or both of the native binding partners Ephrin B2 or Ephrin B3, including any as described above. Thus, in some aspects, a fusogen can be retargeted to display altered tropism. In some embodiments, the binding confers re-targeted binding compared to the binding of a wild-type surface glycoprotein protein in which a new or different binding activity is conferred. In particular embodiments, the binding confers re-targeted binding compared to the binding of a wild-type G protein in which a new or different binding activity is conferred. In some embodiments the fusogen is randomly mutated. In some embodiments the fusogen is rationally mutated. In some embodiments the fusogen is subjected to directed evolution. In some embodiments the fusogen is truncated and only a subset of the peptide is used in the viral vector. In some embodiments, amino acid residues in the measles hemagglutinin protein may be mutated to alter the binding properties of the protein, redirecting fusion (doi:10.1038/nbt942, Molecular Therapy vol. 16 no. 8, 1427-1436 August 2008, doi:10.1038/nbt1060, DOI: 10.1128/JVI.76.7.3558-3563.2002, DOI: 10.1128/JVI.75.17.8016-8020.2001, doi: 10.1073pnas.0604993103).

In some embodiments, protein fusogens may be re-targeted by covalently conjugating a CD8 binding agent to the fusion protein or targeting protein (e.g. the hemagglutinin protein). In some embodiments, the fusogen and CD8 binding agent are covalently conjugated by expression of a chimeric protein comprising the fusogen linked to the CD8 binding agent. In some embodiments, a single-chain variable fragment (scFv) can be conjugated to fusogens to redirect fusion activity towards cells that display the scFv binding target (doi:10.1038/nbt1060, DOI 10.1182/blood-2012-11-468579, doi:10.1038/nmeth.1514, doi:10.1006/mthe.2002.0550, HUMAN GENE THERAPY 11:817-826, doi:10.1038/nbt942, doi:10.1371/journal.pone.0026381, DOI 10.1186/s12896-015-0142-z). In some embodiments, designed ankyrin repeat proteins (DARPin) can be conjugated to fusogens to redirect fusion activity towards cells that display the DARPin binding target (doi:10.1038/mt.2013.16, doi:10.1038/mt.2010.298, doi: 10.4049/jimmunol.1500956), as well as combinations of different DARPins (doi:10.1038/mto.2016.3). In some embodiments, receptor ligands and antigens can be conjugated to fusogens to redirect fusion activity towards cells that display the target receptor (DOI: 10.1089/hgtb.2012.054, DOI: 10.1128/JVI.76.7.3558-3563.2002). In some embodiments, a targeting protein can also include an antibody or an antigen-binding fragment thereof (e.g., Fab, Fab′, F(ab′)2, Fv fragments, scFv antibody fragments, disulfide-linked Fvs (sdFv), a Fd fragment consisting of the VH and CH1 domains, linear antibodies, single domain antibodies such as sdAb (either VL or VH), nanobodies, or camelid VHH domains), an antigen-binding fibronectin type III (Fn3) scaffold such as a fibronectin polypeptide minibody, a ligand, a cytokine, a chemokine, or a T cell receptor (TCRs). In some embodiments, protein fusogens may be re-targeted by non-covalently conjugating a CD8 binding agent to the fusion protein or targeting protein (e.g. the hemagglutinin protein). In some embodiments, the fusion protein can be engineered to bind the Fc region of an antibody that targets an antigen on a target cell, redirecting the fusion activity towards cells that display the antibody's target (DOI: 10.1128/JVI.75.17.8016-8020.2001, doi:10.1038/nml192). In some embodiments, altered and non-altered fusogens may be displayed on the same retroviral vector or VLP (doi: 10.1016/j.biomaterials.2014.01.051).

In some embodiments, a CD8 binding agent comprises a humanized antibody molecule, intact IgA, IgG, IgE or IgM antibody; bi- or multi-specific antibody (e.g., Zybodies®, etc); antibody fragments such as Fab fragments, Fab′ fragments, F(ab′)₂ fragments, Fd′ fragments, Fd fragments, and isolated CDRs or sets thereof; single chain Fvs; polypeptide-Fc fusions; single domain antibodies (e.g., shark single domain antibodies such as IgNAR or fragments thereof); cameloid antibodies; masked antibodies (e.g., Probodies®); Small Modular ImmunoPharmaceuticals (“SMIPsTM”); single chain or Tandem diabodies (TandAb®); VHHs; Anticalins®; Nanobodies®; minibodies; BiTE®s; ankyrin repeat proteins or DARPINs®; Avimers®; DARTs; TCR-like antibodies; Adnectins®; Affilins®; Trans-bodies®; Affibodies®; TrimerX®; MicroProteins; Fynomers®, Centyrins®; and KALBITOR®s.

In some embodiments, the CD8 binding agent is a peptide. In some embodiments, the CD8 binding agent is an antibody, such as a single-chain variable fragment (scFv). In some embodiments, the CD8 binding agent is an antibody, such as a single domain antibody. In some embodiments, the CD8 binding agent is a VHH. In some embodiments, the antibody can be human or humanized. In some embodiments, the antibody or portion thereof is naturally occurring. In some embodiments, the antibody or portion thereof is synthetic.

In some embodiments, the antibody can be generated from phage display libraries to have specificity for a desired target ligand. In some embodiments, the phage display libraries are generated from a VHH repertoire of camelids immunized with various antigens, as described in Arbabi et al., FEBS Letters, 414, 521-526 (1997); Lauwereys et al., EMBO J., 17, 3512-3520 (1998); Decanniere et al., Structure, 7, 361-370 (1999). In some embodiments, the phage display library is generated comprising antibody fragments of a non-immunized camelid. In some embodiments, a library of human single domain antibodies is synthetically generated by introducing diversity into one or more scaffolds.

In some embodiments, the C-terminus of the CD8 binding agent is attached to the C-terminus of the G protein (e.g., fusogen) or biologically active portion thereof. In some embodiments, the N-terminus of the CD8 binding agent is exposed on the exterior surface of the lipid bilayer.

In some embodiments, the CD8 binding agent is the only surface displayed non-viral sequence of the viral vector. In some embodiments, the CD8 binding agent is the only membrane bound non-viral sequence of the viral vector. In some embodiments, the viral vector does not contain a molecule that engages or stimulates T cells other than the CD8 binding agent.

In some embodiments, viral vectors may display CD8 binding agents that are not conjugated to protein fusogens in order to redirect the fusion activity towards a cell that is bound by the targeting moiety, or to affect homing.

In some embodiments, a protein fusogen derived from a virus or organism that do not infect humans does not have a natural fusion targets in patients, and thus has high specificity.

In some embodiments, the G protein or functionally active variant or biologically active portion thereof is linked directly to the binding domain and/or variable domain thereof. In some embodiments, the targeted envelope protein is a fusion protein that has the following structure: (N′-single domain antibody-C′)-(C′-G protein-N′).

In some embodiments, the G protein or functionally active variant or biologically active portion thereof is linked indirectly via a linker to the binding domain and/or variable domain thereof. In some embodiments, the linker is a peptide linker. In some embodiments, the linker is a chemical linker.

In some embodiments, the linker is a peptide linker and the targeted envelope protein is a fusion protein containing the G protein or functionally active variant or biologically active portion thereof linked via a peptide linker to the sdAb variable domain. In some embodiments, the targeted envelope protein is a fusion protein that has the following structure: (N′-single domain antibody-C′)-Linker-(C′-G protein-N′).

In some embodiments, the peptide linker is up to 65 amino acids in length. In some embodiments, the peptide linker comprises from or from about 2 to 65 amino acids, 2 to 60 amino acids, 2 to 56 amino acids, 2 to 52 amino acids, 2 to 48 amino acids, 2 to 44 amino acids, 2 to 40 amino acids, 2 to 36 amino acids, 2 to 32 amino acids, 2 to 28 amino acids, 2 to 24 amino acids, 2 to 20 amino acids, 2 to 18 amino acids, 2 to 14 amino acids, 2 to 12 amino acids, 2 to 10 amino acids, 2 to 8 amino acids, 2 to 6 amino acids, 6 to 65 amino acids, 6 to 60 amino acids, 6 to 56 amino acids, 6 to 52 amino acids, 6 to 48 amino acids, 6 to 44 amino acids, 6 to 40 amino acids, 6 to 36 amino acids, 6 to 32 amino acids, 6 to 28 amino acids, 6 to 24 amino acids, 6 to 20 amino acids, 6 to 18 amino acids, 6 to 14 amino acids, 6 to 12 amino acids, 6 to 10 amino acids, 6 to 8 amino acids, 8 to 65 amino acids, 8 to 60 amino acids, 8 to 56 amino acids, 8 to 52 amino acids, 8 to 48 amino acids, 8 to 44 amino acids, 8 to 40 amino acids, 8 to 36 amino acids, 8 to 32 amino acids, 8 to 28 amino acids, 8 to 24 amino acids, 8 to 20 amino acids, 8 to 18 amino acids, 8 to 14 amino acids, 8 to 12 amino acids, 8 to 10 amino acids, 10 to 65 amino acids, 10 to 60 amino acids, 10 to 56 amino acids, 10 to 52 amino acids, 10 to 48 amino acids, 10 to 44 amino acids, 10 to 40 amino acids, 10 to 36 amino acids, 10 to 32 amino acids, 10 to 28 amino acids, 10 to 24 amino acids, 10 to 20 amino acids, 10 to 18 amino acids, 10 to 14 amino acids, 10 to 12 amino acids, 12 to 65 amino acids, 12 to 60 amino acids, 12 to 56 amino acids, 12 to 52 amino acids, 12 to 48 amino acids, 12 to 44 amino acids, 12 to 40 amino acids, 12 to 36 amino acids, 12 to 32 amino acids, 12 to 28 amino acids, 12 to 24 amino acids, 12 to 20 amino acids, 12 to 18 amino acids, 12 to 14 amino acids, 14 to 65 amino acids, 14 to 60 amino acids, 14 to 56 amino acids, 14 to 52 amino acids, 14 to 48 amino acids, 14 to 44 amino acids, 14 to 40 amino acids, 14 to 36 amino acids, 14 to 32 amino acids, 14 to 28 amino acids, 14 to 24 amino acids, 14 to 20 amino acids, 14 to 18 amino acids, 18 to 65 amino acids, 18 to 60 amino acids, 18 to 56 amino acids, 18 to 52 amino acids, 18 to 48 amino acids, 18 to 44 amino acids, 18 to 40 amino acids, 18 to 36 amino acids, 18 to 32 amino acids, 18 to 28 amino acids, 18 to 24 amino acids, 18 to 20 amino acids, 20 to 65 amino acids, 20 to 60 amino acids, 20 to 56 amino acids, 20 to 52 amino acids, 20 to 48 amino acids, 20 to 44 amino acids, 20 to 40 amino acids, 20 to 36 amino acids, 20 to 32 amino acids, 20 to 28 amino acids, 20 to 26 amino acids, 20 to 24 amino acids, 24 to 65 amino acids, 24 to 60 amino acids, 24 to 56 amino acids, 24 to 52 amino acids, 24 to 48 amino acids, 24 to 44 amino acids, 24 to 40 amino acids, 24 to 36 amino acids, 24 to 32 amino acids, 24 to 30 amino acids, 24 to 28 amino acids, 28 to 65 amino acids, 28 to 60 amino acids, 28 to 56 amino acids, 28 to 52 amino acids, 28 to 48 amino acids, 28 to 44 amino acids, 28 to 40 amino acids, 28 to 36 amino acids, 28 to 34 amino acids, 28 to 32 amino acids, 32 to 65 amino acids, 32 to 60 amino acids, 32 to 56 amino acids, 32 to 52 amino acids, 32 to 48 amino acids, 32 to 44 amino acids, 32 to 40 amino acids, 32 to 38 amino acids, 32 to 36 amino acids, 36 to 65 amino acids, 36 to 60 amino acids, 36 to 56 amino acids, 36 to 52 amino acids, 36 to 48 amino acids, 36 to 44 amino acids, 36 to 40 amino acids, 40 to 65 amino acids, 40 to 60 amino acids, 40 to 56 amino acids, 40 to 52 amino acids, 40 to 48 amino acids, 40 to 44 amino acids, 44 to 65 amino acids, 44 to 60 amino acids, 44 to 56 amino acids, 44 to 52 amino acids, 44 to 48 amino acids, 48 to 65 amino acids, 48 to 60 amino acids, 48 to 56 amino acids, 48 to 52 amino acids, 50 to 65 amino acids, 50 to 60 amino acids, 50 to 56 amino acids, 50 to 52 amino acids, 54 to 65 amino acids, 54 to 60 amino acids, 54 to 56 amino acids, 58 to 65 amino acids, 58 to 60 amino acids, or 60 to 65 amino acids. In some embodiments, the peptide linker is a polypeptide that is 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, or 65 amino acids in length.

In particular embodiments, the linker is a flexible peptide linker. In some such embodiments, the linker is 1-20 amino acids, such as 1-20 amino acids predominantly composed of glycine. In some embodiments, the linker is 1-20 amino acids, such as 1-20 amino acids predominantly composed of glycine and serine. In some embodiments, the linker is a flexible peptide linker containing amino acids Glycine and Serine, referred to as GS-linkers. In some embodiments, the peptide linker includes the sequences GS, GGS, GGGGS (SEQ ID NO:20), GGGGGS (SEQ ID NO:21) or combinations thereof. In some embodiments, the polypeptide linker has the sequence (GGS)n, wherein n is 1 to 10. In some embodiments, the polypeptide linker has the sequence (GGGGS)n, (SEQ ID NO:22) wherein n is 1 to 10. In some embodiments, the polypeptide linker has the sequence (GGGGGS)n (SEQ ID NO:23), wherein n is 1 to 6.

### G. Exogenous Agent

In embodiments, the targeted lipid particle, such as a viral vector, further comprises an agent that is exogenous relative to the source cell (hereinafter also called “cargo” or “payload”). In some embodiments, the exogenous agent is a protein or a nucleic acid (e.g., a DNA, a chromosome (e.g. a human artificial chromosome), an RNA, e.g., an mRNA or miRNA). In some embodiments, the exogenous agent is a nucleic acid that encodes a protein (i.e., a payload gene). The protein can be any protein as is desired for targeted delivery to a target cell. In some embodiments, the protein is a therapeutic agent or a diagnostic agent. In some embodiments, the protein is an antigen receptor for targeting cells expressed by or associated with a disease or condition, for instance a chimeric antigen receptor (CAR) or a T cell receptor (TCR). Reference to the coding sequence of a nucleic acid encoding the protein also is referred to herein as a payload gene. In some embodiments, the exogenous agent or the nucleic acid encoding the exogenous agent are present in the lumen of the non-cell particle.

In some embodiments, the exogenous agent or cargo includes a nucleic acid, e.g., DNA, nDNA (nuclear DNA), mtDNA (mitochondrial DNA), protein coding DNA, gene, operon, chromosome, genome, transposon, retrotransposon, viral genome, intron, exon, modified DNA, mRNA (messenger RNA), tRNA (transfer RNA), modified RNA, microRNA, siRNA (small interfering RNA), tmRNA (transfer messenger RNA), rRNA (ribosomal RNA), mtRNA (mitochondrial RNA), snRNA (small nuclear RNA), small nucleolar RNA (snoRNA), SmY RNA (mRNA trans-splicing RNA), gRNA (guide RNA), TERC (telomerase RNA component), aRNA (antisense RNA), cis-NAT (Cis-natural antisense transcript), CRISPR RNA (crRNA), lncRNA (long noncoding RNA), piRNA (piwi-interacting RNA), shRNA (short hairpin RNA), tasiRNA (trans-acting siRNA), eRNA (enhancer RNA), satellite RNA, pcRNA (protein coding RNA), dsRNA (double stranded RNA), RNAi (interfering RNA), circRNA (circular RNA), reprograming RNAs, aptamers, and any combination thereof. In some embodiments, the nucleic acid is a wild-type nucleic acid. In some embodiments, the protein is a mutant nucleic acid. In some embodiments the nucleic acid is a fusion or chimera of multiple nucleic acid sequences.

In some embodiments, the exogenous agent or cargo may include a nucleic acid. For example, the exogenous agent or cargo may comprise RNA to enhance expression of an endogenous protein, or a siRNA or miRNA that inhibits protein expression of an endogenous protein. For example, the endogenous protein may modulate structure or function in the target cells. In some embodiments, the cargo may include a nucleic acid encoding an engineered protein that modulates structure or function in the target cells. In some embodiments, the exogenous agent or cargo is a nucleic acid that targets a transcriptional activator that modulate structure or function in the target cells.

In some embodiments, the lipid particle comprising a nucleic acid encoding a payload gene. For example, the lipid particle may comprise a nucleic acid that is or encodes an RNA to enhance expression of an endogenous protein, or a siRNA or miRNA that inhibits protein expression of an endogenous protein. For example, the endogenous protein may modulate structure or function in the target cells. In some embodiments, the lipid particle may comprise a nucleic acid that is or encodes an engineered protein that modulates structure or function in the target cells. In some embodiments, the lipid particle may comprise a nucleic acid that is or encodes a transcriptional activator that modulate structure or function in the target cells.

In some embodiments, the lipid described herein comprises a nucleic acid, e.g., RNA or DNA. In some embodiments, the nucleic acid is, comprises, or consists of one or more natural nucleic acid residues. In some embodiments, the nucleic acid is, comprises, or consists of one or more nucleic acid analogs. In some embodiments, the nucleic acid has a nucleotide sequence that encodes a functional gene product such as an RNA or protein. In some embodiments, the nucleic acid includes one or more introns. In some embodiments, nucleic acids are prepared by one or more of isolation from a natural source, enzymatic synthesis by polymerization based on a complementary template (in vivo or in vitro), reproduction in a recombinant cell or system, and chemical synthesis. In some embodiments, the nucleic acid is at least 3, 4, 5, 6, 7, 8, 9, 10, 15, 20, 25, 30, 35, 40, 45, 50, 55, 60, 65, 70, 75, 80, 85, 90, 95, 100, 110, 120, 130, 140, 150, 160, 170, 180, 190, 20, 225, 250, 275, 300, 325, 350, 375, 400, 425, 450, 475, 500, 600, 700, 800, 900, 1000, 1500, 2000, 2500, 3000, 3500, 4000, 4500, 5000 or more residues long. In some embodiments, the nucleic acid is partly or wholly single stranded; in some embodiments, the nucleic acid is partly or wholly double stranded. In some embodiments the nucleic acid has a nucleotide sequence comprising at least one element that encodes, or is the complement of a sequence that encodes, a polypeptide.

In some embodiments, the lipid particle contains a nucleic acid that encodes a payload gene (also referred to as a “heterologous, recombinant, exogenous, or therapeutic gene.”).

In embodiments, the exogenous agent is present at least, or no more than, 10, 20, 50, 100, 200, 500, 1,000, 2,000, 5,000, 10,000, 20,000, 50,000, 100,000, 200,000, 500,000, 1,000,000, 5,000,000, 10,000,000, 50,000,000, 100,000,000, 500,000,000, or 1,000,000,000 copies. In embodiments, the targeted lipid particle has an altered, e.g., increased or decreased level of one or more endogenous molecule, e.g., protein or nucleic acid (e.g., in some embodiments, endogenous relative to the source cell, and in some embodiments, endogenous relative to the target cell), e.g., due to treatment of the source cell, e.g., mammalian source cell with a siRNA or gene editing enzyme. In embodiments, the endogenous molecule is present at least, or no more than, 10, 20, 50, 100, 200, 500, 1,000, 2,000, 5,000, 10,000, 20,000, 50,000, 100,000, 200,000, 500,000, 1,000,000, 5,000,000, 10,000,000, 50,000,000, 100,000,000, 500,000,000, or 1,000,000,000 copies. In embodiments, the endogenous molecule (e.g., an RNA or protein) is present at a concentration of at least 1, 2, 3, 4, 5, 10, 20, 50, 100, 500, 103, 5.0×103, 104, 5.0×104, 105, 5.0×105, 106, 5.0×106, 1.0×107, 5.0×107, or 1.0×108, greater than its concentration in the source cell. In embodiments, the endogenous molecule (e.g., an RNA or protein) is present at a concentration of at least 1, 2, 3, 4, 5, 10, 20, 50, 100, 500, 103, 5.0×103, 104, 5.0×104, 105, 5.0×105, 106, 5.0×106, 1.0×107, 5.0×107, or 1.0×108 less than its concentration in the source cell.

In some embodiments, the targeted lipid particle delivers to a target cell at least 10%, 20%, 30%, 40%, 50%, 60%, 70%, 80%, 90%, 95%, 96%, 97%, 98%, or 99% of the cargo (e.g., a therapeutic agent, e.g., an exogenous therapeutic agent) comprised by the fusosome. In some embodiments, the targeted lipid particle that fuses with the target cell(s) delivers to the target cell an average of at least 10%, 20%, 30%, 40%, 50%, 60%, 70%, 80%, 90%, 95%, 96%, 97%, 98%, or 99% of the cargo (e.g., a therapeutic agent, e.g., an exogenous therapeutic agent) comprised by the lipid particles that fuse with the target cell(s). In some embodiments, the targeted lipid particle composition delivers to a target tissue at least 10%, 20%, 30%, 40%, 50%, 60%, 70%, 80%, 90%, 95%, 96%, 97%, 98%, or 99% of the cargo (e.g., a therapeutic agent, e.g., an exogenous therapeutic agent) comprised by the targeted lipid particle compositions.

In some embodiments, the exogenous agent or cargo is or encodes a polypeptide, e.g., enzymes, structural polypeptides, signaling polypeptides, regulatory polypeptides, transport polypeptides, sensory polypeptides, motor polypeptides, defense polypeptides, storage polypeptides, transcription factors, antibodies, cytokines, hormones, catabolic polypeptides, anabolic polypeptides, proteolytic polypeptides, metabolic polypeptides, kinases, transferases, hydrolases, lyases, isomerases, ligases, enzyme modulator polypeptides, protein binding polypeptides, lipid binding polypeptides, membrane fusion polypeptides, cell differentiation polypeptides, epigenetic polypeptides, cell death polypeptides, nuclear transport polypeptides, nucleic acid binding polypeptides, reprogramming polypeptides, DNA editing polypeptides, DNA repair polypeptides, DNA recombination polypeptides, transposase polypeptides, DNA integration polypeptides, targeted endonucleases (e.g. Zinc-finger nucleases, transcription-activator-like nucleases (TALENs), cas9 and homologs thereof), recombinases, and any combination thereof. In some embodiments the protein targets a protein in the cell for degradation. In some embodiments the protein targets a protein in the cell for degradation by localizing the protein to the proteasome. In some embodiments, the protein is a wild-type protein. In some embodiments, the protein is a mutant protein. In some embodiments the protein is a fusion or chimeric protein.

In some embodiments, the exogenous agent is or encodes a cytosolic protein, e.g., a protein that is produced in the recipient cell and localizes to the recipient cell cytoplasm. In some embodiments, the exogenous agent is or encodes a secreted protein, e.g., a protein that is produced and secreted by the recipient cell. In some embodiments, the exogenous agent is or encodes a nuclear protein, e.g., a protein that is produced in the recipient cell and is imported to the nucleus of the recipient cell. In some embodiments, the exogenous agent is or encodes an organellar protein (e.g., a mitochondrial protein), e.g., a protein that is produced in the recipient cell and is imported into an organelle (e.g., a mitochondrial) of the recipient cell. In some embodiments, the protein is a wild-type protein or a mutant protein. In some embodiments the protein is a fusion or chimeric protein.

In some embodiments, the exogenous agent is capable of being delivered to a hepatocyte or liver cell. In some embodiments, the exogenous agents or cargo can be delivered to treat a disease or disorder in a hepatocyte or liver cell.

In some embodiments, the exogenous agent is encoded by a gene from among OTC, CPS1, NAGS, BCKDHA, BCKDHB, DBT, DLD, MUT, MMAA, MMAB, MMACHC, MMADHC, MCEE, PCCA, PCCB, UGT1A1, ASS1, PAH, PAL, ATP8B1, ABCB11, ABCB4, TJP2, IVD, GCDH, ETFA, ETFB, ETFDH, ASL, D2HGDH, HMGCL, MCCC1, MCCC2, ABCD4, HCFC1, LNBRD1, ARG1, SLC25A15, SLC25A13, ALAD, CPOX, HMBS, PPOX, BTD, HLCS, PC, SLC7A7, CPT2, ACADM, ACADS, ACADVL, AGL, G6PC, GBE1, PHKA1, PHKA2, PHKB, PHKG2, SLC37A4, PMM2, CBS, FAH, TAT, GALT, GALK1, GALE, G6PD, SLC3A1, SLC7A9, MTHFR, MTR, MTRR, ATP7B, HPRT1, HJV, HAMP, JAG1, TTR, AGXT, LIPA, SERPING1, HSD17B4, UROD, HFE, LPL, GRHPR, HOGA1, LDLR, ACAD8, ACADSB, ACAT1, ACSF3, ASPA, AUH, DNAJC19, ETHE1, FBP1, FTCD, GSS, HIBCH, IDH2, L2HGDH, MLYCD, OPA3, OPLAH, OXCT1, POLG, PPM1K, SERAC1, SLC25A1, SUCLA2, SUCLG1, TAZ, AGK, CLPB, TMEM70, ALDH18A1, OAT, CA5A, GLUD1, GLUL, UMPS, SLC22A5, CPT1A, HADHA, HADH, SLC52A1, SLC52A2, SLC52A3, HADHB, GYS2, PYGL, SLC2A2, ALG1, ALG2, ALG3, ALG6, ALG8, ALG9, ALG1l, ALG12, ALG13, ATP6V0A2, B3GLCT, CHST14, COG1, COG2, COG4, COG5, COG6, COG7, COG8, DOLK, DHDDS, DPAGT1, DPM1, DPM2, DPM3, G6PC3, GFPT1, GMPPA, GMPPB, MAGT1, MANIB1, MGAT2, MOGS, MPDU1, MPI, NGLY1, PGM1, PGM3, RFT1, SEC23B, SLC35A1, SLC35A2, SLC35C1, SSR4, SRD5A3, TMEM165, TRIP11, TUSC3, ALG14, B4GALT1, DDOST, NUS1, RPN2, SEC23A, SLC35A3, ST3GAL3, STT3A, STT3B, AGA, ARSA, ARSB, ASAH1, ATP13A2, CLN3, CLN5, CLN6, CLN8, CTNS, CTSA, CTSD, CTSF, CTSK, DNAJC5, FUCA1, GAA, GALC, GALNS, GLA, GLB1, GM2A, GNPTAB, GNPTG, GNS, GRN, GUSB, HEXA, HEXB, HGSNAT, HYAL1, IDS, IDUA, KCTD7, LAMP2, MAN2B1, MANBA, MCOLN1, MFSD8, NAGA, NAGLU, NEU1, NPC1, NPC2, SGSH, PPT1, PSAP, SLC17A5, SMPD1, SUMF1, TPP1, AHCY, GNMT, MAT1A, GCH1, PCBD1, PTS, QDPR, SPR, DNAJC12, ALDH4A1, PRODH, HPD, GBA, HGD, AMN, CD320, CUBN, GIF, TCN1, TCN2, PREPL, PHGDH, PSAT1, PSPH, AMT, GCSH, GLDC, LIAS, NFU1, SLC6A9, SLC2A1, ATP7A, AP1S1, CP, SLC33A1, PEX7, PHYH, AGPS, GNPAT, ABCD1, ACOX1, PEX1, PEX2, PEX3, PEX5, PEX6, PEX10, PEX12, PEX13, PEX14, PEX16, PEX19, PEX26, AMACR, ADA, ADSL, AMPD1, GPHN, MOCOS, MOCS1, PNP, XDH, SUOX, OGDH, SLC25A19, DHTKD1, SLC13A5, FH, DLAT, MPC1, PDHA1, PDHB, PDHX, PDP1, ABCC2, SLCO1B1, SLCO1B3, HFE2, ADAMTS13, PYGM, COL1A2, TNFRSF11B, TSC1, TSC2, DHCR7, PGK1, VLDLR, KYNU, F5, C3, COL4A1, CFH, SLC12A2, GK, SFTPC, CRTAP, P3H1, COL7A1, PKLR, TALDO1, TF, EPCAM, VHL, GC, SERPINA1, ABCC6, F8, F9, ApoB, PCSK9, LDLRAP1, ABCG5, ABCG8, LCAT, SPINK5, or GNE.

In some embodiments, the exogenous agent is encoded by a gene from among OTC, CPS1, NAGS, BCKDHA, BCKDHB, DBT, DLD, MUT, MMAA, MMAB, MMACHC, MMADHC, MCEE, PCCA, PCCB, UGT1A1, ASS1, PAL, PAH, ATP8B1, ABCB11, ABCB4, TJP2, IVD, GCDH, ETFA, ETFB, ETFDH, ASL, D2HGDH, HMGCL, MCCC1, MCCC2, ABCD4, HCFC1, LMBRD1, ARG1, SLC25A15, SLC25A13, ALAD, CPOX, HMBS, PPOX, BTD, HLCS, PC, SLC7A7, CPT2, ACADM, ACADS, ACADVL, AGL, G6PC, GBE1, PHKA1, PHKA2, PHKB, PHKG2, SLC37A4, PMM2, CBS, FAH, TAT, GALT, GALK1, GALE, G6PD, SLC3A1, SLC7A9, MTHFR, MTR, MTRR, ATP7B, HPRT1, HJV, HAMP, JAG1, TTR, AGXT, LIPA, SERPING1, HSD17B4, UROD, HFE, LPL, GRHPR, HOGA1, or LDLR. In some embodiments, the exogenous agent is the enzyme phenylalanine ammonia lyase (PAL).

In some embodiments, the exogenous agents or cargo can be delivered to treat and disease or indication listed in Table 2A. In some embodiments, the indications are specific for a liver cell or hepatocyte.

In some embodiments, the exogenous agent comprises a protein of Table 2A below. In some embodiments, the exogenous agent comprises the wild-type human sequence of any of the proteins of Table 2A, a functional fragment thereof (e.g., an enzymatically active fragment thereof), or a functional variant thereof. In some embodiments, the exogenous agent comprises an amino acid sequence having at least 70%, 75%, 80%, 85%, 90%, 95%, 96%, 97%, 98%, or 99%, identity to an amino acid sequence of Table 2A, e.g., a Uniprot Protein Accession Number sequence of column 4 of Table 2A or an amino acid sequence of column 5 of Table 2A. In some embodiments, the payload gene encoding an exogenous agent encodes an amino acid sequence having at least 70%, 75%, 80%, 85%, 90%, 95%, 96%, 97%, 98%, or 99%, identity to an amino acid sequence of Table 2A. In some embodiments, the payload gene encoding an exogenous agent has a nucleic acid sequence having at least 70%, 75%, 80%, 85%, 90%, 95%, 96%, 97%, 98%, or 99%, identity to a nucleic acid sequence of Table 2A, e.g., an Ensemble Gene Accession Number of column 3 of Table 2A.

Table 2A. The first column lists exogenous agents that can be delivered to treat the indications in the sixth column, according to the methods and uses herein. Each Uniprot accession number of Table 2A is herein incorporated by reference in its entirety. Reference to SEQ ID NO in Table 2A is as contained within WO 2021/202064, which is hereby incorporated by reference in its entirety.

TABLE 2A
Exemplary Exogenous Agents to be Delivered

| | | | | Amino Acid Sequence | | |
|---|---|---|---|---|---|---|
| | | Ensembl Gene(s) | Uniprot | (first Uniprot | | |
| | Entrez | Accession Number | Protein(s) | Accession Number) | | |
| | Accession | (ENSG0000 + | Accession | SEQ ID NO as set | | |
| Gene | Number | number shown) | Number | forth in WO 2021202604 | Disease/Disorder | Category |
| OTC | 5009 | 0036473 | P00480 | 61 | ornithine | Urea cycle disorder |
| | | | | | transcarbamylase | |
| | | | | | (OTC) deficiency | |
| CPS1 | 1373 | 0021826 | P31327, | 62 | carbamoyl | Urea cycle disorder |
| | | | Q6PEK7, | | phosphate | |
| | | | B7ZAW0, | | synthetase I | |
| | | | A0A024R454 | | (CPSI) deficiency | |
| NAGS | 162417 | 0161653 | Q8N159, | 63 | N-acetylglutamate | Urea cycle disorder |
| | | | Q2NKP2 | | synthase (NAGS) | |
| | | | | | deficiency | |
| BCKDHA | 593 | 0248098 | A0A024R0K3, | 64 | maple syrup urine | Organic acidemia |
| | | | P12694, | | disease (MSUD); | |
| | | | Q59EI3 | | Classic Maple | |
| | | | | | Syrup Urine | |
| | | | | | Disease (CMSUD) | |
| BCKDHB | 594 | 0083123 | A0A140VKB3, | 65 | maple syrup urine | Organic acidemia |
| | | | P21953, | | disease (MSUD); | |
| | | | B4E2N3, | | Classic Maple | |
| | | | B7ZB80 | | Syrup Urine | |
| | | | | | Disease (CMSUD) | |
| DBT | 1629 | 0137992 | P11182 | 66 | maple syrup urine | Organic acidemia |
| | | | | | disease (MSUD); | |
| | | | | | Classic Maple | |
| | | | | | Syrup Urine | |
| | | | | | Disease (CMSUD) | |
| DLD | 1738 | 0091140 | A0A024R713, | 67 | maple syrup urine | Urea cycle disorder |
| | | | P09622, | | disease (MSUD) | |
| | | | E9PEX6 | | Dihydrolipoamide | |
| | | | | | dehydrogenase | |
| | | | | | deficiency | |
| MUT | 4594 | 0146085 | A0A024RD82, | 68 | methylmalonic | Organic acidemia |
| | | | B2R6K1, | | acidemia due to | |
| | | | P22033 | | methylmalonyl- | |
| | | | | | CoA mutase | |
| | | | | | deficiency | |
| MMAA | 166785 | 0151611 | Q8IVH4 | 69 | cobalamin A | Organic acidemia |
| | | | | | deficiency | |
| | | | | | (methylmalonic | |
| | | | | | acidemia) | |
| MMAB | 326625 | 0139428 | Q96EY8 | 70 | cobalamin B | Organic acidemia |
| | | | | | deficiency | |
| | | | | | (methylmalonic | |
| | | | | | acidemia) | |
| MMACHC | 25974 | 0132763 | A0A0C4DGU2, | 71 | cobalamin C | Organic acidemia |
| | | | Q9Y4U1 | | deficiency | |
| | | | | | (methylmalonic | |
| | | | | | acidemia); | |
| | | | | | Methylmalonic | |
| | | | | | Acidemia with | |
| | | | | | Homocystinuria | |
| MMADHC | 27249 | 0168288 | Q9H3L0 | 72 | cobalamin D | Organic acidemia |
| | | | | | deficiency | |
| | | | | | (methylmalonic | |
| | | | | | acidemia); | |
| | | | | | Methylmalonic | |
| | | | | | Acidemia with | |
| | | | | | Homocystinuria; | |
| | | | | | Homocystinuria; | |
| | | | | | Cobalamin C | |
| | | | | | Deficiency | |
| MCEE | 84693 | 0124370 | Q96PE7 | 73 | methylmalonic | Organic acidemia |
| | | | | | acidemia; | |
| | | | | | Cobalamin D | |
| | | | | | Deficiency | |
| PCCA | 5095 | 0175198 | P05165 | 74 | propionic acidemia | Organic acidemia |
| PCCB | 5096 | 0114054 | P05166 | 75 | propionic acidemia | Organic acidemia |
| UGT1A1 | 54658 | 0241635 | P22309, | 76 | Crigler-Najjar | |
| | | | Q5DT03 | | syndrome type 1 | |
| | | | | | Crigler-Najjar | |
| | | | | | syndrome type 2, | |
| | | | | | Gilbert syndrome | |
| ASS1 | 445 | 0130707 | P00966, | 77 | citrullinemia type I | Urea cycle disorder |
| | | | Q5T6L4 | | | |
| PAH | 5053 | 0171759 | A0A024RBG4, | 78 | Phenylalanine | Aminoacidopathy |
| | | | P00439 | | hydroxylase | |
| | | | | | deficiency | |
| PAL | | | | 79 | Phenylalanine | Aminoacidopathy |
| | | | | | hydroxylase | |
| | | | | | deficiency | |
| ATP8B1 | 5205 | 0081923 | O43520 | 80 | Progressive | |
| | | | | | familial | |
| | | | | | intrahepatic | |
| | | | | | cholestasis Type 1 | |
| ABCB11 | 8647 | 0073734, | O95342 | 81 | Progressive | |
| | | 0276582 | | | familial | |
| | | | | | intrahepatic | |
| | | | | | cholestasis Type 2; | |
| | | | | | Progressive | |
| | | | | | Familial | |
| | | | | | Intrahepatic | |
| | | | | | Cholestasis Type 3 | |
| ABCB4 | 5244 | 0005471 | P21439 | 82 | Progressive | |
| | | | | | familial | |
| | | | | | intrahepatic | |
| | | | | | cholestasis Type 3; | |
| | | | | | Progressive | |
| | | | | | Familial | |
| | | | | | Intrahepatic | |
| | | | | | Cholestasis Type 2 | |
| TJP2 | 9414 | 0119139 | B7Z2R3, | 83 | Progressive | |
| | | | Q9UDY2, | | familial | |
| | | | B7Z954 | | intrahepatic | |
| | | | | | cholestasis Type 4 | |
| IVD | 3712 | 0128928 | P26440, | 84 | isovaleric | Organic acidemia |
| | | | A0A0A0MT83 | | acidemia (IVD) | |
| GCDH | 2639 | 0105607 | A0A024R7F9, | 85 | glutaric acidemia | Organic acidemia |
| | | | Q92947 | | type I | |
| ETFA | 2108 | 0140374 | A0A0S2Z3L0, | 86 | multiple acyl-CoA | Organic acidemia |
| | | | P13804 | | dehydrogenase | |
| | | | | | deficiency (a.k.a. | |
| | | | | | glutaric aciduria | |
| | | | | | type II) | |
| ETFB | 2109 | 0105379 | P38117 | 87 | multiple acyl-CoA | Organic acidemia |
| | | | | | dehydrogenase | |
| | | | | | deficiency (a.k.a. | |
| | | | | | glutaric aciduria | |
| | | | | | type II) | |
| ETFDH | 2110 | 0171503 | B4DEQ0, | 88 | multiple acyl-CoA | Organic acidemia |
| | | | Q16134 | | dehydrogenase | |
| | | | | | deficiency (a.k.a. | |
| | | | | | glutaric aciduria | |
| | | | | | type II) | |
| ASL | 435 | 0126522 | A0A024RDL8, | 89 | argininosuccinate | Urea cycle disorder |
| | | | P04424, | | lyase (ASL) | |
| | | | A0A0S2Z316 | | deficiency | |
| D2HGDH | 728294 | 0180902 | B3KSR6, | 90 | D-2- | Organic acidemia |
| | | | B4E3K7, | | hydroxyglutaric | |
| | | | B5MCV2, | | aciduria type I | |
| | | | Q8N465 | | | |
| HMGCL | 3155 | 0117305 | P35914 | 91 | 3-hydroxy-3- | Organic academia |
| | | | | | methylglutaryl- | Urea cycle disorder |
| | | | | | CoA lyase | |
| | | | | | (3HMG) | |
| | | | | | deficiency | |
| MCCC1 | 56922 | 0078070 | Q68D27, | 92 | 3-methylcrotonyl- | Organic acidemia |
| | | | Q96RQ3, | | CoA carboxylase | |
| | | | A0A0S2Z693, | | (3MCC) | |
| | | | E9PHF7 | | deficiency | |
| MCCC2 | 64087 | 0131844, | A0A140VK29, | 93 | 3-methylcrotonyl- | Organic acidemia |
| | | 0281742, | Q9HCC0 | | CoA carboxylase | |
| | | 0275300 | | | (3MCC) | |
| | | | | | deficiency | |
| ABCD4 | 5826 | 0119688 | A0A024R6B9, | 94 | methylmalonic | Organic acidemia |
| | | | O14678, | | acidemia with | |
| | | | A0A024R6C8 | | homocystinuria | |
| HCFC1 | 3054 | 0172534 | P51610, | 95 | methylmalonic | Organic acidemia |
| | | | A6NEM2 | | acidemia with | |
| | | | | | homocystinuria | |
| LMBRD1 | 55788 | 0168216 | Q9NUN5 | 96 | methylmalonic | Organic acidemia |
| | | | | | acidemia with | |
| | | | | | homocystinuria | |
| ARG1 | 383 | 0118520 | P05089 | 97 | arginase (ARG1) | Urea cycle disorder |
| | | | | | deficiency | |
| SLC25A15 | 10166 | 0102743 | Q9Y619 | 98 | hyperammonemia- | Urea cycle disorder |
| | | | | | hyperornithinemia- | |
| | | | | | homocitrullinuria | |
| | | | | | (HHH) syndrome | |
| SLC25A13 | 10165 | 0004864 | Q9UJS0 | 99 | citrin deficiency | Urea cycle disorder |
| | | | | | citrullinemia type | |
| | | | | | II | |
| ALAD | 210 | 0148218 | P13716 | 100 | Acute Hepatic | Porphyria |
| | | | | | porphyria | |
| CPOX | 1371 | 0080819 | P36551 | 101 | Acute Hepatic | Porphyria |
| | | | | | porphyria | |
| HMBS | 3145 | 0256269, | P08397 | 102 | Acute Hepatic | Porphyria |
| | | 0281702 | | | porphyria; | |
| | | | | | Acute Intermittent | |
| | | | | | Porphyria | |
| PPOX | 5498 | 0143224 | P50336, | 103 | Acute Hepatic | Porphyria |
| | | | B4DY76 | | porphyria | |
| BTD | 686 | 0169814 | P43251 | 104 | Biotinidase | Organic acidemia |
| | | | | | Deficiency | |
| HLCS | 3141 | 0159267 | P50747 | 105 | Holocarboxylase | Organic acidemia |
| | | | | | Synthetase | |
| | | | | | Deficiency | |
| PC | 5091 | 0173599 | P11498 | 106 | Pyruvate | Urea cycle disorder |
| | | | A0A024R5C5 | | Carboxylase | |
| | | | | | Deficiency | |
| SLC7A7 | 9056 | 0155465 | Q9UM01 | 107 | Lysinuric Protein | Urea cycle disorder |
| | | | A0A0S2Z502 | | Intolerance | |
| CPT2 | 1376 | 0157184 | P23786 | 108 | Carnitine | Fatty Acid Oxidation |
| | | | A0A140VK13 | | Palmitoyltransferase | |
| | | | A0A1B0GTB8 | | Type II (CPT II) | |
| | | | | | Deficiency | |
| ACADM | 34 | 0117054 | P11310 | 109 | Medium Chain | Fatty Acid Oxidation |
| | | | A0A0S2Z366, | | Acyl-CoA | |
| | | | B7Z911, | | Dehydrogenase | |
| | | | Q5HYG7, | | (MCAD) | |
| | | | Q5T4U5, | | Deficiency | |
| | | | B4DJE7 | | | |
| ACADS | 35 | 0122971 | P16219 | 110 | Short Chain Acyl- | Fatty acid oxidation |
| | | | E5KSD5, | | CoA (SCAD) | |
| | | | B4DUH1, | | Dehydrogenase | |
| | | | E9PE82 | | Deficiency | |
| ACADVL | 37 | 0072778 | P49748 | 111 | Very Long Chain | Fatty acid oxidation |
| | | | B3KPA6 | | Acyl-CoA | |
| | | | | | Dehydrogenase | |
| | | | | | (VLCAD) | |
| | | | | | Deficiency | |
| AGL | 178 | 0162688 | P35573 | 112 | GSD III (Cori/ | Liver glycogen storage |
| | | | A0A0S2A4E4 | | Forbe Disease or | disorder |
| | | | | | Debrancher) | |
| G6PC | 2538 | 0131482 | P35575 | 113 | GSDIa (Von | Liver glycogen storage |
| | | | | | Gierke Disease) | disorder |
| GBE1 | 2632 | 0114480 | Q04446 | 114 | GSD IV (Andersen | Liver glycogen storage |
| | | | Q59ET0 | | Disease, Brancher | disorder |
| | | | | | Enzyme) | |
| PHKA1 | 5255 | 0067177 | P46020 | 115 | GSD IXa | |
| PHKA2 | 0044446 | 5256 | P46019 | 116 | GSD IXa | Liver glycogen storage |
| | 5256 | 0044446 | | | | disorder |
| PHKB | 5257 | 0102893 | Q93100 | 117 | GSD IXb | Liver glycogen storage |
| | | | | | | disorder |
| PHKG2 | 5261 | 0156873 | P15735 | 118 | GSD IXc | Liver glycogen storage |
| | | | | | | disorder |
| SLC37A4 | 2542 | 0281500 | O43826 | 119 | GSDIb. c, d | Liver glycogen storage |
| | | 0137700 | A0A024R3H9, | | | disorder |
| | | | A8K0S7, | | | |
| | | | A0A024R3L1, | | | |
| | | | B4DUH2 | | | |
| PMM2 | 5373 | 0140650 | O15305, | 120 | PMM2-CDG | Glycosylation disorder |
| | | | A0A0S2Z4J6, | | | |
| | | | Q59F02 | | | |
| CBS | 102724560, | 0160200 | P35520, | 121 | Cystathionine | Aminoacidopathy |
| | 875 | | P0DN79, | | Beta-Synthase | |
| | | | Q9NTF0, | | Deficiency | |
| | | | B7Z2D6 | | (Classic | |
| | | | | | Homocystinuria); | |
| | | | | | Homocystinuria | |
| FAH | 2184 | 0103876 | P16930 | 122 | Tyrosinemia Type | Aminoacidopathy |
| | | | | | I | |
| TAT | 6898 | 0198650 | P17735, | 123 | Tyrosinemia Type | Aminoacidopathy |
| | | | A0A140VKB7 | | II | |
| | | | | | Tyrosinemia Type | |
| | | | | | III | |
| GALT | 2592 | 0213930 | P07902, | 124 | Galactosemia | Carbohydrate disorder |
| | | | A0A0S2Z3Y7, | | due to galactose-1- | |
| | | | B2RAT6 | | phosphate | |
| | | | | | uridylyltranserase | |
| | | | | | (GALT) | |
| | | | | | deficiency | |
| GALK1 | 2584 | 0108479 | P51570 | 125 | Galactosemia | Carbohydrate disorder |
| GALE | 2582 | 0117308 | Q14376 | 126 | Galactosemia | Carbohydrate disorder |
| G6PD | 2539 | 0160211 | P11413 | 127 | Glucose-6- | Carbohydrate disorder |
| | | | | | Phosphate | |
| | | | | | Dehydrogenase | |
| | | | | | (G6PD) | |
| | | | | | Deficiency | |
| SLC3A1 | 6519 | 0138079 | Q07837, | 128 | Cystinuria | Aminoacidopathy |
| | | | A0A0S2Z4E1, | | | |
| | | | B8ZZK1 | | | |
| SLC7A9 | 11136 | 0021488 | P82251 | 129 | Cystinuria | Aminoacidopathy |
| MTHFR | 4524 | 0177000 | P42898, | 130 | Homocystinuria | Aminoacidopathy |
| | | | Q59GJ6, | | | |
| | | | Q81U67 | | | |
| MTR | 4548 | 0116984 | Q99707 | 131 | Homocystinuria | Aminoacidopathy |
| MTRR | 4552 | 0124275 | Q9UBK8 | 132 | Homocystinuria | Aminoacidopathy |
| ATP7B | 540 | 0123191 | P35670, | 133 | Wilson Disease | Metal transport disorder |
| | | | A0A024RDX3, | | Copper | |
| | | | B7ZLR4, | | Metabolism | |
| | | | B7ZLR3, | | Disorder | |
| | | | E7ET55 | | | |
| HPRT1 | 3251 | 0165704 | P00492, | 134 | Lesch-Nyhan | Purine Metabolism |
| | | | A0A140VJL3 | | Syndrome | Disorder |
| | | | | | Purine Metabolism | |
| | | | | | Disorder | |
| HJV | 148738 | 0168509 | Q6ZVN8 | 135 | Hemochromatosis, | |
| | | | | | Type 2A | |
| HAMP | 57817 | 0105697 | P81172 | 136 | Hemochromatosis | |
| | | | | | Type 2B: Primary | |
| | | | | | Hemochromatosis | |
| JAG1 | 182 | 0101384 | P78504, | 137 | Alagille Syndrome | |
| | | | Q99740 | | 1 | |
| TTR | 7276 | 0118271 | P02766, | 138 | Familial TTR | |
| | | | E9KL36 | | Amyloidoisis; | |
| | | | | | Familial amyloid | |
| | | | | | polyneuropathy | |
| AGXT | 189 | 0172482 | P21549 | 139 | Primary | |
| | | | | | Hyperoxaluria | |
| | | | | | Type I | |
| LIPA | 3988 | 0107798 | P38571 | 140 | Lysosomal Acid | Lyososomal storage |
| | | | A0A0A0MT32 | | Lipase Deficiency | disorder |
| SERPING1 | 710 | 0149131 | P05155, | 141 | Hereditary | |
| | | | A0A0S2Z4J1, | | Angioedma | |
| | | | B2R659, | | | |
| | | | E7EWE5, | | | |
| | | | B3KSP2, | | | |
| | | | G5E9S2 | | | |
| HSD17B4 | 3295 | 0133835 | P51659 | 142 | D-Bifunctional | Peroxisomal disorders |
| | | | | | Protein Deficiency | |
| | | | | | X-linked | |
| | | | | | Adrenoleukodystrophy | |
| UROD | 7389 | 0126088 | P06132 | 143 | Porphyria Cutanea | |
| | | | | | Tarda | |
| HFE | 3077 | 0010704 | Q30201 | 144 | Porphyria Cutanea | |
| | | | | | Tarda | |
| LPL | 4023 | 0175445 | P06858, | 145 | Lipoprotein Lipase | |
| | | | A0A1B1RVA9 | | Deficiency | |
| | | | | | (“hyperlipoproteinemia | |
| | | | | | type Ia; | |
| | | | | | Buerger-Gruetz | |
| | | | | | syndrome, or | |
| | | | | | Familial | |
| | | | | | hyperchylomicronemia) | |
| GRHPR | 9380 | 0137106 | Q9UBQ7 | 146 | Primary | |
| | | | | | Hyperoxaluria | |
| | | | | | Type II | |
| HOGA1 | 112817 | 0241935 | Q86XE5 | 147 | Primary | |
| | | | | | Hyperoxaluria | |
| | | | | | Type III | |
| LDLR | 3949 | 0130164 | P01130, | 148 | Homozygous | |
| | | | A0A024R7D5 | | Familial | |
| | | | | | Hypercholesterolemia | |
| ACAD8 | 27034 | 0151498 | Q9UKU7 | 149 | isobutyryl-CoA | Organic acidemia |
| | | | | | dehydrogenase | |
| | | | | | (IBD) deficiency | |
| ACADSB | 36 | 0196177 | P45954, | 150 | short-branched | Organic acidemia |
| | | | A0A0S2Z3P9 | | chain acyl-CoA | |
| | | | | | dehydrogenase | |
| | | | | | (SBCAD) | |
| | | | | | deficiency | |
| ACAT1 | 38 | 0075239 | A0A140VJX1, | 151 | beta-ketothiolase | Organic acidemia |
| | | | P24752 | | deficiency | |
| ACSF3 | 197322 | 0176715 | Q4G176, | 152 | combined malonic | Organic acidemia |
| | | | F5H5A1 | | and methylmalonic | |
| | | | | | aciduria | |
| ASPA | 443 | 0108381 | P45381, | 153 | Canavan disease | Organic acidemia |
| | | | Q6FH48 | | | |
| AUH | 549 | 0148090 | Q13825, | 154 | 3- | Organic acidemia |
| | | | B4DYI6 | | methylglutaconic | |
| | | | | | acidemia type I | |
| DNAJC19 | 131118 | 0205981 | Q96DA6, | 155 | dilated | Organic acidemia |
| | | | A0A0S2Z5X1 | | cardiomyopathy | |
| | | | | | with ataxia | |
| | | | | | syndrome (causes | |
| | | | | | 3- | |
| | | | | | methylglutaconic | |
| | | | | | aciduria) | |
| ETHE1 | 23474 | 0105755 | A0A0S2Z580, | 156 | ethylmalonic | Organic acidemia |
| | | | O95571, | | encephalopathy | |
| | | | A0A0S2Z5N8, | | | |
| | | | A0A0S2Z5B3, | | | |
| | | | B2RCZ7 | | | |
| FBP1 | 2203 | 0165140 | P09467, | 157 | fructose 1,6- | Organic acidemia |
| | | | Q2TU34 | | Bisphosphatase | |
| | | | | | deficiency | |
| FTCD | 10841 | 0160282, | O95954 | 158 | glutamate | Organic acidemia |
| | | 0281775 | | | formiminotransferase | |
| | | | | | deficiency | |
| | | | | | (FIGLU | |
| GSS | 2937 | 0100983 | P48637, | 159 | glutathione | Organic acidemia |
| | | | V9HWJ1 | | synthetase | |
| | | | | | deficiency | |
| HIBCH | 26275 | 0198130 | A0A140VJL0, | 160 | 3- | Organic acidemia |
| | | | Q6NVY1 | | hyroxyisobutyryl- | |
| | | | | | CoA hydrolase | |
| | | | | | deficiency | |
| IDH2 | 3418 | 0182054 | P48735, | 161 | D-2- | Organic acidemia |
| | | | B4DSZ6 | | hydroxyglutaric | |
| | | | | | aciduria type II | |
| L2HGDH | 79944 | 0087299 | Q9H9P8 | 162 | L-2- | Organic acidemia |
| | | | | | hydroxyglutaric | |
| | | | | | aciduria | |
| MLYCD | 23417 | 0103150 | O95822 | 163 | malonic acidemia | Organic acidemia |
| OPA3 | 80207 | 0125741 | Q9H6K4, | 164 | Costeff syndrome/ | Organic acidemia |
| | | | B4DK77 | | 3- | |
| | | | | | methylglutaconic | |
| | | | | | aciduria type III | |
| OPLAH | 26873 | 0178814 | O14841 | 165 | 5-oxoprolinase | Organic acidemia |
| | | | | | deficiency | |
| OXCT1 | 5019 | 0083720 | A0A024R040, | 166 | SCOT deficiency | Organic acidemia |
| | | | P55809 | | | |
| POLG | 5428 | 0140521 | E5KNU5, | 167 | 3- | Organic acidemia |
| | | | P54098 | | methylglutaconic | |
| | | | | | aciduria | |
| PPM1K | 152926 | 0163644 | Q8N3J5 | 168 | maple syrup urine | Organic acidemia |
| | | | | | disease (MSUD), | |
| | | | | | variant type | |
| SERAC1 | 84947 | 0122335 | Q96JX3 | 169 | Megdel Syndrome | Organic acidemia |
| SLC25A1 | 6576 | 0100075 | D9HTE9, | 170 | D,L-2- | Organic acidemia |
| | | | B4DP62, | | hydroxyglutaric | |
| | | | P53007 | | aciduria | |
| SUCLA2 | 8803 | 0136143 | E5KS60, | 171 | succinate-CoA | Organic acidemia |
| | | | Q9P2R7, | | ligase deficiency, | |
| | | | Q9Y4T0 | | methylmalonic | |
| | | | | | aciduria | |
| SUCLG1 | 8802 | 0163541 | P53597 | 172 | succinate-CoA | Organic acidemia |
| | | | | | ligase deficiency, | |
| | | | | | methylmalonic | |
| | | | | | aciduria | |
| TAZ | 6901 | 0102125 | A0A0S2Z4K0, | 173 | Barth syndrome | Organic acidemia |
| | | | Q16635, | | | |
| | | | A6XNE1, | | | |
| | | | A0A0S2Z4E6, | | | |
| | | | A0A0S2Z4K9, | | | |
| | | | A0A0S2Z4F4 | | | |
| AGK | 55750 | 0006530, | A4D1U5, | 174 | 3- | Organic acidemia |
| | | 0262327 | Q53H12 | | methylglutaconic | |
| | | | | | aciduria | |
| CLPB | 81570 | 0162129 | Q9H078, | 175 | 3- | Organic acidemia |
| | | | A0A140VK11 | | methylglutaconic | |
| | | | | | aciduria | |
| TMEM70 | 54968 | 0175606 | Q9BUB7 | 176 | 3- | Organic acidemia |
| | | | | | methylglutaconic | |
| | | | | | aciduria | |
| ALDH18A1 | 5832 | 0059573 | P54886 | 177 | ALDH18A1- | Urea cycle disorder |
| | | | | | related cutis laxa | |
| OAT | 4942 | 0065154 | A0A140VJQ4, | 178 | gyrate atrophy | Urea cycle disorder |
| | | | P04181 | | (OAT) | |
| CA5A | 763 | 0174990 | P35218 | 179 | carbonic | Urea cycle disorder |
| | | | | | anhydrase | |
| | | | | | deficiency | |
| GLUD1 | 2746 | 0148672 | P00367, | 180 | glutamate | Urea cycle disorder |
| | | | E9KL48 | | dehydrogenase | |
| | | | | | deficiency | |
| GLUL | 2752 | 0135821 | A8YXX4, | 181 | glutamine | Urea cycle disorder |
| | | | P15104 | | synthetase | |
| | | | | | deficienc | |
| UMPS | 7372 | 0114491 | A8K5J1, | 182 | Orotic Aciduria | Urea cycle disorder |
| | | | P11172 | | | |
| SLC22A5 | 6584 | 0197375 | O76082 | 183 | carnitine- | Fatty acid oxidation |
| | | | | | acylcarnitine | |
| | | | | | translocase | |
| | | | | | (CACT) | |
| | | | | | deficiency | |
| CPT1A | 1374 | 0110090 | P50416, | 184 | carnitine | Fatty acid oxidation |
| | | | A0A024R5F4, | | palmitoyltransferase | |
| | | | B2RAQ8, | | type I (CPT I) | |
| | | | Q8WZ48 | | deficiency | |
| HADHA | 3030 | 0084754 | E9KL44, | 185 | long chain 3- | Fatty acid oxidation |
| | | | P40939 | | hydroxyacyl-CoA | |
| | | | | | dehydrogenase | |
| | | | | | (LCHAD) | |
| | | | | | deficiency | |
| HADH | 3033 | 0138796 | Q16836, | 186 | medium/short | Fatty acid oxidation |
| | | | B3KTT6 | | chain acyl-CoA | |
| | | | | | dehydrogenase | |
| | | | | | (M/SCHAD) | |
| | | | | | deficiency | |
| SLC52A1 | 55065 | 0132517 | Q9NWF4 | 187 | Riboflavin | Fatty acid oxidation |
| | | | | | transporter | |
| | | | | | deficiency | |
| SLC52A2 | 79581 | 0185803 | Q9HAB3 | 188 | Riboflavin | Fatty acid oxidation |
| | | | | | transporter | |
| | | | | | deficiency | |
| SLC52A3 | 113278 | 0101276 | K0A6P4, | 189 | Riboflavin | Fatty acid oxidation |
| | | | Q9NQ40 | | transporter | |
| | | | | | deficiency | |
| HADHB | 3032 | 0138029 | P55084, | 190 | Trifunctional | Fatty acid oxidation |
| | | | F5GZQ3 | | protein deficiency | |
| GYS2 | 2998 | 0111713 | P54840 | 191 | GSD 0 (Glycogen | Liver glycogen storage |
| | | | | | synthase, liver | disorder |
| | | | | | isoform) | |
| PYGL | 5836 | 0100504 | P06737 | 192 | GSD VI (Hers | Liver glycogen storage |
| | | | | | disease) | disorder |
| SLC2A2 | 6514 | 0163581 | P11168, | 193 | Fanconi-Bickel | Liver glycogen storage |
| | | | Q6PAU8 | | syndrome | disorder |
| ALG1 | 56052 | 0033011 | Q9BT22 | 194 | ALG1-CDG | Glycosylation disorder |
| ALG2 | 85365 | 0119523 | A0A024R184, | 195 | ALG2-associated | Glycosylation disorder |
| | | | Q9H553 | | myasthenic | |
| | | | | | syndrome | |
| ALG3 | 10195 | 0214160 | Q92685, | 196 | ALG3-CDG | Glycosylation disorder |
| | | | C9J7S5 | | | |
| ALG6 | 29929 | 0088035 | Q9Y672 | 197 | ALG6-CDG | Glycosylation disorder |
| ALG8 | 79053 | 0159063 | Q9BVK2, | 198 | ALG8-CDG | Glycosylation disorder |
| | | | A0A024R5K5 | | | |
| ALG9 | 79796 | 0086848 | Q9H6U8 | 199 | ALG9-CDG | Glycosylation disorder |
| ALG11 | 440138 | 0253710 | Q2TAA5 | 200 | ALG11-CDG | Glycosylation disorder |
| ALG12 | 79087 | 0182858 | A0A024R4V6, | 201 | ALG12-CDG | Glycosylation disorder |
| | | | Q9BV10 | | | |
| ALG13 | 79868 | 0101901 | Q9NP73, | 202 | ALG13-CDG | Glycosylation disorder |
| | | | A0A087WX43, | | | |
| | | | A0A087WT15 | | | |
| ATP6V0A2 | 23545 | 0185344 | Q9Y487 | 203 | ATP6V0A2- | Glycosylation disorder |
| | | | | | associated cutis | |
| | | | | | laxa | |
| B3GLCT | 145173 | 0187676 | Q6Y288 | 204 | B3GLCT-CDG | Glycosylation disorder |
| CHST14 | 113189 | 0169105 | Q8NCH0 | 205 | CHST14-CDG | Glycosylation disorder |
| COG1 | 9382 | 0166685 | Q8WTW3 | 206 | COG1-CDG | Glycosylation disorder |
| COG2 | 22796 | 0135775 | Q14746, | 207 | COG2-CDG | Glycosylation disorder |
| | | | B1ALW7 | | | |
| COG4 | 25839 | 0103051 | A0A0A0MS45, | 208 | COG4-CDG | Glycosylation disorder |
| | | | Q8N8L9, | | | |
| | | | Q9H9E3, | | | |
| | | | J3KNI1 | | | |
| COG5 | 10466 | 0164597, | Q9UP83 | 209 | COG5-CDG | Glycosylation disorder |
| | | 0284369 | | | | |
| COG6 | 57511 | 0133103 | A0A140VJG7, | 210 | COG6-CDG | Glycosylation disorder |
| | | | Q9Y2V7, | | | |
| | | | A0A024RDW5 | | | |
| COG7 | 91949 | 0168434 | A0A0S2Z652, | 211 | COG7-CDG | Glycosylation disorder |
| | | | P83436 | | | |
| COG8 | 84342 | 0272617 | A0A024R6Z6, | 212 | COG8-CDG | Glycosylation disorder |
| | | | Q96MW5 | | | |
| DOLK | 22845 | 0175283 | A0A0S2Z597, | 213 | DOLK-CDG | Glycosylation disorder |
| | | | Q9UPQ8 | | | |
| DHDDS | 79947 | 0117682 | Q86SQ9 | 214 | DHDDS-CDG | Glycosylation disorder |
| DPAGT1 | 1798 | 0172269 | A0A024R3H8, | 215 | DPAGT1-CDG | Glycosylation disorder |
| | | | Q9H3H5 | | | |
| DPM1 | 8813 | 0000419 | O60762, | 216 | DPM1-CDG | Glycosylation disorder |
| | | | Q5QPK2, | | | |
| | | | A0A0S2Z4Y5 | | | |
| DPM2 | 8818 | 0136908 | O94777 | 217 | DPM2-CDG | Glycosylation disorder |
| DPM3 | 54344 | 0179085 | A0A140VJI4, | 218 | DPM3-CDG | Glycosylation disorder |
| | | | Q9P2X0, | | | |
| | | | Q86TM7 | | | |
| G6PC3 | 92579 | 0141349 | Q9BUM1 | 219 | Congenital | Glycosylation disorder |
| | | | | | neutropenia | |
| GFPT1 | 2673 | 0198380 | Q06210 | 220 | Congenital | Glycosylation disorder |
| | | | | | myasthenic | |
| | | | | | syndrome | |
| GMPPA | 29926 | 0144591 | A0A024R482, | 221 | GMPPA-CDG | Glycosylation disorder |
| | | | Q96IJ6 | | | |
| GMPPB | 29925 | 0173540 | Q9Y5P6 | 222 | Congenital | Glycosylation disorder |
| | | | | | muscular | |
| | | | | | dystrophy, | |
| | | | | | congenital | |
| | | | | | myasthenic | |
| | | | | | syndrome, and | |
| | | | | | dystroglycanopathy | |
| MAGT1 | 84061 | 0102158 | A0A087WU53, | 223 | MAGT1-CDG; X- | Glycosylation disorder |
| | | | Q9H0U3 | | linked | |
| | | | | | immunodeficiency | |
| | | | | | with magnesium | |
| | | | | | defect, Epstein- | |
| | | | | | Barr virus | |
| | | | | | infection and | |
| | | | | | neoplasia (XMEN) | |
| | | | | | syndrome | |
| MAN1B1 | 11253 | 0177239 | Q9UKM7 | 224 | MAN1B1-CDG | Glycosylation disorder |
| MGAT2 | 4247 | 0168282 | Q10469 | 225 | MGAT2-CDG | Glycosylation disorder |
| MOGS | 7841 | 0115275 | Q13724, | 226 | MOGS-CDG | Glycosylation disorder |
| | | | Q58F09 | | | |
| MPDU1 | 9526 | 0129255 | J3QW43, | 227 | MPDU1-CDG | Glycosylation disorder |
| | | | O75352, | | | |
| | | | A0A0S2Z4W8, | | | |
| | | | B4DLH7 | | | |
| MPI | 4351 | 0178802 | H3BPP3, | 228 | MPI-CDG | Glycosylation disorder |
| | | | Q8NHZ6, | | | |
| | | | B4DW50, | | | |
| | | | F5GX71, | | | |
| | | | P34949, | | | |
| | | | H3BPB8 | | | |
| NGLY1 | 55768 | 0151092 | Q96IV0 | 229 | NGLY1-CDG | Glycosylation disorder |
| PGM1 | 5236 | 0079739 | B7Z6C2, | 230 | PGM1-CDG | Glycosylation disorder |
| | | | P36871, | | | |
| | | | B4DDQ8 | | | |
| PGM3 | 5238 | 0013375 | O95394, | 231 | PGM3-CDG | Glycosylation disorder |
| | | | A0A087WT27 | | | |
| RFT1 | 91869 | 0163933 | Q96AA3 | 232 | RFT1-CDG | Glycosylation disorder |
| SEC23B | 10483 | 0101310 | Q15437, | 233 | SEC23B-CDG | Glycosylation disorder |
| | | | B4DJW8 | | | |
| SLC35A1 | 10559 | 0164414 | P78382 | 234 | SLC35A1-CDG | Glycosylation disorder |
| SLC35A2 | 7355 | 0102100 | P78381, | 235 | SLC35A2-CDG | Glycosylation disorder |
| | | | A6NFI1, | | | |
| | | | A6NKM8, | | | |
| | | | B4DE15 | | | |
| SLC35C1 | 55343 | 0181830 | Q96A29, | 236 | SLC35C1-CDG | Glycosylation disorder |
| | | | B3KQH0 | | | |
| SSR4 | 6748 | 0180879 | P51571 | 237 | SSR4-CDG | Glycosylation disorder |
| SRD5A3 | 79644 | 0128039 | Q9H8P0 | 238 | SRD5A3-CDG | Glycosylation disorder |
| TMEM165 | 55858 | 0134851 | Q9HC07 | 239 | TMEM165-CDG | Glycosylation disorder |
| TRIP11 | 9321 | 0100815 | Q15643 | 240 | TRIP11-CDG | Glycosylation disorder |
| TUSC3 | 7991 | 0104723 | Q13454 | 241 | TUSC3-CDG | Glycosylation disorder |
| ALG14 | 199857 | 0172339 | Q96F25 | 242 | ALG14-CDG | Glycosylation disorder |
| B4GALT1 | 2683 | 0086062 | P15291, | 243 | B4GALT1-CDG | Glycosylation disorder |
| | | | W6MEN3 | | | |
| DDOST | 1650 | 0244038 | A0A024RAD5, | 244 | DDOST-CDG | Glycosylation disorder |
| | | | P39656 | | | |
| NUS1 | 116150 | 0153989 | Q96E22 | 245 | NUS1-CDG | Glycosylation disorder |
| RPN2 | 6185 | 0118705 | P04844 | 246 | RPN2-CDG | Glycosylation disorder |
| SEC23A | 10484 | 0100934 | Q15436 | 247 | SEC23A-CDG | Glycosylation disorder |
| SLC35A3 | 23443 | 0117620 | Q9Y2D2, | 248 | SLC35A3-CDG | Glycosylation disorder |
| | | | A0A1W2PRT7, | | | |
| | | | A0A1W2PSD1, | | | |
| | | | A0A1W2PQL8 | | | |
| ST3GAL3 | 6487 | 0126091 | Q11203 | 249 | ST3GAL3-CDG | Glycosylation disorder |
| STT3A | 3703 | 0134910 | P46977 | 250 | STT3A-CDG | Glycosylation disorder |
| STT3B | 201595 | 0163527 | Q8TCJ2 | 251 | STT3B-CDG | Glycosylation disorder |
| AGA | 175 | 0038002 | P20933 | 252 | Aspartylglucosaminuria | Lyososomal storage |
| | | | | | | disorder |
| ARSA | 410 | 0100299 | A0A0C4DFZ2, | 253 | Metachromatic | Lyososomal storage |
| | | | B4DVI5, | | leukodystrophy | disorder |
| | | | P15289 | | | |
| ARSB | 411 | 0113273 | A0A024RAJ9, | 254 | Mucopolysaccharidosis | Lyososomal storage |
| | | | P15848, | | type VI | disorder |
| | | | A8K4A0 | | | |
| ASAH1 | 427 | 0104763 | A8K0B6, | 255 | Farber disease | Lyososomal storage |
| | | | Q13510, | | | disorder |
| | | | Q53H01 | | | |
| ATP13A2 | 23400 | 0159363 | Q8N4D4, | 256 | Neuronal ceroid | Lyososomal storage |
| | | | Q9NQ11, | | lipofuscinosis 12 | disorder |
| | | | Q8NBS1 | | (CLN12), Kufor- | |
| | | | | | Rakeb syndrome | |
| | | | | | (KRS) | |
| CLN3 | 1201 | 0188603, | A0A024QZB8, | 257 | Neuronal ceroid | Lyososomal storage |
| | | 0261832 | Q13286, | | lipofuscinosis 3 | disorder |
| | | | B4DMY6, | | (CLN3) | |
| | | | Q2TA70, | | | |
| | | | B4DFF3 | | | |
| CLN5 | 1203 | 0102805 | A0A024R644, | 258 | Neuronal ceroid | Lyososomal storage |
| | | | O75503 | | lipofuscinosis 5 | disorder |
| | | | | | (CLN5) | |
| CLN6 | 54982 | 0128973 | A0A024R601, | 259 | Neuronal ceroid | Lyososomal storage |
| | | | Q9NWW5 | | lipofuscinosis 6 | disorder |
| | | | | | (CLN6) | |
| CLN8 | 2055 | 0182372, | A0A024QZ57, | 260 | Neuronal ceroid | Lyososomal storage |
| | | 0278220 | Q9UBY8 | | lipofuscinosis 8 | disorder |
| | | | | | (CLN8) | |
| CTNS | 1497 | 0040531 | A0A0S2Z3I9, | 261 | cystinosis | Lyososomal storage |
| | | | O60931, | | | disorder |
| | | | A0A0S2Z3K3 | | | |
| CTSA | 5476 | 0064601 | P10619, | 262 | Galactosialidosis | Lyososomal storage |
| | | | X6R8A1, | | | disorder |
| | | | B4E324, | | | |
| | | | X6R5C5 | | | |
| CTSD | 1509 | 0117984 | P07339, | 263 | Neuronal ceroid | Lyososomal storage |
| | | | V9HWI3 | | lipofuscinosis 10 | disorder |
| | | | | | (CLN10) | |
| CTSF | 8722 | 0174080 | Q9UBX1 | 264 | Neuronal ceroid | Lyososomal storage |
| | | | | | lipofuscinosis 13 | disorder |
| | | | | | (CLN13) | |
| CTSK | 1513 | 0143387 | P43235 | 265 | Pycnodysostosis | Lyososomal storage |
| | | | | | | disorder |
| DNAJC5 | 80331 | 0101152 | Q6AHX3, | 266 | Neuronal ceroid | Lyososomal storage |
| | | | Q9H3Z4 | | lipofuscinosis 4 | disorder |
| | | | | | (CLN4) | |
| FUCA1 | 2517 | 0179163 | P04066, | 267 | Fucosidosis | Lyososomal storage |
| | | | B5MDC5 | | | disorder |
| GAA | 2548 | 0171298 | P10253 | 268 | Pompe disease | Lyososomal storage |
| | | | | | | disorder |
| GALC | 2581 | 0054983 | A0A0A0MQV0, | 269 | Krabbe disease | Lyososomal storage |
| | | | P54803 | | | disorder |
| GALNS | 2588 | 0141012 | P34059, | 270 | Mucopolysaccharidosis | Lyososomal storage |
| | | | Q96I49, | | type IVa | disorder |
| | | | Q6YL38 | | | |
| GLA | 2717 | 0102393 | P06280, | 271 | Fabry disease | Lyososomal storage |
| | | | Q53Y83 | | | disorder |
| GLB1 | 2720 | 0170266 | P16278, | 272 | GM1 | Lyososomal storage |
| | | | B7Z6Q5 | | gangliosidosis, | disorder |
| | | | | | Mucopolysaccharidosis | |
| | | | | | IVb | |
| GM2A | 2760 | 0196743 | P17900 | 273 | GM2- | Lyososomal storage |
| | | | | | gangliosidosis, AB | disorder |
| | | | | | variant | |
| GNPTAB | 79158 | 0111670 | Q3T906 | 274 | Mucolipidosis type | Lyososomal storage |
| | | | | | II alpha/beta, | disorder |
| | | | | | Mucolipidosis III | |
| | | | | | alpha/beta | |
| GNPTG | 84572 | 0090581 | Q9UJJ9 | 275 | Mucolipidosis III | Lyososomal storage |
| | | | | | gamma | disorder |
| GNS | 2799 | 0135677 | A0A024RBC5, | 276 | Mucopolysaccharidosis | Lyososomal storage |
| | | | P15586, | | type IIID | disorder |
| | | | Q7Z3X3 | | | |
| GRN | 2896 | 0030582 | P28799 | 277 | Neuronal ceroid | Lyososomal storage |
| | | | | | lipofuscinosis 11 | disorder |
| | | | | | (CLN11), | |
| | | | | | frontotemporal | |
| | | | | | dementia | |
| GUSB | 2990 | 0169919 | P08236 | 278 | Mucopolysaccharidosis | Lyososomal storage |
| | | | | | type VII | disorder |
| HEXA | 3073 | 0213614 | A0A0S2Z3W3, | 279 | Tay-Sachs disease | Lyososomal storage |
| | | | P06865, | | | disorder |
| | | | B4DVA7, | | | |
| | | | H3BP20 | | | |
| HEXB | 3074 | 0049860 | A0A024RAJ6, | 280 | Sandhoff diseaase | Lyososomal storage |
| | | | P07686, | | | disorder |
| | | | Q5URX0 | | | |
| HGSNAT | 138050 | 0165102 | Q68CP4, | 281 | Mucopolysaccharidosis | Lyososomal storage |
| | | | Q8IVU6 | | type IIIC | disorder |
| HYAL1 | 3373 | 0114378 | A0A024R2X3, | 282 | Mucopolysaccharidosis | Lyososomal storage |
| | | | QI2794, | | type IX | disorder |
| | | | B3KUI5, | | | |
| | | | A0A0S2Z3Q0 | | | |
| IDS | 3423 | 0010404 | P22304, | 283 | Mucopolysaccharidosis | Lyososomal storage |
| | | | B4DGD7 | | type II | disorder |
| IDUA | 3425 | 0127415 | P35475 | 284 | Mucopolysaccharidosis | Lyososomal storage |
| | | | | | type I | disorder |
| KCTD7 | 154881 | 0243335 | Q96MP8, | 285 | Neuronal ceroid | Lyososomal storage |
| | | | A0A024RDN7 | | lipofuscinosis 14 | disorder |
| | | | | | (CLN14) | |
| LAMP2 | 3920 | 0005893 | P13473 | 286 | Danon disease | Lyososomal storage |
| | | | | | | disorder |
| MAN2B1 | 4125 | 0104774 | O00754, | 287 | alpha- | Lyososomal storage |
| | | | A8K6A7 | | mannosidosis | disorder |
| MANBA | 4126 | 0109323 | O00462 | 288 | beta-mannosidosis | Lyososomal storage |
| | | | | | | disorder |
| MCOLN1 | 57192 | 0090674 | Q9GZU1 | 289 | Mucolipidosis type | Lyososomal storage |
| | | | | | IV | disorder |
| MFSD8 | 256471 | 0164073 | Q8NHS3 | 290 | Neuronal ceroid | Lyososomal storage |
| | | | | | lipofuscinosis 7 | disorder |
| | | | | | (CLN7) | |
| NAGA | 4668 | 0198951 | A0A024R1Q5, | 291 | Schindler disease | Lyososomal storage |
| | | | P17050 | | | disorder |
| NAGLU | 4669 | 0108784 | A0A140VJE4, | 292 | Mucopolysaccharidosis | Lyososomal storage |
| | | | P54802 | | IIIB | disorder |
| NEU1 | 4758 | 0204386, | Q5JQI0, | 293 | Mucolipidosis type | Lyososomal storage |
| | | 0227315, | Q99519 | | I, Sialidosis I | disorder |
| | | 0227129, | | | | |
| | | 0223957, | | | | |
| | | 0234846, | | | | |
| | | 0184494, | | | | |
| | | 0228691, | | | | |
| | | 0234343 | | | | |
| NPC1 | 4864 | 0141458 | O15118 | 294 | Niemann-Pick | Lyososomal storage |
| | | | | | type C | disorder |
| NPC2 | 10577 | 0119655 | A0A024R6C0, | 295 | Niemann-Pick | Lyososomal storage |
| | | | P61916, | | type C | disorder |
| | | | G3V3E8 | | | |
| SGSH | 6448 | 0181523 | P51688 | 296 | Mucopolysaccharidosis | Lyososomal storage |
| | | | | | IIIA | disorder |
| PPT1 | 5538 | 0131238 | P50897 | 297 | Neuronal ceroid | Lyososomal storage |
| | | | | | lipofuscinosis 1 | disorder |
| | | | | | (CLN1) | |
| PSAP | 5660 | 0197746 | P07602, | 298 | Prosaposin | Lyososomal storage |
| | | | A0A024QZQ2 | | deficiency, SapA | disorder |
| | | | | | deficiency (Krabbe | |
| | | | | | variant), SapB | |
| | | | | | deficiency | |
| | | | | | (MLD variant), | |
| | | | | | SapC deficiency | |
| | | | | | (Gaucher variant) | |
| SLC17A5 | 26503 | 0119899 | Q9NRA2 | 299 | Infantile sialic acid | Lyososomal storage |
| | | | | | storage disease, | disorder |
| | | | | | Salla disease | |
| SMPD1 | 6609 | 0166311 | P17405, | 300 | Niemann Pick | Lyososomal storage |
| | | | Q59EN6, | | types A and B | disorder |
| | | | E9LUE8, | | | |
| | | | Q8IUN0, | | | |
| | | | E9LUE9 | | | |
| SUMF1 | 285362 | 0144455 | Q8NBK3 | 301 | Multiple sulfatase | Lyososomal storage |
| | | | | | deficiency | disorder |
| TPP1 | 1200 | 0166340 | O14773 | 302 | Neuronal ceroid | Lyososomal storage |
| | | | | | lipofuscinosis 2 | disorder |
| | | | | | (CLN2) | |
| AHCY | 191 | 0101444 | P23526, | 303 | Hypermethioninemia | Aminoacidophaty |
| | | | Q1RMG2 | | | |
| GNMT | 27232 | 0124713 | A0A0S2Z5F2, | 304 | Hypermethioninemia | Aminoacidophaty |
| | | | Q14749, | | | |
| | | | V9HW60 | | | |
| MAT1A | 4143 | 0151224 | Q00266 | 305 | Hypermethioninemia | Aminoacidophaty |
| GCH1 | 2643 | 0131979 | A0A024R642, | 306 | BH4 cofactor | Aminoacidophaty |
| | | | P30793, | | deficiency | |
| | | | Q8IZH9 | | | |
| PCBD1 | 5092 | 0166228 | P61457 | 307 | BH4 cofactor | Aminoacidophaty |
| | | | | | deficiency | |
| PTS | 5805 | 0150787 | Q03393 | 308 | BH4 cofactor | Aminoacidophaty |
| | | | | | deficiency | |
| QDPR | 5860 | 0151552 | A0A140VKA9, | 309 | BH4 cofactor | Aminoacidophaty |
| | | | P09417 | | deficiency | |
| SPR | 6697 | 0116096 | P35270 | 310 | BH4 cofactor | Aminoacidophaty |
| | | | | | deficiency | |
| DNAJC12 | 56521 | 0108176 | Q6IAH1, | 311 | Phenylalanine, | Aminoacidophaty |
| | | | Q9UKB3 | | tyrosine, and | |
| | | | | | tryptophan | |
| | | | | | hydroxylases heat | |
| | | | | | shock | |
| | | | | | co-chaperone | |
| | | | | | deficiency | |
| ALDH4A1 | 8659 | 0159423 | P30038, | 312 | Hyperprolinemia | Aminoacidophaty |
| | | | A0A024RAD8 | | | |
| PRODH | 5625 | 0100033 | O43272 | 313 | Hyperprolinemia | Aminoacidophaty |
| HPD | 3242 | 0158104 | P32754 | 314 | Tyrosinemia type | Aminoacidophaty |
| | | | | | II | |
| GBA | 2629 | 0177628, | A0A068F658, | 315 | Gaucher disease | |
| | | 0262446 | P04062, | | | |
| | | | B7Z6S9 | | | |
| HGD | 3081 | 0113924 | Q93099, | 316 | Alkaptonuria | |
| | | | B3KW64 | | | |
| AMN | 81693 | 0166126 | Q9BXJ7, | 317 | Combined | Organic acidemia |
| | | | B3KP64 | | Methylmalonic | |
| | | | | | Acidemia and | |
| | | | | | Homocystinuria | |
| CD320 | 51293 | 0167775 | Q9NPF0 | 318 | Combined | Organic acidemia |
| | | | | | Methylmalonic | |
| | | | | | Acidemia and | |
| | | | | | Homocystinuria | |
| CUBN | 8029 | 0107611 | O60494 | 319 | Combined | Organic acidemia |
| | | | | | Methylmalonic | |
| | | | | | Acidemia and | |
| | | | | | Homocystinuria | |
| GIF | 2694 | 0134812 | P27352 | 320 | Combined | Organic acidemia |
| | | | | | Methylmalonic | |
| | | | | | Acidemia and | |
| | | | | | Homocystinuria | |
| TCN1 | 6947 | 0134827 | P20061 | 321 | Combined | Organic acidemia |
| | | | | | Methylmalonic | |
| | | | | | Acidemia and | |
| | | | | | Homocystinuria | |
| TCN2 | 6948 | 0185339 | P20062 | 322 | Combined | Organic acidemia |
| | | | | | Methylmalonic | |
| | | | | | Acidemia and | |
| | | | | | Homocystinuria | |
| PREPL | 9581 | 0138078 | Q4J6C6 | 323 | Cystinuria | Aminoacidophaty |
| PHGDH | 26227 | 0092621 | O43175 | 324 | Disorders of | Aminoacidophaty |
| | | | | | Serine | |
| | | | | | Biosynthesis | |
| PSAT1 | 29968 | 0135069 | A0A024R280, | 325 | Disorders of | Aminoacidophaty |
| | | | Q9Y617, | | Serine | |
| | | | A0A024R222 | | Biosynthesis | |
| PSPH | 5723 | 0146733 | A0A024RDL3, | 326 | Disorders of | Aminoacidophaty |
| | | | P78330 | | Serine | |
| | | | | | Biosynthesis | |
| AMT | 275 | 0145020 | A0A024R2U7, | 327 | Glycine | Aminoacidophaty |
| | | | P48728 | | Encephalopathy | |
| GCSH | 2653 | 0140905 | P23434 | 328 | Glycine | Aminoacidophaty |
| | | | | | Encephalopathy | |
| GLDC | 2731 | 0178445 | P23378 | 329 | Glycine | Aminoacidophaty |
| | | | | | Encephalopathy | |
| LIAS | 11019 | 0121897 | O43766, | 330 | Glycine | Aminoacidophaty |
| | | | Q6P5Q6, | | Encephalopathy | |
| | | | B4E0L7, | | | |
| | | | A0A024R9W0, | | | |
| | | | A0A1W2PQE9, | | | |
| | | | A0A1X7SBR7 | | | |
| NFU1 | 27247 | 0169599 | Q9UMS0 | 331 | Glycine | Aminoacidophaty |
| | | | | | Encephalopathy | |
| SLC6A9 | 6536 | 0196517 | P48067, | 332 | Glycine | Aminoacidophaty |
| | | | B7Z3W8, | | Encephalopathy | |
| | | | B7Z589 | | | |
| SLC2A1 | 6513 | 0117394 | P11166, | 333 | Glucose | Carbohydrate disorder |
| | | | Q59GX2 | | Transporter Type 1 | |
| | | | | | Deficiency | |
| ATP7A | 538 | 0165240 | B4DRW0, | 334 | ATP7A-Related | Metal transport disorder |
| | | | Q04656, | | Disorders | |
| | | | Q762B6 | | Copper | |
| | | | | | Metabolism | |
| | | | | | Disorder | |
| AP1S1 | 1174 | 0106367 | A0A024QYT6, | 335 | Copper | Metal transport disorder |
| | | | P61966 | | Metabolism | |
| | | | | | Disorder | |
| CP | 1356 | 0047457 | A5PL27, | 336 | Copper | Metal transport disorder |
| | | | P00450 | | Metabolism | |
| | | | | | Disorder | |
| SLC33A1 | 9197 | 0169359 | O00400 | 337 | Copper | Metal transport disorder |
| | | | | | Metabolism | |
| | | | | | Disorder | |
| PEX7 | 5191 | 0112357 | O00628, | 338 | Adult Refsum | Peroxisomal disorders |
| | | | Q6FGN1 | | Disease | |
| | | | | | Rhizomelic | |
| | | | | | Chondrodysplasia | |
| | | | | | Punctata Spectrum | |
| PHYH | 5264 | 0107537 | O14832 | 339 | Adult Refsum | Peroxisomal disorders |
| | | | | | Disease | |
| AGPS | 8540 | 0018510 | O00116, | 340 | Rhizomelic | Peroxisomal disorders |
| | | | B7Z3Q4 | | Chondrodysplasia | |
| | | | | | Punctata Spectrum | |
| GNPAT | 8443 | 0116906 | O15228 | 341 | Rhizomelic | Peroxisomal disorders |
| | | | | | Chondrodysplasia | |
| | | | | | Punctata Spectrum | |
| ABCD1 | 215 | 0101986 | P33897 | 342 | X-linked | Peroxisomal disorders |
| | | | | | Adrenoleukodystrophy | |
| ACOX1 | 51 | 0161533 | Q15067 | 343 | X-linked | Peroxisomal disorders |
| | | | | | Adrenoleukodystrophy | |
| PEX1 | 5189 | 0127980 | O43933, | 344 | X-linked | Peroxisomal disorders |
| | | | A0A0C4DG33, | | Adrenoleukodystrophy | |
| | | | B4DER6 | | | |
| PEX2 | 5828 | 0164751 | P28328 | 345 | X-linked | Peroxisomal disorders |
| | | | | | Adrenoleukodystrophy | |
| PEX3 | 8504 | 0034693 | P56589 | 346 | X-linked | Peroxisomal disorders |
| | | | | | Adrenoleukodystrophy | |
| PEX5 | 5830 | 0139197 | A0A0S2Z480, | 347 | X-linked | Peroxisomal disorders |
| | | | P50542, | | Adrenoleukodystrophy | |
| | | | B4DR50, | | | |
| | | | A0A0S2Z4F3, | | | |
| | | | A0A0S2Z4H1, | | | |
| | | | B4E0T2 | | | |
| PEX6 | 5190 | 0124587 | A0A024RD09, | 348 | X-linked | Peroxisomal disorders |
| | | | Q13608 | | Adrenoleukodystrophy | |
| PEX10 | 5192 | 0157911 | A0A024R068, | 349 | X-linked | Peroxisomal disorders |
| | | | O60683, | | Adrenoleukodystrophy | |
| | | | A0A024R0A4 | | | |
| PEX12 | 5193 | 0108733 | O00623 | 350 | X-linked | Peroxisomal disorders |
| | | | | | Adrenoleukodystrophy | |
| PEX13 | 5194 | 0162928 | Q92968 | 351 | X-linked | Peroxisomal disorders |
| | | | | | Adrenoleukodystrophy | |
| PEX14 | 5195 | 0142655 | O75381 | 352 | X-linked | Peroxisomal disorders |
| | | | | | Adrenoleukodystrophy | |
| PEX16 | 9409 | 0121680 | Q9Y5Y5 | 353 | X-linked | Peroxisomal disorders |
| | | | | | Adrenoleukodystrophy | |
| PEX19 | 5824 | 0162735 | P40855, | 354 | X-linked | Peroxisomal disorders |
| | | | A0A0S2Z497 | | Adrenoleukodystrophy | |
| PEX26 | 55670 | 0215193 | A0A024R100, | 355 | X-linked | Peroxisomal disorders |
| | | | Q7Z412, | | Adrenoleukodystrophy | |
| | | | A0A0S2Z5M7, | | | |
| | | | Q7Z2D7 | | | |
| AMACR | 23600 | 0242110 | Q9UHK6 | 356 | Zellweger | Peroxisomal disorders |
| | | | | | Spectrum Disorder | |
| ADA | 100 | 0196839 | A0A0S2Z381, | 357 | Purine Metabolism | Purine Metabolism |
| | | | P00813, | | Disorder | Disorder |
| | | | F5GWI4 | | | |
| ADSL | 158 | 0239900 | P30566, | 358 | Purine Metabolism | Purine Metabolism |
| | | | X5D8S6, | | Disorder | Disorder |
| | | | X5D7W4, | | | |
| | | | A0A1B0GWJ0 | | | |
| AMPD1 | 270 | 0116748 | P23109 | 359 | Purine Metabolism | Purine Metabolism |
| | | | | | Disorder | Disorder |
| GPHN | 10243 | 0171723 | Q9NQX3 | 360 | Purine Metabolism | Purine Metabolism |
| | | | | | Disorder | Disorder |
| MOCOS | 55034 | 0075643 | Q96EN8 | 361 | Purine Metabolism | Purine Metabolism |
| | | | | | Disorder | Disorder |
| MOCS1 | 4337 | 0124615 | A0A024RD17, | 362 | Purine Metabolism | Purine Metabolism |
| | | | Q9NZB8 | | Disorder | Disorder |
| PNP | 4860 | 0198805 | P00491, | 363 | Purine Metabolism | Purine Metabolism |
| | | | V9HWH6 | | Disorder | Disorder |
| XDH | 7498 | 0158125 | P47989 | 364 | Purine Metabolism | Purine Metabolism |
| | | | | | Disorder | Disorder |
| SUOX | 6821 | 0139531 | A0A024RB79, | 365 | Purine Metabolism | Purine Metabolism |
| | | | P51687 | | Disorder | Disorder |
| OGDH | 4967 | 0105953 | A0A140VJQ5, | 366 | 2-Ketoglutarate | PYRUVATE |
| | | | Q02218, | | Dehydrogenase | METABOLISM AND |
| | | | B4E3E9, | | Deficiency | TRICARBOXYLIC ACID |
| | | | E9PCR7, | | | CYCLE DEFECT |
| | | | E9PDF2 | | | |
| SLC25A19 | 60386 | 0125454 | Q5JPC1, | 367 | 2-Ketoglutarate | PYRUVATE |
| | | | Q9HC21 | | Dehydrogenase | METABOLISM AND |
| | | | | | Deficiency | TRICARBOXYLIC ACID |
| | | | | | | CYCLE DEFECT |
| DHTKD1 | 55526 | 0181192 | Q96HY7 | 368 | 2-Ketoglutarate | PYRUVATE |
| | | | | | Dehydrogenase | METABOLISM AND |
| | | | | | Deficiency | TRICARBOXYLIC ACID |
| | | | | | | CYCLE DEFECT |
| SLC13A5 | 284111 | 0141485 | Q68D44, | 369 | Citrate Transporter | PYRUVATE |
| | | | Q86YT5 | | Deficiency | METABOLISM AND |
| | | | | | | TRICARBOXYLIC ACID |
| | | | | | | CYCLE DEFECT |
| FH | 2271 | 0091483 | A0A0S2Z4C3, | 370 | Fumarase | PYRUVATE |
| | | | P07954 | | Deficiency | METABOLISM AND |
| | | | | | | TRICARBOXYLIC ACID |
| | | | | | | CYCLE DEFECT |
| DLAT | 1737 | 0150768 | P10515, | 371 | Pyruvate | PYRUVATE |
| | | | Q86YI5 | | Dehydrogenase | METABOLISM AND |
| | | | | | Deficiency | TRICARBOXYLIC ACID |
| | | | | | | CYCLE DEFECT |
| MPC1 | 51660 | 0060762 | Q5TI65, | 372 | Pyruvate | PYRUVATE |
| | | | Q9Y5U8 | | Dehydrogenase | METABOLISM AND |
| | | | | | Deficiency | TRICARBOXYLIC ACID |
| | | | | | | CYCLE DEFECT |
| PDHA1 | 5160 | 0131828 | A0A024RBX9, | 373 | Pyruvate | PYRUVATE |
| | | | P08559 | | Dehydrogenase | METABOLISM AND |
| | | | | | Deficiency | TRICARBOXYLIC ACID |
| | | | | | | CYCLE DEFECT |
| PDHB | 5162 | 0168291 | P11177 | 374 | Pyruvate | PYRUVATE |
| | | | | | Dehydrogenase | METABOLISM AND |
| | | | | | Deficiency | TRICARBOXYLIC ACID |
| | | | | | | CYCLE DEFECT |
| PDHX | 8050 | 0110435 | O00330 | 375 | Pyruvate | PYRUVATE |
| | | | | | Dehydrogenase | METABOLISM AND |
| | | | | | Deficiency | TRICARBOXYLIC ACID |
| | | | | | | CYCLE DEFECT |
| PDP1 | 54704 | 0164951 | Q9P0J1, | 376 | Pyruvate | PYRUVATE |
| | | | Q6P1N1, | | Dehydrogenase | METABOLISM AND |
| | | | A0A024R9C0 | | Deficiency | TRICARBOXYLIC ACID |
| | | | | | | CYCLE DEFECT |
| ABCC2 | 1244 | 0023839 | Q92887 | 377 | Dubin-Johnson | |
| | | | | | syndrome | |
| SLCO1B1 | 10599 | 0134538 | A0A024RAU7, | 378 | Rotor Syndrome | |
| | | | Q05CV5, | | | |
| | | | Q9Y6L6 | | | |
| SLCO1B3 | 28234 | 0111700 | B3KP78, | 379 | Rotor Syndrome | |
| | | | Q9NPD5 | | | |
| HFE2 | 148738 | 0168509 | Q6ZVN8, | 380 | Hemochromatosis, | |
| | | | A8K466, | | type 2A | |
| | | | A0A024R4F5 | | | |
| ADAMTS13 | 11093 | 0160323, | Q76LX8 | 381 | Congenital | |
| | | 0281244 | | | thrombotic | |
| | | | | | thrombocytopenic | |
| | | | | | purpura due to | |
| | | | | | ADAMTS-13 | |
| | | | | | deficiency | |
| PYGM | 5837 | 0068976 | P11217 | 382 | McArdle's Disease | |
| COL1A2 | 1278 | 0164692 | A0A0S2Z3H5, | 383 | Ehlers-Danlos | |
| | | | P08123 | | syndrome, cardiac | |
| | | | | | valvular type | |
| TNFRSF11B | 4982 | 0164761 | O00300 | 384 | Juvenile Paget's | |
| | | | | | disease | |
| TSC1 | 7248 | 0165699 | Q86WV8, | 385 | Tuberous sclerosis | |
| | | | Q92574, | | | |
| | | | X5D9D2, | | | |
| | | | Q32NF0 | | | |
| TSC2 | 7249 | 0103197 | P49815, | 386 | Tuberous sclerosis | |
| | | | X5D7Q2, | | | |
| | | | B3KWH7, | | | |
| | | | Q5HYF7, | | | |
| | | | H3BMQ0, | | | |
| | | | X5D2U8 | | | |
| DHCR7 | 1717 | 0172893 | A0A024R5F7, | 387 | Smith-Lemli-Opitz | |
| | | | Q9UBM7 | | Syndrome | |
| PGK1 | 5230 | 0102144 | P00558, | 388 | D- | |
| | | | V9HWF4 | | glycericacidemia | |
| VLDLR | 7436 | 0147852 | P98155, | 389 | Dysequilibrium | |
| | | | Q5VVF5 | | syndrome | |
| KYNU | 8942 | 0115919 | Q16719 | 390 | Encephalopathy | |
| | | | | | due to | |
| | | | | | hydroxykynureninuria | |
| F5 | 2153 | 0198734 | P12259 | 391 | Factor V | |
| | | | | | deficiency | |
| C3 | 718 | 0125730 | B4DR57, | 392 | Atypical hemolytic | |
| | | | P01024, | | uremic syndrome | |
| | | | V9HWA9 | | with C3 anomaly | |
| COL4A1 | 1282 | 0187498 | A5PKV2, | 393 | Autosomal | |
| | | | F5H5K0, | | dominant familial | |
| | | | P02462 | | hematuria - retinal | |
| | | | | | arteriolar | |
| | | | | | tortuosity - | |
| | | | | | contractures | |
| CFH | 3075 | 0000971 | A0A024R962, | 394 | Atypical hemolytic | |
| | | | P08603, | | uremic syndrome | |
| | | | A0A0D9SG88 | | | |
| SLC12A2 | 6558 | 0064651 | P55011, | 395 | Bartter syndrome | |
| | | | Q53ZR1, | | type I (neonatal) | |
| | | | B7ZM24 | | | |
| GK | 2710 | 0198814 | B4DH54, | 396 | Glycerol kinase | |
| | | | P32189 | | deficiency | |
| SFTPC | 6440 | 0168484 | A0A0A0MTC9, | 397 | Chronic | |
| | | | P11686, | | respiratory distress | |
| | | | A0A0S2Z4Q0, | | with surfactant | |
| | | | E5RI64 | | metabolism | |
| | | | | | deficiency | |
| CRTAP | 10491 | 0170275 | O75718 | 398 | Osteogenesis | |
| | | | | | Imperfecta VII | |
| P3H1 | 64175 | 0117385 | Q32P28 | 399 | Osteogenesis | |
| | | | | | Imperfecta VIII | |
| COL7A1 | 1294 | 0114270 | Q02388, | 400 | Autosomal | |
| | | | Q59F16 | | recessive | |
| | | | | | dystrophic | |
| | | | | | epidermolysis | |
| | | | | | bullosa | |
| PKLR | 5313 | 0143627 | P30613 | 401 | Pyruvate Kinase | |
| | | | | | deficiency | |
| TALDO1 | 6888 | 0177156 | A0A140VK56, | 402 | Transaldolase | |
| | | | P37837 | | deficiency | |
| TF | 7018 | 0091513 | A0PJA6, | 403 | Atransferrinemia | |
| | | | P02787, | | (familial | |
| | | | Q06AH7 | | hypotransferrinemia) | |
| EPCAM | 4072 | 0119888 | P16422 | 404 | Intestinal epithelial | |
| | | | | | dysplasia | |
| VHL | 7428 | 0134086 | A0A024R2F2, | 405 | Familial | |
| | | | P40337, | | erythrocytosis type | |
| | | | A0A0S2Z4K1 | | 2; von Hippel | |
| | | | | | Lindau disease | |
| GC | 2638 | 0145321 | P02774 | 406 | Vitamin D | |
| | | | | | deficiency | |
| SERPINA1 | 5265 | 0197249, | E9KL23, | 407 | Alpha-1 | |
| | | 0277377 | P01009 | | antitrypsin | |
| | | | | | deficiency | |
| ABCC6 | 368 | 0091262, | O95255 | 408 | Pseudoxanthoma | |
| | | 0275331 | | | elasticum | |
| F8 | 2157 | 0185010 | P00451 | 409 | Hemophilia A | |
| F9 | 2158 | 0101981 | P00740 | 410 | Hemophilia B | |
| ApoB | 338 | 0084674 | P04114 | 411 | Familial | |
| | | | | | hypercholesterolemia | |
| PCSK9 | 255738 | 0169174 | Q8NBP7 | 412 | Familial | |
| | | | | | hypercholesterolemia | |
| LDLRAP1 | 26119 | 0157978 | B3KR97, | 413 | Familial | |
| | | | Q5SW96 | | hypercholesterolemia | |
| ABCG5 | 64240 | 0138075 | Q9H222 | 414 | Sitosterolemia | |
| ABCG8 | 64241 | 0143921 | Q9H221 | 415 | Sitosterolemia | |
| LCAT | 3931 | 0213398 | A0A140VK24, | 416 | Lecithin | |
| | | | P04180 | | cholesterol | |
| | | | | | acyltransferase | |
| | | | | | deficiency | |
| SPINK5 | 11005 | 0133710 | Q9NQ38 | 417 | Netherton | |
| | | | | | syndrome | |
| GNE | 10020 | 0159921 | Q9Y223 | 418 | Inclusion body | |
| | | | | | myopathy 2 | |

In some embodiments, the payload gene encodes a protein that is ornithine transcarbamylase (OTC), optionally human OTC (hOTC). OTC (also called ornithine carbamoyltransferase or EC 2.1.3.3) is an enzyme that catalyzes the reaction between carbamoyl phosphate and ornithine to form citrulline and phosphate. The functional enzyme consists of three identical subunits. OTC is the last enzyme in the proximal portion of the urea cycle, which consists of the reactions that take place in the mitochondria. OTC deficiency is a disorder that when inherited causes ammonia to accumulate in the blood. Toxic levels of ammonia in the blood can result in neurotoxicity, including to cause coma and death.

In some embodiments, the payload gene encodes a protein that comprises a cytosolic protein, e.g., a protein that is produced in the recipient cell and localizes to the recipient cell cytoplasm. In some embodiments, payload gene encodes a protein that comprises a secreted protein, e.g., a protein that is produced and secreted by the recipient cell. In some embodiments, the payload gene encodes a protein that is a nuclear protein, e.g., a protein that is produced in the recipient cell and is imported to the nucleus of the recipient cell. In some embodiments, the payload gene encodes a protein that comprises an organellar protein (e.g., a mitochondrial protein), e.g., a protein that is produced in the recipient cell and is imported into an organelle (e.g., a mitochondrial) of the recipient cell.

In some embodiments, the payload gene encodes a protein that comprises a membrane protein. In some embodiments, the membrane protein comprises a chimeric antigen receptor (CAR), a T cell receptor, an integrin, an ion channel, a pore forming protein, a Toll-Like Receptor, an interleukin receptor, a cell adhesion protein, or a transport protein.

In some embodiments, the payload gene encodes a protein that is a nuclease for use in gene editing methods. In some embodiments, the nuclease is a zinc-finger nucleases (ZFNs), transcription-activator like effector nucleases (TALENs), or a CRISPR-associated protein-nuclease (Cas). In some embodiments, the Cas is Cas9 from Streptococcus pyogenes. In some embodiments, the Cas is a Cas12a (also known as cpf1) from a Prevotella or Francisella bacteria, or the Cas is a Cas12b from a Bacillus, optionally Bacillus hisashii. In some of any embodiments, the Cas is a Cas3, Cas13, CasMini, or any other Cas protein known in the art. See for example, Wang et al., Biosensors and Bioelectronics (165) 1: 2020, and Wu et al. Nature Reviews Chemistry (4) 441: 2020)

In some embodiments, the provided the lipid particle contains a payload gene that encodes a protein that is a nuclease protein. In some embodiments, the provided the lipid particle contains a protein that is a nuclease protein and the nuclease protein is directly delivered to a target cell Methods of delivering a nuclease protein include those as described, for example, in Cai et al. Elife, 2014, 3:e01911 and International patent publication No. WO2017068077. For instance, the provided lipid particle comprises one or more Cas protein(s), such as Cas9. In some embodiments, the nuclease protein (e.g. Cas, such as Cas 9) is engineered as a chimeric nuclease protein with a viral structural protein (e.g. GAG) for packaging into the lipid particle (e.g. paramyxovirus lipid particles). For instance, a chimeric Cas9-protein fusion with the structural GAG protein can be packaged inside a paramyxovirus lipid particle. In some embodiments, the fusion protein is a cleavable fusion protein between (i) a viral structural protein (e.g. GAG) and (ii) a nuclease protein (e.g. Cas protein, such as Cas 9).

In some embodiments, the lipid particle is a particle which further comprises an encapsulated polypeptide or polynucleotide encoding a payload gene, a therapeutic gene, an exogenous gene, and/or a recombinant gene, such as any recombinant gene, particularly a therapeutic gene.

In some embodiments, the payload gene comprises a nucleic acid (i.e., a heterologous, recombinant, exogenous, or therapeutic gene) that encodes a cytosolic protein, e.g., a protein that is produced in the recipient cell and localizes to the recipient cell cytoplasm. In some embodiments, the payload gene comprises a nucleic acid that encodes a secreted protein, e.g., a protein that is produced and secreted by the recipient cell. In some embodiments, the payload gene comprises a nucleic acid that encodes a nuclear protein, e.g., a protein that is produced in the recipient cell and is imported to the nucleus of the recipient cell. In some embodiments, the payload gene comprises a nucleic acid that encodes an organellar protein (e.g., a mitochondrial protein), e.g., a protein that is produced in the recipient cell and is imported into an organelle (e.g., a mitochondrial) of the recipient cell.

In some embodiments, the payload gene comprises a nucleic acid (i.e., a heterologous, recombinant, exogenous, or therapeutic gene) that encodes a membrane protein. In some embodiments, the membrane protein comprises a nucleic acid that encodes a chimeric antigen receptor (CAR), a T cell receptor, an integrin, an ion channel, a pore forming protein, a Toll-Like Receptor, an interleukin receptor, a cell adhesion protein, or a transport protein. In some embodiments, delivery of the nuclease is by a provided vector encoding the nuclease (e.g. Cas).

In some embodiments, the payload gene is a globin gene. In some embodiments, the payload gene is ADA, IL2RG, JAK3, IL7R, HBB, F8, F9, WAS, CYBA, CYBB, NCF1, NCF2, NCF4, UROS, TCIRG1, CLCN7, MPL, ITGA2B, ITGB3, ITGB2, PKLR, SLC25, A38, RAG1, RAG2, FANCA, FANCC, FANCG, ABCD1, MAN2B1, AGA, LYST, CTNS, LAMP2, GLA, CTSA, GBA, GAA, IDS, IDUA, ISSD, ARSB, GALNS, GLB1, NEU1, GNPTA, SUMF1, SMPD1, NPC1, NPC2, CTSK, GNS, HGSNAT, NAGLU, SGSH, NAGA, GUSB, PSAP, LAL. In some embodiment, the payload gene can be a gene for delivery to a hematopoietic stem cell (HSC). Exemplary payload genes are described in WO2020102485, which is incorporated by reference.

For example, the payload gene can be, but is not limited to antisense ras, antisense myc, antisense raf, antisense erb, antisense src, antisense fins, antisense jun, antisense trk, antisense ret, antisense gsp, antisense hst, antisense bcl, antisense abl, Rb, CFTR, pi 6, p21, p27, p57, p73, C-CAM, APC, CTS-I, zacl, scFV ras, DCC, NF-I, NF-2, WT-I, MEN-I, MEN-II, BRCA1, VHL, MMACl, FCC, MCC, BRCA2, IL-I, IL-2, IL-3, IL-4, IL-5, IL-6, IL-7, IL-8, IL-9, IL-10, IL-I1 IL-12, GM-CSF, G-CSF, thymidine kinase, mda7, fus-1, interferon α, interferon β, interferon γ, ADP, p53, ABLI, BLC1, BLC6, CBFA1, CBL, CSFIR, ERBA, ERBB, EBRB2, ETS1, ETS2, ETV6, FGR, FOX, FYN, HCR, HRAS, JUN, KRAS, LCK, LYN, MDM2, MLL, MYB, MYC, MYCL1, MYCN, NRAS, PIM1, PML, RET, SRC, TALI, TCL3, YES, MADH4, RB1, TP53, WT1, TNF, BDNF, CNTF, NGF, IGF, GMF, aFGF, bFGF, NT3, NT5, ApoAI, ApoAIV, ApoE, Rap1A, cytosine deaminase, Fab, ScFv, BRCA2, zacl, ATM, HIC-I, DPC-4, FHIT, PTEN, ING1, NOEY1, NOEY2, OVCA1, MADR2, 53BP2, IRF-I, Rb, zacl, DBCCR-I, rks-3, COX-I, TFPI, PGS, Dp, E2F, ras, myc, neu, raf, erb, fins, trk, ret, gsp, hst, abl, E1A, p300, VEGF, FGF, thrombospondin, BAI-I, GDAIF, or MCC. In further embodiments of the present invention, the payload gene is a gene encoding an ACP desaturase, an ACP hydroxylase, an ADP-glucose pyrophorylase, an ATPase, an alcohol dehydrogenase, an amylase, an amyloglucosidase, a catalase, a cellulase, a cyclooxygenase, a decarboxylase, a dextrinase, an esterase, a DNA polymerase, an RNA polymerase, a hyaluron synthase, a galactosidase, a glucanase, a glucose oxidase, a GTPase, a helicase, a hemicellulase, a hyaluronidase, an integrase, an invertase, an isomerase, a kinase, a lactase, a lipase, a lipoxygenase, a lyase, a lysozyrne, a pectinesterase, a peroxidase, a phosphatase, a phospholipase, a phosphorylase, a polygalacturonase, a proteinase, a peptidease, a pullanase, a recombinase, a reverse transcriptase, a topoisomerase, a xylanase, a reporter gene, an interleukin, or a cytokine. In other embodiments of the present invention, the payload gene is a gene encoding carbamoyl synthetase I, ornithine transcarbamylase, arginosuccinate synthetase, arginosuccinate lyase, arginase, fumarylacetoacetate hydrolase, phenylalanine hydroxylase, alpha-1 antitrypsin, gmcose-6-phosphatase, low-density-lipoprotein receptor, porphobilinogen deaminase, factor VIII, factor IX, cystathione α-synthase, branched chain ketoacid decarboxylase, albumin, isovaleryl-CoA dehydrogenase, propionyl CoA carboxylase, methyl malonyl CoA mutase, glutaryl CoA dehydrogenase, insulin, beta.-glucosidase, pyruvate carboxylase, hepatic phosphorylase, phosphorylase kinase, glycine decarboxylase, H-protein, T-protein, Menkes disease copper-transporting ATPase, Wilson's disease copper-transporting ATPase, cytosine deaminase, hypoxanthine-guanine phosphoribosyltransferase. galactose-1-phosphate uridyltransferase, phenylalanine hydroxylase, glucocerbrosidase, sphingomyelinase, α-L-iduronidase, glucose-6-phosphate dehydrogenase, HSV thymidine kinase, or human thymidine kinase. Alternatively, the recombinant gene may encode growth hormone, prolactin, placental lactogen, luteinizing hormone, follicle-stimulating hormone, chorionic gonadotropin, thyroid-stimulating hormone, leptin, adrenocorticotropin, angiotensin I, angiotensin II, β-endorphin, β-melanocyte stimulating hormone, cholecystokinin, endothelin I, galanin, gastric inhibitory peptide, glucagon, insulin, lipotropins, neurophysins, somatostatin, calcitonin, calcitonin gene related peptide, β-calcitonin gene related peptide, hypercalcemia of malignancy factor, parathyroid hormone-related protein, parathyroid hormone-related protein, glucagon-like peptide, pancreastatin, pancreatic peptide, peptide YY, PHM, secretin, vasoactive intestinal peptide, oxytocin, vasopressin, vasotocin, enkephalinamide, metorphinamide, alpha melanocyte stimulating hormone, atrial natriuretic factor, amylin, amyloid P component, corticotropin releasing hormone, growth hormone releasing factor, luteinizing hormone-releasing hormone, neuropeptide Y, substance K, substance P, or thyrotropin releasing hormone.

### 1. Chimeric Antigen Receptors

In certain embodiments, the payload gene may comprise an exogenous polynucleotide encoding a CAR. CARs (also known as chimeric immunoreceptors, chimeric T cell receptors, or artificial T cell receptors) are receptor proteins that have been engineered to give host cells (e.g., T cells) the new ability to target a specific protein. The receptors are chimeric because they combine both antigen-binding and T cell activating functions into a single receptor. The polycistronic vector of the present disclosure may be used to express one or more CARs in a host cell (e.g., a T cell) for use in cell-based therapies against various target antigens. The CARs expressed by the one or more expression cassettes may be the same or different. In these embodiments, the CAR may comprise an extracellular binding domain (also referred to as a “binder”) that specifically binds a target antigen, a transmembrane domain, and an intracellular signaling domain. In certain embodiments, the CAR may further comprise one or more additional elements, including one or more signal peptides, one or more extracellular hinge domains, and/or one or more intracellular costimulatory domains. Domains may be directly adjacent to one another, or there may be one or more amino acids linking the domains. The nucleotide sequence encoding a CAR may be derived from a mammalian sequence, for example, a mouse sequence, a primate sequence, a human sequence, or combinations thereof. In the cases where the nucleotide sequence encoding a CAR is non-human, the sequence of the CAR may be humanized. The nucleotide sequence encoding a CAR may also be codon-optimized for expression in a mammalian cell, for example, a human cell. In any of these embodiments, the nucleotide sequence encoding a CAR may be at least 80% identical (e.g., at least 80%, at least 85%, at least 90%, at least 95%, at least 96%, at least 97%, at least 98%, at least 99%, or 100% identical) to any of the nucleotide sequences disclosed herein. The sequence variations may be due to codon-optimization, humanization, restriction enzyme-based cloning scars, and/or additional amino acid residues linking the functional domains, etc.

In certain embodiments, the CAR may comprise a signal peptide at the N-terminus. Non-limiting examples of signal peptides include CD8α signal peptide, IgK signal peptide, and granulocyte-macrophage colony-stimulating factor receptor subunit alpha (GMCSFR-α, also known as colony stimulating factor 2 receptor subunit alpha (CSF2RA)) signal peptide, and variants thereof, the amino acid sequences of which are provided in Table 3 below.

TABLE 3
Exemplary sequences of signal peptides

| SEQ ID | | |
|---|---|---|
| NO: | Sequence | Description |
| 24 | MALPVTALLLPLALLLHAARP | CD8α signal peptide |
| 25 | METDTLLLWVLLLWVPGSTG | IgK signal peptide |
| 26 | MLLLVTSLLLCELPHPAFLLIP | GMCSFR-α (CSF2RA) signal peptide |

In certain embodiments, the extracellular binding domain of the CAR may comprise one or more antibodies specific to one target antigen or multiple target antigens. The antibody may be an antibody fragment, for example, an scFv, or a single-domain antibody fragment, for example, a VHH. In certain embodiments, the scFv may comprise a heavy chain variable region (V_{H}) and a light chain variable region (V_{L}) of an antibody connected by a linker. The V_{H} and the V_{L} may be connected in either order, i.e., V_{H}-linker-V_{L} or V_{L}-linker-V_{H}. Non-limiting examples of linkers include Whitlow linker, (G₄S)ₙ (n can be a positive integer, e.g., 1, 2, 3, 4, 5, 6, etc.) linker, and variants thereof. In certain embodiments, the antigen may be an antigen that is exclusively or preferentially expressed on tumor cells, or an antigen that is characteristic of an autoimmune or inflammatory disease. Exemplary target antigens include, but are not limited to, CD5, CD19, CD20, CD22, CD23, CD30, CD70, Kappa, Lambda, and B cell maturation agent (BCMA), G-protein coupled receptor family C group 5 member D (GPRC5D) (associated with leukemias); CS1/SLAMF7, CD38, CD138, GPRC5D, TACI, and BCMA (associated with myelomas); GD2, HER2, EGFR, EGFRvIII, B7H3, PSMA, PSCA, CAIX, CD171, CEA, CSPG4, EPHA2, FAP, FRα, IL-13Rα, Mesothelin, MUC1, MUC16, and ROR1 (associated with solid tumors). In any of these embodiments, the extracellular binding domain of the CAR can be codon-optimized for expression in a host cell or have variant sequences to increase functions of the extracellular binding domain.

In certain embodiments, the CAR may comprise a hinge domain, also referred to as a spacer. The terms “hinge” and “spacer” may be used interchangeably in the present disclosure. Non-limiting examples of hinge domains include CD8α hinge domain, CD28 hinge domain, IgG4 hinge domain, IgG4 hinge-CH2-CH3 domain, and variants thereof, the amino acid sequences of which are provided in Table 4 below.

TABLE 4
Exemplary sequences of hinge domains

| SEQ ID | | |
|---|---|---|
| NO: | Sequence | Description |
| 27 | TTTPAPRPPTPAPTIASQPLSLRPEACRPAAGGAVH | CD8α hinge domain |
| | TRGLDFACD | |
| 28 | IEVMYPPPYLDNEKSNGTIIHVKGKHLCPSPLFPGP | CD28 hinge domain |
| | SKP | |
| 29 | AAAIEVMYPPPYLDNEKSNGTIIHVKGKHLCPSPLF | CD28 hinge domain |
| | PGPSKP | |
| 30 | ESKYGPPCPPCP | IgG4 hinge domain |
| 31 | ESKYGPPCPSCP | IgG4 hinge domain |
| 32 | ESKYGPPCPPCPAPEFLGGPSVFLFPPKPKDTLMISR | IgG4 hinge-CH2-CH3 |
| | TPEVTCVVVDVSQEDPEVQFNWYVDGVEVHNAK | domain |
| | TKPREEQFNSTYRVVSVLTVLHQDWLNGKEYKCK | |
| | VSNKGLPSSIEKTISKAKGQPREPQVYTLPPSQEEM | |
| | TKNQVSLTCLVKGFYPSDIAVEWESNGQPENNYK | |
| | TTPPVLDSDGSFFLYSRLTVDKSRWQEGNVFSCSV | |
| | MHEALHNHYTQKSLSLSLGK | |

In certain embodiments, the transmembrane domain of the CAR may comprise a transmembrane region of the alpha, beta, or zeta chain of a T cell receptor, CD28, CD3ε, CD45, CD4, CD5, CD8, CD9, CD16, CD22, CD33, CD37, CD64, CD80, CD86, CD134, CD137, CD154, or a functional variant thereof, including the human versions of each of these sequences. In other embodiments, the transmembrane domain may comprise a transmembrane region of CD8α, CD8β, 4-1BB/CD137, CD28, CD34, CD4, FcεRIγ, CD16, OX40/CD134, CD3ζ, CD3ε, CD3γ, CD3δ, TCRα, TCRβ, TCRζ, CD32, CD64, CD64, CD45, CD5, CD9, CD22, CD37, CD80, CD86, CD40, CD40L/CD154, VEGFR2, FAS, and FGFR2B, or a functional variant thereof, including the human versions of each of these sequences. Table 5 provides the amino acid sequences of a few exemplary transmembrane domains.

TABLE 5
Exemplary sequences of transmembrane domains

| SEQ ID | | |
|---|---|---|
| NO: | Sequence | Description |
| 33 | IYIWAPLAGTCGVLLLSLVITLYC | CD8α transmembrane domain |
| 34 | FWVLVVVGGVLACYSLLVTVAFIIFWV | CD28 transmembrane domain |
| 35 | MFWVLVVVGGVLACYSLLVTVAFIIFWV | CD28 transmembrane domain |

In certain embodiments, the intracellular signaling domain and/or intracellular costimulatory domain of the CAR may comprise one or more signaling domains selected from B7-1/CD80, B7-2/CD86, B7-H1/PD-L1, B7-H2, B7-H3, B7-H4, B7-H6, B7-H7, BTLA/CD272, CD28, CTLA-4, Gi24/VISTA/B7-H5, ICOS/CD278, PD-1, PD-L2/B7-DC, PDCD6, 4-1BB/TNFSF9/CD137, 4-1BB Ligand/TNFSF9, BAFF/BLyS/TNFSF13B, BAFF R/TNFRSF13C, CD27/TNFRSF7, CD27 Ligand/TNFSF7, CD30/TNFRSF8, CD30 Ligand/TNFSF8, CD40/TNFRSF5, CD40/TNFSF5, CD40 Ligand/TNFSF5, DR3/TNFRSF25, GITR/TNFRSF18, GITR Ligand/TNFSF18, HVEM/TNFRSF14, LIGHT/TNFSF14, Lymphotoxin-alpha/TNFβ, OX40/TNFRSF4, OX40 Ligand/TNFSF4, RELT/TNFRSF19L, TACI/TNFRSF13B, TL1A/TNFSF15, TNFα, TNF RII/TNFRSF1B, 2B4/CD244/SLAMF4, BLAME/SLAMF8, CD2, CD2F-10/SLAMF9, CD48/SLAMF2, CD58/LFA-3, CD84/SLAMF5, CD229/SLAMF3, CRACC/SLAMF7, NTB-A/SLAMF6, SLAM/CD150, CD2, CD7, CD53, CD82/Kai-1, CD90/Thy1, CD96, CD160, CD200, CD300a/LMIR1, HLA Class I, HLA-DR, Ikaros, Integrin alpha 4/CD49d, Integrin alpha 4 beta 1, Integrin alpha 4 beta 7/LPAM-1, LAG-3, TCL1A, TCL1B, CRTAM, DAP12, Dectin-1/CLEC7A, DPPIV/CD26, EphB6, TIM-1/KIM-1/HAVCR, TIM-4, TSLP, TSLP R, lymphocyte function associated antigen-1 (LFA-1), NKG2C, CD3ζ, an immunoreceptor tyrosine-based activation motif (ITAM), CD27, CD28, 4-1BB, CD134/OX40, CD30, CD40, PD-1, ICOS, lymphocyte function-associated antigen-1 (LFA-1), CD2, CD7, LIGHT, NKG2C, B7-H3, a ligand that specifically binds with CD83, and a functional variant thereof including the human versions of each of these sequences. In some embodiments, the intracellular signaling domain and/or intracellular costimulatory domain comprises one or more signaling domains selected from a CD3ζ domain, an ITAM, a CD28 domain, 4-1BB domain, or a functional variant thereof. Table 6 provides the amino acid sequences of a few exemplary intracellular costimulatory and/or signaling domains. In certain embodiments, as in the case of tisagenlecleucel as described below, the CD3ζ signaling domain of SEQ ID NO:38 may have a mutation, e.g., a glutamine (Q) to lysine (K) mutation, at amino acid position 14 (see SEQ ID NO:39).

TABLE 6
Exemplary sequences of intracellular costimulatory and/or signaling domains

| SEQ ID | | |
|---|---|---|
| NO: | Sequence | Description |
| 36 | KRGRKKLLYIFKQPFMRPVQTTQEEDGCSCR | 4-1BB costimulatory domain |
| | FPEEEEGGCEL | |
| 37 | RSKRSRLLHSDYMNMTPRRPGPTRKHYQPY | CD28 costimulatory domain |
| | APPRDFAAYRS | |
| 38 | RVKFSRSADAPAYQQGQNQLYNELNLGRRE | CD3ζ signaling domain |
| | EYDVLDKRRGRDPEMGGKPRRKNPQEGLYN | |
| | ELQKDKMAEAYSEIGMKGERRRGKGHDGLY | |
| | QGLSTATKDTYDALHMQALPPR | |
| 39 | RVKFSRSADAPAYKQGQNQLYNELNLGRRE | CD3ζ signaling domain (with Q |
| | EYDVLDKRRGRDPEMGGKPRRKNPQEGLYN | to K mutation at position 14) |
| | ELQKDKMAEAYSEIGMKGERRRGKGHDGLY | |
| | QGLSTATKDTYDALHMQALPPR | |

In certain embodiments where the polycistronic vector encodes two or more CARs, the two or more CARs may comprise the same functional domains, or one or more different functional domains, as described. For example, the two or more CARs may comprise different signal peptides, extracellular binding domains, hinge domains, transmembrane domains, costimulatory domains, and/or intracellular signaling domains, in order to minimize the risk of recombination due to sequence similarities. Or, alternatively, the two or more CARs may comprise the same domains. In the cases where the same domain(s) and/or backbone are used, it is optional to introduce codon divergence at the nucleotide sequence level to minimize the risk of recombination.

a. CD19 CAR

In some embodiments, the CAR is a CD19 CAR (“CD19-CAR”), and in these embodiments, the polycistronic vector comprises an expression cassette that contains a nucleotide sequence encoding a CD19 CAR. In some embodiments, the CD19 CAR may comprise a signal peptide, an extracellular binding domain that specifically binds CD19, a hinge domain, a transmembrane domain, an intracellular costimulatory domain, and/or an intracellular signaling domain in tandem.

In some embodiments, the signal peptide of the CD19 CAR comprises a CD8α signal peptide. In some embodiments, the CD8α signal peptide comprises or consists of an amino acid sequence set forth in SEQ ID NO:24 or an amino acid sequence that is at least 80% identical (e.g., at least 80%, at least 85%, at least 90%, at least 95%, at least 96%, at least 97%, at least 98%, at least 99%, or 100% identical) to the amino acid sequence set forth in of SEQ ID NO:24. In some embodiments, the signal peptide comprises an IgK signal peptide. In some embodiments, the IgK signal peptide comprises or consists of an amino acid sequence set forth in SEQ ID NO:25 or an amino acid sequence that is at least 80% identical (e.g., at least 80%, at least 85%, at least 90%, at least 95%, at least 96%, at least 97%, at least 98%, at least 99%, or 100% identical) to the amino acid sequence set forth in of SEQ ID NO:25. In some embodiments, the signal peptide comprises a GMCSFR-α or CSF2RA signal peptide. In some embodiments, the GMCSFR-α or CSF2RA signal peptide comprises or consists of an amino acid sequence set forth in SEQ ID NO:26 or an amino acid sequence that is at least 80% identical (e.g., at least 80%, at least 85%, at least 90%, at least 95%, at least 96%, at least 97%, at least 98%, at least 99%, or 100% identical) to the amino acid sequence set forth in of SEQ ID NO:26.

In some embodiments, the extracellular binding domain of the CD19 CAR is specific to CD19, for example, human CD19. The extracellular binding domain of the CD19 CAR can be codon-optimized for expression in a host cell or to have variant sequences to increase functions of the extracellular binding domain. In some embodiments, the extracellular binding domain comprises an immunogenically active portion of an immunoglobulin molecule, for example, an scFv.

In some embodiments, the extracellular binding domain of the CD19 CAR comprises an scFv derived from the FMC63 monoclonal antibody (FMC63), which comprises the heavy chain variable region (V_{H}) and the light chain variable region (V_{L}) of FMC63 connected by a linker. FMC63 and the derived scFv have been described in Nicholson et al., Mol. Immun. 34(16-17):1157-1165 (1997) and PCT Application Publication No. WO2018/213337, the entire contents of each of which are incorporated by reference herein. In some embodiments, the amino acid sequences of the entire FMC63-derived scFv (also referred to as FMC63 scFv) and its different portions are provided in Table 7 below. In some embodiments, the CD19-specific scFv comprises or consists of an amino acid sequence set forth in SEQ ID NO:40, 41, or 46, or an amino acid sequence that is at least 80% identical (e.g., at least 80%, at least 85%, at least 90%, at least 95%, at least 96%, at least 97%, at least 98%, at least 99%, or 100% identical) to the amino acid sequence set forth in SEQ ID NO:40, 41, or 46. In some embodiments, the CD19-specific scFv may comprise one or more CDRs having amino acid sequences set forth in SEQ ID NOs: 42-44 and 48-50. In some embodiments, the CD19-specific scFv may comprise a light chain with one or more CDRs having amino acid sequences set forth in SEQ ID NOs: 42-44. In some embodiments, the CD19-specific scFv may comprise a heavy chain with one or more CDRs having amino acid sequences set forth in SEQ ID NOs: 48-50. In any of these embodiments, the CD19-specific scFv may comprise one or more CDRs comprising one or more amino acid substitutions, or comprising a sequence that is at least 80% identical (e.g., at least 80%, at least 85%, at least 90%, at least 95%, at least 96%, at least 97%, at least 98%, at least 99%, or 100% identical), to any of the sequences identified. In some embodiments, the extracellular binding domain of the CD19 CAR comprises or consists of the one or more CDRs as described herein.

In some embodiments, the linker linking the V_{H} and the V_{L} portions of the scFv is a Whitlow linker having an amino acid sequence set forth in SEQ ID NO:45. In some embodiments, the Whitlow linker may be replaced by a different linker, for example, a 3×G₄S linker having an amino acid sequence set forth in SEQ ID NO:51, which gives rise to a different FMC63-derived scFv having an amino acid sequence set forth in SEQ ID NO:50. In certain of these embodiments, the CD19-specific scFv comprises or consists of an amino acid sequence set forth in SEQ ID NO:50 or an amino acid sequence that is at least 80% identical (e.g., at least 80%, at least 85%, at least 90%, at least 95%, at least 96%, at least 97%, at least 98%, at least 99%, or 100% identical) to the amino acid sequence set forth in of SEQ ID NO:50.

TABLE 7
Exemplary sequences of anti-CD19 scFv and components

| SEQ ID NO: | Amino Acid Sequence | Description |
|---|---|---|
| 40 | DIQMTQTTSSLSASLGDRVTISCRASQDI | Anti-CD19 FMC63 scFv |
| | SKYLNWYQQKPDGTVKLLIYHTSRLHS | entire sequence, with |
| | GVPSRFSGSGSGTDYSLTISNLEQEDIAT | Whitlow linker |
| | YFCQQGNTLPYTFGGGTKLEITGSTSGS | |
| | GKPGSGEGSTKGEVKLQESGPGLVAPS | |
| | QSLSVTCTVSGVSLPDYGVSWIRQPPRK | |
| | GLEWLGVIWGSETTYYNSALKSRLTIIK | |
| | DNSKSQVFLKMNSLQTDDTAIYYCAKH | |
| | YYYGGSYAMDYWGQGTSVTVSS | |
| 41 | DIQMTQTTSSLSASLGDRVTISCRASQDI | Anti-CD19 FMC63 scFv |
| | SKYLNWYQQKPDGTVKLLIYHTSRLHS | light chain variable region |
| | GVPSRFSGSGSGTDYSLTISNLEQEDIAT | |
| | YFCQQGNTLPYTFGGGTKLEIT | |
| 42 | QDISKY | Anti-CD19 FMC63 scFv |
| | | light chain CDR1 |
| 43 | HTS | Anti-CD19 FMC63 scFv |
| | | light chain CDR2 |
| 44 | QQGNTLPYT | Anti-CD19 FMC63 scFv |
| | | light chain CDR3 |
| 45 | GSTSGSGKPGSGEGSTKG | Whitlow linker |
| 46 | EVKLQESGPGLVAPSQSLSVTCTVSGVS | Anti-CD19 FMC63 scFv |
| | LPDYGVSWIRQPPRKGLEWLGVIWGSE | heavy chain variable region |
| | TTYYNSALKSRLTIIKDNSKSQVFLKMN | |
| | SLQTDDTAIYYCAKHYYYGGSYAMDY | |
| | WGQGTSVTVSS | |
| 47 | GVSLPDYG | Anti-CD19 FMC63 scFv |
| | | heavy chain CDR1 |
| 48 | IWGSETT | Anti-CD19 FMC63 scFv |
| | | heavy chain CDR2 |
| 49 | AKHYYYGGSYAMDY | Anti-CD19 FMC63 scFv |
| | | heavy chain CDR3 |
| 50 | DIQMTQTTSSLSASLGDRVTISCRASQDI | Anti-CD19 FMC63 scFv |
| | SKYLNWYQQKPDGTVKLLIYHTSRLHS | entire sequence, with 3xG₄S |
| | GVPSRFSGSGSGTDYSLTISNLEQEDIAT | linker |
| | YFCQQGNTLPYTFGGGTKLEITGGGGS | |
| | GGGGSGGGGSEVKLQESGPGLVAPSQS | |
| | LSVTCTVSGVSLPDYGVSWIRQPPRKGL | |
| | EWLGVIWGSETTYYNSALKSRLTIIKDN | |
| | SKSQVFLKMNSLQTDDTAIYYCAKHYY | |
| | YGGSYAMDYWGQGTSVTVSS | |
| 51 | GGGGSGGGGSGGGGS | 3xG₄S linker |

In some embodiments, the extracellular binding domain of the CD19 CAR is derived from an antibody specific to CD19, including, for example, SJ25C1 (Bejcek et al., Cancer Res. 55:2346-2351 (1995)), HD37 (Pezutto et al., J. Immunol. 138(9):2793-2799 (1987)), 4G7 (Meeker et al., Hybridoma 3:305-320 (1984)), B43 (Bejcek (1995)), BLY3 (Bejcek (1995)), B4 (Freedman et al., 70:418-427 (1987)), B4 HB12b (Kansas & Tedder, J. Immunol. 147:4094-4102 (1991); Yazawa et al., Proc. Natl. Acad. Sci. USA 102:15178-15183 (2005); Herbst et al., J. Pharmacol. Exp. Ther. 335:213-222 (2010)), BU12 (Callard et al., J. Immunology, 148(10): 2983-2987 (1992)), and CLB-CD19 (De Rie Cell. Immunol. 118:368-381(1989)). In any of these embodiments, the extracellular binding domain of the CD19 CAR can comprise or consist of the V_{H}, the V_{L}, and/or one or more CDRs of any of the antibodies.

In some embodiments, the hinge domain of the CD19 CAR comprises a CD8α hinge domain, for example, a human CD8α hinge domain. In some embodiments, the CD8α hinge domain comprises or consists of an amino acid sequence set forth in SEQ ID NO:27 or an amino acid sequence that is at least 80% identical (e.g., at least 80%, at least 85%, at least 90%, at least 95%, at least 96%, at least 97%, at least 98%, at least 99%, or 100% identical) to the amino acid sequence set forth in of SEQ ID NO:27. In some embodiments, the hinge domain comprises a CD28 hinge domain, for example, a human CD28 hinge domain. In some embodiments, the CD28 hinge domain comprises or consists of an amino acid sequence set forth in SEQ ID NO:28 or an amino acid sequence that is at least 80% identical (e.g., at least 80%, at least 85%, at least 90%, at least 95%, at least 96%, at least 97%, at least 98%, at least 99%, or 100% identical) to the amino acid sequence set forth in of SEQ ID NO:28. In some embodiments, the CD28 hinge domain comprises or consists of an amino acid sequence set forth in SEQ ID NO:29 or an amino acid sequence that is at least 80% identical (e.g., at least 80%, at least 85%, at least 90%, at least 95%, at least 96%, at least 97%, at least 98%, at least 99%, or 100% identical) to the amino acid sequence set forth in of SEQ ID NO:29. In some embodiments, the hinge domain comprises an IgG4 hinge domain, for example, a human IgG4 hinge domain. In some embodiments, the IgG4 hinge domain comprises or consists of an amino acid sequence set forth in SEQ ID NO:31 or SEQ ID NO:30, or an amino acid sequence that is at least 80% identical (e.g., at least 80%, at least 85%, at least 90%, at least 95%, at least 96%, at least 97%, at least 98%, at least 99%, or 100% identical) to the amino acid sequence set forth in of SEQ ID NO:31 or SEQ ID NO:30. In some embodiments, the hinge domain comprises a IgG4 hinge-Ch2-Ch3 domain, for example, a human IgG4 hinge-Ch2-Ch3 domain. In some embodiments, the IgG4 hinge-Ch2-Ch3 domain comprises or consists of an amino acid sequence set forth in SEQ ID NO:32 or an amino acid sequence that is at least 80% identical (e.g., at least 80%, at least 85%, at least 90%, at least 95%, at least 96%, at least 97%, at least 98%, at least 99%, or 100% identical) to the amino acid sequence set forth in of SEQ ID NO:32.

In some embodiments, the transmembrane domain of the CD19 CAR comprises a CD8α transmembrane domain, for example, a human CD8α transmembrane domain. In some embodiments, the CD8α transmembrane domain comprises or consists of an amino acid sequence set forth in SEQ ID NO:33 or an amino acid sequence that is at least 80% identical (e.g., at least 80%, at least 85%, at least 90%, at least 95%, at least 96%, at least 97%, at least 98%, at least 99%, or 100% identical) to the amino acid sequence set forth in SEQ ID NO:33. In some embodiments, the transmembrane domain comprises a CD28 transmembrane domain, for example, a human CD28 transmembrane domain. In some embodiments, the CD28 transmembrane domain comprises or consists of an amino acid sequence set forth in SEQ ID NO:34 or an amino acid sequence that is at least 80% identical (e.g., at least 80%, at least 85%, at least 90%, at least 95%, at least 96%, at least 97%, at least 98%, at least 99%, or 100% identical) to the amino acid sequence set forth in SEQ ID NO:34. In some embodiments, the CD28 transmembrane domain comprises or consists of an amino acid sequence set forth in SEQ ID NO:35 or an amino acid sequence that is at least 80% identical (e.g., at least 80%, at least 85%, at least 90%, at least 95%, at least 96%, at least 97%, at least 98%, at least 99%, or 100% identical) to the amino acid sequence set forth in SEQ ID NO:35.

In some embodiments, the intracellular costimulatory domain of the CD19 CAR comprises a 4-1BB costimulatory domain. 4-1BB, also known as CD137, transmits a potent costimulatory signal to T cells, promoting differentiation and enhancing long-term survival of T lymphocytes. In some embodiments, the 4-1BB costimulatory domain is human. In some embodiments, the 4-1BB costimulatory domain comprises or consists of an amino acid sequence set forth in SEQ ID NO:36 or an amino acid sequence that is at least 80% identical (e.g., at least 80%, at least 85%, at least 90%, at least 95%, at least 96%, at least 97%, at least 98%, at least 99%, or 100% identical) to the amino acid sequence set forth in SEQ ID NO:36. In some embodiments, the intracellular costimulatory domain comprises a CD28 costimulatory domain. CD28 is another co-stimulatory molecule on T cells. In some embodiments, the CD28 costimulatory domain is human.

In some embodiments, the CD28 costimulatory domain comprises or consists of an amino acid sequence set forth in SEQ ID NO:37 or an amino acid sequence that is at least 80% identical (e.g., at least 80%, at least 85%, at least 90%, at least 95%, at least 96%, at least 97%, at least 98%, at least 99%, or 100% identical) to the amino acid sequence set forth in SEQ ID NO:37. In some embodiments, the intracellular costimulatory domain of the CD19 CAR comprises a 4-1BB costimulatory domain and a CD28 costimulatory domain as described.

In some embodiments, the intracellular signaling domain of the CD19 CAR comprises a CD3 zeta (ζ) signaling domain. CD3ζ associates with T cell receptors (TCRs) to produce a signal and contains immunoreceptor tyrosine-based activation motifs (ITAMs). The CD3ζ signaling domain refers to amino acid residues from the cytoplasmic domain of the zeta chain that are sufficient to functionally transmit an initial signal necessary for T cell activation. In some embodiments, the CD3ζ signaling domain is human. In some embodiments, the CD3ζ signaling domain comprises or consists of an amino acid sequence set forth in SEQ ID NO:38 or an amino acid sequence that is at least 80% identical (e.g., at least 80%, at least 85%, at least 90%, at least 95%, at least 96%, at least 97%, at least 98%, at least 99%, or 100% identical) to the amino acid sequence set forth in SEQ ID NO:38.

In some embodiments, the polycistronic vector comprises an expression cassette that contains a nucleotide sequence encoding a CD19 CAR, including, for example, a CD19 CAR comprising the CD19-specific scFv having sequences set forth in SEQ ID NO:40 or SEQ ID NO:41, the CD8α hinge domain of SEQ ID NO:27, the CD8α transmembrane domain of SEQ ID NO:33, the 4-1BB costimulatory domain of SEQ ID NO:36, the CD3ζ signaling domain of SEQ ID NO:38, and/or variants (i.e., having a sequence that is at least 80% identical, for example, at least 80%, at least 85%, at least 90%, at least 95%, at least 96%, at least 97%, at least 98%, or at least 99 identical to the disclosed sequence) thereof. In any of these embodiments, the CD19 CAR may additionally comprise a signal peptide (e.g., a CD8α signal peptide) as described.

In some embodiments, the polycistronic vector comprises an expression cassette that contains a nucleotide sequence encoding a CD19 CAR, including, for example, a CD19 CAR comprising the CD19-specific scFv having sequences set forth in SEQ ID NO:40 or SEQ ID NO:41, the IgG4 hinge domain of SEQ ID NO:30 or SEQ ID NO:31, the CD28 transmembrane domain of SEQ ID NO:34, the 4-1BB costimulatory domain of SEQ ID NO:36, the CD3ζ signaling domain of SEQ ID NO:38, and/or variants (i.e., having a sequence that is at least 80% identical, for example, at least 80%, at least 85%, at least 90%, at least 95%, at least 96%, at least 97%, at least 98%, or at least 99 identical to the disclosed sequence) thereof. In any of these embodiments, the CD19 CAR may additionally comprise a signal peptide (e.g., a CD8α signal peptide) as described.

In some embodiments, the polycistronic vector comprises an expression cassette that contains a nucleotide sequence encoding a CD19 CAR, including, for example, a CD19 CAR comprising the CD19-specific scFv having sequences set forth in SEQ ID NO:40 or SEQ ID NO:41, the CD28 hinge domain of SEQ ID NO:28, the CD28 transmembrane domain of SEQ ID NO:34, the CD28 costimulatory domain of SEQ ID NO:37, the CD3ζ signaling domain of SEQ ID NO:38, and/or variants (i.e., having a sequence that is at least 80% identical, for example, at least 80%, at least 85%, at least 90%, at least 95%, at least 96%, at least 97%, at least 98%, or at least 99 identical to the disclosed sequence) thereof. In any of these embodiments, the CD19 CAR may additionally comprise a signal peptide (e.g., a CD8α signal peptide) as described.

In some embodiments, the polycistronic vector comprises an expression cassette that contains a nucleotide sequence encoding a CD19 CAR as set forth in SEQ ID NO:52 or is at least 80% identical (e.g., at least 80%, at least 85%, at least 90%, at least 95%, at least 96%, at least 97%, at least 98%, at least 99%, or 100% identical) to the nucleotide sequence set forth in SEQ ID NO:52 (see Table 9). The encoded CD19 CAR has a corresponding amino acid sequence set forth in SEQ ID NO:53 or is at least 80% identical (e.g., at least 80%, at least 85%, at least 90%, at least 95%, at least 96%, at least 97%, at least 98%, at least 99%, or 100% identical) to the amino acid sequence set forth in of SEQ ID NO:53, with the following components: CD8α signal peptide, FMC63 scFv (V_{L}—Whitlow linker-V_{H}), CD8α hinge domain, CD8α transmembrane domain, 4-1BB costimulatory domain, and CD3ζ signaling domain.

In some embodiments, the polycistronic vector comprises an expression cassette that contains a nucleotide sequence encoding a commercially available embodiment of CD19 CAR. Non-limiting examples of commercially available embodiments of CD19 CARs expressed and/or encoded by T cells include tisagenlecleucel, lisocabtagene maraleucel, axicabtagene ciloleucel, and brexucabtagene autoleucel.

In some embodiments, the polycistronic vector comprises an expression cassette that contains a nucleotide sequence encoding tisagenlecleucel or portions thereof. Tisagenlecleucel comprises a CD19 CAR with the following components: CD8α signal peptide, FMC63 scFv (V_{L}-3×G₄S linker-V_{H}), CD8α hinge domain, CD8α transmembrane domain, 4-1BB costimulatory domain, and CD3ζ signaling domain. The nucleotide and amino acid sequence of the CD19 CAR in tisagenlecleucel are provided in Table 9, with annotations of the sequences provided in Table 9.

In some embodiments, the polycistronic vector comprises an expression cassette that contains a nucleotide sequence encoding lisocabtagene maraleucel or portions thereof. Lisocabtagene maraleucel comprises a CD19 CAR with the following components: GMCSFR-α or CSF2RA signal peptide, FMC63 scFv (V_{L}—Whitlow linker-V_{H}), IgG4 hinge domain, CD28 transmembrane domain, 4-1BB costimulatory domain, and CD3ζ signaling domain. The nucleotide and amino acid sequence of the CD19 CAR in lisocabtagene maraleucel are provided in Table 8, with annotations of the sequences provided in Table 10.

In some embodiments, the polycistronic vector comprises an expression cassette that contains a nucleotide sequence encoding axicabtagene ciloleucel or portions thereof. Axicabtagene ciloleucel comprises a CD19 CAR with the following components: GMCSFR-α or CSF2RA signal peptide, FMC63 scFv (V_{L}—Whitlow linker-V_{H}), CD28 hinge domain, CD28 transmembrane domain, CD28 costimulatory domain, and CD3ζ signaling domain. The nucleotide and amino acid sequence of the CD19 CAR in axicabtagene ciloleucel are provided in Table 8, with annotations of the sequences provided in Table 11.

In some embodiments, the polycistronic vector comprises an expression cassette that contains a nucleotide sequence encoding brexucabtagene autoleucel or portions thereof. Brexucabtagene autoleucel comprises a CD19 CAR with the following components: GMCSFR-α signal peptide, FMC63 scFv, CD28 hinge domain, CD28 transmembrane domain, CD28 costimulatory domain, and CD3ζ signaling domain.

In some embodiments, the polycistronic vector comprises an expression cassette that contains a nucleotide sequence encoding a CD19 CAR as set forth in SEQ ID NO: 54, 56, or 58, or is at least 80% identical (e.g., at least 80%, at least 85%, at least 90%, at least 95%, at least 96%, at least 97%, at least 98%, at least 99%, or 100% identical) to the nucleotide sequence set forth in SEQ ID NO: 54, 56, or 58. The encoded CD19 CAR has a corresponding amino acid sequence set forth in SEQ ID NO: 55, 57, or 59, respectively, or is at least 80% identical (e.g., at least 80%, at least 85%, at least 90%, at least 95%, at least 96%, at least 97%, at least 98%, at least 99%, or 100% identical) to the amino acid sequence set forth in of SEQ ID NO: 55, 57, or 59, respectively.

TABLE 8
Exemplary sequences of CD19 CARs

| SEQ ID NO: | Sequence | Description |
|---|---|---|
| 52 | atggccttaccagtgaccgccttgctcctgccgctggccttgctgctccac | Exemplary CD19 |
| | gccgccaggccggacatccagatgacacagactacatcctccctgtctgc | CAR nucleotide |
| | ctctctgggagacagagtcaccatcagttgcagggcaagtcaggacatta | sequence |
| | gtaaatatttaaattggtatcagcagaaaccagatggaactgttaaactcct | |
| | gatctaccatacatcaagattacactcaggagtcccatcaaggttcagtgg | |
| | cagtgggtctggaacagattattctctcaccattagcaacctggagcaaga | |
| | agatattgccacttacttttgccaacagggtaatacgcttccgtacacgttcg | |
| | gaggggggaccaagctggagatcacaggctccacctctggatccggca | |
| | agcccggatctggcgagggatccaccaagggcgaggtgaaactgcag | |
| | gagtcaggacctggcctggtggcgccctcacagagcctgtccgtcacat | |
| | gcactgtctcaggggtctcattacccgactatggtgtaagctggattcgcc | |
| | agcctccacgaaagggtctggagtggctgggagtaatatggggtagtga | |
| | aaccacatactataattcagctctcaaatccagactgaccatcatcaagga | |
| | caactccaagagccaagttttcttaaaaatgaacagtctgcaaactgatga | |
| | cacagccatttactactgtgccaaacattattactacggtggtagctatgcta | |
| | tggactactggggccaaggaacctcagtcaccgtctcctcaaccacgac | |
| | gccagcgccgcgaccaccaacaccggcgcccaccatcgcgtcgcagc | |
| | ccctgtccctgcgcccagaggcgtgccggccagcggcggggggcgca | |
| | gtgcacacgagggggctggacttcgcctgtgatatctacatctgggcgcc | |
| | cttggccgggacttgtggggtccttctcctgtcactggttatcaccctttact | |
| | gcaaacggggcagaaagaaactcctgtatatattcaaacaaccatttatga | |
| | gaccagtacaaactactcaagaggaagatggctgtagctgccgatttcca | |
| | gaagaagaagaaggaggatgtgaactgagagtgaagttcagcaggagc | |
| | gcagacgcccccgcgtaccagcagggccagaaccagctctataacgag | |
| | ctcaatctaggacgaagagaggagtacgatgttttggacaagagacgtgg | |
| | ccgggaccctgagatggggggaaagccgagaaggaagaaccctcagg | |
| | aaggcctgtacaatgaactgcagaaagataagatggcggaggcctacag | |
| | tgagattgggatgaaaggcgagcgccggaggggcaaggggcacgatg | |
| | gcctttaccagggtctcagtacagccaccaaggacacctacgacgccctt | |
| | cacatgcaggccctgccccctcgc | |
| 53 | MALPVTALLLPLALLLHAARPDIQMTQTTSSLS | Exemplary CD19 |
| | ASLGDRVTISCRASQDISKYLNWYQQKPDGTV | CAR amino acid |
| | KLLIYHTSRLHSGVPSRFSGSGSGTDYSLTISNL | sequence |
| | EQEDIATYFCQQGNTLPYTFGGGTKLEITGSTS | |
| | GSGKPGSGEGSTKGEVKLQESGPGLVAPSQSLS | |
| | VTCTVSGVSLPDYGVSWIRQPPRKGLEWLGVI | |
| | WGSETTYYNSALKSRLTIIKDNSKSQVFLKMNS | |
| | LQTDDTAIYYCAKHYYYGGSYAMDYWGQGT | |
| | SVTVSSTTTPAPRPPTPAPTIASQPLSLRPEACRP | |
| | AAGGAVHTRGLDFACDIYIWAPLAGTCGVLLL | |
| | SLVITLYCKRGRKKLLYIFKQPFMRPVQTTQEE | |
| | DGCSCRFPEEEEGGCELRVKFSRSADAPAYQQ | |
| | GQNQLYNELNLGRREEYDVLDKRRGRDPEMG | |
| | GKPRRKNPQEGLYNELQKDKMAEAYSEIGMK | |
| | GERRRGKGHDGLYQGLSTATKDTYDALHMQA | |
| | LPPR | |
| 54 | atggccttaccagtgaccgccttgctcctgccgctggccttgctgctccac | Tisagenlecleucel |
| | gccgccaggccggacatccagatgacacagactacatcctccctgtctgc | CD19 CAR |
| | ctctctgggagacagagtcaccatcagttgcagggcaagtcaggacatta | nucleotide sequence |
| | gtaaatatttaaattggtatcagcagaaaccagatggaactgttaaactcct | |
| | gatctaccatacatcaagattacactcaggagtcccatcaaggttcagtgg | |
| | cagtgggtctggaacagattattctctcaccattagcaacctggagcaaga | |
| | agatattgccacttacttttgccaacagggtaatacgcttccgtacacgttcg | |
| | gaggggggaccaagctggagatcacaggtggcggtggctcgggcggt | |
| | ggtgggtcgggtggcggcggatctgaggtgaaactgcaggagtcagga | |
| | cctggcctggtggcgccctcacagagcctgtccgtcacatgcactgtctc | |
| | aggggtctcattacccgactatggtgtaagctggattcgccagcctccacg | |
| | aaagggtctggagtggctgggagtaatatggggtagtgaaaccacatact | |
| | ataattcagctctcaaatccagactgaccatcatcaaggacaactccaaga | |
| | gccaagttttcttaaaaatgaacagtctgcaaactgatgacacagccattta | |
| | ctactgtgccaaacattattactacggtggtagctatgctatggactactgg | |
| | ggccaaggaacctcagtcaccgtctcctcaaccacgacgccagcgccg | |
| | cgaccaccaacaccggcgcccaccatcgcgtcgcagcccctgtccctgc | |
| | gcccagaggcgtgccggccagcggcggggggcgcagtgcacacgag | |
| | ggggctggacttcgcctgtgatatctacatctgggcgcccttggccggga | |
| | cttgtggggtccttctcctgtcactggttatcaccctttactgcaaacggggc | |
| | agaaagaaactcctgtatatattcaaacaaccatttatgagaccagtacaaa | |
| | ctactcaagaggaagatggctgtagctgccgatttccagaagaagaagaa | |
| | ggaggatgtgaactgagagtgaagttcagcaggagcgcagacgccccc | |
| | gcgtacaagcagggccagaaccagctctataacgagctcaatctaggac | |
| | gaagagaggagtacgatgttttggacaagagacgtggccgggaccctga | |
| | gatggggggaaagccgagaaggaagaaccctcaggaaggcctgtaca | |
| | atgaactgcagaaagataagatggcggaggcctacagtgagattgggat | |
| | gaaaggcgagcgccggaggggcaaggggcacgatggcctttaccagg | |
| | gtctcagtacagccaccaaggacacctacgacgcccttcacatgcaggc | |
| | cctgccccctcgc | |
| 55 | MALPVTALLLPLALLLHAARPDIQMTQTTSSLS | Tisagenlecleucel |
| | ASLGDRVTISCRASQDISKYLNWYQQKPDGTV | CD19 CAR amino |
| | KLLIYHTSRLHSGVPSRFSGSGSGTDYSLTISNL | acid sequence |
| | EQEDIATYFCQQGNTLPYTFGGGTKLEITGGGG | |
| | SGGGGSGGGGSEVKLQESGPGLVAPSQSLSVT | |
| | CTVSGVSLPDYGVSWIRQPPRKGLEWLGVIWG | |
| | SETTYYNSALKSRLTIIKDNSKSQVFLKMNSLQ | |
| | TDDTAIYYCAKHYYYGGSYAMDYWGQGTSV | |
| | TVSSTTTPAPRPPTPAPTIASQPLSLRPEACRPA | |
| | AGGAVHTRGLDFACDIYIWAPLAGTCGVLLLS | |
| | LVITLYCKRGRKKLLYIFKQPFMRPVQTTQEED | |
| | GCSCRFPEEEEGGCELRVKFSRSADAPAYKQG | |
| | QNQLYNELNLGRREEYDVLDKRRGRDPEMGG | |
| | KPRRKNPQEGLYNELQKDKMAEAYSEIGMKG | |
| | ERRRGKGHDGLYQGLSTATKDTYDALHMQAL | |
| | PPR | |
| 56 | atgctgctgctggtgaccagcctgctgctgtgcgagctgccccaccccgc | Lisocabtagene |
| | ctttctgctgatccccgacatccagatgacccagaccacctccagcctgag | maraleucel CD19 |
| | cgccagcctgggcgaccgggtgaccatcagctgccgggccagccagg | CAR nucleotide |
| | acatcagcaagtacctgaactggtatcagcagaagcccgacggcaccgt | sequence |
| | caagctgctgatctaccacaccagccggctgcacagcggcgtgcccagc | |
| | cggtttagcggcagcggctccggcaccgactacagcctgaccatctcca | |
| | acctggaacaggaagatatcgccacctacttttgccagcagggcaacaca | |
| | ctgccctacacctttggcggcggaacaaagctggaaatcaccggcagca | |
| | cctccggcagcggcaagcctggcagcggcgagggcagcaccaaggg | |
| | cgaggtgaagctgcaggaaagcggccctggcctggtggcccccagcca | |
| | gagcctgagcgtgacctgcaccgtgagcggcgtgagcctgcccgacta | |
| | cggcgtgagctggatccggcagccccccaggaagggcctggaatggct | |
| | gggcgtgatctggggcagcgagaccacctactacaacagcgccctgaa | |
| | gagccggctgaccatcatcaaggacaacagcaagagccaggtgttcctg | |
| | aagatgaacagcctgcagaccgacgacaccgccatctactactgcgcca | |
| | agcactactactacggcggcagctacgccatggactactggggccaggg | |
| | caccagcgtgaccgtgagcagcgaatctaagtacggaccgccctgcccc | |
| | ccttgccctatgttctgggtgctggtggtggtcggaggcgtgctggcctgc | |
| | tacagcctgctggtcaccgtggccttcatcatcttttgggtgaaacggggc | |
| | agaaagaaactcctgtatatattcaaacaaccatttatgagaccagtacaaa | |
| | ctactcaagaggaagatggctgtagctgccgatttccagaagaagaagaa | |
| | ggaggatgtgaactgcgggtgaagttcagcagaagcgccgacgcccct | |
| | gcctaccagcagggccagaatcagctgtacaacgagctgaacctgggc | |
| | agaagggaagagtacgacgtcctggataagcggagaggccgggaccc | |
| | tgagatgggcggcaagcctcggcggaagaacccccaggaaggcctgta | |
| | taacgaactgcagaaagacaagatggccgaggcctacagcgagatcgg | |
| | catgaagggcgagcggaggcggggcaagggccacgacggcctgtatc | |
| | agggcctgtccaccgccaccaaggatacctacgacgccctgcacatgca | |
| | ggccctgcccccaagg | |
| 57 | MLLLVTSLLLCELPHPAFLLIPDIQMTQTTSSLS | Lisocabtagene |
| | ASLGDRVTISCRASQDISKYLNWYQQKPDGTV | maraleucel CD19 |
| | KLLIYHTSRLHSGVPSRFSGSGSGTDYSLTISNL | CAR amino acid |
| | EQEDIATYFCQQGNTLPYTFGGGTKLEITGSTS | sequence |
| | GSGKPGSGEGSTKGEVKLQESGPGLVAPSQSLS | |
| | VTCTVSGVSLPDYGVSWIRQPPRKGLEWLGVI | |
| | WGSETTYYNSALKSRLTIIKDNSKSQVFLKMNS | |
| | LQTDDTAIYYCAKHYYYGGSYAMDYWGQGT | |
| | SVTVSSESKYGPPCPPCPMFWVLVVVGGVLAC | |
| | YSLLVTVAFIIFWVKRGRKKLLYIFKQPFMRPV | |
| | QTTQEEDGCSCRFPEEEEGGCELRVKFSRSADA | |
| | PAYQQGQNQLYNELNLGRREEYDVLDKRRGR | |
| | DPEMGGKPRRKNPQEGLYNELQKDKMAEAYS | |
| | EIGMKGERRRGKGHDGLYQGLSTATKDTYDA | |
| | LHMQALPPR | |
| 58 | atgcttctcctggtgacaagccttctgctctgtgagttaccacacccagcatt | Axicabtagene |
| | cctcctgatcccagacatccagatgacacagactacatcctccctgtctgc | ciloleucel CD19 |
| | ctctctgggagacagagtcaccatcagttgcagggcaagtcaggacatta | CAR nucleotide |
| | gtaaatatttaaattggtatcagcagaaaccagatggaactgttaaactcct | sequence |
| | gatctaccatacatcaagattacactcaggagtcccatcaaggttcagtgg | |
| | cagtgggtctggaacagattattctctcaccattagcaacctggagcaaga | |
| | agatattgccacttacttttgccaacagggtaatacgcttccgtacacgttcg | |
| | gaggggggactaagttggaaataacaggctccacctctggatccggcaa | |
| | gcccggatctggcgagggatccaccaagggcgaggtgaaactgcagg | |
| | agtcaggacctggcctggtggcgccctcacagagcctgtccgtcacatg | |
| | cactgtctcaggggtctcattacccgactatggtgtaagctggattcgcca | |
| | gcctccacgaaagggtctggagtggctgggagtaatatggggtagtgaa | |
| | accacatactataattcagctctcaaatccagactgaccatcatcaaggac | |
| | aactccaagagccaagttttcttaaaaatgaacagtctgcaaactgatgac | |
| | acagccatttactactgtgccaaacattattactacggtggtagctatgctat | |
| | ggactactggggtcaaggaacctcagtcaccgtctcctcagcggccgca | |
| | attgaagttatgtatcctcctccttacctagacaatgagaagagcaatggaa | |
| | ccattatccatgtgaaagggaaacacctttgtccaagtcccctatttcccgg | |
| | accttctaagcccttttgggtgctggtggtggttgggggagtcctggcttgc | |
| | tatagcttgctagtaacagtggcctttattattttctgggtgaggagtaagag | |
| | gagcaggctcctgcacagtgactacatgaacatgactccccgccgcccc | |
| | gggcccacccgcaagcattaccagccctatgccccaccacgcgacttcg | |
| | cagcctatcgctccagagtgaagttcagcaggagcgcagacgcccccg | |
| | cgtaccagcagggccagaaccagctctataacgagctcaatctaggacg | |
| | aagagaggagtacgatgttttggacaagagacgtggccgggaccctgag | |
| | atggggggaaagccgagaaggaagaaccctcaggaaggcctgtacaat | |
| | gaactgcagaaagataagatggcggaggcctacagtgagattgggatga | |
| | aaggcgagcgccggaggggcaaggggcacgatggcctttaccagggt | |
| | ctcagtacagccaccaaggacacctacgacgcccttcacatgcaggccc | |
| | tgccccctcgc | |
| 59 | MLLLVTSLLLCELPHPAFLLIPDIQMTQTTSSLS | Axicabtagene |
| | ASLGDRVTISCRASQDISKYLNWYQQKPDGTV | ciloleucel CD19 |
| | KLLIYHTSRLHSGVPSRFSGSGSGTDYSLTISNL | CAR amino acid |
| | EQEDIATYFCQQGNTLPYTFGGGTKLEITGSTS | sequence |
| | GSGKPGSGEGSTKGEVKLQESGPGLVAPSQSLS | |
| | VTCTVSGVSLPDYGVSWIRQPPRKGLEWLGVI | |
| | WGSETTYYNSALKSRLTIIKDNSKSQVFLKMNS | |
| | LQTDDTAIYYCAKHYYYGGSYAMDYWGQGT | |
| | SVTVSSAAAIEVMYPPPYLDNEKSNGTIIHVKG | |
| | KHLCPSPLFPGPSKPFWVLVVVGGVLACYSLL | |
| | VTVAFIIFWVRSKRSRLLHSDYMNMTPRRPGPT | |
| | RKHYQPYAPPRDFAAYRSRVKFSRSADAPAYQ | |
| | QGQNQLYNELNLGRREEYDVLDKRRGRDPEM | |
| | GGKPRRKNPQEGLYNELQKDKMAEAYSEIGM | |
| | KGERRRGKGHDGLYQGLSTATKDTYDALHMQ | |
| | ALPPR | |

TABLE 9
Annotation of tisagenlecleucel CD19 CAR sequences

| | Nucleotide | Amino Acid |
|---|---|---|
| | Sequence | Sequence |
| Feature | Position | Position |
| CD8α signal peptide | 1-63 | 1-21 |
| FMC63 scFv | 64-789 | 22-263 |
| (V_{L}-3xG₄S linker-V_{H}) | | |
| CD8α hinge domain | 790-924 | 264-308 |
| CD8α transmembrane domain | 925-996 | 309-332 |
| 4-1BB costimulatory domain | 997-1122 | 333-374 |
| CD3ζ signaling domain | 1123-1458 | 375-486 |

TABLE 10
Annotation of lisocabtagene maraleucel CD19 CAR sequences

| | Nucleotide | Amino Acid |
|---|---|---|
| | Sequence | Sequence |
| Feature | Position | Position |
| GMCSFR-α signal peptide | 1-66 | 1-22 |
| FMC63 scFv | 67-801 | 23-267 |
| (V_{L}-Whitlow linker-V_{H}) | | |
| IgG4 hinge domain | 802-837 | 268-279 |
| CD28 transmembrane domain | 838-921 | 280-307 |
| 4-1BB costimulatory domain | 922-1047 | 308-349 |
| CD3ζ signaling domain | 1048-1383 | 350-461 |

TABLE 11
Annotation of axicabtagene ciloleucel CD19 CAR sequences

| | Nucleotide | Amino Acid |
|---|---|---|
| | Sequence | Sequence |
| Feature | Position | Position |
| CSF2RA signal peptide | 1-66 | 1-22 |
| FMC63 scFv | 67-801 | 23-267 |
| (V_{L}-Whitlow linker-V_{H}) | | |
| CD28 hinge domain | 802-927 | 268-309 |
| CD28 transmembrane domain | 928-1008 | 310-336 |
| CD28 costimulatory domain | 1009-1131 | 337-377 |
| CD3ζ signaling domain | 1132-1467 | 378-489 |

In some embodiments, the polycistronic vector comprises an expression cassette that contains a nucleotide sequence encoding CD19 CAR as set forth in SEQ ID NO: 54, 56, or 58, or at least 80% identical (e.g., at least 80%, at least 85%, at least 90%, at least 95%, at least 96%, at least 97%, at least 98%, at least 99%, or 100% identical) to the nucleotide sequence set forth in SEQ ID NO: 54, 56, or 58. The encoded CD19 CAR has a corresponding amino acid sequence set forth in SEQ ID NO: 55, 57, or 59, respectively, is at least 80% identical (e.g., at least 80%, at least 85%, at least 90%, at least 95%, at least 96%, at least 97%, at least 98%, at least 99%, or 100% identical) to the amino acid sequence set forth in of SEQ ID NO: 55, 57, or 59, respectively.

b. CD20 CAR

In some embodiments, the CAR is a CD20 CAR (“CD20-CAR”), and in these embodiments, the polycistronic vector comprises an expression cassette that contains a nucleotide sequence encoding a CD20 CAR. CD20 is an antigen found on the surface of B cells as early at the pro-B phase and progressively at increasing levels until B cell maturity, as well as on the cells of most B-cell neoplasms. CD20 positive cells are also sometimes found in cases of Hodgkins disease, myeloma, and thymoma. In some embodiments, the CD20 CAR may comprise a signal peptide, an extracellular binding domain that specifically binds CD20, a hinge domain, a transmembrane domain, an intracellular costimulatory domain, and/or an intracellular signaling domain in tandem.

In some embodiments, the signal peptide of the CD20 CAR comprises a CD8α signal peptide. In some embodiments, the CD8α signal peptide comprises or consists of an amino acid sequence set forth in SEQ ID NO:24 or an amino acid sequence that is at least 80% identical (e.g., at least 80%, at least 85%, at least 90%, at least 95%, at least 96%, at least 97%, at least 98%, at least 99%, or 100% identical) to the amino acid sequence set forth in of SEQ ID NO:24. In some embodiments, the signal peptide comprises an IgK signal peptide. In some embodiments, the IgK signal peptide comprises or consists of an amino acid sequence set forth in SEQ ID NO:25 or an amino acid sequence that is at least 80% identical (e.g., at least 80%, at least 85%, at least 90%, at least 95%, at least 96%, at least 97%, at least 98%, at least 99%, or 100% identical) to the amino acid sequence set forth in of SEQ ID NO:25. In some embodiments, the signal peptide comprises a GMCSFR-α or CSF2RA signal peptide. In some embodiments, the GMCSFR-α or CSF2RA signal peptide comprises or consists of an amino acid sequence set forth in SEQ ID NO:26 or an amino acid sequence that is at least 80% identical (e.g., at least 80%, at least 85%, at least 90%, at least 95%, at least 96%, at least 97%, at least 98%, at least 99%, or 100% identical) to the amino acid sequence set forth in of SEQ ID NO:26.

In some embodiments, the extracellular binding domain of the CD20 CAR is specific to CD20, for example, human CD20. The extracellular binding domain of the CD20 CAR can be codon-optimized for expression in a host cell or to have variant sequences to increase functions of the extracellular binding domain. In some embodiments, the extracellular binding domain comprises an immunogenically active portion of an immunoglobulin molecule, for example, an scFv.

In some embodiments, the extracellular binding domain of the CD20 CAR is derived from an antibody specific to CD20, including, for example, Leu16, IFS, 1.5.3, rituximab, obinutuzumab, ibritumomab, ofatumumab, tositumumab, odronextamab, veltuzumab, ublituximab, and ocrelizumab. In any of these embodiments, the extracellular binding domain of the CD20 CAR can comprise or consist of the V_{H}, the V_{L}, and/or one or more CDRs of any of the antibodies.

In some embodiments, the extracellular binding domain of the CD20 CAR comprises an scFv derived from the Leu16 monoclonal antibody, which comprises the heavy chain variable region (V_{H}) and the light chain variable region (V_{L}) of Leu16 connected by a linker. See Wu et al., Protein Engineering. 14(12):1025-1033 (2001). In some embodiments, the linker is a 3×G₄S linker. In other embodiments, the linker is a Whitlow linker as described herein. In some embodiments, the amino acid sequences of different portions of the entire Leu16-derived scFv (also referred to as Leu16 scFv) and its different portions are provided in Table 12 below. In some embodiments, the CD20-specific scFv comprises or consists of an amino acid sequence set forth in SEQ ID NO:60, 61, or 65, or an amino acid sequence that is at least 80% identical (e.g., at least 80%, at least 85%, at least 90%, at least 95%, at least 96%, at least 97%, at least 98%, at least 99%, or 100% identical) to the amino acid sequence set forth in of SEQ ID NO:60, 61, or 65 In some embodiments, the CD20-specific scFv may comprise one or more CDRs having amino acid sequences set forth in SEQ ID NOs: 62-64, 66, 67, and 68. In some embodiments, the CD20-specific scFv may comprise a light chain with one or more CDRs having amino acid sequences set forth in SEQ ID NOs: 62-64. In some embodiments, the CD20-specific scFv may comprise a heavy chain with one or more CDRs having amino acid sequences set forth in SEQ ID NOs: 66, 67, and 68. In any of these embodiments, the CD20-specific scFv may comprise one or more CDRs comprising one or more amino acid substitutions, or comprising a sequence that is at least 80% identical (e.g., at least 80%, at least 85%, at least 90%, at least 95%, at least 96%, at least 97%, at least 98%, at least 99%, or 100% identical), to any of the sequences identified. In some embodiments, the extracellular binding domain of the CD20 CAR comprises or consists of the one or more CDRs as described herein.

TABLE 12
Exemplary sequences of anti-CD20 scFv and components

| SEQ ID NO: | Amino Acid Sequence | Description |
|---|---|---|
| 60 | DIVLTQSPAILSASPGEKVTMTCRASSS | Anti-CD20 Leu16 scFv |
| | VNYMDWYQKKPGSSPKPWIYATSNLA | entire sequence, with |
| | SGVPARFSGSGSGTSYSLTISRVEAEDA | Whitlow linker |
| | ATYYCQQWSFNPPTFGGGTKLEIKGSTS | |
| | GSGKPGSGEGSTKGEVQLQQSGAELVK | |
| | PGASVKMSCKASGYTFTSYNMHWVKQ | |
| | TPGQGLEWIGAIYPGNGDTSYNQKFKG | |
| | KATLTADKSSSTAYMQLSSLTSEDSAD | |
| | YYCARSNYYGSSYWFFDVWGAGTTVT | |
| | VSS | |
| 61 | DIVLTQSPAILSASPGEKVTMTCRASSS | Anti-CD20 Leu16 scFv |
| | VNYMDWYQKKPGSSPKPWIYATSNLA | light chain variable region |
| | SGVPARFSGSGSGTSYSLTISRVEAEDA | |
| | ATYYCQQWSFNPPTFGGGTKLEIK | |
| 62 | RASSSVNYMD | Anti-CD20 Leu16 scFv |
| | | light chain CDR1 |
| 63 | ATSNLAS | Anti-CD20 Leu16 scFv |
| | | light chain CDR2 |
| 64 | QQWSFNPPT | Anti-CD20 Leu16 scFv |
| | | light chain CDR3 |
| 65 | EVQLQQSGAELVKPGASVKMSCKASG | Anti-CD20 Leu16 scFv |
| | YTFTSYNMHWVKQTPGQGLEWIGAIYP | heavy chain |
| | GNGDTSYNQKFKGKATLTADKSSSTAY | |
| | MQLSSLTSEDSADYYCARSNYYGSSYW | |
| | FFDVWGAGTTVTVSS | |
| 66 | SYNMH | Anti-CD20 Leu16 scFv |
| | | heavy chain CDR1 |
| 67 | AIYPGNGDTSYNQKFKG | Anti-CD20 Leu16 scFv |
| | | heavy chain CDR2 |
| 68 | SNYYGSSYWFFDV | Anti-CD20 Leu16 scFv |
| | | heavy chain CDR3 |

In some embodiments, the hinge domain of the CD20 CAR comprises a CD8α hinge domain, for example, a human CD8α hinge domain. In some embodiments, the CD8α hinge domain comprises or consists of an amino acid sequence set forth in SEQ ID NO:27 or an amino acid sequence that is at least 80% identical (e.g., at least 80%, at least 85%, at least 90%, at least 95%, at least 96%, at least 97%, at least 98%, at least 99%, or 100% identical) to the amino acid sequence set forth in of SEQ ID NO:27. In some embodiments, the hinge domain comprises a CD28 hinge domain, for example, a human CD28 hinge domain. In some embodiments, the CD28 hinge domain comprises or consists of an amino acid sequence set forth in SEQ ID NO:28 or an amino acid sequence that is at least 80% identical (e.g., at least 80%, at least 85%, at least 90%, at least 95%, at least 96%, at least 97%, at least 98%, at least 99%, or 100% identical) to the amino acid sequence set forth in of SEQ ID NO:28. In some embodiments, the hinge domain comprises an IgG4 hinge domain, for example, a human IgG4 hinge domain. In some embodiments, the IgG4 hinge domain comprises or consists of an amino acid sequence set forth in SEQ ID NO:30 or SEQ ID NO:31, or an amino acid sequence that is at least 80% identical (e.g., at least 80%, at least 85%, at least 90%, at least 95%, at least 96%, at least 97%, at least 98%, at least 99%, or 100% identical) to the amino acid sequence set forth in of SEQ ID NO:30 or SEQ ID NO:31. In some embodiments, the hinge domain comprises a IgG4 hinge-Ch2-Ch3 domain, for example, a human IgG4 hinge-Ch2-Ch3 domain. In some embodiments, the IgG4 hinge-Ch2-Ch3 domain comprises or consists of an amino acid sequence set forth in SEQ ID NO:32 or an amino acid sequence that is at least 80% identical (e.g., at least 80%, at least 85%, at least 90%, at least 95%, at least 96%, at least 97%, at least 98%, at least 99%, or 100% identical) to the amino acid sequence set forth in of SEQ ID NO:32.

In some embodiments, the transmembrane domain of the CD20 CAR comprises a CD8α transmembrane domain, for example, a human CD8α transmembrane domain. In some embodiments, the CD8α transmembrane domain comprises or consists of an amino acid sequence set forth in SEQ ID NO:33 or an amino acid sequence that is at least 80% identical (e.g., at least 80%, at least 85%, at least 90%, at least 95%, at least 96%, at least 97%, at least 98%, at least 99%, or 100% identical) to the amino acid sequence set forth in SEQ ID NO:33. In some embodiments, the transmembrane domain comprises a CD28 transmembrane domain, for example, a human CD28 transmembrane domain. In some embodiments, the CD28 transmembrane domain comprises or consists of an amino acid sequence set forth in SEQ ID NO:35 or an amino acid sequence that is at least 80% identical (e.g., at least 80%, at least 85%, at least 90%, at least 95%, at least 96%, at least 97%, at least 98%, at least 99%, or 100% identical) to the amino acid sequence set forth in SEQ ID NO:35.

In some embodiments, the intracellular costimulatory domain of the CD20 CAR comprises a 4-1BB costimulatory domain, for example, a human 4-1BB costimulatory domain. In some embodiments, the 4-1BB costimulatory domain comprises or consists of an amino acid sequence set forth in SEQ ID NO:36 or an amino acid sequence that is at least 80% identical (e.g., at least 80%, at least 85%, at least 90%, at least 95%, at least 96%, at least 97%, at least 98%, at least 99%, or 100% identical) to the amino acid sequence set forth in SEQ ID NO:36. In some embodiments, the intracellular costimulatory domain comprises a CD28 costimulatory domain, for example, a human CD28 costimulatory domain. In some embodiments, the CD28 costimulatory domain comprises or consists of an amino acid sequence set forth in SEQ ID NO:37 or an amino acid sequence that is at least 80% identical (e.g., at least 80%, at least 85%, at least 90%, at least 95%, at least 96%, at least 97%, at least 98%, at least 99%, or 100% identical) to the amino acid sequence set forth in SEQ ID NO:37.

In some embodiments, the intracellular signaling domain of the CD20 CAR comprises a CD3 zeta (ζ) signaling domain, for example, a human CD3ζ signaling domain. In some embodiments, the CD3ζ signaling domain comprises or consists of an amino acid sequence set forth in SEQ ID NO:38 or an amino acid sequence that is at least 80% identical (e.g., at least 80%, at least 85%, at least 90%, at least 95%, at least 96%, at least 97%, at least 98%, at least 99%, or 100% identical) to the amino acid sequence set forth in SEQ ID NO:38.

In some embodiments, the polycistronic vector comprises an expression cassette that contains a nucleotide sequence encoding a CD20 CAR, including, for example, a CD20 CAR comprising the CD20-specific scFv having sequences set forth in SEQ ID NO:60, the CD8α hinge domain of SEQ ID NO:27, the CD8α transmembrane domain of SEQ ID NO:33, the 4-1BB costimulatory domain of SEQ ID NO:36, the CD3ζ signaling domain of SEQ ID NO:38, and/or variants (i.e., having a sequence that is at least 80% identical, for example, at least 80%, at least 85%, at least 90%, at least 95%, at least 96%, at least 97%, at least 98%, or at least 99 identical to the disclosed sequence) thereof.

In some embodiments, the polycistronic vector comprises an expression cassette that contains a nucleotide sequence encoding a CD20 CAR, including, for example, a CD20 CAR comprising the CD20-specific scFv having sequences set forth in SEQ ID NO:60, the CD28 hinge domain of SEQ ID NO:27, the CD8α transmembrane domain of SEQ ID NO:33, the 4-1BB costimulatory domain of SEQ ID NO:36, the CD3ζ signaling domain of SEQ ID NO:38, and/or variants (i.e., having a sequence that is at least 80% identical, for example, at least 80%, at least 85%, at least 90%, at least 95%, at least 96%, at least 97%, at least 98%, or at least 99 identical to the disclosed sequence) thereof.

In some embodiments, the polycistronic vector comprises an expression cassette that contains a nucleotide sequence encoding a CD20 CAR, including, for example, a CD20 CAR comprising the CD20-specific scFv having sequences set forth in SEQ ID NO:60, the IgG4 hinge domain of SEQ ID NO:30 or SEQ ID NO:31, the CD8α transmembrane domain of SEQ ID NO:33, the 4-1BB costimulatory domain of SEQ ID NO:36, the CD3ζ signaling domain of SEQ ID NO:37, and/or variants (i.e., having a sequence that is at least 80% identical, for example, at least 80%, at least 85%, at least 90%, at least 95%, at least 96%, at least 97%, at least 98%, or at least 99 identical to the disclosed sequence) thereof.

In some embodiments, the polycistronic vector comprises an expression cassette that contains a nucleotide sequence encoding a CD20 CAR, including, for example, a CD20 CAR comprising the CD20-specific scFv having sequences set forth in SEQ ID NO:60, the CD8α hinge domain of SEQ ID NO:27, the CD28 transmembrane domain of SEQ ID NO:29, the 4-1BB costimulatory domain of SEQ ID NO:36, the CD3ζ signaling domain of SEQ ID NO:37, and/or variants (i.e., having a sequence that is at least 80% identical, for example, at least 80%, at least 85%, at least 90%, at least 95%, at least 96%, at least 97%, at least 98%, or at least 99 identical to the disclosed sequence) thereof.

In some embodiments, the polycistronic vector comprises an expression cassette that contains a nucleotide sequence encoding a CD20 CAR, including, for example, a CD20 CAR comprising the CD20-specific scFv having sequences set forth in SEQ ID NO:60, the CD28 hinge domain of SEQ ID NO:29, the CD28 transmembrane domain of SEQ ID NO:35, the 4-1BB costimulatory domain of SEQ ID NO:36, the CD3ζ signaling domain of SEQ ID NO:37, and/or variants (i.e., having a sequence that is at least 80% identical, for example, at least 80%, at least 85%, at least 90%, at least 95%, at least 96%, at least 97%, at least 98%, or at least 99 identical to the disclosed sequence) thereof.

In some embodiments, the polycistronic vector comprises an expression cassette that contains a nucleotide sequence encoding a CD20 CAR, including, for example, a CD20 CAR comprising the CD20-specific scFv having sequences set forth in SEQ ID NO:60, the IgG4 hinge domain of SEQ ID NO:30 or SEQ ID NO:31, the CD28 transmembrane domain of SEQ ID NO:34, the 4-1BB costimulatory domain of SEQ ID NO:36, the CD3ζ signaling domain of SEQ ID NO:37, and/or variants (i.e., having a sequence that is at least 80% identical, for example, at least 80%, at least 85%, at least 90%, at least 95%, at least 96%, at least 97%, at least 98%, or at least 99 identical to the disclosed sequence) thereof.

c. CD22 CAR

In some embodiments, the CAR is a CD22 CAR (“CD22-CAR”), and in these embodiments, the polycistronic vector comprises an expression cassette that contains a nucleotide sequence encoding a CD22 CAR. CD22, which is a transmembrane protein found mostly on the surface of mature B cells that functions as an inhibitory receptor for B cell receptor (BCR) signaling. CD22 is expressed in 60-70% of B cell lymphomas and leukemias (e.g., B-chronic lymphocytic leukemia, hairy cell leukemia, acute lymphocytic leukemia (ALL), and Burkitt's lymphoma) and is not present on the cell surface in early stages of B cell development or on stem cells. In some embodiments, the CD22 CAR may comprise a signal peptide, an extracellular binding domain that specifically binds CD22, a hinge domain, a transmembrane domain, an intracellular costimulatory domain, and/or an intracellular signaling domain in tandem.

In some embodiments, the signal peptide of the CD22 CAR comprises a CD8α signal peptide. In some embodiments, the CD8α signal peptide comprises or consists of an amino acid sequence set forth in SEQ ID NO:24 or an amino acid sequence that is at least 80% identical (e.g., at least 80%, at least 85%, at least 90%, at least 95%, at least 96%, at least 97%, at least 98%, at least 99%, or 100% identical) to the amino acid sequence set forth in of SEQ ID NO:24. In some embodiments, the signal peptide comprises an IgK signal peptide. In some embodiments, the IgK signal peptide comprises or consists of an amino acid sequence set forth in SEQ ID NO:25 or an amino acid sequence that is at least 80% identical (e.g., at least 80%, at least 85%, at least 90%, at least 95%, at least 96%, at least 97%, at least 98%, at least 99%, or 100% identical) to the amino acid sequence set forth in of SEQ ID NO: 25. In some embodiments, the signal peptide comprises a GMCSFR-α or CSF2RA signal peptide. In some embodiments, the GMCSFR-α or CSF2RA signal peptide comprises or consists of an amino acid sequence set forth in SEQ ID NO:26 or an amino acid sequence that is at least 80% identical (e.g., at least 80%, at least 85%, at least 90%, at least 95%, at least 96%, at least 97%, at least 98%, at least 99%, or 100% identical) to the amino acid sequence set forth in of SEQ ID NO:26.

In some embodiments, the extracellular binding domain of the CD22 CAR is specific to CD22, for example, human CD22. The extracellular binding domain of the CD22 CAR can be codon-optimized for expression in a host cell or to have variant sequences to increase functions of the extracellular binding domain. In some embodiments, the extracellular binding domain comprises an immunogenically active portion of an immunoglobulin molecule, for example, an scFv.

In some embodiments, the extracellular binding domain of the CD22 CAR is derived from an antibody specific to CD22, including, for example, SM03, inotuzumab, epratuzumab, moxetumomab, and pinatuzumab. In any of these embodiments, the extracellular binding domain of the CD22 CAR can comprise or consist of the V_{H}, the V_{L}, and/or one or more CDRs of any of the antibodies.

In some embodiments, the extracellular binding domain of the CD22 CAR comprises an scFv derived from the m971 monoclonal antibody (m971), which comprises the heavy chain variable region (V_{H}) and the light chain variable region (V_{L}) of m971 connected by a linker. In some embodiments, the linker is a 3×G₄S linker. In other embodiments, the Whitlow linker may be used instead. In some embodiments, the amino acid sequences of the entire m971-derived scFv (also referred to as m971 scFv) and its different portions are provided in Table 14 below. In some embodiments, the CD22-specific scFv comprises or consists of an amino acid sequence set forth in SEQ ID NO:69, 70, or 74, or an amino acid sequence that is at least 80% identical (e.g., at least 80%, at least 85%, at least 90%, at least 95%, at least 96%, at least 97%, at least 98%, at least 99%, or 100% identical) to the amino acid sequence set forth in of SEQ ID NO: 69, 70, or 74. In some embodiments, the CD22-specific scFv may comprise one or more CDRs having amino acid sequences set forth in SEQ ID NOs: 130-132 and 134-136. In some embodiments, the CD22-specific scFv may comprise a heavy chain with one or more CDRs having amino acid sequences set forth in SEQ ID NOs: 71-73. In some embodiments, the CD22-specific scFv may comprise a light chain with one or more CDRs having amino acid sequences set forth in SEQ ID NOs: 75-77. In any of these embodiments, the CD22-specific scFv may comprise one or more CDRs comprising one or more amino acid substitutions, or comprising a sequence that is at least 80% identical (e.g., at least 80%, at least 85%, at least 90%, at least 95%, at least 96%, at least 97%, at least 98%, at least 99%, or 100% identical), to any of the sequences identified. In some embodiments, the extracellular binding domain of the CD22 CAR comprises or consists of the one or more CDRs as described herein.

In some embodiments, the extracellular binding domain of the CD22 CAR comprises an scFv derived from m971-L7, which is an affinity matured variant of m971 with significantly improved CD22 binding affinity compared to the parental antibody m971 (improved from about 2 nM to less than 50 pM). In some embodiments, the scFv derived from m971-L7 comprises the V_{H} and the V_{L} of m971-L7 connected by a 3×G₄S linker. In other embodiments, the Whitlow linker may be used instead. In some embodiments, the amino acid sequences of the entire m971-L7-derived scFv (also referred to as m971-L7 scFv) and its different portions are provided in Table 13 below. In some embodiments, the CD22-specific scFv comprises or consists of an amino acid sequence set forth in SEQ ID NO:137, 138, or 142, or an amino acid sequence that is at least 80% identical (e.g., at least 80%, at least 85%, at least 90%, at least 95%, at least 96%, at least 97%, at least 98%, at least 99%, or 100% identical) to the amino acid sequence set forth in of SEQ ID NO:78, 79, or 83. In some embodiments, the CD22-specific scFv may comprise one or more CDRs having amino acid sequences set forth in SEQ ID NOs: 80-82, 83-86. In some embodiments, the CD22-specific scFv may comprise a heavy chain with one or more CDRs having amino acid sequences set forth in SEQ ID NOs: 80-82. In some embodiments, the CD22-specific scFv may comprise a light chain with one or more CDRs having amino acid sequences set forth in SEQ ID NOs: 83-86. In any of these embodiments, the CD22-specific scFv may comprise one or more CDRs comprising one or more amino acid substitutions, or comprising a sequence that is at least 80% identical (e.g., at least 80%, at least 85%, at least 90%, at least 95%, at least 96%, at least 97%, at least 98%, at least 99%, or 100% identical), to any of the sequences identified. In some embodiments, the extracellular binding domain of the CD22 CAR comprises or consists of the one or more CDRs as described herein.

TABLE 13
Exemplary sequences of anti-CD22 scFv and components

| SEQ ID NO: | Amino Acid Sequence | Description |
|---|---|---|
| 69 | QVQLQQSGPGLVKPSQTLSLTCAISGDS | Anti-CD22 m971 scFv |
| | VSSNSAAWNWIRQSPSRGLEWLGRTYY | entire sequence, with |
| | RSKWYNDYAVSVKSRITINPDTSKNQFS | 3xG4S linker |
| | LQLNSVTPEDTAVYYCAREVTGDLEDA | |
| | FDIWGQGTMVTVSSGGGGSGGGGSGG | |
| | GGSDIQMTQSPSSLSASVGDRVTITCRA | |
| | SQTIWSYLNWYQQRPGKAPNLLIYAAS | |
| | SLQSGVPSRFSGRGSGTDFTLTISSLQAE | |
| | DFATYYCQQSYSIPQTFGQGTKLEIK | |
| 70 | QVQLQQSGPGLVKPSQTLSLTCAISGDS | Anti-CD22 m971 scFv |
| | VSSNSAAWNWIRQSPSRGLEWLGRTYY | heavy chain variable |
| | RSKWYNDYAVSVKSRITINPDTSKNQFS | region |
| | LQLNSVTPEDTAVYYCAREVTGDLEDA | |
| | FDIWGQGTMVTVSS | |
| 71 | GDSVSSNSAA | Anti-CD22 m971 scFv |
| | | heavy chain CDR1 |
| 72 | TYYRSKWYN | Anti-CD22 m971 scFv |
| | | heavy chain CDR2 |
| 73 | AREVTGDLEDAFDI | Anti-CD22 m971 scFv |
| | | heavy chain CDR3 |
| 74 | DIQMTQSPSSLSASVGDRVTITCRASQTI | Anti-CD22 m971 scFv |
| | WSYLNWYQQRPGKAPNLLIYAASSLQS | light chain |
| | GVPSRFSGRGSGTDFTLTISSLQAEDFAT | |
| | YYCQQSYSIPQTFGQGTKLEIK | |
| 75 | QTIWSY | Anti-CD22 m971 scFv |
| | | light chain CDR1 |
| 76 | AAS | Anti-CD22 m971 scFv |
| | | light chain CDR2 |
| 77 | QQSYSIPQT | Anti-CD22 m971 scFv |
| | | light chain CDR3 |
| 78 | QVQLQQSGPGMVKPSQTLSLTCAISGD | Anti-CD22 m971-L7 scFv |
| | SVSSNSVAWNWIRQSPSRGLEWLGRTY | entire sequence, with |
| | YRSTWYNDYAVSMKSRITINPDTNKNQ | 3xG4S linker |
| | FSLQLNSVTPEDTAVYYCAREVTGDLE | |
| | DAFDIWGQGTMVTVSSGGGGSGGGGS | |
| | GGGGSDIQMIQSPSSLSASVGDRVTITC | |
| | RASQTIWSYLNWYRQRPGEAPNLLIYA | |
| | ASSLQSGVPSRFSGRGSGTDFTLTISSLQ | |
| | AEDFATYYCQQSYSIPQTFGQGTKLEIK | |
| 79 | QVQLQQSGPGMVKPSQTLSLTCAISGD | Anti-CD22 m971-L7 scFv |
| | SVSSNSVAWNWIRQSPSRGLEWLGRTY | heavy chain variable |
| | YRSTWYNDYAVSMKSRITINPDTNKNQ | region |
| | FSLQLNSVTPEDTAVYYCAREVTGDLE | |
| | DAFDIWGQGTMVTVSS | |
| 80 | GDSVSSNSVA | Anti-CD22 m971-L7 scFv |
| | | heavy chain CDR1 |
| 81 | TYYRSTWYN | Anti-CD22 m971-L7 scFv |
| | | heavy chain CDR2 |
| 82 | AREVTGDLEDAFDI | Anti-CD22 m971-L7 scFv |
| | | heavy chain CDR3 |
| 83 | DIQMIQSPSSLSASVGDRVTITCRASQTI | Anti-CD22 m971-L7 scFv |
| | WSYLNWYRQRPGEAPNLLIYAASSLQS | light chain variable region |
| | GVPSRFSGRGSGTDFTLTISSLQAEDFAT | |
| | YYCQQSYSIPQTFGQGTKLEIK | |
| 84 | QTIWSY | Anti-CD22 m971-L7 scFv |
| | | light chain CDR1 |
| 85 | AAS | Anti-CD22 m971-L7 scFv |
| | | light chain CDR2 |
| 86 | QQSYSIPQT | Anti-CD22 m971-L7 scFv |
| | | light chain CDR3 |

In some embodiments, the extracellular binding domain of the CD22 CAR comprises immunotoxins HA22 or BL22. Immunotoxins BL22 and HA22 are therapeutic agents that comprise an scFv specific for CD22 fused to a bacterial toxin, and thus can bind to the surface of the cancer cells that express CD22 and kill the cancer cells. BL22 comprises a dsFv of an anti-CD22 antibody, RFB4, fused to a 38-kDa truncated form of Pseudomonas exotoxin A (Bang et al., Clin. Cancer Res., 11:1545-50 (2005)). HA22 (CAT8015, moxetumomab pasudotox) is a mutated, higher affinity version of BL22 (Ho et al., J. Biol. Chem., 280(1): 607-17 (2005)). Suitable sequences of antigen binding domains of HA22 and BL22 specific to CD22 are disclosed in, for example, U.S. Pat. Nos. 7,541,034; 7,355,012; and 7,982,011, which are hereby incorporated by reference in their entirety.

In some embodiments, the hinge domain of the CD22 CAR comprises a CD8α hinge domain, for example, a human CD8α hinge domain. In some embodiments, the CD8α hinge domain comprises or consists of an amino acid sequence set forth in SEQ ID NO:24 or an amino acid sequence that is at least 80% identical (e.g., at least 80%, at least 85%, at least 90%, at least 95%, at least 96%, at least 97%, at least 98%, at least 99%, or 100% identical) to the amino acid sequence set forth in of SEQ ID NO:24. In some embodiments, the hinge domain comprises a CD28 hinge domain, for example, a human CD28 hinge domain. In some embodiments, the CD28 hinge domain comprises or consists of an amino acid sequence set forth in SEQ ID NO:28 or SEQ ID NO: 29 or an amino acid sequence that is at least 80% identical (e.g., at least 80%, at least 85%, at least 90%, at least 95%, at least 96%, at least 97%, at least 98%, at least 99%, or 100% identical) to the amino acid sequence set forth in of SEQ ID NO:28 or SEQ ID NO: 29. In some embodiments, the hinge domain comprises an IgG4 hinge domain, for example, a human IgG4 hinge domain. In some embodiments, the IgG4 hinge domain comprises or consists of an amino acid sequence set forth in SEQ ID NO:30 or SEQ ID NO:31, or an amino acid sequence that is at least 80% identical (e.g., at least 80%, at least 85%, at least 90%, at least 95%, at least 96%, at least 97%, at least 98%, at least 99%, or 100% identical) to the amino acid sequence set forth in of SEQ ID NO:30 or SEQ ID NO:31. In some embodiments, the hinge domain comprises a IgG4 hinge-Ch2-Ch3 domain, for example, a human IgG4 hinge-Ch2-Ch3 domain. In some embodiments, the IgG4 hinge-Ch2-Ch3 domain comprises or consists of an amino acid sequence set forth in SEQ ID NO:32 or an amino acid sequence that is at least 80% identical (e.g., at least 80%, at least 85%, at least 90%, at least 95%, at least 96%, at least 97%, at least 98%, at least 99%, or 100% identical) to the amino acid sequence set forth in of SEQ ID NO:32.

In some embodiments, the transmembrane domain of the CD22 CAR comprises a CD8α transmembrane domain, for example, a human CD8α transmembrane domain. In some embodiments, the CD8α transmembrane domain comprises or consists of an amino acid sequence set forth in SEQ ID NO:33 or an amino acid sequence that is at least 80% identical (e.g., at least 80%, at least 85%, at least 90%, at least 95%, at least 96%, at least 97%, at least 98%, at least 99%, or 100% identical) to the amino acid sequence set forth in SEQ ID NO:33. In some embodiments, the transmembrane domain comprises a CD28 transmembrane domain, for example, a human CD28 transmembrane domain. In some embodiments, the CD28 transmembrane domain comprises or consists of an amino acid sequence set forth in SEQ ID NO:35 or an amino acid sequence that is at least 80% identical (e.g., at least 80%, at least 85%, at least 90%, at least 95%, at least 96%, at least 97%, at least 98%, at least 99%, or 100% identical) to the amino acid sequence set forth in SEQ ID NO:35.

In some embodiments, the intracellular costimulatory domain of the CD22 CAR comprises a 4-1BB costimulatory domain, for example, a human 4-1BB costimulatory domain. In some embodiments, the 4-1BB costimulatory domain comprises or consists of an amino acid sequence set forth in SEQ ID NO:36 or an amino acid sequence that is at least 80% identical (e.g., at least 80%, at least 85%, at least 90%, at least 95%, at least 96%, at least 97%, at least 98%, at least 99%, or 100% identical) to the amino acid sequence set forth in SEQ ID NO:36. In some embodiments, the intracellular costimulatory domain comprises a CD28 costimulatory domain, for example, a human CD28 costimulatory domain. In some embodiments, the CD28 costimulatory domain comprises or consists of an amino acid sequence set forth in SEQ ID NO:37 or an amino acid sequence that is at least 80% identical (e.g., at least 80%, at least 85%, at least 90%, at least 95%, at least 96%, at least 97%, at least 98%, at least 99%, or 100% identical) to the amino acid sequence set forth in SEQ ID NO:37.

In some embodiments, the intracellular signaling domain of the CD22 CAR comprises a CD3 zeta (ζ) signaling domain, for example, a human CD3ζ signaling domain. In some embodiments, the CD3ζ signaling domain comprises or consists of an amino acid sequence set forth in SEQ ID NO:38 or an amino acid sequence that is at least 80% identical (e.g., at least 80%, at least 85%, at least 90%, at least 95%, at least 96%, at least 97%, at least 98%, at least 99%, or 100% identical) to the amino acid sequence set forth in SEQ ID NO:38. In some embodiments, the CD3ζ signaling domain comprises or consists of an amino acid sequence set forth in SEQ ID NO:39 or an amino acid sequence that is at least 80% identical (e.g., at least 80%, at least 85%, at least 90%, at least 95%, at least 96%, at least 97%, at least 98%, at least 99%, or 100% identical) to the amino acid sequence set forth in SEQ ID NO:39.

In some embodiments, the polycistronic vector comprises an expression cassette that contains a nucleotide sequence encoding a CD22 CAR, including, for example, a CD22 CAR comprising the CD22-specific scFv having sequences set forth in SEQ ID NO:128 or SEQ ID NO:137, the CD8α hinge domain of SEQ ID NO:88, the CD8α transmembrane domain of SEQ ID NO:94, the 4-1BB costimulatory domain of SEQ ID NO:97, the CD3ζ signaling domain of SEQ ID NO:99, and/or variants (i.e., having a sequence that is at least 80% identical, for example, at least 80%, at least 85%, at least 90%, at least 95%, at least 96%, at least 97%, at least 98%, or at least 99 identical to the disclosed sequence) thereof.

In some embodiments, the polycistronic vector comprises an expression cassette that contains a nucleotide sequence encoding a CD22 CAR, including, for example, a CD22 CAR comprising the CD22-specific scFv having sequences set forth in SEQ ID NO:128 or SEQ ID NO:137, the CD28 hinge domain of SEQ ID NO:89, the CD8α transmembrane domain of SEQ ID NO:94, the 4-1BB costimulatory domain of SEQ ID NO:97, the CD3ζ signaling domain of SEQ ID NO:99, and/or variants (i.e., having a sequence that is at least 80% identical, for example, at least 80%, at least 85%, at least 90%, at least 95%, at least 96%, at least 97%, at least 98%, or at least 99 identical to the disclosed sequence) thereof.

In some embodiments, the polycistronic vector comprises an expression cassette that contains a nucleotide sequence encoding a CD22 CAR, including, for example, a CD22 CAR comprising the CD22-specific scFv having sequences set forth in SEQ ID NO:128 or SEQ ID NO:137, the IgG4 hinge domain of SEQ ID NO:91 or SEQ ID NO:92, the CD8α transmembrane domain of SEQ ID NO:94, the 4-1BB costimulatory domain of SEQ ID NO:97, the CD3ζ signaling domain of SEQ ID NO:99, and/or variants (i.e., having a sequence that is at least 80% identical, for example, at least 80%, at least 85%, at least 90%, at least 95%, at least 96%, at least 97%, at least 98%, or at least 99 identical to the disclosed sequence) thereof.

In some embodiments, the polycistronic vector comprises an expression cassette that contains a nucleotide sequence encoding a CD22 CAR, including, for example, a CD22 CAR comprising the CD22-specific scFv having sequences set forth in SEQ ID NO:128 or SEQ ID NO:137, the CD8α hinge domain of SEQ ID NO:8, the CD28 transmembrane domain of SEQ ID NO:95, the 4-1BB costimulatory domain of SEQ ID NO:97, the CD3ζ signaling domain of SEQ ID NO:99, and/or variants (i.e., having a sequence that is at least 80% identical, for example, at least 80%, at least 85%, at least 90%, at least 95%, at least 96%, at least 97%, at least 98%, or at least 99 identical to the disclosed sequence) thereof.

In some embodiments, the polycistronic vector comprises an expression cassette that contains a nucleotide sequence encoding a CD22 CAR, including, for example, a CD22 CAR comprising the CD22-specific scFv having sequences set forth in SEQ ID NO:128 or SEQ ID NO:137, the CD28 hinge domain of SEQ ID NO:89, the CD28 transmembrane domain of SEQ ID NO:95, the 4-1BB costimulatory domain of SEQ ID NO:97, the CD3ζ signaling domain of SEQ ID NO:99, and/or variants (i.e., having a sequence that is at least 80% identical, for example, at least 80%, at least 85%, at least 90%, at least 95%, at least 96%, at least 97%, at least 98%, or at least 99 identical to the disclosed sequence) thereof.

In some embodiments, the polycistronic vector comprises an expression cassette that contains a nucleotide sequence encoding a CD22 CAR, including, for example, a CD22 CAR comprising the CD22-specific scFv having sequences set forth in SEQ ID NO:128 or SEQ ID NO:137, the IgG4 hinge domain of SEQ ID NO:91 or SEQ ID NO:92, the CD28 transmembrane domain of SEQ ID NO:95, the 4-1BB costimulatory domain of SEQ ID NO:97, the CD3ζ signaling domain of SEQ ID NO:99, and/or variants (i.e., having a sequence that is at least 80% identical, for example, at least 80%, at least 85%, at least 90%, at least 95%, at least 96%, at least 97%, at least 98%, or at least 99 identical to the disclosed sequence) thereof.

d. BCMA CAR

In some embodiments, the CAR is a BCMA CAR (“BCMA-CAR”), and in these embodiments, the polycistronic vector comprises an expression cassette that contains a nucleotide sequence encoding a BCMA CAR. BCMA is a tumor necrosis family receptor (TNFR) member expressed on cells of the B cell lineage, with the highest expression on terminally differentiated B cells or mature B lymphocytes. BCMA is involved in mediating the survival of plasma cells for maintaining long-term humoral immunity. The expression of BCMA has been recently linked to a number of cancers, such as multiple myeloma, Hodgkin's and non-Hodgkin's lymphoma, various leukemias, and glioblastoma. In some embodiments, the BCMA CAR may comprise a signal peptide, an extracellular binding domain that specifically binds BCMA, a hinge domain, a transmembrane domain, an intracellular costimulatory domain, and/or an intracellular signaling domain in tandem.

In some embodiments, the signal peptide of the BCMA CAR comprises a CD8α signal peptide. In some embodiments, the CD8α signal peptide comprises or consists of an amino acid sequence set forth in SEQ ID NO:85 or an amino acid sequence that is at least 80% identical (e.g., at least 80%, at least 85%, at least 90%, at least 95%, at least 96%, at least 97%, at least 98%, at least 99%, or 100% identical) to the amino acid sequence set forth in of SEQ ID NO:85. In some embodiments, the signal peptide comprises an IgK signal peptide. In some embodiments, the IgK signal peptide comprises or consists of an amino acid sequence set forth in SEQ ID NO:86 or an amino acid sequence that is at least 80% identical (e.g., at least 80%, at least 85%, at least 90%, at least 95%, at least 96%, at least 97%, at least 98%, at least 99%, or 100% identical) to the amino acid sequence set forth in of SEQ ID NO:86. In some embodiments, the signal peptide comprises a GMCSFR-α or CSF2RA signal peptide. In some embodiments, the GMCSFR-α or CSF2RA signal peptide comprises or consists of an amino acid sequence set forth in SEQ ID NO:87 or an amino acid sequence that is at least 80% identical (e.g., at least 80%, at least 85%, at least 90%, at least 95%, at least 96%, at least 97%, at least 98%, at least 99%, or 100% identical) to the amino acid sequence set forth in of SEQ ID NO:87.

In some embodiments, the extracellular binding domain of the BCMA CAR is specific to BCMA, for example, human BCMA. The extracellular binding domain of the BCMA CAR can be codon-optimized for expression in a host cell or to have variant sequences to increase functions of the extracellular binding domain.

In some embodiments, the extracellular binding domain comprises an immunogenically active portion of an immunoglobulin molecule, for example, an scFv. In some embodiments, the extracellular binding domain of the BCMA CAR is derived from an antibody specific to BCMA, including, for example, belantamab, erlanatamab, teclistamab, LCAR-B38M, and ciltacabtagene. In any of these embodiments, the extracellular binding domain of the BCMA CAR can comprise or consist of the V_{H}, the V_{L}, and/or one or more CDRs of any of the antibodies.

In some embodiments, the extracellular binding domain of the BCMA CAR comprises an scFv derived from C11D5.3, a murine monoclonal antibody as described in Carpenter et al., Clin. Cancer Res. 19(8):2048-2060 (2013). See also PCT Application Publication No. WO2010/104949. The C11D5.3-derived scFv may comprise the heavy chain variable region (V_{H}) and the light chain variable region (V_{L}) of C11D5.3 connected by the Whitlow linker, the amino acid sequences of which is provided in Table 15 below. In some embodiments, the BCMA-specific extracellular binding domain comprises or consists of an amino acid sequence set forth in SEQ ID NO:146, 147, or 151, or an amino acid sequence that is at least 80% identical (e.g., at least 80%, at least 85%, at least 90%, at least 95%, at least 96%, at least 97%, at least 98%, at least 99%, or 100% identical) to the amino acid sequence set forth in of SEQ ID NO:146, 147, or 151. In some embodiments, the BCMA-specific extracellular binding domain may comprise one or more CDRs having amino acid sequences set forth in SEQ ID NOs: 148-150 and 152-154. In some embodiments, the BCMA-specific extracellular binding domain may comprise a light chain with one or more CDRs having amino acid sequences set forth in SEQ ID NOs: 148-150. In some embodiments, the BCMA-specific extracellular binding domain may comprise a heavy chain with one or more CDRs having amino acid sequences set forth in SEQ ID NOs: 152-154. In any of these embodiments, the BCMA-specific scFv may comprise one or more CDRs comprising one or more amino acid substitutions, or comprising a sequence that is at least 80% identical (e.g., at least 80%, at least 85%, at least 90%, at least 95%, at least 96%, at least 97%, at least 98%, at least 99%, or 100% identical), to any of the sequences identified. In some embodiments, the extracellular binding domain of the BCMA CAR comprises or consists of the one or more CDRs as described herein.

In some embodiments, the extracellular binding domain of the BCMA CAR comprises an scFv derived from another murine monoclonal antibody, C12A3.2, as described in Carpenter et al., Clin. Cancer Res. 19(8):2048-2060 (2013) and PCT Application Publication No. WO2010/104949, the amino acid sequence of which is also provided in Table 14 below. In some embodiments, the BCMA-specific extracellular binding domain comprises or consists of an amino acid sequence set forth in SEQ ID NO:155, 156, or 160, or an amino acid sequence that is at least 80% identical (e.g., at least 80%, at least 85%, at least 90%, at least 95%, at least 96%, at least 97%, at least 98%, at least 99%, or 100% identical) to the amino acid sequence set forth in of SEQ ID NO:155, 156, or 160. In some embodiments, the BCMA-specific extracellular binding domain may comprise one or more CDRs having amino acid sequences set forth in SEQ ID NOs: 157-159 and 161-163. In some embodiments, the BCMA-specific extracellular binding domain may comprise a light chain with one or more CDRs having amino acid sequences set forth in SEQ ID NOs: 157-159. In some embodiments, the BCMA-specific extracellular binding domain may comprise a heavy chain with one or more CDRs having amino acid sequences set forth in SEQ ID NOs: 161-163. In any of these embodiments, the BCMA-specific scFv may comprise one or more CDRs comprising one or more amino acid substitutions, or comprising a sequence that is at least 80% identical (e.g., at least 80%, at least 85%, at least 90%, at least 95%, at least 96%, at least 97%, at least 98%, at least 99%, or 100% identical), to any of the sequences identified. In some embodiments, the extracellular binding domain of the BCMA CAR comprises or consists of the one or more CDRs as described herein.

In some embodiments, the extracellular binding domain of the BCMA CAR comprises a murine monoclonal antibody with high specificity to human BCMA, referred to as BB2121 in Friedman et al., Hum. Gene Ther. 29(5):585-601 (2018)). See also, PCT Application Publication No. WO2012163805.

In some embodiments, the extracellular binding domain of the BCMA CAR comprises single variable fragments of two heavy chains (VHH) that can bind to two epitopes of BCMA as described in Zhao et al., J. Hematol. Oncol. 11(1):141 (2018), also referred to as LCAR-B38M. See also, PCT Application Publication No. WO2018/028647.

In some embodiments, the extracellular binding domain of the BCMA CAR comprises a fully human heavy-chain variable domain (FHVH) as described in Lam et al., Nat. Commun. 11(1):283 (2020), also referred to as FHVH33. See also, PCT Application Publication No. WO2019/006072. The amino acid sequences of FHVH33 and its CDRs are provided in Table 14 below. In some embodiments, the BCMA-specific extracellular binding domain comprises or consists of an amino acid sequence set forth in SEQ ID NO:164 or an amino acid sequence that is at least 80% identical (e.g., at least 80%, at least 85%, at least 90%, at least 95%, at least 96%, at least 97%, at least 98%, at least 99%, or 100% identical) to the amino acid sequence set forth in of SEQ ID NO:164. In some embodiments, the BCMA-specific extracellular binding domain may comprise one or more CDRs having amino acid sequences set forth in SEQ ID NOs: 165-167. In any of these embodiments, the BCMA-specific extracellular binding domain may comprise one or more CDRs comprising one or more amino acid substitutions, or comprising a sequence that is at least 80% identical (e.g., at least 80%, at least 85%, at least 90%, at least 95%, at least 96%, at least 97%, at least 98%, at least 99%, or 100% identical), to any of the sequences identified. In some embodiments, the extracellular binding domain of the BCMA CAR comprises or consists of the one or more CDRs as described herein.

In some embodiments, the extracellular binding domain of the BCMA CAR comprises an scFv derived from CT103A (or CAR0085) as described in U.S. Pat. No. 11,026,975 B2, the amino acid sequence of which is provided in Table 14 below. In some embodiments, the BCMA-specific extracellular binding domain comprises or consists of an amino acid sequence set forth in SEQ ID NO:168, 169, or 173, or an amino acid sequence that is at least 80% identical (e.g., at least 80%, at least 85%, at least 90%, at least 95%, at least 96%, at least 97%, at least 98%, at least 99%, or 100% identical) to the amino acid sequence set forth in of SEQ ID NO: 168, 169, or 173. In some embodiments, the BCMA-specific extracellular binding domain may comprise one or more CDRs having amino acid sequences set forth in SEQ ID NOs: 170-172 and 174-176. In some embodiments, the BCMA-specific extracellular binding domain may comprise a light chain with one or more CDRs having amino acid sequences set forth in SEQ ID NOs: 170-172. In some embodiments, the BCMA-specific extracellular binding domain may comprise a heavy chain with one or more CDRs having amino acid sequences set forth in SEQ ID NOs: 174-176. In any of these embodiments, the BCMA-specific scFv may comprise one or more CDRs comprising one or more amino acid substitutions, or comprising a sequence that is at least 80% identical (e.g., at least 80%, at least 85%, at least 90%, at least 95%, at least 96%, at least 97%, at least 98%, at least 99%, or 100% identical), to any of the sequences identified. In some embodiments, the extracellular binding domain of the BCMA CAR comprises or consists of the one or more CDRs as described herein.

Additionally, CARs and binders directed to BCMA have been described in U.S. Application Publication Nos. 2020/0246381 A1 and 2020/0339699 A1, the entire contents of each of which are incorporated by reference herein.

TABLE 14
Exemplary sequences of anti-BCMA binder and components

| SEQ ID NO: | Amino Acid Sequence | Description |
|---|---|---|
| 87 | DIVLTQSPASLAMSLGKRATISCRASES | Anti-BCMA C11D5.3 |
| | VSVIGAHLIHWYQQKPGQPPKLLIYLAS | scFv entire sequence, with |
| | NLETGVPARFSGSGSGTDFTLTIDPVEE | Whitlow linker |
| | DDVAIYSCLQSRIFPRTFGGGTKLEIKGS | |
| | TSGSGKPGSGEGSTKGQIQLVQSGPELK | |
| | KPGETVKISCKASGYTFTDYSINWVKR | |
| | APGKGLKWMGWINTETREPAYAYDFR | |
| | GRFAFSLETSASTAYLQINNLKYEDTAT | |
| | YFCALDYSYAMDYWGQGTSVTVSS | |
| 88 | DIVLTQSPASLAMSLGKRATISCRASES | Anti-BCMA C11D5.3 |
| | VSVIGAHLIHWYQQKPGQPPKLLIYLAS | scFv light chain variable |
| | NLETGVPARFSGSGSGTDFTLTIDPVEE | region |
| | DDVAIYSCLQSRIFPRTFGGGTKLEIK | |
| 89 | RASESVSVIGAHLIH | Anti-BCMA C11D5.3 |
| | | scFv light chain CDR1 |
| 90 | LASNLET | Anti-BCMA C11D5.3 |
| | | scFv light chain CDR2 |
| 91 | LQSRIFPRT | Anti-BCMA C11D5.3 |
| | | scFv light chain CDR3 |
| 92 | QIQLVQSGPELKKPGETVKISCKASGYT | Anti-BCMA C11D5.3 |
| | FTDYSINWVKRAPGKGLKWMGWINTE | scFv heavy chain variable |
| | TREPAYAYDFRGRFAFSLETSASTAYLQ | region |
| | INNLKYEDTATYFCALDYSYAMDYWG | |
| | QGTSVTVSS | |
| 93 | DYSIN | Anti-BCMA C11D5.3 |
| | | scFv heavy chain CDR1 |
| 94 | WINTETREPAYAYDFRG | Anti-BCMA C11D5.3 |
| | | scFv heavy chain CDR2 |
| 95 | DYSYAMDY | Anti-BCMA C11D5.3 |
| | | scFv heavy chain CDR3 |
| 96 | DIVLTQSPPSLAMSLGKRATISCRASESV | Anti-BCMA C12A3.2 |
| | TILGSHLIYWYQQKPGQPPTLLIQLASN | scFv entire sequence, with |
| | VQTGVPARFSGSGSRTDFTLTIDPVEED | Whitlow linker |
| | DVAVYYCLQSRTIPRTFGGGTKLEIKGS | |
| | TSGSGKPGSGEGSTKGQIQLVQSGPELK | |
| | KPGETVKISCKASGYTFRHYSMNWVK | |
| | QAPGKGLKWMGRINTESGVPIYADDFK | |
| | GRFAFSVETSASTAYLVINNLKDEDTAS | |
| | YFCSNDYLYSLDFWGQGTALTVSS | |
| 97 | DIVLTQSPPSLAMSLGKRATISCRASESV | Anti-BCMA C12A3.2 |
| | TILGSHLIYWYQQKPGQPPTLLIQLASN | scFv light chain variable |
| | VQTGVPARFSGSGSRTDFTLTIDPVEED | region |
| | DVAVYYCLQSRTIPRTFGGGTKLEIK | |
| 98 | RASESVTILGSHLIY | Anti-BCMA C12A3.2 |
| | | scFv light chain CDR1 |
| 99 | LASNVQT | Anti-BCMA C12A3.2 |
| | | scFv light chain CDR2 |
| 100 | LQSRTIPRT | Anti-BCMA C12A3.2 |
| | | scFv light chain CDR3 |
| 101 | QIQLVQSGPELKKPGETVKISCKASGYT | Anti-BCMA C12A3.2 |
| | FRHYSMNWVKQAPGKGLKWMGRINTE | scFv heavy chain variable |
| | SGVPIYADDFKGRFAFSVETSASTAYLV | region |
| | INNLKDEDTASYFCSNDYLYSLDFWGQ | |
| | GTALTVSS | |
| 102 | HYSMN | Anti-BCMA C12A3.2 |
| | | scFv heavy chain CDR1 |
| 103 | RINTESGVPIYADDFKG | Anti-BCMA C12A3.2 |
| | | scFv heavy chain CDR2 |
| 104 | DYLYSLDF | Anti-BCMA C12A3.2 |
| | | scFv heavy chain CDR3 |
| 105 | EVQLLESGGGLVQPGGSLRLSCAASGF | Anti-BCMA FHVH33 |
| | TFSSYAMSWVRQAPGKGLEWVSSISGS | entire sequence |
| | GDYIYYADSVKGRFTISRDISKNTLYLQ | |
| | MNSLRAEDTAVYYCAKEGTGANSSLA | |
| | DYRGQGTLVTVSS | |
| 106 | GFTFSSYA | Anti-BCMA FHVH33 |
| | | CDR1 |
| 107 | ISGSGDYI | Anti-BCMA FHVH33 |
| | | CDR2 |
| 108 | AKEGTGANSSLADY | Anti-BCMA FHVH33 |
| | | CDR3 |
| 109 | DIQMTQSPSSLSASVGDRVTITCRASQSI | Anti-BCMA CT103A |
| | SSYLNWYQQKPGKAPKLLIYAASSLQS | scFv entire sequence, with |
| | GVPSRFSGSGSGTDFTLTISSLQPEDFAT | Whitlow linker |
| | YYCQQKYDLLTFGGGTKVEIKGSTSGS | |
| | GKPGSGEGSTKGQLQLQESGPGLVKPS | |
| | ETLSLTCTVSGGSISSSSYYWGWIRQPP | |
| | GKGLEWIGSISYSGSTYYNPSLKSRVTIS | |
| | VDTSKNQFSLKLSSVTAADTAVYYCAR | |
| | DRGDTILDVWGQGTMVTVSS | |
| 110 | DIQMTQSPSSLSASVGDRVTITCRASQSI | Anti-BCMA CT103A |
| | SSYLNWYQQKPGKAPKLLIYAASSLQS | scFv light chain variable |
| | GVPSRFSGSGSGTDFTLTISSLQPEDFAT | region |
| | YYCQQKYDLLTFGGGTKVEIK | |
| 111 | QSISSY | Anti-BCMA CT103A |
| | | scFv light chain CDR1 |
| 112 | AAS | Anti-BCMA CT103A |
| | | scFv light chain CDR2 |
| 113 | QQKYDLLT | Anti-BCMA CT103A |
| | | scFv light chain CDR3 |
| 114 | QLQLQESGPGLVKPSETLSLTCTVSGGS | Anti-BCMA CT103A |
| | ISSSSYYWGWIRQPPGKGLEWIGSISYS | scFv heavy chain variable |
| | GSTYYNPSLKSRVTISVDTSKNQFSLKL | region |
| | SSVTAADTAVYYCARDRGDTILDVWG | |
| | QGTMVTVSS | |
| 115 | GGSISSSSYY | Anti-BCMA CT103A |
| | | scFv heavy chain CDR1 |
| 116 | ISYSGST | Anti-BCMA CT103A |
| | | scFv heavy chain CDR2 |
| 117 | ARDRGDTILDV | Anti-BCMA CT103A |
| | | scFv heavy chain CDR3 |

In some embodiments, the hinge domain of the BCMA CAR comprises a CD8α hinge domain, for example, a human CD8α hinge domain. In some embodiments, the CD8α hinge domain comprises or consists of an amino acid sequence set forth in SEQ ID NO:88 or an amino acid sequence that is at least 80% identical (e.g., at least 80%, at least 85%, at least 90%, at least 95%, at least 96%, at least 97%, at least 98%, at least 99%, or 100% identical) to the amino acid sequence set forth in of SEQ ID NO:88. In some embodiments, the hinge domain comprises a CD28 hinge domain, for example, a human CD28 hinge domain. In some embodiments, the CD28 hinge domain comprises or consists of an amino acid sequence set forth in SEQ ID NO:89 or an amino acid sequence that is at least 80% identical (e.g., at least 80%, at least 85%, at least 90%, at least 95%, at least 96%, at least 97%, at least 98%, at least 99%, or 100% identical) to the amino acid sequence set forth in of SEQ ID NO:89. In some embodiments, the hinge domain comprises an IgG4 hinge domain, for example, a human IgG4 hinge domain. In some embodiments, the IgG4 hinge domain comprises or consists of an amino acid sequence set forth in SEQ ID NO:91 or SEQ ID NO:92, or an amino acid sequence that is at least 80% identical (e.g., at least 80%, at least 85%, at least 90%, at least 95%, at least 96%, at least 97%, at least 98%, at least 99%, or 100% identical) to the amino acid sequence set forth in of SEQ ID NO:91 or SEQ ID NO:92. In some embodiments, the hinge domain comprises a IgG4 hinge-Ch2-Ch3 domain, for example, a human IgG4 hinge-Ch2-Ch3 domain. In some embodiments, the IgG4 hinge-Ch2-Ch3 domain comprises or consists of an amino acid sequence set forth in SEQ ID NO:93 or an amino acid sequence that is at least 80% identical (e.g., at least 80%, at least 85%, at least 90%, at least 95%, at least 96%, at least 97%, at least 98%, at least 99%, or 100% identical) to the amino acid sequence set forth in of SEQ ID NO:93.

In some embodiments, the transmembrane domain of the BCMA CAR comprises a CD8α transmembrane domain, for example, a human CD8α transmembrane domain. In some embodiments, the CD8α transmembrane domain comprises or consists of an amino acid sequence set forth in SEQ ID NO:94 or an amino acid sequence that is at least 80% identical (e.g., at least 80%, at least 85%, at least 90%, at least 95%, at least 96%, at least 97%, at least 98%, at least 99%, or 100% identical) to the amino acid sequence set forth in SEQ ID NO:94. In some embodiments, the transmembrane domain comprises a CD28 transmembrane domain, for example, a human CD28 transmembrane domain. In some embodiments, the CD28 transmembrane domain comprises or consists of an amino acid sequence set forth in SEQ ID NO:95 or an amino acid sequence that is at least 80% identical (e.g., at least 80%, at least 85%, at least 90%, at least 95%, at least 96%, at least 97%, at least 98%, at least 99%, or 100% identical) to the amino acid sequence set forth in SEQ ID NO:95.

In some embodiments, the intracellular costimulatory domain of the BCMA CAR comprises a 4-1BB costimulatory domain, for example, a human 4-1BB costimulatory domain. In some embodiments, the 4-1BB costimulatory domain comprises or consists of an amino acid sequence set forth in SEQ ID NO:97 or an amino acid sequence that is at least 80% identical (e.g., at least 80%, at least 85%, at least 90%, at least 95%, at least 96%, at least 97%, at least 98%, at least 99%, or 100% identical) to the amino acid sequence set forth in SEQ ID NO:97. In some embodiments, the intracellular costimulatory domain comprises a CD28 costimulatory domain, for example, a human CD28 costimulatory domain. In some embodiments, the CD28 costimulatory domain comprises or consists of an amino acid sequence set forth in SEQ ID NO:98 or an amino acid sequence that is at least 80% identical (e.g., at least 80%, at least 85%, at least 90%, at least 95%, at least 96%, at least 97%, at least 98%, at least 99%, or 100% identical) to the amino acid sequence set forth in SEQ ID NO:98.

In some embodiments, the intracellular signaling domain of the BCMA CAR comprises a CD3 zeta (ζ) signaling domain, for example, a human CD3ζ signaling domain. In some embodiments, the CD3ζ signaling domain comprises or consists of an amino acid sequence set forth in SEQ ID NO:99 or an amino acid sequence that is at least 80% identical (e.g., at least 80%, at least 85%, at least 90%, at least 95%, at least 96%, at least 97%, at least 98%, at least 99%, or 100% identical) to the amino acid sequence set forth in SEQ ID NO:99.

In some embodiments, the polycistronic vector comprises an expression cassette that contains a nucleotide sequence encoding a BCMA CAR, including, for example, a BCMA CAR comprising any of the BCMA-specific extracellular binding domains as described, the CD8α hinge domain of SEQ ID NO:88, the CD8α transmembrane domain of SEQ ID NO:94, the 4-1BB costimulatory domain of SEQ ID NO:97, the CD3ζ signaling domain of SEQ ID NO:99, and/or variants (i.e., having a sequence that is at least 80% identical, for example, at least 80%, at least 85%, at least 90%, at least 95%, at least 96%, at least 97%, at least 98%, or at least 99 identical to the disclosed sequence) thereof. In any of these embodiments, the BCMA CAR may additionally comprise a signal peptide (e.g., a CD8α signal peptide) as described.

In some embodiments, the polycistronic vector comprises an expression cassette that contains a nucleotide sequence encoding a BCMA CAR, including, for example, a BCMA CAR comprising any of the BCMA-specific extracellular binding domains as described, the CD8α hinge domain of SEQ ID NO:88, the CD8α transmembrane domain of SEQ ID NO:94, the CD28 costimulatory domain of SEQ ID NO:98, the CD3ζ signaling domain of SEQ ID NO:99, and/or variants (i.e., having a sequence that is at least 80% identical, for example, at least 80%, at least 85%, at least 90%, at least 95%, at least 96%, at least 97%, at least 98%, or at least 99 identical to the disclosed sequence) thereof. In any of these embodiments, the BCMA CAR may additionally comprise a signal peptide as described.

In some embodiments, the polycistronic vector comprises an expression cassette that contains a nucleotide sequence encoding a BCMA CAR as set forth in SEQ ID NO:177 or is at least 80% identical (e.g., at least 80%, at least 85%, at least 90%, at least 95%, at least 96%, at least 97%, at least 98%, at least 99%, or 100% identical) to the nucleotide sequence set forth in SEQ ID NO:177 (see Table 15). The encoded BCMA CAR has a corresponding amino acid sequence set forth in SEQ ID NO: 178 or is at least 80% identical (e.g., at least 80%, at least 85%, at least 90%, at least 95%, at least 96%, at least 97%, at least 98%, at least 99%, or 100% identical) to the amino acid sequence set forth in of SEQ ID NO:178, with the following components: CD8α signal peptide, CT103A scFv (V_{L}—Whitlow linker-V_{H}), CD8α hinge domain, CD8α transmembrane domain, 4-1BB1 costimulatory domain, and CD3ζ signaling domain.

In some embodiments, the polycistronic vector comprises an expression cassette that contains a nucleotide sequence encoding a commercially available embodiment of BCMA CAR, including, for example, idecabtagene vicleucel (ide-cel, also called bb212I). In some embodiments, the polycistronic vector comprises an expression cassette that contains a nucleotide sequence encoding idecabtagene vicleucel or portions thereof. Idecabtagene vicleucel comprises a BCMA CAR with the following components: the BB2121 binder, CD8α hinge domain, CD8α transmembrane domain, 4-1BB1 costimulatory domain, and CD3ζ signaling domain.

TABLE 15
Exemplary sequences of BCMA CARs

| SEQ ID NO: | Sequence | Description |
|---|---|---|
| 118 | atggccttaccagtgaccgccttgctcctgccgctggccttgctgctcca | Exemplary BCMA |
| | cgccgccaggccggacatccagatgacccagtctccatcctccctgtct | CAR nucleotide |
| | gcatctgtaggagacagagtcaccatcacttgccgggcaagtcagagc | sequence |
| | attagcagctatttaaattggtatcagcagaaaccagggaaagcccctaa | |
| | gctcctgatctatgctgcatccagtttgcaaagtggggtcccatcaaggtt | |
| | cagtggcagtggatctgggacagatttcactctcaccatcagcagtctgc | |
| | aacctgaagattttgcaacttactactgtcagcaaaaatacgacctcctca | |
| | cttttggcggagggaccaaggttgagatcaaaggcagcaccagcggct | |
| | ccggcaagcctggctctggcgagggcagcacaaagggacagctgca | |
| | gctgcaggagtcgggcccaggactggtgaagccttcggagaccctgtc | |
| | cctcacctgcactgtctctggtggctccatcagcagtagtagttactactg | |
| | gggctggatccgccagcccccagggaaggggctggagtggattggg | |
| | agtatctcctatagtgggagcacctactacaacccgtccctcaagagtcg | |
| | agtcaccatatccgtagacacgtccaagaaccagttctccctgaagctga | |
| | gttctgtgaccgccgcagacacggcggtgtactactgcgccagagatc | |
| | gtggagacaccatactagacgtatggggtcagggtacaatggtcaccgt | |
| | cagctcattcgtgcccgtgttcctgcccgccaaacctaccaccacccctg | |
| | cccctagacctcccaccccagccccaacaatcgccagccagcctctgt | |
| | ctctgcggcccgaagcctgtagacctgctgccggcggagccgtgcaca | |
| | ccagaggcctggacttcgcctgcgacatctacatctgggcccctctggc | |
| | cggcacctgtggcgtgctgctgctgagcctggtgatcaccctgtactgc | |
| | aaccaccggaacaaacggggcagaaagaaactcctgtatatattcaaa | |
| | caaccatttatgagaccagtacaaactactcaagaggaagatggctgta | |
| | gctgccgatttccagaagaagaagaaggaggatgtgaactgagagtga | |
| | agttcagcagatccgccgacgcccctgcctaccagcagggacagaac | |
| | cagctgtacaacgagctgaacctgggcagacgggaagagtacgacgt | |
| | gctggacaagcggagaggccgggaccccgagatgggcggaaagcc | |
| | cagacggaagaacccccaggaaggcctgtataacgaactgcagaaag | |
| | acaagatggccgaggcctacagcgagatcggcatgaagggcgagcg | |
| | gaggcgcggcaagggccacgatggcctgtaccagggcctgagcacc | |
| | gccaccaaggacacctacgacgccctgcacatgcaggccctgccccc | |
| | caga | |
| 119 | MALPVTALLLPLALLLHAARPDIQMTQSPSSL | Exemplary BCMA |
| | SASVGDRVTITCRASQSISSYLNWYQQKPGKA | CAR amino acid |
| | PKLLIYAASSLQSGVPSRFSGSGSGTDFTLTISS | sequence |
| | LQPEDFATYYCQQKYDLLTFGGGTKVEIKGST | |
| | SGSGKPGSGEGSTKGQLQLQESGPGLVKPSET | |
| | LSLTCTVSGGSISSSSYYWGWIRQPPGKGLEWI | |
| | GSISYSGSTYYNPSLKSRVTISVDTSKNQFSLK | |
| | LSSVTAADTAVYYCARDRGDTILDVWGQGT | |
| | MVTVSSFVPVFLPAKPTTTPAPRPPTPAPTIAS | |
| | QPLSLRPEACRPAAGGAVHTRGLDFACDIYIW | |
| | APLAGTCGVLLLSLVITLYCNHRNKRGRKKLL | |
| | YIFKQPFMRPVQTTQEEDGCSCRFPEEEEGGC | |
| | ELRVKFSRSADAPAYQQGQNQLYNELNLGRR | |
| | EEYDVLDKRRGRDPEMGGKPRRKNPQEGLY | |
| | NELQKDKMAEAYSEIGMKGERRRGKGHDGL | |
| | YQGLSTATKDTYDALHMQALPPR | |

### 2. Gene Editing Enzymes

In some embodiments, the exogenous agent is or comprises a genome editing technology. In some embodiments, the exogenous agent is or comprises a heterologous protein that is associated with a genome editing technology. Any of a variety of agents associated with gene editing technologies can be included as the exogenous agent and/or heterologous protein, such as for delivery of gene editing machinery to a cell. In some embodiments, the gene editing technology can include systems involving nuclease, nickase, homing, integrase, transposase, recombinase, and/or reverse transcriptase activity. In some embodiments, the gene editing technologies can be used for knock-out or knock-down of genes. In some embodiments, the gene-editing technologies can be used for knock-in or integration of DNA into a region of the genome. In some embodiments, the exogenous agent and/or heterologous protein mediates single-strand breaks (SSB). In some embodiments, the exogenous agent and/or heterologous protein mediates double-strand breaks (DSB), including in connection with non-homologous end-joining (NHEJ) or homology-directed repair (HDR). In some embodiments, the exogenous agent and/or heterologous protein does not mediate SSB. In some embodiments, the exogenous agent and/or heterologous protein does not mediate DSB. In some embodiments, the exogenous agent and/or heterologous protein can be used for DNA base editing or prime-editing. In some embodiments, the exogenous agent and/or heterologous protein can be used for Programmable Addition via Site-specific Targeting Elements (PASTE).

In some embodiments, the exogenous agent is a nuclease for use in gene editing methods. In some embodiments, the nuclease is a zinc-finger nucleases (ZFNs), transcription-activator like effector nucleases (TALENs), or a CRISPR-associated protein-nuclease (Cas). In some embodiments, the Cas protein is selected from the group consisting of Cas3, Cas9, Cas10, Cas12, and Cas13. In some embodiments, the Cas is a Cas12a (also known as cpf1) from a Prevotella, Francisella novicida, Acidaminococcus sp., Lachnospiraceae bacterium, or Francisella bacteria. In some embodiments, the Cas is Cas9 from Streptococcus pyogenes. In some embodiments, the Cas is Cas9 from Streptococcus pyogenes (SpCas). In some embodiments, the Cas9 is from Staphylococcus aureus (SaCas9). In some embodiments, the Cas9 is from Neisseria meningitidis (NmeCas9). In some embodiments, the Cas9 is from Campylobacter jejuni (CjCas9). In some embodiments, the Cas9 is from Streptococcus thermophilis (StCas9). In some embodiments, the Cas is a Cas12a (also known as Cpf1) from a Prevotella or Francisella bacteria, or the Cas is a Cas12b from a Bacillus, optionally Bacillus hisashii. In some embodiments, the Cas is a Cas12a (also known as cpf1) from a Prevotella, Francisella novicida, Acidaminococcus sp., Lachnospiraceae bacterium, or Francisella bacteria. In some embodiments, the nuclease is MAD7 or CasX. In some of any embodiments, the Cas is a Cas3, Cas13, CasMini, or any other Cas protein known in the art. See for example, Wang et al., Biosensors and Bioelectronics (165) 1: 2020, and Wu et al. Nature Reviews Chemistry (4) 441: 2020). The Cas9 nuclease can, in some embodiments, be a Cas9 or functional fragment thereof from any bacterial species. See, e.g., Makarova et al. Nature Reviews, Microbiology, 9: 467-477 (2011), including supplemental information, hereby incorporated by reference in its entirety.

In some embodiments, delivery of the nuclease is by a provided vector encoding the nuclease (e.g. Cas).

In some embodiments, the provided viral vector particles contain a nuclease protein and the nuclease protein is directly delivered to a target cell. Methods of delivering a nuclease protein include those as described, for example, in Cai et al. Elife, 2014, 3:e01911 and International patent publication No. WO2017068077. For instance, provided viral vector particles comprise one or more Cas protein(s), such as Cas9. In some embodiments, the nuclease protein (e.g. Cas, such as Cas 9) is engineered as a chimeric nuclease protein with a viral structural protein (e.g. GAG) for packaging into the viral vector particle (e.g. lentiviral vector particle). For instance, a chimeric Cas9-protein fusion with the structural GAG protein can be packaged inside a lentiviral vector particle. In some embodiments, the fusion protein is a cleavable fusion protein between (i) a viral structural protein (e.g. GAG) and (ii) a nuclease protein (e.g. Cas protein, such as Cas 9).

In some embodiments, the Cas is wild-type Cas9, which can site-specifically cleave double-stranded DNA, resulting in the activation of the double-strand break (DSB) repair machinery. DSBs can be repaired by the cellular Non-Homologous End Joining (NHEJ) pathway (Overballe-Petersen et al., 2013, Proc Natl Acad Sci USA, Vol. 110: 19860-19865), resulting in insertions and/or deletions (indels) which disrupt the targeted locus. Alternatively, if a donor template with homology to the targeted locus is supplied, the DSB may be repaired by the homology-directed repair (HDR) pathway allowing for precise replacement mutations to be made (Overballe-Petersen et al., 2013, Proc Natl Acad Sci USA, Vol. 110: 19860-19865; Gong et al., 2005, Nat. Struct Mol Biol, Vol. 12: 304-312). In some embodiments, the Cas is mutant form, known as Cas9 D10A, with only nickase activity. This means that Cas9D10A cleaves only one DNA strand, and does not activate NHEJ. Instead, when provided with a homologous repair template, DNA repairs are conducted via the high-fidelity HDR pathway only, resulting in reduced indel mutations (Cong et al., 2013, Science, Vol. 339: 819-823; Jinek et al., 2012, Science, Vol. 337: 816-821; Qi et al., 2013 Cell, Vol. 152: 1173-1183). Cas9D10A is even more appealing in terms of target specificity when loci are targeted by paired Cas9 complexes designed to generate adjacent DNA nicks (Ran et al., 2013, Cell, Vol. 154: 1380-1389). In some embodiments, the Cas is a nuclease-deficient Cas9 (Qi et al., 2013 Cell, Vol. 152: 1173-1183). For instance, mutations H840A in the HNH domain and D10A in the RuvC domain inactivate cleavage activity, but do not prevent DNA binding. Therefore, this variant can be used to target in a sequence-specific manner any region of the genome without cleavage. Instead, by fusing with various effector domains, dCas9 can be used either as a gene silencing or activation tools. Furthermore, it can be used as a visualization tool by coupling the guide RNA or the Cas9 protein to a fluorophore or a fluorescent protein. In some embodiments, the Cas protein comprises one or more mutations such that the Cas protein is converted into a nickase that is able to cleave only one strand of a double stranded DNA molecule (e.g., a SSB). In some embodiments, the Cas protein is selected from the group consisting of Cas3, Cas4, Cas5, Cas8a, Cas8b, Cas8c, Cas9, Cas10, Cas12, Cas12a (Cpf1), Cas12b (C2cl), Cas12c (C2c3), Cas12d (CasY), Cas12e (CasX), Cas12f (C2c10), Cas12g, Cas12 h, Cas12i, Cas12k (C2c5), Cas13, Cas13a (C2c2), Cas13b, Cas13c, Cas13d, C2c4, C2c8, C2c9, Cmr5, Cse1, Cse2, Csf1, Csm2, Csn2, Csx10, Csx11, Csy1, Csy2, Csy3, and Mad7. In some embodiments, the Cas protein is Cas9. In some embodiments, the Cas9 is from a bacteria selected from the group consisting of Streptococcus pyogenes, Staphylococcus aureus, Neisseria meningitides, Campylobacter jejuni, and Streptococcus thermophilis. In some embodiments, the Cas9 is from Streptococcus pyogenes. In some embodiments, the Cas9 is from Streptococcus pyogenes and comprises one or more mutations in the RuvC I, RuvC II, or RuvC III motifs. In some embodiments, the Cas9 is from Streptococcus pyogenes and comprises a D10A mutation in the RuvC I motif. In some embodiments, the Cas9 is from Streptococcus pyogenes and comprises one or more mutations in the HNH catalytic domain. In some embodiments, the Cas9 is from Streptococcus pyogenes and comprises one or more mutations in the HNH catalytic domain selected from the group consisting of H840A, H854A, and H863A. In some embodiments, the Cas9 is from Streptococcus pyogenes and comprises a H840A mutation in the HNH catalytic domain. In some embodiments, the Cas9 is from Streptococcus pyogenes and comprises a mutation selected from the group consisting of DOA, H840A, H854A, and H863A.

In some embodiments, the Cas protein is selected from the group consisting of Cas3, Cas9, Cas10, Cas12, and Cas13. In particular embodiments, the nuclease is a Cas nuclease, such as Cas9. In some embodiments, delivery of the CRISPR/Cas can be used to introduce single point mutations (deletions or insertions) in a particular target gene, via a single gRNA. Using a pair of gRNA-directed Cas9 nucleases instead, it is also possible to induce large deletions or genomic rearrangements, such as inversions or translocations. In some embodiments, the one or more agent(s) (e.g., the heterologous protein) capable of inducing a DSB comprise Cas9 or a functional fragment thereof, and a first guide RNA, e.g., a first sgRNA, and a second guide RNA, e.g., a second sgRNA. The guide RNA, e.g., the first guide RNA or the second guide RNA, in some embodiments, binds to the recombinant nuclease and targets the recombinant nuclease to a specific location within the target gene such as at a location within the sense strand or the antisense strand of the target gene that is or includes the cleavage site. In some embodiments, the recombinant nuclease is a Cas protein from any bacterial species, or is a functional fragment thereof. In some embodiments, the Cas protein is Cas9 nuclease. Cas9 can, in some embodiments, be a Cas9 or functional fragment thereof from any bacterial species. See, e.g., Makarova et al. Nature Reviews, Microbiology, 9: 467-477 (2011), including supplemental information, hereby incorporated by reference in its entirety. In some embodiments, the Cas9 is from Streptococcus pyogenes (SpCas9). In some embodiments, the Cas9 is from Staphylococcus aureus (SaCas9). In some embodiments, the Cas9 is from Neisseria meningitidis (NmeCas9). In some embodiments, the Cas9 is from Campylobacter jejuni (CjCas9). In some embodiments, the Cas9 is from Streptococcus thermophilis (StCas9).

In some embodiments, the Cas9 is from Streptococcus pyogenes (SpCas9) and comprises one or more mutations in the RuvC catalytic domain or the HNH catalytic domain. In some embodiments, the one or more mutations in the RuvC catalytic domain or the HNH catalytic domain inactivates the catalytic activity of the domain. In some embodiments, the recombinant nuclease has RuvC activity but does not have HNH activity. In some embodiments, the recombinant nuclease does not have RuvC activity but does have HNH activity. In some embodiments, the Cas9 is from Streptococcus pyogenes (SpCas9) and comprises one or more mutations selected from the group consisting of D10A, H840A, H854A, and H863A. In some embodiments, the Cas9 is from Streptococcus pyogenes (SpCas9) and comprises one or more mutations in the RuvC I, RuvC II, or RuvC III motifs. In some embodiments, the Cas9 is from Streptococcus pyogenes (SpCas9) and comprises a mutation in the RuvC I motif. In some embodiments, the Cas9 is from Streptococcus pyogenes (SpCas9) and comprises a D10A mutation in the RuvC I motif. In some embodiments, the Cas9 is from Streptococcus pyogenes (SpCas9) and comprises one or more mutations in the HNH catalytic domain. In some embodiments, the one or more mutations in the HNH catalytic domain is selected from the group consisting of H840A, H854A, and H863A. In some embodiments, the Cas9 is from Streptococcus pyogenes (SpCas9) and comprises a H840A mutation in the HNH catalytic domain. In some embodiments, the Cas9 is from Streptococcus pyogenes (SpCas9) and comprises a H840A mutation. In some embodiments, the Cas9 is from Streptococcus pyogenes (SpCas9) and comprises a D10A mutation. In some embodiments, the Cas9 is from Streptococcus pyogenes (SpCas9) and comprises one or more mutations selected from the group consisting of N497A, R661A, Q695A, and Q926A. In some embodiments, the Cas9 is from Streptococcus pyogenes (SpCas9) and comprises one or more mutations selected from the group consisting of R780A, K810A, K855A, H982A, K1003A, R1060A, and K848A. In some embodiments, the Cas9 is from Streptococcus pyogenes (SpCas9) and comprises one or more mutations selected from the group consisting of N692A, M694A, Q695A, and H698A. In some embodiments, the Cas9 is from Streptococcus pyogenes (SpCas9) and comprises one or more mutations selected from the group consisting of M495V, Y515N, K526E, and R661Q. In some embodiments, the Cas9 is from Streptococcus pyogenes (SpCas9) and comprises one or more mutations selected from the group consisting of F539S, M763I, and K890N. In some embodiments, the Cas9 is from Streptococcus pyogenes (SpCas9) and comprises one or more mutations selected from the group consisting of E480K, E543D, E1219V, A262T, S4091, M6941, E108G, S217A.

In some embodiments, the Cas9 is from Streptococcus pyogenes (SaCas9). In some embodiments, the SaCas9 is wild type SaCas9. In some embodiments, the SaCas9 comprises one or more mutations in REC3 domain. In some embodiments, the SaCas9 comprises one or more mutations in REC1 domain. In some embodiments, the SaCas9 comprises one or more mutations selected from the group consisting of N260D, N260Q, N260E, Q414A, Q414L. In some embodiments, the SaCas9 comprises one or more mutations in the recognition lobe. In some embodiments, the SaCas9 comprises one or more mutations selected from the group consisting of R245A, N413A, N419A. In some embodiments, the SaCas9 comprises one or more mutations in the RuvC-III domain. In some embodiments, the SaCas9 comprises a R654A mutation.

In some embodiments, the Cas protein is Cas12. In some embodiments, the Cas protein is Cas12a (i.e. cpf1). In some embodiments, the Cas12a is from the group consisting of Francisella novicida U112 (FnCas12a), Acidaminococcus sp. BV3L6 (AsCas12a), Moraxella bovoculi AAX11_00205 (Mb3Cas12a), Lachnospiraceae bacterium ND2006 (LbCas12a), Thiomicrospira sp. Xs5 (TsCas12a), Moraxella bovoculi AAX08_00205 (Mb2Cas12a), and Butyrivibrio sp. NC3005 (BsCas12a). In some embodiments, the Cas12a recognizes a T-rich 5′ protospacer adjacent motif (PAM). In some embodiments, the Cas12a processes its own crRNA without requiring a transactivating crRNA (tracrRNA). In some embodiments, the Cas12a processes both RNase and DNase activity. In some embodiments, the Cas12a is a split Cas12a platform, consisting of N-terminal and C-terminal fragments of Cas12a. In some embodiments, the split Cas12a platform is from Lachnospiraceae bacterium.

In some embodiments, the lipid particle further comprises a polynucleotide per se, i.e. a polynucleotide that does not encode for a heterologous protein. In some embodiments, the polynucleotide per se is associated with a gene editing system. For example, a lipid particle may comprise a guide RNA (gRNA), such as a single guide RNA (sgRNA).

In some embodiments, the one or more agent(s) (e.g., one or more exogenous agent and/or heterologous protein) comprise, or are used in combination with, a guide RNA, e.g., single guide RNA (sgRNA), for inducing a DSB at the cleavage site. In some embodiments, the one or more agent(s) comprise, or are used in combination with, more than one guide RNA, e.g., a first sgRNA and a second sgRNA, for inducing a DSB at the cleavage site through a SSB on each strand. In some embodiments, the one or more agent(s) (e.g., the heterologous protein) can be used in combination with a donor template, e.g., a single-stranded DNA oligonucleotide (ssODN), for HDR-mediated integration of the donor template into the target gene, such as at the targeting sequence. In some embodiments, the one or more agent(s) (e.g., one or more exogenous agent and/or heterologous protein) can be used in combination with a donor template, e.g., an ssODN, and a guide RNA, e.g., a sgRNA, for HDR-mediated integration of the donor template into the target gene, such as at the targeting sequence. In some embodiments, the one or more agent(s) (e.g., one or more exogenous agent and/or heterologous protein) can be used in combination with a donor template, e.g., an ssODN, and a first guide RNA, e.g., a first sgRNA, and a second guide RNA, e.g., a second sgRNA, for HDR-mediated integration of the donor template into the target gene, such as at the targeting sequence.

In particular embodiments, the genome-modifying agent is a Cas protein, such as Cas9. In some embodiments, delivery of the CRISPR/Cas can be used to introduce single point mutations (deletions or insertions) in a particular target gene, via a single gRNA. Using a pair of gRNA-directed Cas9 nucleases instead, it is also possible to induce large deletions or genomic rearrangements, such as inversions or translocations. In some embodiments, a dCas9 version of the CRISPR/Cas9 system can be used to target protein domains for transcriptional regulation, epigenetic modification, and microscopic visualization of specific genome loci.

In some embodiments, the genome-modifying agent, e.g., Cas9, is targeted to the cleavage site by interacting with a guide RNA, e.g., sgRNA, that hybridizes to a DNA sequence that immediately precedes a Protospacer Adjacent Motif (PAM) sequence. In general, a guide RNA, e.g., sgRNA, is any nucleotide sequence comprising a sequence, e.g., a crRNA sequence, that has sufficient complementarity with a target gene sequence to hybridize with the target gene sequence at the cleavage site and direct sequence-specific binding of the recombinant nuclease to a portion of the target gene that includes the cleavage site. Full complementarity (100%) is not necessarily required, so long as there is sufficient complementarity to cause hybridization and promote formation of a complex, e.g., CRISPR complex, that includes the recombinant nuclease, e.g., Cas9, and the guide RNA, e.g., sgRNA. In some embodiments, the cleavage site is situated at a site within the target gene that is homologous to the sequence of the guide RNA, e.g., sgRNA. In some embodiments, the cleavage site is situated approximately 3 nucleotides upstream of the PAM sequence. In some embodiments, the cleavage site is situated approximately 3 nucleotides upstream of the juncture between the guide RNA and the PAM sequence. In some embodiments, the cleavage site is situated 3 nucleotides upstream of the PAM sequence. In some embodiments, the cleavage site is situated 4 nucleotides upstream of the PAM sequence.

In some embodiments, the one or more agent(s) (e.g., one or more exogenous agent and/or heterologous protein) capable of inducing a DSB comprise a fusion protein comprising a DNA binding domain and a DNA cleavage domain. In some embodiments, the DNA cleavage domain is or comprises a recombinant nuclease. In some embodiments, the fusion protein is a TALEN comprising a DNA binding domain and a DNA cleavage domain. In some embodiments, the DNA binding domain is a transcription activator-like (TAL) effector DNA binding domain. In some embodiments, the TAL effector DNA binding domain is from Xanthomonas bacteria. In some embodiments, the DNA cleavage domain is a Fokl nuclease domain. In some embodiments, the TAL effector DNA binding domain is engineered to target a specific target sequence, e.g., a portion of a target gene that includes a cleavage site.

In some embodiments, the fusion protein is a zinc finger nuclease (ZFN) comprising a zinc finger DNA binding domain and a DNA cleavage domain. In some embodiments, the DNA cleavage domain is a Fokl nuclease domain. In some embodiments, the zinc finger DNA binding domain is engineered to target a specific target sequence, e.g., a portion of a target gene, that includes a cleavage site, such as the targeting sequence.

In some embodiments, the provided lipid particles can be for use in a method to deliver an exogenous agent which involves introducing, into a cell, one or more agent(s) (e.g., one or more exogenous agent and/or heterologous protein) capable of inducing a SSB at a cleavage site within the sense strand and a SSB at a cleavage site within the antisense strand of an endogenous target gene in the cell.

In some embodiments, the cleavage site in the sense strand is less than 400, less than 350, less than 300, less than 250, less than 200, less than 175, less than 150, less than 125, less than 100, less than 90, less than 80, less than 75, less than 70, less than 65, less than 60, less than 55, less than 50, less than 45, less than 40, or less than 35 nucleotides from the nucleotide that is complementary to the cleavage site in the antisense strand. In some embodiments, the cleavage site in the antisense strand is less than 400, less than 350, less than 300, less than 250, less than 200, less than 175, less than 150, less than 125, less than 100, less than 90, less than 80, less than 75, less than 70, less than 65, less than 60, less than 55, less than 50, less than 45, less than 40, or less than 35 nucleotides from the nucleotide that is complementary to the cleavage site in the sense strand. In some embodiments, the cleavage site in the sense strand is between 20 and 400, 20 and 350, 20 and 300, 20 and 250, 20 and 200, 20 and 150, 20 and 125, 20 and 100, 20 and 90, 20 and 80, 20 and 70, 30 and 400, 30 and 350, 30 and 300, 30 and 250, 30 and 200, 30 and 150, 30 and 125, 30 and 100, 30 and 90, 30 and 80, 30 and 70, 40 and 400, 40 and 350, 40 and 300, 40 and 250, 40 and 200, 40 and 150, 40 and 125, 40 and 100, 40 and 90, 40 and 80, or 40 and 70 nucleotides from the nucleotide that is complementary to the cleavage site in the antisense strand. In some embodiments, the cleavage site in the antisense strand is between 20 and 400, 20 and 350, 20 and 300, 20 and 250, 20 and 200, 20 and 150, 20 and 125, 20 and 100, 20 and 90, 20 and 80, 20 and 70, 30 and 400, 30 and 350, 30 and 300, 30 and 250, 30 and 200, 30 and 150, 30 and 125, 30 and 100, 30 and 90, 30 and 80, 30 and 70, 40 and 400, 40 and 350, 40 and 300, 40 and 250, 40 and 200, 40 and 150, 40 and 125, 40 and 100, 40 and 90, 40 and 80, or 40 and 70 nucleotides from the nucleotide that is complementary to the cleavage site in the sense strand.

In some embodiments, the one or more agent(s) (e.g., one or more exogenous agent and/or heterologous protein) capable of inducing a SSB at a cleavage site within the sense strand and a SSB at a cleavage site within the antisense strand comprise a recombinant nuclease. In some embodiments, the recombinant nuclease includes a recombinant nuclease that induces the SSB in the sense strand, and a recombinant nuclease that induced the SSB in the antisense strand, and both of which recombinant nucleases are referred to as the recombinant nuclease. Accordingly, in some embodiments, the method involves introducing, into a cell, one or more agent(s) (e.g., the one or more exogenous agent and/or heterologous protein) comprising a recombinant nuclease for inducing a SSB at a cleavage site in the sense strand and a SSB at a cleavage site in the antisense strand within an endogenous target gene in the cell. Although, in some embodiments, it is described that “a” “the” recombinant nuclease induces a SSB in the antisense strand a SSB in the sense strand, it is to be understood that this includes situations where two of the same recombinant nuclease is used, such that one of the recombinant nuclease induces the SSB in the sense strand and the other recombinant nuclease induces the SSB in the antisense strand. In some embodiments, the recombinant nuclease that induces the SSB lacks the ability to induce a DSB by cleaving both strands of double stranded DNA.

In some embodiments, the one or more agent(s) capable of inducing a SSB comprise a recombinant nuclease and a first guide RNA, e.g., a first sgRNA, and a second guide RNA, e.g., a second sgRNA.

In some embodiments, the genome-modifying agent is a Cas protein, a transcription activator-like effector nuclease (TALEN), or a zinc finger nuclease (ZFN). In some embodiments, the recombinant nuclease is a Cas nuclease. In some embodiments, the recombinant nuclease is a TALEN. In some embodiments, the recombinant nuclease is a ZFN.

In some embodiments, the one or more agent(s) capable of inducing a SSB at a cleavage site within the sense strand and a SSB at a cleavage site within the antisense strand comprise a fusion protein comprising a DNA binding domain and a DNA cleavage domain. In some embodiments, the DNA cleavage domain is or comprises a recombinant nuclease. In some embodiments, the fusion protein is a TALEN comprising a DNA binding domain and a DNA cleavage domain. In some embodiments, the DNA binding domain is a transcription activator-like (TAL) effector DNA binding domain. In some embodiments, the TAL effector DNA binding domain is from Xanthomonas bacteria. In some embodiments, the DNA cleavage domain is a Fokl nuclease domain. In some embodiments, the TAL effector DNA binding domain is engineered to target a specific target sequence, e.g., a portion of a target gene that includes a cleavage site. In some embodiments, the fusion protein is a zinc finger nuclease (ZFN) comprising a zinc finger DNA binding domain and a DNA cleavage domain. In some embodiments, the DNA cleavage domain is a Fokl nuclease domain. In some embodiments, the zinc finger DNA binding domain is engineered to target a specific target sequence, e.g., a portion of a target gene that includes a cleavage site, such as the targeting sequence.

In some embodiments, the one or more agent(s) capable of inducing a SSB at a cleavage site within the sense strand and a SSB at a cleavage site within the antisense strand involve use of the CRISPR/Cas gene editing system. In some embodiments, the one or more agent(s) comprise a recombinant nuclease.

In some embodiments, the genome-modifying agent is a Cas protein. In some embodiments, the Cas protein comprises one or more mutations such that the Cas protein is converted into a nickase that lacks the ability to cleave both strands of a double stranded DNA molecule. In some embodiments, the Cas protein comprises one or more mutations such that the Cas protein is converted into a nickase that is able to cleave only one strand of a double stranded DNA molecule. For example, Cas9, which is normally capable of inducing a double strand break, can be converted into a Cas9 nickase, which is capable of inducing a single strand break, by mutating one of two Cas9 catalytic domains: the RuvC domain, which comprises the RuvC I, RuvC II, and RuvC III motifs, or the NHN domain. In some embodiments, the Cas protein comprises one or more mutations in the RuvC catalytic domain or the HNH catalytic domain. In some embodiments, the genome-modifying protein is a recombinant nuclease that has been modified to have nickase activity. In some embodiments, the recombinant nuclease cleaves the strand to which the guide RNA, e.g., sgRNA, hybridizes, but does not cleave the strand that is complementary to the strand to which the guide RNA, e.g., sgRNA, hybridizes. In some embodiments, the recombinant nuclease does not cleave the strand to which the guide RNA, e.g., sgRNA, hybridizes, but does cleave the strand that is complementary to the strand to which the guide RNA, e.g., sgRNA, hybridizes.

In some embodiments, the lipid particle further comprises a guide RNA (gRNA), such as a single guide RNA (sgRNA). Thus, in some embodiments, the heterologous agent comprises a guide RNA (gRNA). In some embodiments, the gRNA is a single guide RNA (sgRNA).

In some embodiments, the genome-modifying protein, e.g., Cas9, is targeted to the cleavage site by interacting with a guide RNA, e.g., a first guide RNA, such as a first sgRNA, or a second guide RNA, such as a second sgRNA, that hybridizes to a DNA sequence on the sense strand or the antisense strand that immediately precedes a Protospacer Adjacent Motif (PAM) sequence.

In some embodiments, the genome-modifying agent, e.g., Cas9, is targeted to the cleavage site on the sense strand by interacting with a first guide RNA, e.g., first sgRNA, that hybridizes to a sequence on the sense strand that immediately precedes a PAM sequence. In some embodiments, the genome-modifying agent, e.g., Cas9, is targeted to the cleavage site on the antisense strand by interacting with a second guide RNA, e.g., second sgRNA, that hybridizes to a sequence on the antisense strand that immediately precedes a PAM sequence.

In some embodiments, the first guide RNA, e.g., first sgNA, that is specific to the sense strand of a target gene of interest is used to target the recombinant nuclease, e.g., Cas9, to induce a SSB at a cleavage site within the sense strand of the target gene. In some embodiments, the first guide RNA, e.g., first sgNA, that is specific to the antisense strand of a target gene of interest is used to target the recombinant nuclease, e.g., Cas9, to induce a SSB at a cleavage site within the antisense strand of the target gene.

In some embodiments, the second guide RNA, e.g., second sgNA, that is specific to the sense strand of a target gene of interest used to target the recombinant nuclease, e.g., Cas9, to induce a SSB at a cleavage site within the sense strand of the target gene. In some embodiments, the second guide RNA, e.g., second sgNA, that is specific to the antisense strand of a target gene of interest is used to target the recombinant nuclease, e.g., Cas9, to induce a SSB at a cleavage site within the antisense strand of the target gene.

In some embodiments, the first guide RNA, e.g., first sgNA, that is specific to the sense strand of a target gene of interest is used to target the recombinant nuclease, e.g., Cas9, to induce a SSB at a cleavage site within the sense strand of the target gene; and the second guide RNA, e.g., second sgNA, that is specific to the antisense strand of a target gene of interest is used to target the recombinant nuclease, e.g., Cas9, to induce a SSB at a cleavage site within the antisense strand of the target gene.

In some embodiments, the first guide RNA, e.g., first sgNA, that is specific to the antisense strand of a target gene of interest is used to target the recombinant nuclease, e.g., Cas9, to induce a SSB at a cleavage site within the antisense strand of the target gene; and the second guide RNA, e.g., second sgNA, that is specific to the sense strand of a target gene of interest is used to target the recombinant nuclease, e.g., Cas9, to induce a SSB at a cleavage site within the sense strand of the target gene. In general, a guide RNA, e.g., a first guide RNA, such as a first sgRNA, or a second guide RNA, such as a second sgRNA, is any nucleotide sequence comprising a sequence, e.g., a crRNA sequence, that has sufficient complementarity with a target gene sequence to hybridize with the target gene sequence at the cleavage site and direct sequence-specific binding of the recombinant nuclease to a portion of the target gene that includes the cleavage site. Full complementarity (100%) is not necessarily required, so long as there is sufficient complementarity to cause hybridization and promote formation of a complex, e.g., CRISPR complex, that includes the recombinant nuclease, e.g., Cas9, and the guide RNA, e.g., the first guide RNA, such as the first sgRNA, or the second guide RNA, such as the second sgRNA.

In some embodiments, the cleavage site is situated at a site within the target gene that is homologous to a sequence comprised within the guide RNA, e.g., sgRNA. In some embodiments, the cleavage site of the sense strand is situated at a site within the sense strand of the target gene that is homologous to a sequence comprised within the first guide RNA, e.g., the first sgRNA. In some embodiments, the cleavage site of the antisense strand is situated at a site within the antisense strand of the target gene that is homologous to a sequence comprised within the first guide RNA, e.g., the first sgRNA. In some embodiments, the cleavage site of the sense strand is situated at a site within the sense strand of the target gene that is homologous to a sequence comprised within the second guide RNA, e.g., the second sgRNA. In some embodiments, the cleavage site of the antisense strand is situated at a site within the antisense strand of the target gene that is homologous to a sequence comprised within the second guide RNA, e.g., the second sgRNA. In some embodiments, the cleavage site of the sense strand is situated at a site within the sense strand of the target gene that is homologous to a sequence comprised within the first guide RNA, e.g., the first sgRNA; and the cleavage site of the antisense strand is situated at a site within the antisense strand of the target gene that is homologous to a sequence comprised within the second guide RNA, e.g., the second sgRNA. In some embodiments, the cleavage site of the antisense strand is situated at a site within the antisense strand of the target gene that is homologous to a sequence comprised within the first guide RNA, e.g., the first sgRNA; and the cleavage site of the sense strand is situated at a site within the sense strand of the target gene that is homologous to a sequence comprised within the second guide RNA, e.g., the second sgRNA. In some embodiments, the cleavage site of the antisense strand is situated at a site within the antisense strand of the target gene that is homologous to a sequence comprised within the second guide RNA, e.g., the second sgRNA; and the cleavage site of the sense strand is situated at a site within the sense strand of the target gene that is homologous to a sequence comprised within the first guide RNA, e.g., the first sgRNA.

In some embodiments, the sense strand comprises the targeting sequence, and the targeting sequence includes the SNP and a protospacer adjacent motif (PAM) sequence. In some embodiments, the sense strand comprises the targeting sequence, and the targeting sequence includes the SNP and a protospacer adjacent motif (PAM) sequence; and the antisense strand comprises a sequence that is complementary to the targeting sequence and includes a PAM sequence. In some embodiments, the antisense strand comprises the targeting sequence, and the targeting sequence includes the SNP and a protospacer adjacent motif (PAM) sequence. In some embodiments, the antisense strand comprises the targeting sequence, and the targeting sequence includes the SNP and a protospacer adjacent motif (PAM) sequence; and the sense strand comprises a sequence that is complementary to the targeting sequence and includes a PAM sequence.

In some embodiments, the cleavage site on the sense strand and/or the antisense strand is situated approximately 3 nucleotides upstream of the PAM sequence. In some embodiments, the cleavage site on the sense strand and/or the antisense strand is situated approximately 3 nucleotides upstream of the juncture between the guide RNA and the PAM sequence. In some embodiments, the cleavage site on the sense strand and/or the antisense strand is situated 3 nucleotides upstream of the PAM sequence. In some embodiments, the cleavage site on the sense strand and/or the antisense strand is situated 4 nucleotides upstream of the PAM sequence.

In some embodiments, the PAM sequence that is recognized by a recombinant nuclease is in the sense strand. In some embodiments, the PAM sequence that is recognized by a recombinant nuclease is in the antisense strand. In some embodiments, the PAM sequence that is recognized by a recombinant nuclease is in the sense strand and is in the antisense strand. In some embodiments, the PAM sequence on the sense strand and the PAM sequence on the antisense strand are outwardly facing. In some embodiments, the PAM sequence on the sense strand and the PAM sequence on the antisense strand comprise the same nucleic acid sequence, which can be any PAM sequence disclosed herein. In some embodiments, the PAM sequence on the sense strand and the PAM sequence on the antisense strand each comprise a different nucleic acid sequence, each of which can be any of the PAM sequences disclosed herein.

In some embodiments, the PAM sequence that is recognized by a recombinant nuclease, e.g., Cas9, differs depending on the particular recombinant nuclease and the bacterial species it is from

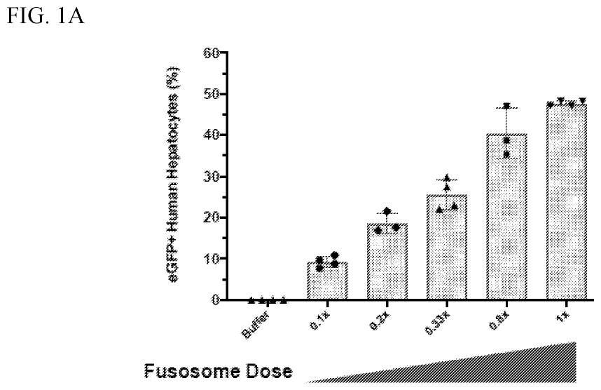

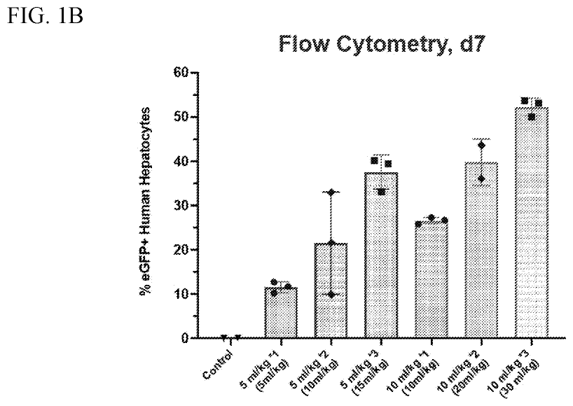

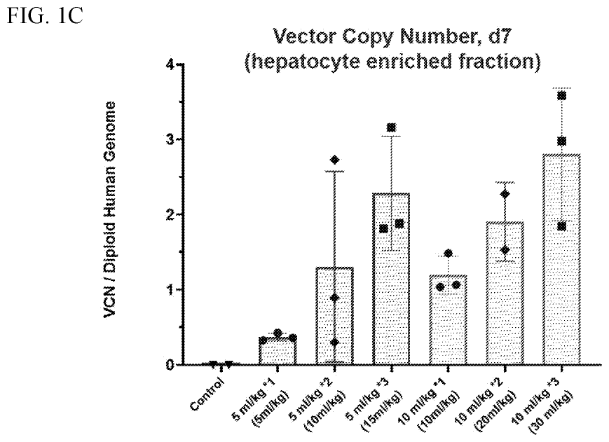

Methods for designing guide RNAs, e.g., sgRNAs, and their exemplary targeting sequences, e.g., crRNA sequences, can include those described in, e.g., International PCT Pub. Nos. WO2015/161276, WO2017/193107, and WO2017/093969. Exemplary guide RNA structures, including particular domains, are described in WO2015/161276, e.g., in FIGS. 1A-1G therein. Since guide RNA is an RNA molecule, it will comprise the base uracil (U), while any DNA encoding the guide RNA molecule will comprise the base thymine (T). In some embodiments, the guide RNA, e.g., sgRNA, comprises a CRISPR targeting RNA sequence (crRNA) and a trans-activating crRNA sequence (tracrRNA). In some embodiments, the first guide RNA, e.g., the first sgRNA, and the second guide RNA, e.g., the second sgRNA, each comprise a crRNA and a tracrRNA. In some embodiments, the guide RNA, e.g., sgRNA, is an RNA comprising, from 5′ to 3′: a crRNA sequence and a tracrRNA sequence. In some embodiments, each of the first guide RNA, e.g., first sgRNA, and the second guide RNA, e.g., second sgRNA, is an RNA comprising, from 5′ to 3′: a crRNA sequence and a tracrRNA sequence. In some embodiments, the crRNA and tracrRNA do not naturally occur together in the same sequence.

In some embodiments, the crRNA comprises a nucleotide sequence that is homologous, e.g., is at least 80%, 85%, 90%, 95%, 96%, 97%, 98%, or 99% homologous, or is 100% homologous, to a portion of the target gene that includes the cleavage site. In some embodiments, the crRNA comprises a nucleotide sequence that is 100% homologous to a portion of the target gene that includes the cleavage site. In some embodiments, the portion of the target gene that includes the cleavage site is a portion of the sense strand of the target gene that includes the cleavage site. In some embodiments, the portion of the target gene that includes the cleavage site is a portion of the antisense strand of the target gene that includes the cleavage site.

In some embodiments, the sgRNA comprises a crRNA sequence that is homologous to a sequence in the target gene that includes the cleavage site. In some embodiments, the first sgRNA comprises a crRNA sequence that is homologous to a sequence in the sense strand of the target gene that includes the cleavage site; and/or the second sgRNA comprises a crRNA sequence that is homologous to a sequence in the antisense strand of the target gene that includes the cleavage site. In some embodiments, the first sgRNA comprises a crRNA sequence that is homologous to a sequence in the antisense strand of the target gene that includes the cleavage site; and/or the second sgRNA comprises a crRNA sequence that is homologous to a sequence in the sense strand of the target gene that includes the cleavage site.

In some embodiments, the crRNA sequence has 100% sequence identity to a sequence in the target gene that includes the cleavage site. In some embodiments, the crRNA sequence of the first sgRNA has 100% sequence identity to a sequence in the sense strand of the target gene that includes the cleavage site; and/or the crRNA sequence of the second sgRNA has 100% sequence identity to a sequence in the antisense strand of the target gene that includes the cleavage site. In some embodiments, the crRNA sequence of the first sgRNA has 100% sequence identity to a sequence in the antisense strand of the target gene that includes the cleavage site; and/or the crRNA sequence of the second sgRNA has 100% sequence identity to a sequence in the sense strand of the target gene that includes the cleavage site.

Guidance on the selection of crRNA sequences can be found, e.g., in Fu Y et al., Nat Biotechnol 2014 (doi: 10.1038/nbt.2808) and Sternberg S H et al., Nature 2014 (doi: 10.1038/naturel3011). Examples of the placement of crRNA sequences within the guide RNA, e.g., sgRNA, structure include those described in WO2015/161276, e.g., in FIGS. 1A-1G therein.

Reference to “the crRNA” is to be understood as also including reference to the crRNA of the first sgRNA and the crRNA of the second sgRNA, each independently. Thus, embodiments referring to “the crRNA” is to be understood as independently referring to embodiments of (i) the crRNA, (ii) the crRNA of the first sgRNA, and (iii) the crRNA of the second sgRNA. In some embodiments, the crRNA is 15-27 nucleotides in length, i.e., the crRNA is 15, 16, 17, 18, 19, 20, 21, 22, 23, 24, 25, 26, or 27 nucleotides in length. In some embodiments, the crRNA is 18-22 nucleotides in length. In some embodiments, the crRNA is 19-21 nucleotides in length. In some embodiments, the crRNA is 20 nucleotides in length.

In some embodiments, the crRNA is homologous to a portion of a target gene that includes the cleavage site. In some embodiments, the crRNA is homologous to a portion of the sense strand of the target gene that includes the cleavage site. In some embodiments, the crRNA is homologous to a portion of the antisense strand of the target gene that includes the cleavage site. In some embodiments, the crRNA of the first sgRNA is homologous to a portion of the sense strand of the target gene that includes the cleavage site; and the crRNA of the second sgRNA is homologous to a portion of the antisense strand of the target gene that includes the cleavage site.

In some embodiments, the crRNA is homologous to a portion of the antisense strand of a target gene that includes the cleavage site. In some embodiments, the crRNA is homologous to a portion of the sense strand of the target gene that includes the cleavage site. In some embodiments, the crRNA of the first sgRNA is homologous to a portion of the antisense strand of the target gene that includes the cleavage site; and the crRNA of the second sgRNA is homologous to a portion of the sense strand of the target gene that includes the cleavage site.

In some embodiments, the crRNA is homologous to a portion of a target gene that includes the cleavage site, and is 15-27 nucleotides in length, i.e., the crRNA is 15, 16, 17, 18, 19, 20, 21, 22, 23, 24, 25, 26, or 27 nucleotides in length. In some embodiments, the portion of the target gene that includes the cleavage site is on the sense strand. In some embodiments, the portion of the target gene that includes the cleavage site is on the antisense strand.

In some embodiments, the crRNA is homologous to a portion, i.e., sequence, in the sense strand or the antisense strand of the target gene that includes the cleavage site and is immediately upstream of the PAM sequence.

In some embodiments, the tracrRNA sequence may be or comprise any sequence for tracrRNA that is used in any CRISPR/Cas9 system known in the art. Reference to “the tracrRNA” is to be understood as also including reference to the tracrRNA of the first sgRNA and the tracrRNA of the second sgRNA, each independently. Thus, embodiments referring to “the tracrRNA” is to be understood as independently referring to embodiments of (i) the tracrRNA, (ii) the tracrRNA of the first sgRNA, and (iii) the tracrRNA of the second sgRNA. Exemplary CRISPR/Cas9 systems, sgRNA, crRNA, and tracrRNA, and their manufacturing process and use include those described in, e.g., International PCT Pub. Nos. WO2015/161276, WO2017/193107 and WO2017/093969, and those described in, e.g., U.S. Patent Application Publication Nos. 20150232882, 20150203872, 20150184139, 20150079681, 20150073041, 20150056705, 20150031134, 20150020223, 20140357530, 20140335620, 20140310830, 20140273234, 20140273232, 20140273231, 20140256046, 20140248702, 20140242700, 20140242699, 20140242664, 20140234972, 20140227787, 20140189896, 20140186958, 20140186919, 20140186843, 20140179770, 20140179006, 20140170753, 20140093913, and 20140080216.

In some embodiments, the heterologous protein is associated with base editing. Base editors (BEs) are typically fusions of a Cas (“CRISPR-associated”) domain and a nucleobase modification domain (e.g., a natural or evolved deaminase, such as a cytidine deaminase that include APOBEC1 (“apolipoprotein B mRNA editing enzyme, catalytic polypeptide 1”), CDA (“cytidine deaminase”), and AID (“activation-induced cytidine deaminase”)) domains. In some cases, base editors may also include proteins or domains that alter cellular DNA repair processes to increase the efficiency and/or stability of the resulting single-nucleotide change.

In some aspects, currently available base editors include cytidine base editors (e.g., BE4) that convert target C·G to T·A and adenine base editors (e.g., ABE7.10) that convert target A·T to G·C. In some aspects, Cas9-targeted deamination was first demonstrated in connection with a Base Editor (BE) system designed to induce base changes without introducing double-strand DNA breaks. Further Rat deaminase APOBEC1 (rAPOBEC1) fused to deactivated Cas9 (dCas9) was used to successfully convert cytidines to thymidines upstream of the PAM of the sgRNA. In some aspects, this first BE system was optimized by changing the dCas9 to a “nickase” Cas9 D10A, which nicks the strand opposite the deaminated cytidine. Without being bound by theory, this is expected to initiate long-patch base excision repair (BER), where the deaminated strand is preferentially used to template the repair to produce a U:A base pair, which is then converted to T:A during DNA replication.

In some embodiments, the exogenous agent and/or heterologous protein is or encodes a base editor (e.g., a nucleobase editor). In some embodiments, the exogenous agent and/or heterologous protein is a nucleobase editor containing a first DNA binding protein domain that is catalytically inactive, a domain having base editing activity, and a second DNA binding protein domain having nickase activity, where the DNA binding protein domains are expressed on a single fusion protein or are expressed separately (e.g., on separate expression vectors). In some embodiments, the base editor is a fusion protein comprising a domain having base editing activity (e.g., cytidine deaminase or adenosine deaminase), and two nucleic acid programmable DNA binding protein domains (napDNAbp), a first comprising nickase activity and a second napDNAbp that is catalytically inactive, wherein at least the two napDNAbp are joined by a linker. In some embodiments, the base editor is a fusion protein that comprises a DNA domain of a CRISPR-Cas (e.g., Cas9) having nickase activity (nCas; nCas9), a catalytically inactive domain of a CRISPR-Cas protein (e.g., Cas9) having nucleic acid programmable DNA binding activity (dCas; e.g., dCas9), and a deaminase domain, wherein the dCas is joined to the nCas by a linker, and the dCas is immediately adjacent to the deaminase domain. In some embodiments, the base editor is a adenine-to-thymine or “ATBE” (or thymine-to-adenine or “TABE”) transversion base editors. Exemplary base editor and base editor systems include any as described in patent publication Nos. US20220127622, US20210079366, US20200248169, US20210093667, US20210071163, WO2020181202, WO2021158921, WO2019126709, WO2020181178, WO2020181195, WO2020214842, WO2020181193, which are hereby incorporated in their entirety.

In some embodiments, the exogenous agent and/or heterologous protein is one for use in target-primed reverse transcription (TPRT) or “prime editing”. In some embodiments, prime editing mediates targeted insertions, deletions, all 12 possible base-to-base conversions, and combinations thereof in human cells without requiring DSBs or donor DNA templates.

Prime editing is a genome editing method that directly writes new genetic information into a specified DNA site using a nucleic acid programmable DNA binding protein (“napDNAbp”) working in association with a polymerase (i.e., in the form of a fusion protein or otherwise provided in trans with the napDNAbp), wherein the prime editing system is programmed with a prime editing (PE) guide RNA (“PEgRNA”) that both specifies the target site and templates the synthesis of the desired edit in the form of a replacement DNA strand by way of an extension (either DNA or RNA) engineered onto a guide RNA (e.g., at the 5′ or 3′ end, or at an internal portion of a guide RNA). The replacement strand containing the desired edit (e.g., a single nucleobase substitution) shares the same sequence as the endogenous strand of the target site to be edited (with the exception that it includes the desired edit). Through DNA repair and/or replication machinery, the endogenous strand of the target site is replaced by the newly synthesized replacement strand containing the desired edit. In some cases, prime editing may be thought of as a “search-and-replace” genome editing technology since the prime editors search and locate the desired target site to be edited, and encode a replacement strand containing a desired edit which is installed in place of the corresponding target site endogenous DNA strand at the same time. For example, prime editing can be adapted for conducting precision CRISPR/Cas-based genome editing in order to bypass double stranded breaks. In some embodiments, the heterologous protein is or encodes for a Cas protein-reverse transcriptase fusions or related systems to target a specific DNA sequence with a guide RNA, generate a single strand nick at the target site, and use the nicked DNA as a primer for reverse transcription of an engineered reverse transcriptase template that is integrated with the guide RNA. In some embodiments, the prime editor protein is paired with two prime editing guide RNAs (pegRNAs) that template the synthesis of complementary DNA flaps on opposing strands of genomic DNA, resulting in the replacement of endogenous DNA sequence between the PE-induced nick sites with pegRNA-encoded sequences.

In some embodiments, the exogenous agent and/or heterologous protein is or encodes for a primer editor that is a reverse transcriptase, or any DNA polymerase known in the art. Thus, in one aspect, the prime editor may comprise Cas9 (or an equivalent napDNAbp) which is programmed to target a DNA sequence by associating it with a specialized guide RNA (i.e., PEgRNA) containing a spacer sequence that anneals to a complementary protospacer in the target DNA. Such methods include any disclosed in Anzalone et al., (doi.org/10.1038/s41586-019-1711-4), or in PCT publication Nos. WO2020191248, WO2021226558, or WO2022067130, which are hereby incorporated in their entirety.

In some embodiments, the exogenous agent and/or heterologous protein is for use in Programmable Addition via Site-specific Targeting Elements (PASTE). In some aspects, PASTE is platform in which genomic insertion is directed via a CRISPR-Cas9 nickase fused to both a reverse transcriptase and serine integrase. As described in Ioannidi et al. (doi.org/10.1101/2021.11.01.466786), PASTE does not generate double stranded breaks, but allowed for integration of sequences as large as ˜36 kb. In some embodiments, the serine integrase can be any known in the art. In some embodiments, the serine integrase has sufficient orthogonality such that PASTE can be used for multiplexed gene integration, simultaneously integrating at least two different genes at least two genomic loci. In some embodiments, PASTE has editing efficiencies comparable to or better than those of homology directed repair or non-homologous end joining based integration, with activity in nondividing cells and fewer detectable off-target events.

In some embodiments, the exogenous agent and/or heterologous protein is or encodes one or more polypeptides having an activity selected from the group consisting of: nuclease activity (e.g., programmable nuclease activity); nickase activity (e.g., programmable nickase activity); homing activity (e.g., programmable DNA binding activity); nucleic acid polymerase activity (e.g., DNA polymerase or RNA polymerase activity); integrase activity; recombinase activity; or base editing activity (e.g., cytidine deaminase or adenosine deaminase activity).

In some embodiments, delivery of the nuclease is by a provided vector encoding the nuclease (e.g. Cas).

In some embodiments, the provided lipid particles contain a nuclease protein and the nuclease protein is directly delivered to a target cell. Methods of delivering a nuclease protein include those as described, for example, in Cai et al. Elife, 2014, 3:e01911 and International patent publication No. WO2017068077. For instance, provided lipid particles comprise one or more Cas protein(s), such as Cas9. In some embodiments, the nuclease protein (e.g. Cas, such as Cas 9) is engineered as a chimeric nuclease protein with a viral structural protein (e.g. GAG) for packaging into the lipid particle (e.g. lentiviral vector particle, VLP, or gesicle). For instance, a chimeric Cas9-protein fusion with the structural GAG protein can be packaged inside a lipid particle. In some embodiments, the fusion protein is a cleavable fusion protein between (i) a viral structural protein (e.g. GAG) and (ii) a nuclease protein (e.g. Cas protein, such as Cas9). In some embodiments, the fusion protein is a cleavable fusion protein between (i) a viral matrix (MA) protein and (ii) a nuclease protein (e.g. Cas protein, such as Cas9). In some embodiments, the particle contains a nuclease protein (e.g., Cas protein, such as Cas 9) immediately downstream of the gag start codon.

In some embodiments, the provided lipid particles contain mRNA encoding a Cas nuclease (e.g., Cas9). In some embodiments, the provided lipid particles contain guide RNA (gRNA), such as a single guide RNA (sgRNA).

In some embodiments, a dCas9 version of the CRISPR/Cas9 system can be used to target protein domains for transcriptional regulation, epigenetic modification, and microscopic visualization of specific genome loci.

In some embodiments, the provided virus particles (e.g. lentiviral particles) containing a Cas nuclease (e.g. Cas9) further comprise, or is further complexed with, one or more CRISPR-Cas system guide RNA(s) for targeting a desired target gene. In some embodiments, the CRISPR guide RNAs are efficiently encapsulated in the CAS-containing viral particles. In some embodiments, the provided virus particles (e.g. lentiviral particles) further comprises, or is further complexed with a targeting nucleic acid.

### IV. EXEMPLARY EMBODIMENTS

Among the provided embodiments are:

1. A method of delivering an exogenous agent to a subject, the method comprising:
administering to a subject a first dose of a targeted lipid particle comprising an exogenous agent, and administering to the subject a second dose of the targeted lipid particle, wherein
the targeted lipid particle comprises one or more Paramyxovirus envelope proteins, and wherein
the first and second doses are administered within one month (e.g., within four weeks, within three weeks, within two weeks, within seven days, within six days, within five days, within four days, within three days, within two days, or within one day) of each other.

2. A method of delivering an exogenous agent to a subject, the method comprising:
administering to a subject a first dose of a targeted lipid particle comprising an exogenous agent, and administering to the subject a second dose of the targeted lipid particle, wherein
the targeted lipid particle comprises one or more Paramyxovirus envelope proteins, and wherein
the second dose is administered (i) on the first day following the first dose or (ii) on the second, third, fourth, fifth, sixth, seventh, fourteenth, twenty-first, or twenty-eighth day following the first dose.

3. The method of embodiment 1 or 2, wherein the one or more Paramyxovirus envelope proteins have fusogenic activity.

4. The method of any one of embodiments 1-3, wherein the native binding tropism of the one or more of the Paramyxovirus envelope proteins is reduced.

5. The method of any one of embodiment 1-4, wherein the one or more Paramyxovirus envelope proteins is derived from an H protein molecule or a biologically active portion thereof from a Paramyxovirus and/or an HN protein molecule or a biologically active portion thereof from a Paramyxovirus.

6. The method of any one of embodiments 1-4, wherein the one or more Paramyxovirus envelope proteins comprises an F protein molecule or a biologically active portion thereof and/or a glycoprotein G (G protein) or a biologically active portion thereof.

7. The method of embodiment 1-6, wherein the one or more Paramyxovirus envelope proteins comprises an F protein molecule or a biologically active portion thereof from a Paramyxovirus and a glycoprotein G (G protein) or a biologically active portion thereof from a Paramyxovirus.

8. The method of any of embodiments 1-7, wherein the paramyxovirus is a henipavirus.

9. The method of any of embodiments 1-8, wherein the paramyxovirus is Measles morbillivirus.

10. The method of any of embodiments 1-8, wherein the paramyxovirus is a Hendra virus.

11. The method of any of embodiments 1-8, wherein the paramyxovirus is Nipah virus.

12. The method of any of embodiments 6-8 and 11, wherein the F protein or the biologically active portion thereof is a wild-type Nipah virus F (NiV-F) protein or is a functionally active variant or biologically active portion thereof.

13. The method of any of embodiments 6-8, 11 or 12 wherein the F protein molecule or a biologically active portion thereof is a NiV-F protein that has the amino acid sequence set forth in SEQ ID NO: 7 or an amino acid sequence having at or about 80%, at least at or about 81%, at least at or about 82%, at least at or about 83%, at or about 84%, at least at or about 85%, at least at or about 86%, or at least at or about 87%, at least at or about 88%, or at least at or about 89%, at least at or about 90%, at least at or about 91%, at least at or about 92%, at least at or about 93%, at least at or about 94%, at least at or about 95%, at or about 96%, at least at or about 97%, at least at or about 98%, or at least at or about 99% sequence identity to SEQ ID NO:7.

14. The method of any of embodiments 6-8 and 11-13, wherein the NiV-F protein is a biologically active portion that is truncated and lacks up to 40 contiguous amino acid residues at or near the C-terminus of the wild-type NiV-F protein (SEQ ID NO:7).

15. The method of any of embodiments 6-8 and 11-14, wherein the NiV-F protein is a biologically active portion that is truncated at the C-terminus of wild-type NiV-F and has the sequence set forth in any of SEQ ID NOS: 1-10 or an amino acid sequence having at least at or about 80%, at least at or about 81%, at least at or about 82%, at least at or about 83%, at least at or about 84%, at least at or about 85%, at least at or about 86%, at least at or about 87%, at least at or about 88%, at least at or about 89%, at least at or about 90%, at least at or about 91%, at least at or about 92%, at least at or about 93%, at least at or about 94%, at least at or about 95%, at or about 96%, at least at or about 97%, at least at or about 98%, or at least at or about 99% sequence identity to SEQ ID NOs: 1-10.

16. The method of any of embodiments 6-8 and 11-14, wherein the NiV-F protein is a biologically active portion that has a truncation at or near the C-terminus of the wild-type NiV-F selected from the group consisting of a 5 amino acid truncation at or near the C-terminus of the wild-type NiV-F protein, a 10 amino acid truncation at or near the C-terminus of the wild-type NiV-F protein, a 15 amino acid truncation at or near the C-terminus, a 20 amino acid truncation at or near the C-terminus of the wild-type NiV-F protein, a 22 amino acid truncation at or near the C-terminus of the wild-type NiV-F protein, or a 25 amino acid truncation at or near the C-terminus of the wild-type NiV-F protein, optionally wherein the wild-type NiV-F protein is set forth in SEQ ID NO:7.

17. The method of any of embodiments 6-8 and 11-15, wherein the F protein is a NiV-F protein that is a biologically active portion that has a 20 amino acid truncation at or near the C-terminus of the wild-type NiV-F protein (SEQ ID NO:7).

18. The method of embodiment 17, wherein the NiV-F protein has an amino acid sequence set forth in SEQ ID NO:2 or an amino acid sequence having at or about 80%, at least at or about 81%, at least at or about 82%, at least at or about 83%, at or about 84%, at least at or about 85%, at least at or about 86%, or at least at or about 87%, at least at or about 88%, or at least at or about 89%, at least at or about 90%, at least at or about 91%, at least at or about 92%, at least at or about 93%, at least at or about 94%, at least at or about 95%, at or about 96%, at least at or about 97%, at least at or about 98%, or at least at or about 99% sequence identity to SEQ ID NO: 2.

19. The method of any of embodiments 6-8 and 11-16, wherein the F protein is a NiV-F protein that is a biologically active portion thereof that has a 22 amino acid truncation at or near the C-terminus of the wild-type NiV-F protein (SEQ ID NO:7).

20. The method of embodiment 19, wherein the NiV-F protein has an amino acid sequence set forth in SEQ ID NO:11 or a sequence having at or about 80%, at least at or about 81%, at least at or about 82%, at least at or about 83%, at or about 84%, at least at or about 85%, at least at or about 86%, or at least at or about 87%, at least at or about 88%, or at least at or about 89%, at least at or about 90%, at least at or about 91%, at least at or about 92%, at least at or about 93%, at least at or about 94%, at least at or about 95%, at or about 96%, at least at or about 97%, at least at or about 98%, or at least at or about 99% sequence identity to SEQ ID NO: 11.

21. The method of embodiment 19 or embodiment 20, wherein the NiV-F protein has an amino acid sequence set forth in SEQ ID NO:12 or a sequence having at or about 80%, at least at or about 81%, at least at or about 82%, at least at or about 83%, at or about 84%, at least at or about 85%, at least at or about 86%, or at least at or about 87%, at least at or about 88%, or at least at or about 89%, at least at or about 90%, at least at or about 91%, at least at or about 92%, at least at or about 93%, at least at or about 94%, at least at or about 95%, at or about 96%, at least at or about 97%, at least at or about 98%, or at least at or about 99% sequence identity to SEQ ID NO: 12.

22. The method of any of embodiments 5-8 and 11-21, wherein the F protein or the biologically active portion thereof is a NiV-F protein that is a functionally active variant that comprises a point mutation on an N-linked glycosylation site of the wild-type NiV-F protein (SEQ ID NO:7) or a biologically active portion thereof.

23. The method of any of embodiments 5-8 and 11-21, wherein the F protein or the biologically active portion thereof is a NiV-F protein that is a functionally active variant that comprises:
i) a 20 amino acid truncation at or near the C-terminus of the wild-type NiV-F protein (SEQ ID NO:7); and/or
ii) a point mutation on an N-linked glycosylation site.

24. The method of embodiment 23, wherein the NiV-F protein has an amino acid sequence set forth in SEQ ID NO:11 or an amino acid sequence having at or about 80%, at least at or about 81%, at least at or about 82%, at least at or about 83%, at or about 84%, at least at or about 85%, at least at or about 86%, or at least at or about 87%, at least at or about 88%, or at least at or about 89%, at least at or about 90%, at least at or about 91%, at least at or about 92%, at least at or about 93%, at least at or about 94%, at least at or about 95%, at or about 96%, at least at or about 97%, at least at or about 98%, or at least at or about 99% sequence identity to SEQ ID NO: 11.

25. The method of any of embodiments 5-8 and 10-24, wherein the G protein or the biologically active portion thereof is a wild-type Nipah virus G (NiV-G) protein or a Hendra virus G protein or is a functionally active variant or biologically active portion thereof.

26. The method of any of embodiments 5-8 and 11-25, wherein the G protein or the biologically active portion thereof is a wild-type NiV-G protein or a functionally active variant or biologically active portion thereof.

27. The method of any of embodiments 5-8 and 11-26, wherein the G protein or the biologically active portion thereof is a mutant NiV-G protein that is modified to exhibit reduced native binding tropism.

28. The method of any of embodiments 5-8 and 11-27, wherein the G protein or the biologically active portion thereof is a mutant NiV-G protein that exhibits reduced binding to Ephrin B2 or Ephrin B3.

29. The method of any of embodiments 5-8 and 11-28, wherein the G protein or the biologically active portion thereof is a mutant NiV-G protein comprising one or more amino acid substitutions corresponding to amino acid substitutions selected from the group consisting of E501A, W504A, Q530A and E533A with reference to numbering set forth in SEQ ID NO:14.

30. The method of any of embodiments 5-8 and 11-29, wherein the NiV-G protein is a biologically active portion that is truncated and lacks up to 40 contiguous amino acid residues at or near the N-terminus of the wild-type NiV-G protein (SEQ ID NO:14).

31. The method of any of embodiments 5-8 and 11-30, wherein the NiV-G protein is a biologically active portion that has a truncation at or near the N-terminus of the wild-type NiV-G selected from the group consisting of a 5 amino acid truncation at or near the N-terminus of the wild-type NiV-G protein, a 10 amino acid truncation at or near the N-terminus of the wild-type NiV-G protein, a 15 amino acid truncation at or near the N-terminus of the wild-type NiV-G protein, a 20 amino acid truncation at or near the N-terminus of the wild-type NiV-G protein, a 25 amino acid truncation at or near the N-terminus of the wild-type NiV-G protein, a 30 amino acid truncation at or near the N-terminus of the wild-type NiV-G protein, or a 34 amino acid truncation at or near the N-terminus of the wild-type NiV-G protein, optionally wherein the wild-type NiV-G protein is set forth in SEQ ID NO:13, SEQ ID NO:14, or SEQ ID NO: 19.

32. The method of any of embodiments 6-8 and 11-31, wherein the NiV-G protein is a biologically active portion that is truncated at the N-terminus of wild-type NiV-G and has the sequence set forth in any of SEQ ID NOS: 13, 14, or 19 or an amino acid sequence having at least at or about 80%, at least at or about 81%, at least at or about 82%, at least at or about 83%, at least at or about 84%, at least at or about 85%, at least at or about 86%, at least at or about 87%, at least at or about 88%, at least at or about 89%, at least at or about 90%, at least at or about 91%, at least at or about 92%, at least at or about 93%, at least at or about 94%, at least at or about 95%, at or about 96%, at least at or about 97%, at least at or about 98%, or at least at or about 99% sequence identity to SEQ ID NOs 13, 14, or 19.

33. The method of any of embodiments 6-8 and 11-32, wherein the G protein molecule or a biologically active portion thereof NiV-G protein has the amino acid sequence set forth in SEQ ID NO: 13 or an amino acid sequence having at or about 80%, at least at or about 81%, at least at or about 82%, at least at or about 83%, at or about 84%, at least at or about 85%, at least at or about 86%, or at least at or about 87%, at least at or about 88%, or at least at or about 89%, at least at or about 90%, at least at or about 91%, at least at or about 92%, at least at or about 93%, at least at or about 94%, at least at or about 95%, at or about 96%, at least at or about 97%, at least at or about 98%, or at least at or about 99% sequence identity to SEQ ID NO:13.

34. The method of any of embodiments 6-8 and 11-32, wherein the G protein or the biologically active portion thereof is a mutant NiV-G protein having the amino acid sequence set forth in SEQ ID NO: 14 or an amino acid sequence having at or about 80%, at least at or about 81%, at least at or about 82%, at least at or about 83%, at or about 84%, at least at or about 85%, at least at or about 86%, or at least at or about 87%, at least at or about 88%, or at least at or about 89%, at least at or about 90%, at least at or about 91%, at least at or about 92%, at least at or about 93%, at least at or about 94%, at least at or about 95%, at or about 96%, at least at or about 97%, at least at or about 98%, or at least at or about 99% sequence identity to SEQ ID NO:14.

35. The method of any of embodiments 6-8 and 11-32, wherein the G protein or the biologically active portion thereof is a mutant NiV-G protein having the amino acid sequence set forth in SEQ ID NO:19 or an amino acid sequence having at or about 80%, at least at or about 81%, at least at or about 82%, at least at or about 83%, at or about 84%, at least at or about 85%, at least at or about 86%, or at least at or about 87%, at least at or about 88%, or at least at or about 89%, at least at or about 90%, at least at or about 91%, at least at or about 92%, at least at or about 93%, at least at or about 94%, at least at or about 95%, at or about 96%, at least at or about 97%, at least at or about 98%, or at least at or about 99% sequence identity to SEQ ID NO:19.

36. The method of any of embodiments 6-8, 11-34, wherein the F protein comprises the sequence set forth in SEQ ID NO. 12 and the G protein comprises the sequence set forth in SEQ ID NO. 19.

37. The method of any of embodiments 1-36, wherein at least one of the one or more Paramyxovirus envelope proteins are linked to a secondary moiety that is a targeting domain or a functional domain.

38. The method of embodiment 37, wherein the at least one of the one or more Paramyxovirus is a glycoprotein G (G protein) or a biologically active portion thereof and the G protein or biologically active portion thereof is linked to the secondary moiety.

39. The method of embodiment 37 or embodiment 38, wherein the secondary moiety is a functional domain and the functional domain is selected from a cytokine, growth factor, hormone, neurotransmitter, receptor, or apoptosis ligand.

40. The method of embodiment 37 or embodiment 38, wherein the secondary moiety is a targeting domain and the targeting domain is specific for a cell surface receptor on a target cell.

41. The method of any of embodiment 37, wherein the targeting domain is a Design ankyrin repeat proteins (DARPin), a single domain antibody (sdAb), a single chain variable fragment (scFv), or an antigen-binding fibronectin type III (Fn3) scaffold.

42. The method of any one of embodiments 37-41, wherein the at least one of the one or more Paramyxovirus envelope proteins and the secondary moiety are directly linked.

43. The method of any one of embodiments 37-41, wherein the at least one of the one or more Paramyxovirus envelope proteins and secondary moiety are indirectly linked via a linker.

44. The method of embodiment 43, wherein the linker is a peptide linker.

45. The method of embodiment 44, wherein the peptide linker is (GmS)ₙ (SEQ ID NO: 11), wherein each of m and n is an integer between 1 to 4, inclusive.

46. The method of any of embodiments 1-45, wherein the exogenous agent is a nucleic acid or a polypeptide.

47. The method of any of embodiments 1-46, wherein the exogenous agent is a nucleic acid encoding a payload gene, optionally wherein the nucleic acid encodes a chimeric antigen receptor (CAR).

48. The method of embodiment 40, wherein the target cell is one or more of a monocyte, macrophage, neutrophil, dendritic cell, eosinophil, mast cell, platelet, large granular lymphocyte, Langerhans' cell, natural killer (NK) cell, T lymphocyte (e.g., T cell), a Gamma delta T cell, B lymphocyte (e.g., B cell), CD3+ T cell, a CD4+ T cell, a CD8+ T cell, a hepatocyte, a hematopoietic stem cell, a CD34+ hematopoietic stem cell, a CD105+ hematopoietic stem cell, a CD117+ hematopoietic stem cell, a CD105+ endothelial cell, a B cell, a CD20+ B cell, a CD19+ B cell, a cancer cell, a CD133+ cancer cell, an EpCAM+ cancer cell, a CD19+ cancer cell, a Her2/Neu+ cancer cell, a GluA2+ neuron, a GluA4+ neuron, a NKG2D+ natural killer cell, a SLC1A3+ astrocyte, a SLC7A10+ adipocyte, or a CD30+ lung epithelial cell.

49. The method of any of embodiments 1-48, wherein the first and second dose are administered ex vivo to the subject.

50. The method of any of embodiments 1-48, wherein the first and second dose are administered to the subject intravenously.

51. The method of any of embodiments 1-50, wherein the time period between the first and second dose is no more than one month.

52. The method of any of embodiments 1-50, wherein the time period between the first and second dose is no more than one week.

53. The method of any of embodiments 1-50, wherein the time period between the first and second dose is no more than three days.

54. The method of any of embodiments 1-53, wherein the method further comprises administration of a third dose of the targeted lipid particle.

55. The method of embodiment 54, wherein the third dose is administered within one month (e.g., within four weeks, within three weeks, within two weeks, within seven days, within six days, within five days, within four days, within three days, within two days, or within one day) of the second dose.

56. The method of embodiment 54, wherein the third dose is administered (i) on the first day following the second dose or (ii) on the second, third, fourth, fifth, sixth, seventh, fourteenth, twenty-first, or twenty-eighth day following the second dose.

57. The method of any of embodiments 54-56, wherein the third dose is administered ex vivo to the subject.

58. The method of any of embodiments 54-56, wherein the third dose is administered to the subject intravenously.

59. The method of any of embodiments 54-56, wherein the time period between the first and third dose is no more than one month.

60. The method of any of embodiments 54-59, wherein the time period between the first and third dose is no more than one week.

61. The method of any of embodiments 54-60, wherein the time period between the first and third dose is no more than three days.

62. A targeted lipid particle comprising one or more Paramyxovirus envelope proteins for use in the method of any of embodiments 1-61.

63. The targeted lipid particle of embodiment 61, wherein the lipid particle is a viral vector and/or viral-like particle.

64. The targeted lipid particle of embodiment 62 or embodiment 63, wherein the lipid particle is a viral vector, optionally wherein the lipid particle is a lentiviral vector.

### V. EXAMPLES

The following examples are included for illustrative purposes only and are not intended to limit the scope of the invention.

### Example I Repeat Dosing of Pseudotyned Lentiviral Vectors

A method of dosing of a liver-(hepatocyte) targeted lentiviral vectors by repeated administration of the lentiviral vector at successive doses was tested in vivo in the FRG (Fah−/− Rag2−/—Il2rg−/−) humanized liver mouse model.

For viral vector production, HEK293 producer cells were transfected with plasmids expressing viral vector proteins (gag/pol, rev) and a transfer plasmid encoding a GFP reporter. Envelope proteins were provided as plasmids expressing Nipah F protein and a ASGR1-retargeted Nipah G protein. Following viral vector production, the cell culture was centrifuged to pellet the cells and the supernatant containing virus was collected, and concentrated by ultracentrifugation prior to in vivo dosing.

To first assay the transduction potential of the viral vectors, mice were administered a single dose of GFP expressing vector ranging from 6¹¹-6¹² VGs/kg (vector genomes/kilogram body weight). Flow cytometric analysis on Day 7 demonstrated that the re-targeted vector transduced in a dose-dependent manner in vivo (FIG. 1A).

To test the effect of repeat dosing, mice were next administered one, two, or three successive GFP-expressing vector doses. Vector was administered up to three times at two different dose levels (5 ml/kg-8.3e12 VGs/kg and 10 ml/kg-1.7e13 VGs/kg; vector genomes/kilogram body weight) 24 hours apart. Administrations were well-tolerated, and the H&E stains of the liver sections were comparable to saline-treated animals. Animals were sacrificed seven days post final administration of vector dose. Livers were dissociated for analysis by flow cytometry and vector copy number (VCN). A stepwise increase in number of GFP+ cells was observed with repeat dosing, as shown in FIG. 1B. Similar increases in VCN was observed with repeat dosing in FIG. 1C. Qualitative immunohistochemistry confirmed a dose-dependent increase in hepatocyte transduction following repeat dosing.

The results show that a similarly high level of hepatocyte transduction in the FRG model could be achieved by repeat dosing a lower potency vector. Taken together, these results indicate that this strategy (e.g., repeated dosing of pseudotyped vector) can result in increased gene delivery in vivo, including for vectors with lower potency, and supports a repeat dosing strategy for vector administration.

### Example 2 Lentiviral Vector Mediated Delivery of a Therapeutic Gene

In a further experiment, the same lentiviral vector pseudotyped with Nipah F protein and an ASGR1-retargeted Nipah G protein as described above was used to deliver human ornithine transcarbamylase enzyme (hOTC) in vivo.

Viral vector was produced as described above. Briefly, HEK293 producer cells were transfected with plasmids expressing viral vector proteins (gag/pol, rev) and a transfer plasmid encoding a codon optimized hOTC with a hepatocyte specific promoter. Envelope proteins were provided as plasmids expressing Nipah F protein and a ASGR1-retargeted Nipah G protein. The supernatant was ultracentrifuged to concentrate the vector roughly ˜300× before dosing mice. Following this viral vector production, the cell culture was centrifuged to pellet the cells and the supernatant containing the fusosome was collected.

To test the delivery of this therapeutic gene, FRG Hu-liver model mice as detailed in Example 1 above were administered 3.7¹² VGs/kg (vector genomes/kilogram body weight) of OTC-expressing vector. Administrations were well-tolerated, and animals were sacrificed fourteen days post administration of vector dose. Livers were analyzed for OTC expression, vector copy number (VCN), and histology.

To assay expression of OTC enzyme, liver lysate was incubated with ornithine and carbamyl phosphate (i.e., OTC substrates). OTC present in the liver lysate converts the provided substrates into the product citrulline, which was measured by an HPLC-based assay.

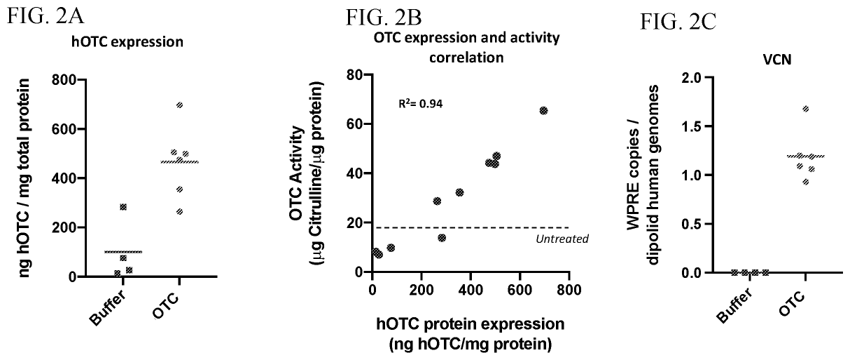

As shown in FIG. 2A, an average five-fold increase in hOTC expression was observed in animals treated with the exemplary ASGR1-retargeted vector. In some aspects, the hOTC expression observed exceeds the estimated per cell requirement for a therapeutic benefit in an OTC deficient model. This increase in expression was also shown to be correlated with functional OTC activity (FIG. 2B). VCN was also shown to be significantly elevated in vector-treated animals (FIG. 2C). Fluorescence in situ hybridization of liver samples was also performed, and confirmed broad distribution of the OTC transgene-expressing hepatocytes.

To test the effect of repeat dosing, mice are administered one, two, or three successive OTC-expressing vector doses as described in Example 1 above. The results show an increase in number of OTC+ cells is observed with repeat dosing. This strategy (e.g., repeated dosing of pseudotyped vector) can result in increased gene delivery in vivo, including for delivery of therapeutic genes such as OTC, and supports a repeat dosing strategy for vector administration.

### Example 3 Repeat Dosing of BaEVTR LV Boosts Transduction In Vivo

This Example demonstrates the effect of repeat dosing of a lentiviral vector (LV) pseudotyped with BaEVTR on transduction. NBSGW mice were humanized with cord-blood derived CD34+ cells (Jackson Labs strain Hu-NSG-NBSGW-CD34, 702662) and were 10 weeks after humanization at study start. Baseline bleeds were performed at weeks 2, 4, 6, and 9 to analyze the composition of the cell types of the humanized mouse blood by flow cytometry. At D0 of CD34+ dosing, mice were also administered 10 mL/kg lentiviral vector pseudotyped with BaEVTR further comprising a green fluorescent protein (GFP) transgene.

LV+VECTOFUSIN-1 in vivo administration occurred 75 min after AMD3100 dosing. LV was thawed and kept on ice. LV was pre-mixed with Vectofusin-1 (Miltenyi Biotec, Cat #130-111-163) at a final Vectofusin-1 concentration of 12 μg/ml) for 10 minutes. Immediately following the 10-minute LV+VECTOFUSIN-1 mixture incubation, the LV+VECTOFUSIN-1 mix was injected into mice intravenously.

Following the primary administration of GFP LV, mice were administered agents for mobilization in two stages: 1) D-4 through D-1: subcutaneous G-CSF injections (5 μg/day; 24 h between each injection) and 2) D0: subcutaneous AMD3100 (5 mg/kg). G-CSF (Peprotech; Recombinant Human, Cat #300-23) was resuspended in ultrapure water (Invitrogen 10977-015) and filter-sterilized (0.2 um). G-CSF solution was prepared three days prior to first injection and aliquots were stored at 4° C. for the duration of the study. AMD3100 (Sigma Aldrich Cat #A5602-5MG) was resuspended in ultrapure water (Invitrogen 10977-015) and sterile-filtered shortly prior to in vivo administration. AMD3100 was administered subcutaneously 24 h after the last dose of G-CSF. The non-mobilized control mouse cohort received a subcutaneous saline injection (Hospira/Pfizer, 00409-4888-20) at the same dosing schedule as the mice that were dosed the G-CSF and AMD3100 mobilizing agents.

Mice received three further doses of a similar BaEVTR LV with a RFP transgene on week 10 D0, and again on week 10 D1 or D2: a dose of LV (10 mL/kg)+VECTOFUSIN-1 (BaEVTR) The dose was about 9.2e7 TU/mouse of the BaEVTR RFP LV. For each administration, VECTOFUSIN-1 was administered intravenously 120 min after AMD3100 administration.

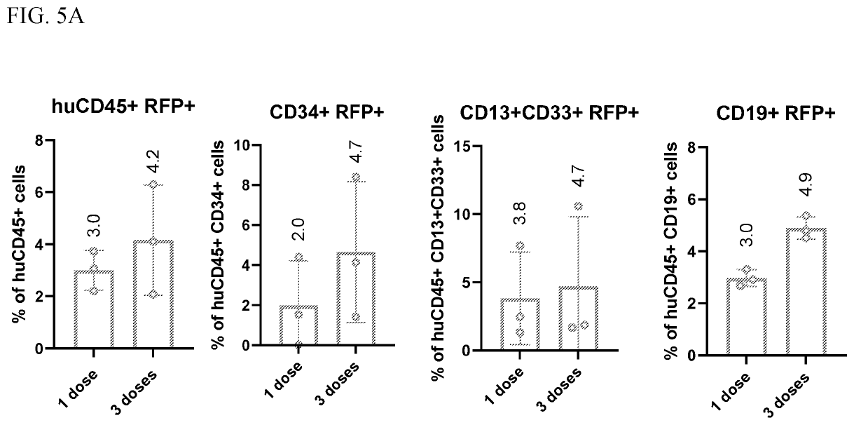

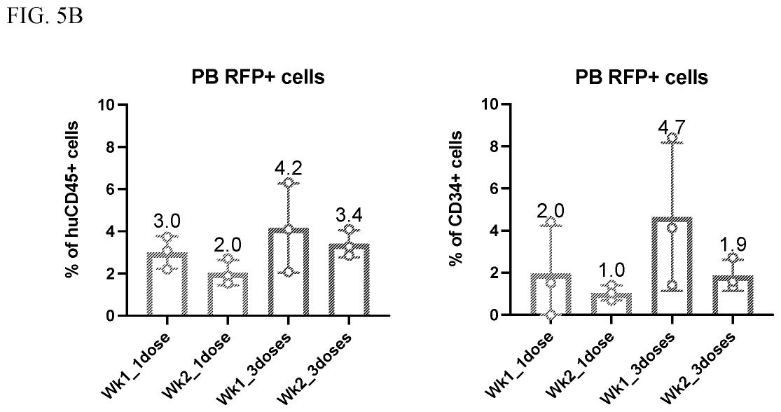

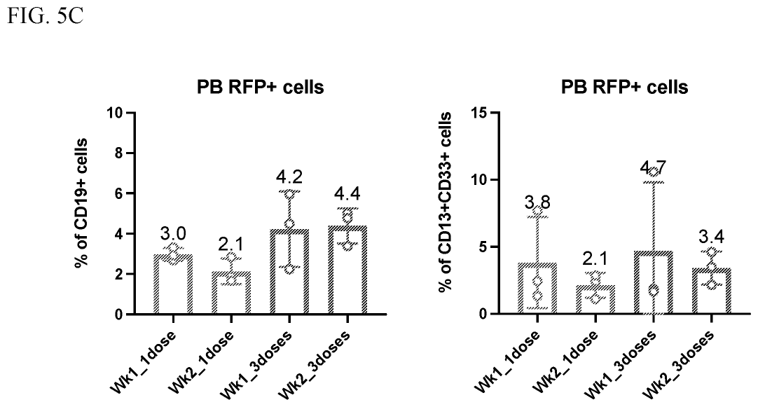

CD45+ cells collected from the Day 7 bleed were analyzed by flow cytometry for expression of the GFP transgene from the initial dose, as well as the RFP transgene from the subsequent doses, in order to determine whether the BaEVTR+RFP LV was capable of transducing cells that have already been transduced by BaEVTR+GFP LV. It was observed that BaEVTR transduction was much greater at 3 doses than when a single dose was administered, as evidenced by an increase in RFP+ cells (FIG. 5A). The percentage of RFP+ cells also was similarly increased after multiple doses in samples collected on D12, including for hcCD34 and CD34+ cells (FIG. 5B) and CD19+ and CD13+/CD33+ cells (FIG. 5C).

The present invention is not intended to be limited in scope to the particular disclosed embodiments, which are provided, for example, to illustrate various aspects of the invention. Various modifications to the compositions and methods described will become apparent from the description and teachings herein. Such variations may be practiced without departing from the true scope and spirit of the disclosure and are intended to fall within the scope of the present disclosure.

VI. SEQUENCES

| SEQ ID NO | SEQUENCE | DESCRIPTION |
|---|---|---|
| 1 | MATQEVRLKCLLCGIIVLVLSLEGLGILHYEKLSKIGLVKGI | Hendra F |
| | TRKYKIKSNPLTKDIVIKMIPNVSNVSKCTGTVMENYKSRL | |
| | TGILSPIKGAIELYNNNTHDLVGDVKLAGVVMAGIAIGIAT | |
| | AAQITAGVALYEAMKNADNINKLKSSIESTNEAVVKLQET | |
| | AEKTVYVLTALQDYINTNLVPTIDQISCKQTELALDLALSK | |
| | YLSDLLFVFGPNLQDPVSNSMTIQAISQAFGGNYETLLRTL | |
| | GYATEDFDDLLESDSIAGQIVYVDLSSYYIIVRVYFPILTEIQ | |
| | QAYVQELLPVSFNNDNSEWISIVPNFVLIRNTLISNIEVKYC | |
| | LITKKSVICNQDYATPMTASVRECLTGSTDKCPRELVVSSH | |
| | VPRFALSGGVLFANCISVTCQCQTTGRAISQSGEQTLLMID | |
| | NTTCTTVVLGNIIISLGKYLGSINYNSESIAVGPPVYTDKVDI | |
| | SSQISSMNQSLQQSKDYIKEAQKILDTVNPSLISMLSMIILYV | |
| | LSIAALCIGLITFISFVIVEKKRGNYSRLDDRQVRPVSNGDL | |
| | YYIGT | |
| 2 | MVVILDKRCYCNLLILILMISECSVGILHYEKLSKIGLVKGV | Nipah FO |
| | TRKYKIKSNPLTKDIVIKMIPNVSNMSQCTGSVMENYKTRL | |
| | NGILTPIKGALEIYKNNTHDLVGDVRLAGVIMAGVAIGIAT | |
| | AAQITAGVALYEAMKNADNINKLKSSIESTNEAVVKLQET | |
| | AEKTVYVLTALQDYINTNLVPTIDKISCKQTELSLDLALSK | |
| | YLSDLLFVFGPNLQDPVSNSMTIQAISQAFGGNYETLLRTL | |
| | GYATEDFDDLLESDSITGQIIYVDLSSYYIIVRVYFPILTEIQQ | |
| | AYIQELLPVSFNNDNSEWISIVPNFILVRNTLISNIEIGFCLIT | |
| | KRSVICNQDYATPMTNNMRECLTGSTEKCPRELVVSSHVP | |
| | RFALSNGVLFANCISVTCQCQTTGRAISQSGEQTLLMIDNTT | |
| | CPTAVLGNVIISLGKYLGSVNYNSEGIAIGPPVFTDKVDISS | |
| | QISSMNQSLQQSKDYIKEAQRLLDTVNPSLISMLSMIILYVL | |
| | SIASLCIGLITFISFIIVEKKRNTYSRLEDRRVRPTSSGDLYYI | |
| | GT | |
| 3 | MSNKRTTVLIIISYTLFYLNNAAIVGFDFDKLNKIGVVQGRV | CedV F |
| | LNYKIKGDPMTKDLVLKFIPNIVNITECVREPLSRYNETVRR | |
| | LLLPIHNMLGLYLNNTNAKMTGLMIAGVIMGGIAIGIATAA | |
| | QITAGFALYEAKKNTENIQKLTDSIMKTQDSIDKLTDSVGT | |
| | SILILNKLQTYINNQLVPNLELLSCRQNKIEFDLMLTKYLVD | |
| | LMTVIGPNINNPVNKDMTIQSLSLLFDGNYDIMMSELGYTP | |
| | QDFLDLIESKSITGQIIYVDMENLYVVIRTYLPTLIEVPDAQI | |
| | YEFNKITMSSNGGEYLSTIPNFILIRGNYMSNIDVATCYMTK | |
| | ASVICNQDYSLPMSQNLRSCYQGETEYCPVEAVIASHSPRF | |
| | ALTNGVIFANCINTICRCQDNGKTITQNINQFVSMIDNSTCN | |
| | DVMVDKFTIKVGKYMGRKDINNINIQIGPQIIIDKVDLSNEI | |
| | NKMNQSLKDSIFYLREAKRILDSVNISLISPSVQLFLIIISVLS | |
| | FIILLIIIVYLYCKSKHSYKYNKFIDDPDYYNDYKRERINGK | |
| | ASKSNNIYYVGD | |
| 4 | MALNKNMFSSLFLGYLLVYATTVQSSIHYDSLSKVGVIKGL | Mojiang F |
| | TYNYKIKGSPSTKLMVVKLIPNIDSVKNCTQKQYDEYKNL | |
| | VRKALEPVKMAIDTMLNNVKSGNNKYRFAGAIMAGVALG | |
| | VATAATVTAGIALHRSNENAQAIANMKSAIQNTNEAVKQL | |
| | QLANKQTLAVIDTIRGEINNNIIPVINQLSCDTIGLSVGIRLT | |
| | QYYSEIITAFGPALQNPVNTRITIQAISSVENGNFDELLKIMG | |
| | YTSGDLYEILHSELIRGNIIDVDVDAGYIALEIEFPNLTLVPN | |
| | AVVQELMPISYNIDGDEWVTLVPRFVLTRTTLLSNIDTSRC | |
| | TITDSSVICDNDYALPMSHELIGCLQGDTSKCAREKVVSSY | |
| | VPKFALSDGLVYANCLNTICRCMDTDTPISQSLGATVSLLD | |
| | NKRCSVYQVGDVLISVGSYLGDGEYNADNVELGPPIVIDKI | |
| | DIGNQLAGINQTLQEAEDYIEKSEEFLKGVNPSIITLGSMVV | |
| | LYIFMILIAIVSVIALVLSIKLTVKGNVVRQQFTYTQHVPSM | |
| | ENINYVSH | |
| 5 | MKKKTDNPTISKRGHNHSRGIKSRALLRETDNYSNGLIVEN | Bat PV F |
| | LVRNCHHPSKNNLNYTKTQKRDSTIPYRVEERKGHYPKIK | |
| | HLIDKSYKHIKRGKRRNGHNGNIITIILLLILILKTQMSEGAI | |
| | HYETLSKIGLIKGITREYKVKGTPSSKDIVIKLIPNVTGLNKC | |
| | TNISMENYKEQLDKILIPINNIIELYANSTKSAPGNARFAGVI | |
| | IAGVALGVAAAAQITAGIALHEARQNAERINLLKDSISATN | |
| | NAVAELQEATGGIVNVITGMQDYINTNLVPQIDKLQCSQIK | |
| | TALDISLSQYYSEILTVFGPNLQNPVTTSMSIQAISQSFGGNI | |
| | DLLLNLLGYTANDLLDLLESKSITGQITYINLEHYFMVIRVY | |
| | YPIMTTISNAYVQELIKISFNVDGSEWVSLVPSYILIRNSYLS | |
| | NIDISECLITKNSVICRHDFAMPMSYTLKECLTGDTEKCPRE | |
| | AVVTSYVPRFAISGGVIYANCLSTTCQCYQTGKVIAQDGSQ | |
| | TLMMIDNQTCSIVRIEEILISTGKYLGSQEYNTMHVSVGNPV | |
| | FTDKLDITSQISNINQSIEQSKFYLDKSKAILDKINLNLIGSVP | |
| | ISILFIIAILSLILSIITFVIVMIIVRRYNKYTPLINSDPSSRRSTIQ | |
| | DVYIIPNPGEHSIRSAARSIDRDRD | |
| 6 | ILHYEKLSKIGLVKGITRKYKIKSNPLTKDIVIKMIPNVSNVS | Hendra virus F |
| | KCTGTVMENYKSRLTGILSPIKGAIELYNNNTHDLVGDVKL | Protein, Without |
| | AGVVMAGIAIGIATAAQITAGVALYEAMKNADNINKLKSSI | signal sequence |
| | ESTNEAVVKLQETAEKTVYVLTALQDYINTNLVPTIDQISC | |
| | KQTELALDLALSKYLSDLLFVFGPNLQDPVSNSMTIQAISQ | |
| | AFGGNYETLLRTLGYATEDFDDLLESDSIAGQIVYVDLSSY | |
| | YIIVRVYFPILTEIQQAYVQELLPVSFNNDNSEWISIVPNFVLI | |
| | RNTLISNIEVKYCLITKKSVICNQDYATPMTASVRECLTGST | |
| | DKCPRELVVSSHVPRFALSGGVLFANCISVTCQCQTTGRAI | |
| | SQSGEQTLLMIDNTTCTTVVLGNIIISLGKYLGSINYNSESIA | |
| | VGPPVYTDKVDISSQISSMNQSLQQSKDYIKEAQKILDTVN | |
| | PSLISMLSMIILYVLSIAALCIGLITFISFVIVEKKRGNYSRLD | |
| | DRQVRPVSNGDLYYIGT | |
| 7 | ILHYEKLSKIGLVKGVTRKYKIKSNPLTKDIVIKMIPNVSNM | Nipah virus F |
| | SQCTGSVMENYKTRLNGILTPIKGALEIYKNNTHDLVGDVR | Protein, without |
| | LAGVIMAGVAIGIATAAQITAGVALYEAMKNADNINKLKS | signal sequence |
| | SIESTNEAVVKLQETAEKTVYVLTALQDYINTNLVPTIDKIS | |
| | CKQTELSLDLALSKYLSDLLFVFGPNLQDPVSNSMTIQAISQ | |
| | AFGGNYETLLRTLGYATEDFDDLLESDSITGQIIYVDLSSYY | |
| | IIVRVYFPILTEIQQAYIQELLPVSFNNDNSEWISIVPNFILVR | |
| | NTLISNIEIGFCLITKRSVICNQDYATPMTNNMRECLTGSTE | |
| | KCPRELVVSSHVPRFALSNGVLFANCISVTCQCQTTGRAIS | |
| | QSGEQTLLMIDNTTCPTAVLGNVIISLGKYLGSVNYNSEGIA | |
| | IGPPVFTDKVDISSQISSMNQSLQQSKDYIKEAQRLLDTVNP | |
| | SLISMLSMIILYVLSIASLCIGLITFISFIIVEKKRNTYSRLEDR | |
| | RVRPTSSGDLYYIGT | |
| 8 | TVLIIISYTLFYLNNAAIVGFDFDKLNKIGVVQGRVLNYKIK | Cedar Virus F |
| | GDPMTKDLVLKFIPNIVNITECVREPLSRYNETVRRLLLPIH | Protein, without |
| | NMLGLYLNNTNAKMTGLMIAGVIMGGIAIGIATAAQITAG | signal sequence |
| | FALYEAKKNTENIQKLTDSIMKTQDSIDKLTDSVGTSILILN | |
| | KLQTYINNQLVPNLELLSCRQNKIEFDLMLTKYLVDLMTVI | |
| | GPNINNPVNKDMTIQSLSLLFDGNYDIMMSELGYTPQDFLD | |
| | LIESKSITGQIIYVDMENLYVVIRTYLPTLIEVPDAQIYEFNKI | |
| | TMSSNGGEYLSTIPNFILIRGNYMSNIDVATCYMTKASVICN | |
| | QDYSLPMSQNLRSCYQGETEYCPVEAVIASHSPRFALTNGV | |
| | IFANCINTICRCQDNGKTITQNINQFVSMIDNSTCNDVMVD | |
| | KFTIKVGKYMGRKDINNINIQIGPQIIIDKVDLSNEINKMNQS | |
| | LKDSIFYLREAKRILDSVNISLISPSVQLFLIIISVLSFIILLIIIV | |
| | YLYCKSKHSYKYNKFIDDPDYYNDYKRERINGKASKSNNI | |
| | YYVGD | |
| 9 | IHYDSLSKVGVIKGLTYNYKIKGSPSTKLMVVKLIPNIDSVK | Mojiang virus, |
| | NCTQKQYDEYKNLVRKALEPVKMAIDTMLNNVKSGNNK | Tongguan 1 F |
| | YRFAGAIMAGVALGVATAATVTAGIALHRSNENAQAIAN | Protein, without |
| | MKSAIQNTNEAVKQLQLANKQTLAVIDTIRGEINNNIIPVIN | signal sequence |
| | QLSCDTIGLSVGIRLTQYYSEIITAFGPALQNPVNTRITIQAIS | |
| | SVFNGNFDELLKIMGYTSGDLYEILHSELIRGNIIDVDVDAG | |
| | YIALEIEFPNLTLVPNAVVQELMPISYNIDGDEWVTLVPRFV | |
| | LTRTTLLSNIDTSRCTITDSSVICDNDYALPMSHELIGCLQG | |
| | DTSKCAREKVVSSYVPKFALSDGLVYANCLNTICRCMDTD | |
| | TPISQSLGATVSLLDNKRCSVYQVGDVLISVGSYLGDGEYN | |
| | ADNVELGPPIVIDKIDIGNQLAGINQTLQEAEDYIEKSEEFLK | |
| | GVNPSIITLGSMVVLYIFMILIAIVSVIALVLSIKLTVKGNVV | |
| | RQQFTYTQHVPSMENINYVSH | |
| 10 | SRALLRETDNYSNGLIVENLVRNCHHPSKNNLNYTKTQKR | Bat |
| | DSTIPYRVEERKGHYPKIKHLIDKSYKHIKRGKRRNGHNGN | Paramyxovirus F |
| | IITIILLLILILKTQMSEGAIHYETLSKIGLIKGITREYKVKGTP | Protein, without |
| | SSKDIVIKLIPNVTGLNKCTNISMENYKEQLDKILIPINNIIEL | signal sequence |
| | YANSTKSAPGNARFAGVIIAGVALGVAAAAQITAGIALHEA | |
| | RQNAERINLLKDSISATNNAVAELQEATGGIVNVITGMQDY | |
| | INTNLVPQIDKLQCSQIKTALDISLSQYYSEILTVFGPNLQNP | |
| | VTTSMSIQAISQSFGGNIDLLLNLLGYTANDLLDLLESKSIT | |
| | GQITYINLEHYFMVIRVYYPIMTTISNAYVQELIKISFNVDGS | |
| | EWVSLVPSYILIRNSYLSNIDISECLITKNSVICRHDFAMPMS | |
| | YTLKECLTGDTEKCPREAVVTSYVPRFAISGGVIYANCLST | |
| | TCQCYQTGKVIAQDGSQTLMMIDNQTCSIVRIEEILISTGKY | |
| | LGSQEYNTMHVSVGNPVFTDKLDITSQISNINQSIEQSKFYL | |
| | DKSKAILDKINLNLIGSVPISILFIIAILSLILSIITFVIVMIIVRR | |
| | YNKYTPLINSDPSSRRSTIQDVYIIPNPGEHSIRSAARSIDRD | |
| | RD | |
| 11 | MVVILDKRCY CNLLILILMI SECSVGILHY EKLSKIGLVK | Truncated NiV |
| | GVTRKYKIKS NPLTKDIVIK MIPNVSNMSQ | fusion |
| | CTGSVMENYK TRLNGILTPI KGALEIYKNN | glycoprotein |
| | THDLVGDVRL AGVIMAGVAI GIATAAQITA | (FcDelta22) at |
| | GVALYEAMKN ADNINKLKSS IESTNEAVVK | cytoplasmic tail |
| | LQETAEKTVY VLTALQDYIN TNLVPTIDKI SCKQTELSLD | (with signal |
| | LALSKYLSDL LFVFGPNLQD PVSNSMTIQA ISQAFGGNYE | sequence) |
| | TLLRTLGYAT EDFDDLLESD SITGQIIYVD LSSYYIIVRV | |
| | YFPILTEIQQ AYIQELLPVS FNNDNSEWIS IVPNFILVRN | |
| | TLISNIEIGF CLITKRSVIC NQDYATPMTN NMRECLTGST | |
| | EKCPRELVVS SHVPRFALSN GVLFANCISV TCQCQTTGRA | |
| | ISQSGEQTLL MIDNTTCPTA VLGNVIISLG KYLGSVNYNS | |
| | EGIAIGPPVF TDKVDISSQI SSMNQSLQQS KDYIKEAQRL | |
| | LDTVNPSLIS MLSMIILYVL SIASLCIGLI TFISFIIVEK KRNT | |
| 12 | ILHY EKLSKIGLVK GVTRKYKIKS NPLTKDIVIK | Truncated mature |
| | MIPNVSNMSQ CTGSVMENYK TRLNGILTPI KGALEIYKNN | NiV fusion |
| | THDLVGDVRL AGVIMAGVAI GIATAAQITA | glycoprotein |
| | GVALYEAMKN ADNINKLKSS IESTNEAVVK | (FcDelta22) at |
| | LQETAEKTVY VLTALQDYIN TNLVPTIDKI SCKQTELSLD | cytoplasmic tail |
| | LALSKYLSDL LFVFGPNLQD PVSNSMTIQA ISQAFGGNYE | |
| | TLLRTLGYAT EDFDDLLESD SITGQIIYVD LSSYYIIVRV | |
| | YFPILTEIQQ AYIQELLPVS FNNDNSEWIS IVPNFILVRN | |
| | TLISNIEIGF CLITKRSVIC NQDYATPMTN NMRECLTGST | |
| | EKCPRELVVS SHVPRFALSN GVLFANCISV TCQCQTTGRA | |
| | ISQSGEQTLL MIDNTTCPTA VLGNVIISLG KYLGSVNYNS | |
| | EGIAIGPPVF TDKVDISSQI SSMNQSLQQS KDYIKEAQRL | |
| | LDTVNPSLIS MLSMIILYVL SIASLCIGLI TFISFIIVEK KRNT | |
| 13 | MMADSKLVSLNNNLSGKIKDQGKVIKNYYGTMDIKKIND | Hendra virus G |
| | GLLDSKILGAFNTVIALLGSIIIIVMNIMIIQNYTRTTDNQALI | protein Uniprot |
| | KESLQSVQQQIKALTDKIGTEIGPKVSLIDTSSTITIPANIGLL | O89343 |
| | GSKISQSTSSINENVNDKCKFTLPPLKIHECNISCPNPLPFRE | |
| | YRPISQGVSDLVGLPNQICLQKTTSTILKPRLISYTLPINTRE | |
| | GVCITDPLLAVDNGFFAYSHLEKIGSCTRGIAKQRIIGVGEV | |
| | LDRGDKVPSMFMTNVWTPPNPSTIHHCSSTYHEDFYYTLC | |
| | AVSHVGDPILNSTSWTESLSLIRLAVRPKSDSGDYNQKYIAI | |
| | TKVERGKYDKVMPYGPSGIKQGDTLYFPAVGFLPRTEFQY | |
| | NDSNCPIIHCKYSKAENCRLSMGVNSKSHYILRSGLLKYNL | |
| | SLGGDIILQFIEIADNRLTIGSPSKIYNSLGQPVFYQASYSWD | |
| | TMIKLGDVDTVDPLRVQWRNNSVISRPGQSQCPRFNVCPE | |
| | VCWEGTYNDAFLIDRLNWVSAGVYLNSNQTAENPVFAVF | |
| | KDNEILYQVPLAEDDTNAQKTITDCFLLENVIWCISLVEIYD | |
| | TGDSVIRPKLFAVKIPAQCSES | |
| 14 | MPAENKKVRFENTTSDKGKIPSKVIKSYYGTMDIKKINEGL | gb: AF212302\| |
| | LDSKILSAFNTVIALLGSIVIIVMNIMIIQNYTRSTDNQAVIK | Organism: Nipah |
| | DALQGIQQQIKGLADKIGTEIGPKVSLIDTSSTITIPANIGLLG | virus\|Strain |
| | SKISQSTASINENVNEKCKFTLPPLKIHECNISCPNPLPFREY | Name: UNKNOWN- |
| | RPQTEGVSNLVGLPNNICLQKTSNQILKPKLISYTLPVVGQS | AF212302\|Protein |
| | GTCITDPLLAMDEGYFAYSHLERIGSCSRGVSKQRIIGVGEV | Name: attachment |
| | LDRGDEVPSLFMTNVWTPPNPNTVYHCSAVYNNEFYYVL | glycoprotein\|Gene |
| | CAVSTVGDPILNSTYWSGSLMMTRLAVKPKSNGGGYNQH | Symbol: G |
| | QLALRSIEKGRYDKVMPYGPSGIKQGDTLYFPAVGFLVRTE | Uniprot Q9IH62 |
| | FKYNDSNCPITKCQYSKPENCRLSMGIRPNSHYILRSGLLK | |
| | YNLSDGENPKVVFIEISDQRLSIGSPSKIYDSLGQPVFYQASF | |
| | SWDTMIKFGDVLTVNPLVVNWRNNTVISRPGQSQCPRFNT | |
| | CPEICWEGVYNDAFLIDRINWISAGVFLDSNQTAENPVFTV | |
| | FKDNEILYRAQLASEDTNAQKTITNCFLLKNKIWCISLVEIY | |
| | DTGDNVIRPKLFAVKIPEQCT | |
| 15 | MLSQLQKNYLDNSNQQGDKMNNPDKKLSVNFNPLELDKG | gb: JQ001776: |
| | QKDLNKSYYVKNKNYNVSNLLNESLHDIKFCIYCIFSLLIIIT | 0817- |
| | IINIITISIVITRLKVHEENNGMESPNLQSIQDSLSSLTNMINTE | 10275\|Organism: |
| | ITPRIGILVTATSVTLSSSINYVGTKTNQLVNELKDYITKSCG | Cedar virus\|Strain |
| | FKVPELKLHECNISCADPKISKSAMYSTNAYAELAGPPKIFC | Name: CG1a\|Protein |
| | KSVSKDPDFRLKQIDYVIPVQQDRSICMNNPLLDISDGFFTY | Name: attachment |
| | IHYEGINSCKKSDSFKVLLSHGEIVDRGDYRPSLYLLSSHYH | glycoprotein\|Gene |
| | PYSMQVINCVPVTCNQSSFVFCHISNNTKTLDNSDYSSDEY | Symbol: G |
| | YITYFNGIDRPKTKKIPINNMTADNRYIHFTFSGGGGVCLGE | |
| | EFIIPVTTVINTDVFTHDYCESFNCSVQTGKSLKEICSESLRS | |
| | PTNSSRYNLNGIMIISQNNMTDFKIQLNGITYNKLSFGSPGR | |
| | LSKTLGQVLYYQSSMSWDTYLKAGFVEKWKPFTPNWMN | |
| | NTVISRPNQGNCPRYHKCPEICYGGTYNDIAPLDLGKDMY | |
| | VSVILDSDQLAENPEITVENSTTILYKERVSKDELNTRSTTTS | |
| | CFLFLDEPWCISVLETNRFNGKSIRPEIYSYKIPKYC | |
| 16 | MPQKTVEFINMNSPLERGVSTLSDKKTLNQSKITKQGYFGL | gb: NC_025256: |
| | GSHSERNWKKQKNQNDHYMTVSTMILEILVVLGIMFNLIV | 9117- |
| | LTMVYYQNDNINQRMAELTSNITVLNLNLNQLTNKIQREII | 11015\|Organism: |
| | PRITLIDTATTITIPSAITYILATLTTRISELLPSINQKCEFKTPT | Bat |
| | LVLNDCRINCTPPLNPSDGVKMSSLATNLVAHGPSPCRNFS | Paramyxovirus |
| | SVPTIYYYRIPGLYNRTALDERCILNPRLTISSTKFAYVHSEY | Eid_hel/GH- |
| | DKNCTRGFKYYELMTFGEILEGPEKEPRMFSRSFYSPTNAV | M74a/GHA/2009\| |
| | NYHSCTPIVTVNEGYFLCLECTSSDPLYKANLSNSTFHLVIL | Strain |
| | RHNKDEKIVSMPSFNLSTDQEYVQIIPAEGGGTAESGNLYF | Name: BatPV/Eid_ |
| | PCIGRLLHKRVTHPLCKKSNCSRTDDESCLKSYYNQGSPQH | hel/GH- |
| | QVVNCLIRIRNAQRDNPTWDVITVDLTNTYPGSRSRIFGSFS | M74a/GHA/2009\| |
| | KPMLYQSSVSWHTLLQVAEITDLDKYQLDWLDTPYISRPG | Protein |
| | GSECPFGNYCPTVCWEGTYNDVYSLTPNNDLFVTVYLKSE | Name: glycoprotein\| |
| | QVAENPYFAIFSRDQILKEFPLDAWISSARTTTISCFMFNNEI | Gene Symbol: G |
| | WCIAALEITRLNDDIIRPIYYSFWLPTDCRTPYPHTGKMTRV | |
| | PLRSTYNY | |
| 17 | MATNRDNTITSAEVSQEDKVKKYYGVETAEKVADSISGNK | gb: NC_025352: |
| | VFILMNTLLILTGAIITITLNITNLTAAKSQQNMLKIIQDDVN | 8716- |
| | AKLEMFVNLDQLVKGEIKPKVSLINTAVSVSIPGQISNLQTK | 11257\|Organism: |
| | FLQKYVYLEESITKQCTCNPLSGIFPTSGPTYPPTDKPDDDT | Mojiang |
| | TDDDKVDTTIKPIEYPKPDGCNRTGDHFTMEPGANFYTVPN | virus\|Strain |
| | LGPASSNSDECYTNPSFSIGSSIYMFSQEIRKTDCTAGEILSIQ | Name: Tongguan1\| |
| | IVLGRIVDKGQQGPQASPLLVWAVPNPKIINSCAVAAGDE | Protein |
| | MGWVLCSVTLTAASGEPIPHMFDGFWLYKLEPDTEVVSYR | Name: attachment |
| | ITGYAYLLDKQYDSVFIGKGGGIQKGNDLYFQMYGLSRNR | glycoprotein\|Gene |
| | QSFKALCEHGSCLGTGGGGYQVLCDRAVMSFGSEESLITN | Symbol: G |
| | AYLKVNDLASGKPVIIGQTFPPSDSYKGSNGRMYTIGDKYG | |
| | LYLAPSSWNRYLRFGITPDISVRSTTWLKSQDPIMKILSTCT | |
| | NTDRDMCPEICNTRGYQDIFPLSEDSEYYTYIGITPNNGGTK | |
| | NFVAVRDSDGHIASIDILQNYYSITSATISCFMYKDEIWCIAI | |
| | TEGKKQKDNPQRIYAHSYKIRQMCYNMKSATVTVGNAKN | |
| | ITIRRY | |
| 18 | MLSQLQKNYLDNSNQQGDKMNNPDKKLSVNFNPLELDKG | gb: JQ001776: |
| | QKDLNKSYYVKNKNYNVSNLLNESLHDIKFCIYCIFSLLIIIT | 8170- |
| | IINIITISIVITRLKVHEENNGMESPNLQSIQDSLSSLTNMINTE | 10275\|Organism: |
| | ITPRIGILVTATSVTLSSSINYVGTKTNQLVNELKDYITKSCG | Cedar virus\|Strain |
| | FKVPELKLHECNISCADPKISKSAMYSTNAYAELAGPPKIFC | Name: CG1a\|Protein |
| | KSVSKDPDFRLKQIDYVIPVQQDRSICMNNPLLDISDGFFTY | Name: attachment |
| | IHYEGINSCKKSDSFKVLLSHGEIVDRGDYRPSLYLLSSHYH | glycoprotein\|Gene |
| | PYSMQVINCVPVTCNQSSFVFCHISNNTKTLDNSDYSSDEY | Symbol: G |
| | YITYFNGIDRPKTKKIPINNMTADNRYIHFTFSGGGGVCLGE | |
| | EFIIPVTTVINTDVFTHDYCESFNCSVQTGKSLKEICSESLRS | |
| | PTNSSRYNLNGIMIISQNNMTDFKIQLNGITYNKLSFGSPGR | |
| | LSKTLGQVLYYQSSMSWDTYLKAGFVEKWKPFTPNWMN | |
| | NTVISRPNQGNCPRYHKCPEICYGGTYNDIAPLDLGKDMY | |
| | VSVILDSDQLAENPEITVFNSTTILYKERVSKDELNTRSTTTS | |
| | CFLFLDEPWCISVLETNRFNGKSIRPEIYSYKIPKYC | |
| 19 | MKKINE GLLDSKILSA FNTVIALLGS IVIIVMNIMI | (E501 A, W504A, |
| | IQNYTRSTDN QAVIKDALQG IQQQIKGLAD KIGTEIGPKV | Q530A, E533A) |
| | SLIDTSSTIT IPANIGLLGS KISQSTASIN ENVNEKCKFT | NiV G protein (Gc |
| | LPPLKIHECN ISCPNPLPFR EYRPQTEGVS NLVGLPNNIC | Δ 34) |
| | LQKTSNQILK PKLISYTLPV VGQSGTCITD PLLAMDEGYF | |
| | AYSHLERIGS CSRGVSKQRI IGVGEVLDRG DEVPSLFMTN | |
| | VWTPPNPNTV YHCSAVYNNE FYYVLCAVST | |
| | VGDPILNSTY WSGSLMMTRL AVKPKSNGGG | |
| | YNQHQLALRS IEKGRYDKVM PYGPSGIKQG | |
| | DTLYFPAVGF LVRTEFKYND SNCPITKCQY SKPENCRLSM | |
| | GIRPNSHYIL RSGLLKYNLS DGENPKVVFI EISDQRLSIG | |
| | SPSKIYDSLG QPVFYQASFS WDTMIKFGDV | |
| | LTVNPLVVNW RNNTVISRPG QSQCPRFNTC | |
| | PAICAEGVYN DAFLIDRINW ISAGVFLDSN ATAANPVFTV | |
| | FKDNEILYRA QLASEDTNAQ KTITNCFLLK NKIWCISLVE | |
| | IYDTGDNVIR PKLFAVKIPE QC | |
| 20 | GGGGS | Linker |
| 21 | GGGGGS | Linker |
| 22 | (GGGGS)n | Linker |
| 23 | (GGGGGS)n | Linker |
| 24 | MALPVTALLLPLALLLHAARP | CD8α signal |
| | | peptide |
| 25 | METDTLLLWVLLLWVPGSTG | IgK signal peptide |
| 26 | MLLLVTSLLLCELPHPAFLLIP | GMCSFR-α |
| | | (CSF2RA) signal |
| | | peptide |
| 27 | TTTPAPRPPTPAPTIASQPLSLRPEACRPAAGGAVHTRGLDF | CD8α hinge |
| | ACD | domain |
| 28 | IEVMYPPPYLDNEKSNGTIIHVKGKHLCPSPLFPGPSKP | CD28 hinge |
| | | domain |
| 29 | AAAIEVMYPPPYLDNEKSNGTIIHVKGKHLCPSPLFPGPSKP | CD28 hinge |
| | | domain |
| 30 | ESKYGPPCPPCP | IgG4 hinge |
| | | domain |
| 31 | ESKYGPPCPSCP | IgG4 hinge |
| | | domain |
| 32 | ESKYGPPCPPCPAPEFLGGPSVFLFPPKPKDTLMISRTPEVTC | IgG4 hinge-CH2- |
| | VVVDVSQEDPEVQFNWYVDGVEVHNAKTKPREEQFNSTY | CH3 domain |
| | RVVSVLTVLHQDWLNGKEYKCKVSNKGLPSSIEKTISKAK | |
| | GQPREPQVYTLPPSQEEMTKNQVSLTCLVKGFYPSDIAVE | |
| | WESNGQPENNYKTTPPVLDSDGSFFLYSRLTVDKSRWQEG | |
| | NVFSCSVMHEALHNHYTQKSLSLSLGK | |
| 33 | IYIWAPLAGTCGVLLLSLVITLYC | CD8α |
| | | transmembrane |
| | | domain |
| 34 | FWVLVVVGGVLACYSLLVTVAFIIFWV | CD28 |
| | | transmembrane |
| | | domain |
| 35 | MFWVLVVVGGVLACYSLLVTVAFIIFWV | CD28 |
| | | transmembrane |
| | | domain |
| 36 | KRGRKKLLYIFKQPFMRPVQTTQEEDGCSCRFPEEEEGGCE | 4-1BB |
| | L | costimulatory |
| | | domain |
| 37 | RSKRSRLLHSDYMNMTPRRPGPTRKHYQPYAPPRDFAAYR | CD28 |
| | S | costimulatory |
| | | domain |
| 38 | RVKFSRSADAPAYQQGQNQLYNELNLGRREEYDVLDKRR | CD3ζ signaling |
| | GRDPEMGGKPRRKNPQEGLYNELQKDKMAEAYSEIGMKG | domain |
| | ERRRGKGHDGLYQGLSTATKDTYDALHMQALPPR | |
| 39 | RVKFSRSADAPAYKQGQNQLYNELNLGRREEYDVLDKRR | CD3ζ signaling |
| | GRDPEMGGKPRRKNPQEGLYNELQKDKMAEAYSEIGMKG | domain (with Q to |
| | ERRRGKGHDGLYQGLSTATKDTYDALHMQALPPR | K mutation at |
| | | position 14) |
| 40 | DIQMTQTTSSLSASLGDRVTISCRASQDISKYLNWYQQKPD | Anti-CD19 |
| | GTVKLLIYHTSRLHSGVPSRFSGSGSGTDYSLTISNLEQEDI | FMC63 scFv |
| | ATYFCQQGNTLPYTFGGGTKLEITGSTSGSGKPGSGEGSTK | entire sequence, |
| | GEVKLQESGPGLVAPSQSLSVTCTVSGVSLPDYGVSWIRQP | with Whitlow |
| | PRKGLEWLGVIWGSETTYYNSALKSRLTIIKDNSKSQVFLK | linker |
| | MNSLQTDDTAIYYCAKHYYYGGSYAMDYWGQGTSVTVS | |
| | S | |
| 41 | DIQMTQTTSSLSASLGDRVTISCRASQDISKYLNWYQQKPD | Anti-CD19 |
| | GTVKLLIYHTSRLHSGVPSRFSGSGSGTDYSLTISNLEQEDI | FMC63 scFv light |
| | ATYFCQQGNTLPYTFGGGTKLEIT | chain variable |
| | | region |
| 42 | QDISKY | Anti-CD19 |
| | | FMC63 scFv light |
| | | chain CDR1 |
| 43 | HTS | Anti-CD19 |
| | | FMC63 scFv light |
| | | chain CDR2 |
| 44 | QQGNTLPYT | Anti-CD19 |
| | | FMC63 scFv light |
| | | chain CDR3 |
| 45 | GSTSGSGKPGSGEGSTKG | Whitlow linker |
| 46 | EVKLQESGPGLVAPSQSLSVTCTVSGVSLPDYGVSWIRQPP | Anti-CD19 |
| | RKGLEWLGVIWGSETTYYNSALKSRLTIIKDNSKSQVFLKM | FMC63 scFv |
| | NSLQTDDTAIYYCAKHYYYGGSYAMDYWGQGTSVTVSS | heavy chain |
| | | variable region |
| 47 | GVSLPDYG | Anti-CD19 |
| | | FMC63 scFv |
| | | heavy chain |
| | | CDR1 |
| 48 | IWGSETT | Anti-CD19 |
| | | FMC63 scFv |
| | | heavy chain |
| | | CDR2 |
| 49 | AKHYYYGGSYAMDY | Anti-CD19 |
| | | FMC63 scFv |
| | | heavy chain |
| | | CDR3 |
| 50 | DIQMTQTTSSLSASLGDRVTISCRASQDISKYLNWYQQKPD | Anti-CD19 |
| | GTVKLLIYHTSRLHSGVPSRFSGSGSGTDYSLTISNLEQEDI | FMC63 scFv |
| | ATYFCQQGNTLPYTFGGGTKLEITGGGGSGGGGSGGGGSE | entire sequence, |
| | VKLQESGPGLVAPSQSLSVTCTVSGVSLPDYGVSWIRQPPR | with 3xG4S linker |
| | KGLEWLGVIWGSETTYYNSALKSRLTIIKDNSKSQVFLKM | |
| | NSLQTDDTAIYYCAKHYYYGGSYAMDYWGQGTSVTVSS | |
| 51 | GGGGSGGGGSGGGGS | 3xG4S linker |
| 52 | atggccttaccagtgaccgccttgctcctgccgctggccttgctgctccacgccgccaggccg | Exemplary CD19 |
| | gacatccagatgacacagactacatcctccctgtctgcctctctgggagacagagtcaccatc | CAR nucleotide |
| | agttgcagggcaagtcaggacattagtaaatatttaaattggtatcagcagaaaccagatggaa | sequence |
| | ctgttaaactcctgatctaccatacatcaagattacactcaggagtcccatcaaggttcagtggc | |
| | agtgggtctggaacagattattctctcaccattagcaacctggagcaagaagatattgccactta | |
| | cttttgccaacagggtaatacgcttccgtacacgttcggaggggggaccaagctggagatca | |
| | caggctccacctctggatccggcaagcccggatctggcgagggatccaccaagggcgagg | |
| | tgaaactgcaggagtcaggacctggcctggtggcgccctcacagagcctgtccgtcacatgc | |
| | actgtctcaggggtctcattacccgactatggtgtaagctggattcgccagcctccacgaaag | |
| | ggtctggagtggctgggagtaatatggggtagtgaaaccacatactataattcagctctcaaat | |
| | ccagactgaccatcatcaaggacaactccaagagccaagttttcttaaaaatgaacagtctgca | |
| | aactgatgacacagccatttactactgtgccaaacattattactacggtggtagctatgctatgg | |
| | actactggggccaaggaacctcagtcaccgtctcctcaaccacgacgccagcgccgcgacc | |
| | accaacaccggcgcccaccatcgcgtcgcagcccctgtccctgcgcccagaggcgtgccg | |
| | gccagcggcggggggcgcagtgcacacgagggggctggacttcgcctgtgatatctacatc | |
| | tgggcgcccttggccgggacttgtggggtccttctcctgtcactggttatcaccctttactgcaa | |
| | acggggcagaaagaaactcctgtatatattcaaacaaccatttatgagaccagtacaaactact | |
| | caagaggaagatggctgtagctgccgatttccagaagaagaagaaggaggatgtgaactga | |
| | gagtgaagttcagcaggagcgcagacgcccccgcgtaccagcagggccagaaccagctct | |
| | ataacgagctcaatctaggacgaagagaggagtacgatgttttggacaagagacgtggccg | |
| | ggaccctgagatggggggaaagccgagaaggaagaaccctcaggaaggcctgtacaatg | |
| | aactgcagaaagataagatggcggaggcctacagtgagattgggatgaaaggcgagcgcc | |
| | ggaggggcaaggggcacgatggcctttaccagggtctcagtacagccaccaaggacacct | |
| | acgacgcccttcacatgcaggccctgccccctcgc | |
| 53 | MALPVTALLLPLALLLHAARPDIQMTQTTSSLSASLGDRVT | Exemplary CD19 |
| | ISCRASQDISKYLNWYQQKPDGTVKLLIYHTSRLHSGVPSR | CAR amino acid |
| | FSGSGSGTDYSLTISNLEQEDIATYFCQQGNTLPYTFGGGTK | sequence |
| | LEITGSTSGSGKPGSGEGSTKGEVKLQESGPGLVAPSQSLSV | |
| | TCTVSGVSLPDYGVSWIRQPPRKGLEWLGVIWGSETTYYN | |
| | SALKSRLTIIKDNSKSQVFLKMNSLQTDDTAIYYCAKHYYY | |
| | GGSYAMDYWGQGTSVTVSSTTTPAPRPPTPAPTIASQPLSL | |
| | RPEACRPAAGGAVHTRGLDFACDIYIWAPLAGTCGVLLLS | |
| | LVITLYCKRGRKKLLYIFKQPFMRPVQTTQEEDGCSCRFPE | |
| | EEEGGCELRVKFSRSADAPAYQQGQNQLYNELNLGRREEY | |
| | DVLDKRRGRDPEMGGKPRRKNPQEGLYNELQKDKMAEA | |
| | YSEIGMKGERRRGKGHDGLYQGLSTATKDTYDALHMQAL | |
| | PPR | |
| 54 | atggccttaccagtgaccgccttgctcctgccgctggccttgctgctccacgccgccaggccg | Tisagenlecleucel |
| | gacatccagatgacacagactacatcctccctgtctgcctctctgggagacagagtcaccatc | CD19 CAR |
| | agttgcagggcaagtcaggacattagtaaatatttaaattggtatcagcagaaaccagatggaa | nucleotide |
| | ctgttaaactcctgatctaccatacatcaagattacactcaggagtcccatcaaggttcagtggc | sequence |
| | agtgggtctggaacagattattctctcaccattagcaacctggagcaagaagatattgccactta | |
| | cttttgccaacagggtaatacgcttccgtacacgttcggaggggggaccaagctggagatca | |
| | caggtggcggtggctcgggcggtggtgggtcgggtggcggcggatctgaggtgaaactgc | |
| | aggagtcaggacctggcctggtggcgccctcacagagcctgtccgtcacatgcactgtctca | |
| | ggggtctcattacccgactatggtgtaagctggattcgccagcctccacgaaagggtctggag | |
| | tggctgggagtaatatggggtagtgaaaccacatactataattcagctctcaaatccagactga | |
| | ccatcatcaaggacaactccaagagccaagttttcttaaaaatgaacagtctgcaaactgatga | |
| | cacagccatttactactgtgccaaacattattactacggtggtagctatgctatggactactggg | |
| | gccaaggaacctcagtcaccgtctcctcaaccacgacgccagcgccgcgaccaccaacac | |
| | cggcgcccaccatcgcgtcgcagcccctgtccctgcgcccagaggcgtgccggccagcgg | |
| | cggggggcgcagtgcacacgagggggctggacttcgcctgtgatatctacatctgggcgcc | |
| | cttggccgggacttgtggggtccttctcctgtcactggttatcaccctttactgcaaacggggca | |
| | gaaagaaactcctgtatatattcaaacaaccatttatgagaccagtacaaactactcaagagga | |
| | agatggctgtagctgccgatttccagaagaagaagaaggaggatgtgaactgagagtgaagt | |
| | tcagcaggagcgcagacgcccccgcgtacaagcagggccagaaccagctctataacgagc | |
| | tcaatctaggacgaagagaggagtacgatgttttggacaagagacgtggccgggaccctga | |
| | gatggggggaaagccgagaaggaagaaccctcaggaaggcctgtacaatgaactgcagaa | |
| | agataagatggcggaggcctacagtgagattgggatgaaaggcgagcgccggaggggca | |
| | aggggcacgatggcctttaccagggtctcagtacagccaccaaggacacctacgacgccctt | |
| | cacatgcaggccctgccccctegc | |
| 55 | MALPVTALLLPLALLLHAARPDIQMTQTTSSLSASLGDRVT | Tisagenlecleucel |
| | ISCRASQDISKYLNWYQQKPDGTVKLLIYHTSRLHSGVPSR | CD19 CAR amino |
| | FSGSGSGTDYSLTISNLEQEDIATYFCQQGNTLPYTFGGGTK | acid sequence |
| | LEITGGGGSGGGGSGGGGSEVKLQESGPGLVAPSQSLSVTC | |
| | TVSGVSLPDYGVSWIRQPPRKGLEWLGVIWGSETTYYNSA | |
| | LKSRLTIIKDNSKSQVFLKMNSLQTDDTAIYYCAKHYYYG | |
| | GSYAMDYWGQGTSVTVSSTTTPAPRPPTPAPTIASQPLSLR | |
| | PEACRPAAGGAVHTRGLDFACDIYIWAPLAGTCGVLLLSL | |
| | VITLYCKRGRKKLLYIFKQPFMRPVQTTQEEDGCSCRFPEE | |
| | EEGGCELRVKFSRSADAPAYKQGQNQLYNELNLGRREEYD | |
| | VLDKRRGRDPEMGGKPRRKNPQEGLYNELQKDKMAEAYS | |
| | EIGMKGERRRGKGHDGLYQGLSTATKDTYDALHMQALPP | |
| | R | |
| 56 | atgctgctgctggtgaccagcctgctgctgtgcgagctgccccaccccgcctttctgctgatcc | Lisocabtagene |
| | ccgacatccagatgacccagaccacctccagcctgagcgccagcctgggcgaccgggtga | maraleucel CD19 |
| | ccatcagctgccgggccagccaggacatcagcaagtacctgaactggtatcagcagaagcc | CAR nucleotide |
| | cgacggcaccgtcaagctgctgatctaccacaccagccggctgcacagcggcgtgcccag | sequence |
| | ccggtttagcggcagcggctccggcaccgactacagcctgaccatctccaacctggaacag | |
| | gaagatatcgccacctacttttgccagcagggcaacacactgccctacacctttggcggogga | |
| | acaaagctggaaatcaccggcagcacctccggcagcggcaagcctggcagcggcgaggg | |
| | cagcaccaagggcgaggtgaagctgcaggaaagcggccctggcctggtggcccccagcc | |
| | agagcctgagcgtgacctgcaccgtgagcggcgtgagcctgcccgactacggcgtgagct | |
| | ggatccggcagccccccaggaagggcctggaatggctgggcgtgatctggggcagcgag | |
| | accacctactacaacagegccctgaagagccggctgaccatcatcaaggacaacagcaaga | |
| | gccaggtgttcctgaagatgaacagcctgcagaccgacgacaccgccatctactactgcgcc | |
| | aagcactactactacggcggcagctacgccatggactactggggccagggcaccagcgtga | |
| | ccgtgagcagcgaatctaagtacggaccgccctgccccccttgccctatgttctgggtgctgg | |
| | tggtggtcggaggcgtgctggcctgctacagcctgctggtcaccgtggccttcatcatcttttg | |
| | ggtgaaacggggcagaaagaaactcctgtatatattcaaacaaccatttatgagaccagtaca | |
| | aactactcaagaggaagatggctgtagctgccgatttccagaagaagaagaaggaggatgtg | |
| | aactgcgggtgaagttcagcagaagcgccgacgcccctgcctaccagcagggccagaatc | |
| | agctgtacaacgagctgaacctgggcagaagggaagagtacgacgtcctggataagcgga | |
| | gaggccgggaccctgagatgggcggcaagcctcggcggaagaacccccaggaaggcct | |
| | gtataacgaactgcagaaagacaagatggccgaggcctacagcgagatcggcatgaaggg | |
| | cgagcggaggcggggcaagggccacgacggcctgtatcagggcctgtccaccgccacca | |
| | aggatacctacgacgccctgcacatgcaggccctgcccccaagg | |
| 57 | MLLLVTSLLLCELPHPAFLLIPDIQMTQTTSSLSASLGDRVTI | Lisocabtagene |
| | SCRASQDISKYLNWYQQKPDGTVKLLIYHTSRLHSGVPSRF | maraleucel CD19 |
| | SGSGSGTDYSLTISNLEQEDIATYFCQQGNTLPYTFGGGTKL | CAR amino acid |
| | EITGSTSGSGKPGSGEGSTKGEVKLQESGPGLVAPSQSLSVT | sequence |
| | CTVSGVSLPDYGVSWIRQPPRKGLEWLGVIWGSETTYYNS | |
| | ALKSRLTIIKDNSKSQVFLKMNSLQTDDTAIYYCAKHYYY | |
| | GGSYAMDYWGQGTSVTVSSESKYGPPCPPCPMFWVLVVV | |
| | GGVLACYSLLVTVAFIIFWVKRGRKKLLYIFKQPFMRPVQT | |
| | TQEEDGCSCRFPEEEEGGCELRVKFSRSADAPAYQQGQNQ | |
| | LYNELNLGRREEYDVLDKRRGRDPEMGGKPRRKNPQEGL | |
| | YNELQKDKMAEAYSEIGMKGERRRGKGHDGLYQGLSTAT | |
| | KDTYDALHMQALPPR | |
| 58 | atgcttctcctggtgacaagccttctgctctgtgagttaccacacccagcattcctcctgatccca | Axicabtagene |
| | gacatccagatgacacagactacatcctccctgtctgcctctctgggagacagagtcaccatc | ciloleucel CD19 |
| | agttgcagggcaagtcaggacattagtaaatatttaaattggtatcagcagaaaccagatggaa | |
| | ctgttaaactcctgattaccatacatcaagattacactcaggagtcccatcaaggttcagtggc | CAR nucleotide |
| | agtgggtctggaacagattattctctcaccattagcaacctggagcaagaagatattgccactta | sequence |
| | cttttgccaacagggtaatacgcttccgtacacgttcggaggggggactaagttggaaataac | |
| | aggctccacctctggatccggcaagcccggatctggcgagggatccaccaagggcgaggt | |
| | gaaactgcaggagtcaggacctggcctggtggcgccctcacagagcctgtccgtcacatgc | |
| | actgtctcaggggtctcattacccgactatggtgtaagctggattcgccagcctccacgaaag | |
| | ggtctggagtggctgggagtaatatggggtagtgaaaccacatactataattcagctctcaaat | |
| | ccagactgaccatcatcaaggacaactccaagagccaagttttcttaaaaatgaacagtctgca | |
| | aactgatgacacagccatttactactgtgccaaacattattactacggtggtagctatgctatgg | |
| | actactggggtcaaggaacctcagtcaccgtctcctcagcggccgcaattgaagttatgtatcc | |
| | tcctccttacctagacaatgagaagagcaatggaaccattatccatgtgaaagggaaacacctt | |
| | tgtccaagtcccctatttcccggaccttctaagcccttttgggtgctggtggtggttgggggagt | |
| | cctggcttgctatagcttgctagtaacagtggcctttattattttctgggtgaggagtaagaggag | |
| | caggctcctgcacagtgactacatgaacatgactccccgccgccccgggcccacccgcaag | |
| | cattaccagccctatgccccaccacgcgacttcgcagcctatcgctccagagtgaagttcagc | |
| | aggagcgcagacgcccccgcgtaccagcagggccagaaccagctctataacgagctcaat | |
| | ctaggacgaagagaggagtacgatgttttggacaagagacgtggccgggaccctgagatgg | |
| | ggggaaagccgagaaggaagaaccctcaggaaggcctgtacaatgaactgcagaaagata | |
| | agatggcggaggcctacagtgagattgggatgaaaggcgagcgccggaggggcaagggg | |
| | cacgatggcctttaccagggtctcagtacagccaccaaggacacctacgacgcccttcacat | |
| | gcaggccctgccccctcgc | |
| 59 | MLLLVTSLLLCELPHPAFLLIPDIQMTQTTSSLSASLGDRVTI | Axicabtagene |
| | SCRASQDISKYLNWYQQKPDGTVKLLIYHTSRLHSGVPSRF | ciloleucel CD19 |
| | SGSGSGTDYSLTISNLEQEDIATYFCQQGNTLPYTFGGGTKL | CAR amino acid |
| | EITGSTSGSGKPGSGEGSTKGEVKLQESGPGLVAPSQSLSVT | sequence |
| | CTVSGVSLPDYGVSWIRQPPRKGLEWLGVIWGSETTYYNS | |
| | ALKSRLTIIKDNSKSQVFLKMNSLQTDDTAIYYCAKHYYY | |
| | GGSYAMDYWGQGTSVTVSSAAAIEVMYPPPYLDNEKSNG | |
| | TIIHVKGKHLCPSPLFPGPSKPFWVLVVVGGVLACYSLLVT | |
| | VAFIIFWVRSKRSRLLHSDYMNMTPRRPGPTRKHYQPYAPP | |
| | RDFAAYRSRVKFSRSADAPAYQQGQNQLYNELNLGRREE | |
| | YDVLDKRRGRDPEMGGKPRRKNPQEGLYNELQKDKMAE | |
| | AYSEIGMKGERRRGKGHDGLYQGLSTATKDTYDALHMQA | |
| | LPPR | |
| 60 | DIVLTQSPAILSASPGEKVTMTCRASSSVNYMDWYQKKPG | Anti-CD20 Leu16 |
| | SSPKPWIYATSNLASGVPARFSGSGSGTSYSLTISRVEAEDA | scFv entire |
| | ATYYCQQWSFNPPTFGGGTKLEIKGSTSGSGKPGSGEGSTK | sequence, with |
| | GEVQLQQSGAELVKPGASVKMSCKASGYTFTSYNMHWVK | Whitlow linker |
| | QTPGQGLEWIGAIYPGNGDTSYNQKFKGKATLTADKSSST | |
| | AYMQLSSLTSEDSADYYCARSNYYGSSYWFFDVWGAGTT | |
| | VTVSS | |
| 61 | DIVLTQSPAILSASPGEKVTMTCRASSSVNYMDWYQKKPG | Anti-CD20 Leu16 |
| | SSPKPWIYATSNLASGVPARFSGSGSGTSYSLTISRVEAEDA | scFv light chain |
| | ATYYCQQWSFNPPTFGGGTKLEIK | variable region |
| 62 | RASSSVNYMD | Anti-CD20 Leu16 |
| | | scFv light chain |
| | | CDR1 |
| 63 | ATSNLAS | Anti-CD20 Leu16 |
| | | scFv light chain |
| | | CDR2 |
| 64 | QQWSFNPPT | Anti-CD20 Leu16 |
| | | scFv light chain |
| | | CDR3 |
| 65 | EVQLQQSGAELVKPGASVKMSCKASGYTFTSYNMHWVKQ | Anti-CD20 Leu16 |
| | TPGQGLEWIGAIYPGNGDTSYNQKFKGKATLTADKSSSTA | scFv heavy chain |
| | YMQLSSLTSEDSADYYCARSNYYGSSYWFFDVWGAGTTV | |
| | TVSS | |
| 66 | SYNMH | Anti-CD20 Leu16 |
| | | scFv heavy chain |
| | | CDR1 |
| 67 | AIYPGNGDTSYNQKFKG | Anti-CD20 Leu16 |
| | | scFv heavy chain |
| | | CDR2 |
| 68 | SNYYGSSYWFFDV | Anti-CD20 Leu16 |
| | | scFv heavy chain |
| | | CDR3 |
| 69 | QVQLQQSGPGLVKPSQTLSLTCAISGDSVSSNSAAWNWIRQ | Anti-CD22 m971 |
| | SPSRGLEWLGRTYYRSKWYNDYAVSVKSRITINPDTSKNQ | scFv entire |
| | FSLQLNSVTPEDTAVYYCAREVTGDLEDAFDIWGQGTMVT | sequence, with |
| | VSSGGGGSGGGGSGGGGSDIQMTQSPSSLSASVGDRVTITC | 3xG4S linker |
| | RASQTIWSYLNWYQQRPGKAPNLLIYAASSLQSGVPSRFSG | |
| | RGSGTDFTLTISSLQAEDFATYYCQQSYSIPQTFGQGTKLEI | |
| | K | |
| 70 | QVQLQQSGPGLVKPSQTLSLTCAISGDSVSSNSAAWNWIRQ | Anti-CD22 m971 |
| | SPSRGLEWLGRTYYRSKWYNDYAVSVKSRITINPDTSKNQ | scFv heavy chain |
| | FSLQLNSVTPEDTAVYYCAREVTGDLEDAFDIWGQGTMVT | variable region |
| | VSS | |
| 71 | GDSVSSNSAA | Anti-CD22 m971 |
| | | scFv heavy chain |
| | | CDR1 |
| 72 | TYYRSKWYN | Anti-CD22 m971 |
| | | scFv heavy chain |
| | | CDR2 |
| 73 | AREVTGDLEDAFDI | Anti-CD22 m971 |
| | | scFv heavy chain |
| | | CDR3 |
| 74 | DIQMTQSPSSLSASVGDRVTITCRASQTIWSYLNWYQQRPG | Anti-CD22 m971 |
| | KAPNLLIYAASSLQSGVPSRFSGRGSGTDFTLTISSLQAEDF | scFv light chain |
| | ATYYCQQSYSIPQTFGQGTKLEIK | |
| 75 | QTIWSY | Anti-CD22 m971 |
| | | scFv light chain |
| | | CDR1 |
| 76 | AAS | Anti-CD22 m971 |
| | | scFv light chain |
| | | CDR2 |
| 77 | QQSYSIPQT | Anti-CD22 m971 |
| | | scFv light chain |
| | | CDR3 |
| 78 | QVQLQQSGPGMVKPSQTLSLTCAISGDSVSSNSVAWNWIR | Anti-CD22 m971- |
| | QSPSRGLEWLGRTYYRSTWYNDYAVSMKSRITINPDTNKN | L7 scFv entire |
| | QFSLQLNSVTPEDTAVYYCAREVTGDLEDAFDIWGQGTMV | sequence, with |
| | TVSSGGGGSGGGGSGGGGSDIQMIQSPSSLSASVGDRVTIT | 3xG₄S linker |
| | CRASQTIWSYLNWYRQRPGEAPNLLIYAASSLQSGVPSRFS | |
| | GRGSGTDFTLTISSLQAEDFATYYCQQSYSIPQTFGQGTKLE | |
| | IK | |
| 79 | QVQLQQSGPGMVKPSQTLSLTCAISGDSVSSNSVAWNWIR | Anti-CD22 m971- |
| | QSPSRGLEWLGRTYYRSTWYNDYAVSMKSRITINPDTNKN | L7 scFv heavy |
| | QFSLQLNSVTPEDTAVYYCAREVTGDLEDAFDIWGQGTMV | chain variable |
| | TVSS | region |
| 80 | GDSVSSNSVA | Anti-CD22 m971- |
| | | L7 scFv heavy |
| | | chain CDR1 |
| 81 | TYYRSTWYN | Anti-CD22 m971- |
| | | L7 scFv heavy |
| | | chain CDR2 |
| 82 | AREVTGDLEDAFDI | Anti-CD22 m971- |
| | | L7 scFv heavy |
| | | chain CDR3 |
| 83 | DIQMIQSPSSLSASVGDRVTITCRASQTIWSYLNWYRQRPG | Anti-CD22 m971- |
| | EAPNLLIYAASSLQSGVPSRFSGRGSGTDFTLTISSLQAEDF | L7 scFv light |
| | ATYYCQQSYSIPQTFGQGTKLEIK | chain variable |
| | | region |
| 84 | QTIWSY | Anti-CD22 m971- |
| | | L7 scFv light |
| | | chain CDR1 |
| 85 | AAS | Anti-CD22 m971- |
| | | L7 scFv light |
| | | chain CDR2 |
| 86 | QQSYSIPQT | Anti-CD22 m971- |
| | | L7 scFv light |
| | | chain CDR3 |
| 87 | DIVLTQSPASLAMSLGKRATISCRASESVSVIGAHLIHWYQ | Anti-BCMA |
| | QKPGQPPKLLIYLASNLETGVPARFSGSGSGTDFTLTIDPVE | C11D5.3 scFv |
| | EDDVAIYSCLQSRIFPRTFGGGTKLEIKGSTSGSGKPGSGEG | entire sequence, |
| | STKGQIQLVQSGPELKKPGETVKISCKASGYTFTDYSINWV | with Whitlow |
| | KRAPGKGLKWMGWINTETREPAYAYDFRGRFAFSLETSAS | linker |
| | TAYLQINNLKYEDTATYFCALDYSYAMDYWGQGTSVTVS | |
| | S | |
| 88 | DIVLTQSPASLAMSLGKRATISCRASESVSVIGAHLIHWYQ | Anti-BCMA |
| | QKPGQPPKLLIYLASNLETGVPARFSGSGSGTDFTLTIDPVE | C11D5.3 scFv |
| | EDDVAIYSCLQSRIFPRTFGGGTKLEIK | light chain |
| | | variable region |
| 89 | RASESVSVIGAHLIH | Anti-BCMA |
| | | C11D5.3 scFv |
| | | light chain CDR1 |
| 90 | LASNLET | Anti-BCMA |
| | | C11D5.3 scFv |
| | | light chain CDR2 |
| 91 | LOSRIFPRT | Anti-BCMA |
| | | C11D5.3 scFv |
| | | light chain CDR3 |
| 92 | QIQLVQSGPELKKPGETVKISCKASGYTFTDYSINWVKRAP | Anti-BCMA |
| | GKGLKWMGWINTETREPAYAYDFRGRFAFSLETSASTAYL | C11D5.3 scFv |
| | QINNLKYEDTATYFCALDYSYAMDYWGQGTSVTVSS | heavy chain |
| | | variable region |
| 93 | DYSIN | Anti-BCMA |
| | | C11D5.3 scFv |
| | | heavy chain |
| | | CDR1 |
| 94 | WINTETREPAYAYDFRG | Anti-BCMA |
| | | C11D5.3 scFv |
| | | heavy chain |
| | | CDR2 |
| 95 | DYSYAMDY | Anti-BCMA |
| | | C11D5.3 scFv |
| | | heavy chain |
| | | CDR3 |
| 96 | DIVLTQSPPSLAMSLGKRATISCRASESVTILGSHLIYWYQQ | Anti-BCMA |
| | KPGQPPTLLIQLASNVQTGVPARFSGSGSRTDFTLTIDPVEE | C12A3.2 scFv |
| | DDVAVYYCLQSRTIPRTFGGGTKLEIKGSTSGSGKPGSGEG | entire sequence, |
| | STKGQIQLVQSGPELKKPGETVKISCKASGYTFRHYSMNW | with Whitlow |
| | VKQAPGKGLKWMGRINTESGVPIYADDFKGRFAFSVETSA | linker |
| | STAYLVINNLKDEDTASYFCSNDYLYSLDFWGQGTALTVS | |
| | S | |
| 97 | DIVLTQSPPSLAMSLGKRATISCRASESVTILGSHLIYWYQQ | Anti-BCMA |
| | KPGQPPTLLIQLASNVQTGVPARFSGSGSRTDFTLTIDPVEE | C12A3.2 scFv |
| | DDVAVYYCLQSRTIPRTFGGGTKLEIK | light chain |
| | | variable region |
| 98 | RASESVTILGSHLIY | Anti-BCMA |
| | | C12A3.2 scFv |
| | | light chain CDR1 |
| 99 | LASNVQT | Anti-BCMA |
| | | C12A3.2 scFv |
| | | light chain CDR2 |
| 100 | LQSRTIPRT | Anti-BCMA |
| | | C12A3.2 scFv |
| | | light chain CDR3 |
| 101 | QIQLVQSGPELKKPGETVKISCKASGYTFRHYSMNWVKQA | Anti-BCMA |
| | PGKGLKWMGRINTESGVPIYADDFKGRFAFSVETSASTAYL | C12A3.2 scFv |
| | VINNLKDEDTASYFCSNDYLYSLDFWGQGTALTVSS | heavy chain |
| | | variable region |
| 102 | HYSMN | Anti-BCMA |
| | | C12A3.2 scFv |
| | | heavy chain |
| | | CDR1 |
| 103 | RINTESGVPIYADDFKG | Anti-BCMA |
| | | C12A3.2 scFv |
| | | heavy chain |
| | | CDR2 |
| 104 | DYLYSLDF | Anti-BCMA |
| | | C12A3.2 scFv |
| | | heavy chain |
| | | CDR3 |
| 105 | EVQLLESGGGLVQPGGSLRLSCAASGFTFSSYAMSWVRQA | Anti-BCMA |
| | PGKGLEWVSSISGSGDYIYYADSVKGRFTISRDISKNTLYLQ | FHVH33 entire |
| | MNSLRAEDTAVYYCAKEGTGANSSLADYRGQGTLVTVSS | sequence |
| 106 | GFTFSSYA | Anti-BCMA |
| | | FHVH33 CDR1 |
| 107 | ISGSGDYI | Anti-BCMA |
| | | FHVH33 CDR2 |
| 108 | AKEGTGANSSLADY | Anti-BCMA |
| | | FHVH33 CDR3 |
| 109 | DIQMTQSPSSLSASVGDRVTITCRASQSISSYLNWYQQKPG | Anti-BCMA |
| | KAPKLLIYAASSLQSGVPSRFSGSGSGTDFTLTISSLQPEDFA | CT103A scFv |
| | TYYCQQKYDLLTFGGGTKVEIKGSTSGSGKPGSGEGSTKG | entire sequence, |
| | QLQLQESGPGLVKPSETLSLTCTVSGGSISSSSYYWGWIRQP | with Whitlow |
| | PGKGLEWIGSISYSGSTYYNPSLKSRVTISVDTSKNQFSLKL | linker |
| | SSVTAADTAVYYCARDRGDTILDVWGQGTMVTVSS | |
| 110 | DIQMTQSPSSLSASVGDRVTITCRASQSISSYLNWYQQKPG | Anti-BCMA |
| | KAPKLLIYAASSLQSGVPSRFSGSGSGTDFTLTISSLQPEDFA | CT103A scFv |
| | TYYCQQKYDLLTFGGGTKVEIK | light chain |
| | | variable region |
| 111 | QSISSY | Anti-BCMA |
| | | CT103A scFv |
| | | light chain CDR1 |
| 112 | AAS | Anti-BCMA |
| | | CT103A scFv |
| | | light chain CDR2 |
| 113 | QQKYDLLT | Anti-BCMA |
| | | CT103A scFv |
| | | light chain CDR3 |
| 114 | QLQLQESGPGLVKPSETLSLTCTVSGGSISSSSYYWGWIRQP | Anti-BCMA |
| | PGKGLEWIGSISYSGSTYYNPSLKSRVTISVDTSKNQFSLKL | CT103A scFv |
| | SSVTAADTAVYYCARDRGDTILDVWGQGTMVTVSS | heavy chain |
| | | variable region |
| 115 | GGSISSSSYY | Anti-BCMA |
| | | CT103A scFv |
| | | heavy chain |
| | | CDR1 |
| 116 | ISYSGST | Anti-BCMA |
| | | CT103A scFv |
| | | heavy chain |
| | | CDR2 |
| 117 | ARDRGDTILDV | Anti-BCMA |
| | | CT103A scFv |
| | | heavy chain |
| | | CDR3 |
| 118 | atggccttaccagtgaccgccttgctcctgccgctggccttgctgctccacgccgccaggccg | Exemplary BCMA |
| | gacatccagatgacccagtctccatcctccctgtctgcatctgtaggagacagagtcaccatca | CAR nucleotide |
| | cttgccgggcaagtcagagcattagcagctatttaaattggtatcagcagaaaccagggaaag | sequence |
| | cccctaagctcctgatctatgctgcatccagtttgcaaagtggggtcccatcaaggttcagtgg | |
| | cagtggatctgggacagatttcactctcaccatcagcagtctgcaacctgaagattttgcaactt | |
| | actactgtcagcaaaaatacgacctcctcacttttggcggagggaccaaggttgagatcaaag | |
| | gcagcaccagcggctccggcaagcctggctctggcgagggcagcacaaagggacagctg | |
| | cagctgcaggagtcgggcccaggactggtgaagccttcggagaccctgtccctcacctgca | |
| | ctgtctctggtggctccatcagcagtagtagttactactggggctggatccgccagcccccag | |
| | ggaaggggctggagtggattgggagtatctcctatagtgggagcacctactacaacccgtcc | |
| | ctcaagagtcgagtcaccatatccgtagacacgtccaagaaccagttctccctgaagctgagtt | |
| | ctgtgaccgccgcagacacggcggtgtactactgcgccagagatcgtggagacaccatact | |
| | agacgtatggggtcagggtacaatggtcaccgtcagctcattcgtgcccgtgttcctgcccgc | |
| | caaacctaccaccacccctgcccctagacctcccaccccagccccaacaatcgccagccag | |
| | cctctgtctctgcggcccgaagcctgtagacctgctgccggcggagccgtgcacaccagag | |
| | gcctggacttcgcctgcgacatctacatctgggcccctctggccggcacctgtggcgtgctgc | |
| | tgctgagcctggtgatcaccctgtactgcaaccaccggaacaaacggggcagaaagaaact | |
| | cctgtatatattcaaacaaccatttatgagaccagtacaaactactcaagaggaagatggctgta | |
| | gctgccgatttccagaagaagaagaaggaggatgtgaactgagagtgaagttcagcagatcc | |
| | gccgacgcccctgcctaccagcagggacagaaccagctgtacaacgagctgaacctgggc | |
| | agacgggaagagtacgacgtgctggacaagcggagaggccgggaccccgagatgggcg | |
| | gaaagcccagacggaagaacccccaggaaggcctgtataacgaactgcagaaagacaag | |
| | atggccgaggcctacagcgagatcggcatgaagggcgagcggaggcgcggcaagggcc | |
| | acgatggcctgtaccagggcctgagcaccgccaccaaggacacctacgacgccctgcacat | |
| | gcaggccctgccccccaga | |
| 119 | MALPVTALLLPLALLLHAARPDIQMTQSPSSLSASVGDRVT | Exemplary BCMA |
| | ITCRASQSISSYLNWYQQKPGKAPKLLIYAASSLQSGVPSRF | CAR amino acid |
| | SGSGSGTDFTLTISSLQPEDFATYYCQQKYDLLTFGGGTKV | sequence |
| | EIKGSTSGSGKPGSGEGSTKGQLQLQESGPGLVKPSETLSLT | |
| | CTVSGGSISSSSYYWGWIRQPPGKGLEWIGSISYSGSTYYNP | |
| | SLKSRVTISVDTSKNQFSLKLSSVTAADTAVYYCARDRGDT | |
| | ILDVWGQGTMVTVSSFVPVFLPAKPTTTPAPRPPTPAPTIAS | |
| | QPLSLRPEACRPAAGGAVHTRGLDFACDIYIWAPLAGTCG | |
| | VLLLSLVITLYCNHRNKRGRKKLLYIFKQPFMRPVQTTQEE | |
| | DGCSCRFPEEEEGGCELRVKFSRSADAPAYQQGQNQLYNE | |
| | LNLGRREEYDVLDKRRGRDPEMGGKPRRKNPQEGLYNEL | |
| | QKDKMAEAYSEIGMKGERRRGKGHDGLYQGLSTATKDTY | |
| | DALHMQALPPR | |
| 120 | QVQLVQSGAEVKKPGASVKVSCKASGGTFSSYAISWVRQA | CD8_1 VH |
| | PGQGLEWMGIIDPSDGNTNYAQNFQGRVTMTRDTSTSTVY | |
| | MELSSLRSEDTAVYYCAKERAAAGYYYYMDVWGQGTTV | |
| | TVSS | |
| 121 | DIQMTQSPSSLSASVGDRVTITCRASQSISSYLNWYQQKPG | CD8_1 VL |
| | KAPKLLIYAASSLQSGVPSRFSGSGSGTDFTLTISSLQPEDFA | |
| | TYYCQQSYSTPLTFGGGTKVEIKR | |
| 122 | QVQLVQSGAEVKKPGASVKVSCKASGYTFTDYYIQWVRQ | CD8_2 VH |
| | APGQGLEWMGWINPNSGGTSYAQKFQGRVTMTRDTSTST | |
| | VYMELSSLRSEDTAVYYCAKEGDYYYGMDAWGQGTMVT | |
| | VSS | |
| 123 | DIVMTQSPLSLPVTPGEPASISCRSSQSLLHSNGYNYLDWY | CD8_2 VL |
| | LQKPGQSPQLLIYLGSNRASGVPDRFSGSGSGTDFTLKISRV | |
| | EAEDVGVYYCMQGLQTPHTFGQGTKVEIKR | |
| 124 | QVQLVQSGAEVKKPGASVKVSCKASGYTFTSYYMHWVRQ | CD8_3 VH |
| | APGQGLEWMGGFDPEDGETIYAQKFQGRVTMTRDTSTSTV | |
| | YMELSSLRSEDTAVYYCARDQGWGMDVWGQGTTVTVSS | |
| 125 | DIQMTQSPSSLSASVGDRVTITCRASQSISSYLNWYQQKPG | CD8_3 VL |
| | KAPKLLIYAASSLQSGVPSRFSGSGSGTDFTLTISSLQPEDFA | |
| | TYYCQQTYSTPYTFGQGTKLEIKR | |
| 126 | QVQLVQSGAEVKKPGASVKVSCKASGYTFTNHYMHWVR | CD8_4 VH |
| | QAPGQGLEWMGWMNPNSGNTGYAQKFQGRVTMTRDTST | |
| | STVYMELSSLRSEDTAVYYCASSESGSDLDYWGQGTLVTV | |
| | SS | |
| 127 | DIQMTQSPSSLSASVGDRVTITCRASQTIGNYVNWYQQKPG | CD8_4 VL |
| | KAPKLLIYGASNLHTGVPSRFSGSGSGTDFTLTISSLQPEDF | |
| | ATYYCQQTYSAPLTFGGGTKVEIKR | |
| 128 | QVQLVESGGGLVQAGGSLRLSCAASGRTFSGYVMGWFRQ | CD8_VHH |
| | APGKQRKFVAAISRGGLSTSYADSVKGRFTISRDNAKNTVF | |
| | LQMNTLKPEDTAVYYCAADRSDLYEITAASNIDSWGQGTL | |
| | VTVSS | |

1. A method of delivering an exogenous agent to a subject, the method comprising:
administering to a subject a first dose of a targeted lipid particle comprising an exogenous agent, and administering to the subject a second dose of the targeted lipid particle, wherein
the targeted lipid particle comprises one or more Paramyxovirus envelope proteins, and wherein
the first and second doses are administered within one month (e.g., within four weeks, within three weeks, within two weeks, within seven days, within six days, within five days, within four days, within three days, within two days, or within one day) of each other.

2. A method of delivering an exogenous agent to a subject, the method comprising:
administering to a subject a first dose of a targeted lipid particle comprising an exogenous agent, and administering to the subject a second dose of the targeted lipid particle, wherein
the targeted lipid particle comprises one or more Paramyxovirus envelope proteins, and wherein
the second dose is administered (i) on the first day following the first dose or (ii) on the second, third, fourth, fifth, sixth, seventh, fourteenth, twenty-first, or twenty-eighth day following the first dose

3. The method of claim 1 or 2, wherein the one or more Paramyxovirus envelope proteins have fusogenic activity.

4. The method of any one of claims 1-3, wherein the native binding tropism of the one or more of the Paramyxovirus envelope proteins is reduced.

5. The method of any one of claim 1-4, wherein the one or more Paramyxovirus envelope proteins is derived from an H protein molecule or a biologically active portion thereof from a Paramyxovirus and/or an HN protein molecule or a biologically active portion thereof from a Paramyxovirus.

6. The method of any one of claims 1-4, wherein the one or more Paramyxovirus envelope proteins comprises an F protein molecule or a biologically active portion thereof and/or a glycoprotein G (G protein) or a biologically active portion thereof.

7. The method of claim 1-6, wherein the one or more Paramyxovirus envelope proteins comprises an F protein molecule or a biologically active portion thereof from a Paramyxovirus and a glycoprotein G (G protein) or a biologically active portion thereof from a Paramyxovirus.

8. The method of any of claims 1-7, wherein the paramyxovirus is a henipavirus.

9. The method of any of claims 1-8, wherein the paramyxovirus is Measles morbillivirus.

10. The method of any of claims 1-8, wherein the paramyxovirus is a Hendra virus.

11. The method of any of claims 1-8, wherein the paramyxovirus is Nipah virus.

12. The method of any of claims 6-8 and 11, wherein the F protein or the biologically active portion thereof is a wild-type Nipah virus F (NiV-F) protein or is a functionally active variant or biologically active portion thereof.

13. The method of any of claims 6-8, 11 or 12 wherein the F protein molecule or a biologically active portion thereof is a NiV-F protein that has the amino acid sequence set forth in SEQ ID NO: 7 or an amino acid sequence having at or about 80%, at least at or about 81%, at least at or about 82%, at least at or about 83%, at or about 84%, at least at or about 85%, at least at or about 86%, or at least at or about 87%, at least at or about 88%, or at least at or about 89%, at least at or about 90%, at least at or about 91%, at least at or about 92%, at least at or about 93%, at least at or about 94%, at least at or about 95%, at or about 96%, at least at or about 97%, at least at or about 98%, or at least at or about 99% sequence identity to SEQ ID NO:7.

14. The method of any of claims 6-8 and 11-13, wherein the NiV-F protein is a biologically active portion that is truncated and lacks up to 40 contiguous amino acid residues at or near the C-terminus of the wild-type NiV-F protein (SEQ ID NO:7).

15. The method of any of claims 6-8 and 11-14, wherein the NiV-F protein is a biologically active portion that is truncated at the C-terminus of wild-type NiV-F and has the sequence set forth in any of SEQ ID NOS: 1-10 or an amino acid sequence having at least at or about 80%, at least at or about 81%, at least at or about 82%, at least at or about 83%, at least at or about 84%, at least at or about 85%, at least at or about 86%, at least at or about 87%, at least at or about 88%, at least at or about 89%, at least at or about 90%, at least at or about 91%, at least at or about 92%, at least at or about 93%, at least at or about 94%, at least at or about 95%, at or about 96%, at least at or about 97%, at least at or about 98%, or at least at or about 99% sequence identity to SEQ ID NOs: 1-10.

16. The method of any of claims 6-8 and 11-14, wherein the NiV-F protein is a biologically active portion that has a truncation at or near the C-terminus of the wild-type NiV-F selected from the group consisting of a 5 amino acid truncation at or near the C-terminus of the wild-type NiV-F protein, a 10 amino acid truncation at or near the C-terminus of the wild-type NiV-F protein, a 15 amino acid truncation at or near the C-terminus, a 20 amino acid truncation at or near the C-terminus of the wild-type NiV-F protein, a 22 amino acid truncation at or near the C-terminus of the wild-type NiV-F protein, or a 25 amino acid truncation at or near the C-terminus of the wild-type NiV-F protein, optionally wherein the wild-type NiV-F protein is set forth in SEQ ID NO:7.

17. The method of any of claims 6-8 and 11-15, wherein the F protein is a NiV-F protein that is a biologically active portion that has a 20 amino acid truncation at or near the C-terminus of the wild-type NiV-F protein (SEQ ID NO:7).

18. The method of claim 17, wherein the NiV-F protein has an amino acid sequence set forth in SEQ ID NO:2 or an amino acid sequence having at or about 80%, at least at or about 81%, at least at or about 82%, at least at or about 83%, at or about 84%, at least at or about 85%, at least at or about 86%, or at least at or about 87%, at least at or about 88%, or at least at or about 89%, at least at or about 90%, at least at or about 91%, at least at or about 92%, at least at or about 93%, at least at or about 94%, at least at or about 95%, at or about 96%, at least at or about 97%, at least at or about 98%, or at least at or about 99% sequence identity to SEQ ID NO: 2.

19. The method of any of claims 6-8 and 11-16, wherein the F protein is a NiV-F protein that is a biologically active portion thereof that has a 22 amino acid truncation at or near the C-terminus of the wild-type NiV-F protein (SEQ ID NO:7).

20. The method of claim 19, wherein the NiV-F protein has an amino acid sequence set forth in SEQ ID NO:11 or a sequence having at or about 80%, at least at or about 81%, at least at or about 82%, at least at or about 83%, at or about 84%, at least at or about 85%, at least at or about 86%, or at least at or about 87%, at least at or about 88%, or at least at or about 89%, at least at or about 90%, at least at or about 91%, at least at or about 92%, at least at or about 93%, at least at or about 94%, at least at or about 95%, at or about 96%, at least at or about 97%, at least at or about 98%, or at least at or about 99% sequence identity to SEQ ID NO: 11.

21. The method of claim 19 or claim 20, wherein the NiV-F protein has an amino acid sequence set forth in SEQ ID NO:12 or a sequence having at or about 80%, at least at or about 81%, at least at or about 82%, at least at or about 83%, at or about 84%, at least at or about 85%, at least at or about 86%, or at least at or about 87%, at least at or about 88%, or at least at or about 89%, at least at or about 90%, at least at or about 91%, at least at or about 92%, at least at or about 93%, at least at or about 94%, at least at or about 95%, at or about 96%, at least at or about 97%, at least at or about 98%, or at least at or about 99% sequence identity to SEQ ID NO: 12.

22. The method of any of claims 5-8 and 11-21, wherein the F protein or the biologically active portion thereof is a NiV-F protein that is a functionally active variant that comprises a point mutation on an N-linked glycosylation site of the wild-type NiV-F protein (SEQ ID NO:7) or a biologically active portion thereof.

23. The method of any of claims 5-8 and 11-21, wherein the F protein or the biologically active portion thereof is a NiV-F protein that is a functionally active variant that comprises:
i) a 20 amino acid truncation at or near the C-terminus of the wild-type NiV-F protein (SEQ ID NO:7); and/or
ii) a point mutation on an N-linked glycosylation site.

24. The method of claim 23, wherein the NiV-F protein has an amino acid sequence set forth in SEQ ID NO:11 or an amino acid sequence having at or about 80%, at least at or about 81%, at least at or about 82%, at least at or about 83%, at or about 84%, at least at or about 85%, at least at or about 86%, or at least at or about 87%, at least at or about 88%, or at least at or about 89%, at least at or about 90%, at least at or about 91%, at least at or about 92%, at least at or about 93%, at least at or about 94%, at least at or about 95%, at or about 96%, at least at or about 97%, at least at or about 98%, or at least at or about 99% sequence identity to SEQ ID NO: 11.

25. The method of any of claims 5-8 and 10-24, wherein the G protein or the biologically active portion thereof is a wild-type Nipah virus G (NiV-G) protein or a Hendra virus G protein or is a functionally active variant or biologically active portion thereof.

26. The method of any of claims 5-8 and 11-25, wherein the G protein or the biologically active portion thereof is a wild-type NiV-G protein or a functionally active variant or biologically active portion thereof.

27. The method of any of claims 5-8 and 11-26, wherein the G protein or the biologically active portion thereof is a mutant NiV-G protein that is modified to exhibit reduced native binding tropism.

28. The method of any of claims 5-8 and 11-27, wherein the G protein or the biologically active portion thereof is a mutant NiV-G protein that exhibits reduced binding to Ephrin B2 or Ephrin B3.

29. The method of any of claims 5-8 and 11-28, wherein the G protein or the biologically active portion thereof is a mutant NiV-G protein comprising one or more amino acid substitutions corresponding to amino acid substitutions selected from the group consisting of E501A, W504A, Q530A and E533A with reference to numbering set forth in SEQ ID NO:14.

30. The method of any of claims 5-8 and 11-29, wherein the NiV-G protein is a biologically active portion that is truncated and lacks up to 40 contiguous amino acid residues at or near the N-terminus of the wild-type NiV-G protein (SEQ ID NO:14).

31. The method of any of claims 5-8 and 11-30, wherein the NiV-G protein is a biologically active portion that has a truncation at or near the N-terminus of the wild-type NiV-G selected from the group consisting of a 5 amino acid truncation at or near the N-terminus of the wild-type NiV-G protein, a 10 amino acid truncation at or near the N-terminus of the wild-type NiV-G protein, a 15 amino acid truncation at or near the N-terminus of the wild-type NiV-G protein, a 20 amino acid truncation at or near the N-terminus of the wild-type NiV-G protein, a 25 amino acid truncation at or near the N-terminus of the wild-type NiV-G protein, a 30 amino acid truncation at or near the N-terminus of the wild-type NiV-G protein, or a 34 amino acid truncation at or near the N-terminus of the wild-type NiV-G protein, optionally wherein the wild-type NiV-G protein is set forth in SEQ ID NO:13, SEQ ID NO:14, or SEQ ID NO: 19.

32. The method of any of claims 6-8 and 11-31, wherein the NiV-G protein is a biologically active portion that is truncated at the N-terminus of wild-type NiV-G and has the sequence set forth in any of SEQ ID NOS: 13, 14, or 19 or an amino acid sequence having at least at or about 80%, at least at or about 81%, at least at or about 82%, at least at or about 83%, at least at or about 84%, at least at or about 85%, at least at or about 86%, at least at or about 87%, at least at or about 88%, at least at or about 89%, at least at or about 90%, at least at or about 91%, at least at or about 92%, at least at or about 93%, at least at or about 94%, at least at or about 95%, at or about 96%, at least at or about 97%, at least at or about 98%, or at least at or about 99% sequence identity to SEQ ID NOs 13, 14, or 19.

33. The method of any of claims 6-8 and 11-32, wherein the G protein molecule or a biologically active portion thereof NiV-G protein has the amino acid sequence set forth in SEQ ID NO: 13 or an amino acid sequence having at or about 80%, at least at or about 81%, at least at or about 82%, at least at or about 83%, at or about 84%, at least at or about 85%, at least at or about 86%, or at least at or about 87%, at least at or about 88%, or at least at or about 89%, at least at or about 90%, at least at or about 91%, at least at or about 92%, at least at or about 93%, at least at or about 94%, at least at or about 95%, at or about 96%, at least at or about 97%, at least at or about 98%, or at least at or about 99% sequence identity to SEQ ID NO:13.

34. The method of any of claims 6-8 and 11-32, wherein the G protein or the biologically active portion thereof is a mutant NiV-G protein having the amino acid sequence set forth in SEQ ID NO: 14 or an amino acid sequence having at or about 80%, at least at or about 81%, at least at or about 82%, at least at or about 83%, at or about 84%, at least at or about 85%, at least at or about 86%, or at least at or about 87%, at least at or about 88%, or at least at or about 89%, at least at or about 90%, at least at or about 91%, at least at or about 92%, at least at or about 93%, at least at or about 94%, at least at or about 95%, at or about 96%, at least at or about 97%, at least at or about 98%, or at least at or about 99% sequence identity to SEQ ID NO:14.

35. The method of any of claims 6-8 and 11-32, wherein the G protein or the biologically active portion thereof is a mutant NiV-G protein having the amino acid sequence set forth in SEQ ID NO:19 or an amino acid sequence having at or about 80%, at least at or about 81%, at least at or about 82%, at least at or about 83%, at or about 84%, at least at or about 85%, at least at or about 86%, or at least at or about 87%, at least at or about 88%, or at least at or about 89%, at least at or about 90%, at least at or about 91%, at least at or about 92%, at least at or about 93%, at least at or about 94%, at least at or about 95%, at or about 96%, at least at or about 97%, at least at or about 98%, or at least at or about 99% sequence identity to SEQ ID NO:19.

36. The method of any of claims 6-8, 11-34, wherein the F protein comprises the sequence set forth in SEQ ID NO. 12 and the G protein comprises the sequence set forth in SEQ ID NO. 19.

37. The method of any of claims 1-36, wherein at least one of the one or more Paramyxovirus envelope proteins are linked to a secondary moiety that is a targeting domain or a functional domain.

38. The method of claim 37, wherein the at least one of the one or more Paramyxovirus is a glycoprotein G (G protein) or a biologically active portion thereof and the G protein or biologically active portion thereof is linked to the secondary moiety.

39. The method of claim 37 or claim 38, wherein the secondary moiety is a functional domain and the functional domain is selected from a cytokine, growth factor, hormone, neurotransmitter, receptor, or apoptosis ligand.

40. The method of claim 37 or claim 38, wherein the secondary moiety is a targeting domain and the targeting domain is specific for a cell surface receptor on a target cell.

41. The method of any of claim 37, wherein the targeting domain is a Design ankyrin repeat proteins (DARPin), a single domain antibody (sdAb), a VHH fragment, a single chain variable fragment (scFv), or an antigen-binding fibronectin type III (Fn3) scaffold.

42. The method of any one of claims 37-41, wherein the at least one of the one or more Paramyxovirus envelope proteins and the secondary moiety are directly linked.

43. The method of any one of claims 37-41, wherein the at least one of the one or more Paramyxovirus envelope proteins and secondary moiety are indirectly linked via a linker.

44. The method of claim 43, wherein the linker is a peptide linker.

45. The method of claim 44, wherein the peptide linker is (GmS)ₙ (SEQ ID NO: 11), wherein each of m and n is an integer between 1 to 4, inclusive.

46. The method of any of claims 1-45, wherein the exogenous agent is a nucleic acid or a polypeptide.

47. The method of any of claims 1-46, wherein the exogenous agent is a nucleic acid encoding a payload gene, optionally wherein the nucleic acid encodes a chimeric antigen receptor (CAR).

48. The method of claim 40, wherein the target cell is one or more of a monocyte, macrophage, neutrophil, dendritic cell, eosinophil, mast cell, platelet, large granular lymphocyte, Langerhans' cell, natural killer (NK) cell, T lymphocyte (e.g., T cell), a Gamma delta T cell, B lymphocyte (e.g., B cell), CD3+ T cell, a CD4+ T cell, a CD8+ T cell, a hepatocyte, a hematopoietic stem cell, a CD34+ hematopoietic stem cell, a CD105+ hematopoietic stem cell, a CD117+ hematopoietic stem cell, a CD105+ endothelial cell, a B cell, a CD20+ B cell, a CD19+ B cell, a cancer cell, a CD133+ cancer cell, an EpCAM+ cancer cell, a CD19+ cancer cell, a Her2/Neu+ cancer cell, a GluA2+ neuron, a GluA4+ neuron, a NKG2D+ natural killer cell, a SLC1A3+ astrocyte, a SLC7A10+ adipocyte, or a CD30+ lung epithelial cell.

49. The method of any of claims 1-48, wherein the first and second dose are administered ex vivo to the subject.

50. The method of any of claims 1-48, wherein the first and second dose are administered to the subject intravenously.

51. The method of any of claims 1-50, wherein the time period between the first and second dose is no more than one month.

52. The method of any of claims 1-50, wherein the time period between the first and second dose is no more than one week.

53. The method of any of claims 1-50, wherein the time period between the first and second dose is no more than three days.

54. The method of any of claims 1-53, wherein the method further comprises administration of a third dose of the targeted lipid particle.

55. The method of claim 54, wherein the third dose is administered within one month (e.g., within four weeks, within three weeks, within two weeks, within seven days, within six days, within five days, within four days, within three days, within two days, or within one day) of the second dose.

56. The method of claim 54, wherein the third dose is administered (i) on the first day following the second dose or (ii) on the second, third, fourth, fifth, sixth, seventh, fourteenth, twenty-first, or twenty-eighth day following the second dose.

57. The method of any of claims 54-56, wherein the third dose is administered ex vivo to the subject.

58. The method of any of claims 54-56, wherein the third dose is administered to the subject intravenously.

59. The method of any of claims 54-56, wherein the time period between the first and third dose is no more than one month.

60. The method of any of claims 54-59, wherein the time period between the first and third dose is no more than one week.

61. The method of any of claims 54-60, wherein the time period between the first and third dose is no more than three days.

62. A targeted lipid particle comprising one or more Paramyxovirus envelope proteins for use in the method of any of claims 1-61.

63. The targeted lipid particle of claim 61, wherein the lipid particle is a viral vector and/or viral-like particle.

64. The targeted lipid particle of claim 62 or claim 63, wherein the lipid particle is a viral vector, optionally wherein the lipid particle is a lentiviral vector.
